SEQUENCE LISTING (28 sequences)

SEQ ID NO: 1 - DNA, 21 bp, artificial sequence
  primer
taaattttggagggtctcggc

SEQ ID NO: 2 - DNA, 21 bp, artificial sequence
  primer
ttaattgccgcgcaaaatgcg

SEQ ID NO: 3 - DNA, 29 bp, artificial sequence
  primer
gcgatggagtaccacttggcgatttttgg

SEQ ID NO: 4 - DNA, 28 bp, artificial sequence
  primer
accgtgattggactgttttcagtgcacc

SEQ ID NO: 5 - DNA, 28 bp, artificial sequence
  primer
gctttgaacccgcagacactaagattgg

SEQ ID NO: 6 - DNA, 30 bp, artificial sequence
  primer
gttagccttccaacctgaatagagaacagg

SEQ ID NO: 7 - DNA, 21 bp, artificial sequence
  primer
ggaaaaacgacacgactatgg

SEQ ID NO: 8 - DNA, 22 bp, artificial sequence
  primer
atgcgacgaaatcactactcgg

SEQ ID NO: 9 - DNA, 29 bp, artificial sequence
  primer
gctagaaatgggtgaaatcggtcttccgg

SEQ ID NO: 10 - DNA, 28 bp, artificial sequence
  primer
accgtgattggactgttttcagtgcacc

SEQ ID NO: 11 - DNA, 28 bp, artificial sequence
  primer
tgaactgacgaaactgggaggataaccg

SEQ ID NO: 12 - DNA, 30 bp, artificial sequence; identical to SEQ ID NO: 6
  primer

SEQ ID NO: 13 - DNA, 50 bp, artificial sequence
  primer
tttaaaatcaaatttctcaggtaatgcgggattggccaaaggacccaaag

SEQ ID NO: 14 - DNA, 50 bp, artificial sequence
  primer
tatccgaccacactaccatcagaatgcgggattggccaaaggacccaaag

SEQ ID NO: 15 - DNA, 49 bp, artificial sequence
  primer
aattcctatcagttgtcgggtaatgcgggattggccaaaggacccaaag

SEQ ID NO: 16 - DNA, 48 bp, artificial sequence
  primer
ttatttatagttgtttttcagatgcgggattggccaaaggacccaaag

SEQ ID NO: 17 - DNA, 49 bp, artificial sequence
  primer
tcttgatgttctattttgcagaatgcgggattggccaaaggacccaaag

SEQ ID NO: 18 - DNA, 50 bp, artificial sequence
  primer
gtaataaattttggcaataaatcatgcgggattggccaaaggacccaaag

SEQ ID NO: 19 - DNA, 50 bp, Artificial Sequence
  primer
ctttgggtcctttggccaatcccgcattacctgagaaatttgattttaaa

SEQ ID NO: 20 - DNA, 50 bp, Artificial Sequence
  primer
ctttgggtcctttggccaatcccgcattctgatggtagtgtggtcggata

SEQ ID NO: 21 - DNA, 49 bp, Artificial Sequence
  primer
ctttgggtcctttggccaatcccgcattacccgacaactgataggaatt

SEQ ID NO: 22 - DNA, 48 bp, Artificial Sequence
  primer
ctttgggtcctttggccaatcccgcatctgaaaaacaactataaataa

SEQ ID NO: 23 - DNA, 49 bp, Artificial sequence
  primer
ctttgggtcctttggccaatcccgcattctgcaaaatagaacatcaaga

SEQ ID NO: 24 - DNA, 50 bp, Artificial Sequence
  primer
ctttgggtcctttggccaatcccgcatgatttattgccaaaatttattac

SEQ ID NO: 25 - DNA, 20 bp, artificial sequence
  primer
gccgcatagttaagccagcc

SEQ ID NO: 26 - DNA, 24 bp, Artificial Sequence
  primer
cgggattggccaaaggacccaaag

SEQ ID NO: 27 - DNA, 24 bp, Artificial Sequence
  primer
ctttgggtcctttggccaatcccg

SEQ ID NO: 28 - DNA, 24 bp, Artificial Sequence
  primer
ccgcttacagacaagctgtgaccg

(19) United States
(12) Patent Application Publication
(10) Pub. No.: US 2006/0178292 A1
(43) Pub. Date: Aug. 10, 2006

(54) Methods and compositions of ecdysozoan molt inhibition

(75) Inventors: Gary Ruvkun, Newton, MA (US); Alison Frand, Cambridge, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/540,445

(22) PCT Filed: Dec. 31, 2003
(86) PCT No.: PCT/US03/41788

§ 371 Date: Dec. 16, 2005

Related U.S. Application Data
(60) Provisional application No. 60/437,235, filed on Dec. 31, 2002.

(51) Int. Cl. A01N 37/18; C12Q 1/68; G01N 33/567; G01N 33/53

(52) U.S. Cl. 514/2; 435/6; 435/7.2

(57) ABSTRACT

In general, this invention relates to nucleic acid and amino acid sequences involved in molting and the use of these sequences as targets for the development of compounds that disrupt Ecdysozoan molting, and are useful as insecticides, nematicides, and anti-parasitic agents.

### STATEMENT AS TO FEDERALLY SPONSORED RESEARCH

This work was supported in part by the National Institutes of Health (NIH GM 44619). The government may have certain rights in this invention.

### BACKGROUND OF THE INVENTION

In general, the invention features methods and compositions that disrupt molting and are therefore useful targets for pesticides.

Nematodes represent one out of every five animals on the planet, and virtually all plant and animal species are targeted by at least one parasitic nematode. Plant-parasitic nematodes reduce the yield of the world's 40 major food staples resulting in losses of approximately 12.3% annually. Parasitic nematodes also damage human and domestic animal health. Lymphatic filariasis and elephantiasis are among the most devastating human tropical diseases. The World Health Organization estimated that these diseases affected 120 million people worldwide in 1992.

The impact of nematodes on human, animal, and plant health has resulted in the search for effective nematicides. Benzimidazoles and avermectins are two common nematicides, which target microtubule assembly and muscle activity, respectively. Unfortunately, resistance to these compounds is increasingly common. In addition, these compounds can have toxic effects on humans and other animals. Moreover, these nematicides are not effective against all nematodes. Thus more effective and specific nematicides are required.

### SUMMARY OF THE INVENTION

The present invention features improved methods and compositions for inhibiting molting in Ecdysozoans, including nematodes, parasitic nematodes, and insects.

In one aspect, the invention provides a method for identifying a candidate compound that disrupts molting in an Ecdysozoan (e.g., an insect or nematode). The method includes the steps of: (a) providing a cell expressing a mlt nucleic acid molecule or an ortholog of a mlt nucleic acid molecule; (b) contacting the cell with a candidate compound; and (c) comparing the expression of the mlt nucleic acid molecule in the cell contacted with the candidate compound with the expression of the nucleic acid molecule in a control cell not contacted with said candidate compound, where an alteration in expression identifies the candidate compound as a candidate compound that disrupts molting.

In a related aspect, the invention provides another method for identifying a candidate compound that disrupts molting in a nematode. The method includes the steps of: (a) providing a nematode cell expressing a mlt nucleic acid molecule; (b) contacting the nematode cell with a candidate compound; and (c) comparing the expression of the mlt nucleic acid molecule in the cell contacted with the candidate compound with the expression of the nucleic acid molecule in a control cell not contacted with said candidate compound, where an alteration in expression identifies the candidate compound as a candidate compound that modulates molting.

In various embodiments of the previous aspects, the method identifies a compound that increases or decreases transcription of a mlt nucleic acid molecule. In other embodiments of the previous aspects, the method identifies a compound that increases or decreases translation of an mRNA transcribed from the mlt nucleic acid molecule. In still other embodiments of the identification methods described herein, the compound is a member of a chemical library. In preferred embodiments, the cell is in a nematode.

Typically, a compound that decreases transcription or translation of a mlt nucleic acid molecule is useful in the invention. For some applications, however, a compound that increases transcription or translation of a mlt nucleic acid molecule is useful, for example, a mlt nucleic acid (e.g., W08F4.6, F09B12.1, or W01F3.3) that when overexpressed leads to larval arrest or death, or a mlt nucleic acid (e.g., C17G1.6, CD4.6, C42D8.5, F08C6.1) that encodes a secreted protease, which degrades Ecdysozoan cuticle and leads to larval arrest or death.

In a related aspect, the invention provides yet another method for identifying a candidate compound that disrupts molting in an Ecdysozoan. The method involves (a) providing a cell expressing a MLT polypeptide; (b) contacting the cell with a candidate compound; and (c) comparing the biological activity of the MLT polypeptide in the cell contacted with the candidate compound to a control cell not contacted with said candidate compound, where an alteration in the biological activity of the MLT polypeptide identifies the candidate compound as a candidate compound that disrupts molting.

In various embodiments, the cell is a nematode cell or a mammalian cell. In other embodiments, the MLT polypeptide is a protease. In still other embodiments, the biological activity of MLT polypeptide is monitored with an enzymatic assay or an immunological assay. In other preferred embodiments, the cell is in a nematode and the biological activity is monitored by detecting molting.

In another related aspect, the invention provides yet another method for identifying a candidate compound that disrupts molting. The method includes the steps of: (a) contacting a nematode with a candidate compound; and (b) comparing molting in the nematode contacted with the candidate compound to a control nematode not contacted with said candidate compound, where an alteration in molting identifies the candidate compound as a candidate compound that disrupts molting.

In yet another related aspect, the invention provides a yet further method of identifying a candidate compound that disrupts Ecdysozoan molting. The method includes the steps of: (a) contacting a cell containing a mlt nucleic acid regulatory region fused to a detectable reporter gene with a candidate compound; (b) detecting the expression of the reporter gene; and (c) comparing the reporter gene expression in the cell contacted with the candidate compound with a control cell not contacted with the candidate compound, where an alteration in the expression of the reporter gene identifies the candidate compound as a candidate compound that disrupts molting.

In various embodiments of the previous aspect, the alteration is an alteration in the timing of reporter gene expression of at least 10%, 20%, 30%, 40%, 50%, 60%, or even 70%, 80%, 90%, 95%, or 99% relative to the timing of expression in a control nematode not contacted with the candidate compound. In another embodiment, the alteration is an alteration in the level of expression of the reporter gene of at least 10%, 20%, 30%, 40%, 50%, 60%, or even 70%, 80%, 90%, 95%, or 99% relative to the level of expression in a control nematode not contacted with the candidate compound. In another embodiment, the alteration is an alteration in the cellular expression pattern of the reporter gene relative to the cellular expression pattern in a control nematode not contacted with the candidate compound.

In another related aspect, the invention provides a method for identifying a candidate compound that disrupts Ecdysozoan molting. The method includes the steps of: (a) contacting a MLT polypeptide with a candidate compound; and (b) detecting binding of said candidate compound to said MLT polypeptide, wherein said binding identifies said candidate compound as a candidate compound that disrupts molting.

In other aspects, the invention generally features an isolated RNA mlt nucleic acid inhibitor comprising at least a portion of a naturally occurring mlt nucleic acid molecule of an organism, or its complement, where the mlt nucleic acid is selected from the group consisting of any or all of the following B0024.14, C09G5.6, C11H1.3, C23F12.1, B0272.5, C34G6.6, C37C3.3, C42D8.5, CD4.4, CD4.6, D1054.15, F08C6.1, F09B12.1, F16H9.2, F18A1.3, F20G4.1, F25B4.6, F33A8.1, F33C8.3, F38H4.9, F40G9.1, F41C3.4, F41H10.7, F45G2.5, F49C12.12, F52B11.3, F53B8.1, F54A5.1, F54C9.2, F57B9.2, H04M03.4, H19M22.1, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T14F9.1, T19B10.2, T23F2.1, T24H7.2, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK430.8, ZK686.3, ZK783.1, ZK970.4, C09F12.1, C09H10.2, C17H12.14, C37C3.2, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y38F2AL.3, Y41D4B.21, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of any or all of these mlt nucleic acid molecules, where the RNA mlt nucleic acid inhibitor comprises at least a portion of a naturally occurring mlt nucleic acid inhibitor, or is capable of hybridizing to a naturally occurring mlt nucleic acid molecule, and decreases expression from a naturally occurring mlt nucleic acid molecule in the organism. In some embodiments, the naturally occurring mlt nucleic acid had been previously identified as functioning in molting, but had not been identified as the target for a nematicide, insecticide, or other compound that inhibits molting (e.g., C01H6.5, C17G1.6, C45B2.7, F11C1.6, F18C12.2, F29D11.1, F53G12.3, F56C11.1, K04F10.4, T05C12.10, T27F2.1, Y23H5A.7, and ZK270.1). In other embodiments, the naturally occurring mlt nucleic acid encodes a component of a secretory pathway (e.g., ZK1014.1, H15N14.1, F26H9.6, Y63D3A.5, C56C10.3, ZK180.4, F57H12.1, C39F7.4, Y113G7A.3, R160.1, C02C6.1, E03H4.8, F59E10.3, K12H4.4, D1014.3, C13B9.3, F43D9.3). In other embodiments, the naturally occurring mlt nucleic acid encodes a protein that functions in protein synthesis (e.g., B0336.10, B0393.1, C04F12.4, C23G10.3, D1007.6, F28D1.7, F35H10.4, F37C12.11, F37C12.9, F40F11.1, F53A3.3, T01C3.6, T05F1.3, Y45F10D.12). In still other embodiments, the inactivation or inhibition of a naturally occurring mlt nucleic acid produces mlt defects in less than 5% of larvae (e.g., C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1).

In preferred embodiments, the naturally occurring mlt nucleic acid molecule is an ortholog of a mlt nucleic acid molecule. The ortholog is selected from the group consisting of any one or all of the following M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709 AA161577, CAAC01000031, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

In other preferred embodiments, the naturally occurring mlt nucleic acid molecule is a Drosophila ortholog of a mlt nucleic acid molecule. The Drosophila ortholog is selected from the group consisting of any one or all of the following ref|NM_{—}079167, gb|M90806, ref|NM_{—}079419, ref|NM_{—}080092, gb|AY075331, ref NM_{—}057698, ref|NM_{—}132335, ref|NM_{—}134871, gb|AAF51201, ref|NM_{—}136653, ref|NM_{—}057520, ref|NM_{—}080132, gb|AY094832, emb|AJ487018, ref|NM_{—}080072, emb|AJ011925, ref|NM_{—}078644, ref|NM_{—}132550, ref|NM_{—}079972, gb|AY089504, emb|X78577, gb|AY118647, gb|AY071265, ref|NM_{—}140652, ref|NM_{—}078577, emb|X58374, ref|NM_{—}134578, gb|AY058709, gb|AY060235, gb|AY052122, AY060893, gb|AY113364, ref|NM_{—}135238, ref|NM_{—}057621, ref|NM_{—}136498, ref|NM_{—}143476, ref|NM_{—}137449, gb|M16152, ref|NM_{—}057268, ref|NM_{—}139674, gb|L02793, gb|AY060635, gb|AC008339.

In other preferred embodiments of the previous aspects, the RNA mlt nucleic acid inhibitor is a double stranded RNA molecule that decreases expression in the organism by at least 10%, 20%, 30%, 40%, 50%, 60%, or even 70%, 80%, 90%, 95%, or 99% from a naturally occurring mlt nucleic acid molecule. In other preferred embodiments, the RNA mlt nucleic acid inhibitor is an antisense RNA molecule that is complementary to at least six, seven, eight, nine, ten, fifteen, twenty, twenty-five, thirty, forty, fifty, seventy-five, or one hundred nucleotides of the mlt nucleic acid molecule and decreases expression in the organism by at least 10%, 20%, 30%, 40%, 50%, 60%, or even 70%, 80%, 90%, 95%, or 99% from a nucleic acid molecule to which it is complementary. In other preferred embodiments, the RNA mlt nucleic acid inhibitor is an siRNA molecule that comprises at least fifteen, sixteen, seventeen, eighteen, nineteen, twenty, twenty-one, twenty-two, twenty-three, twenty-four, twenty-five, or twenty-six nucleic acids of a mlt nucleic acid molecule and decreases expression in said organism by at least 10%, 20%, 30%, 40%, 50%, 60%, or even 70%, 80%, 90%, 95%, or 99%.

In related aspects, the invention features a vector comprising a mlt nucleic acid that encodes a MLT polypeptide or a nucleic acid encoding an RNA mlt nucleic acid inhibitor (e.g., double-stranded RNA, antisense RNA, or siRNA), positioned for expression, and a host cell (e.g., plant, animal, or bacterial cell) containing the vector. For some applications, the vector used is a vector described in Fraser et al. (Nature, 408:325-30, 2000), hereby incorporated by reference.

In another aspect, the invention provides a method for reducing or ameliorating a parasitic nematode infection in an organism (e.g., a human or domestic mammal, such as a cow, sheep, goat, pig, horse, dog, or cat). The method includes contacting the organism with a mlt nucleic acid or an RNA mlt nucleic acid inhibitor (e.g., double-stranded RNA, antisense RNA, or siRNA).

In a related aspect, the invention provides a method for reducing or ameliorating a parasitic nematode infection in an organism (e.g., a human or domestic mammal, such as a cow, sheep, goat, pig, horse, dog, or cat). The method includes contacting the organism with a MLT polypeptide.

In other related aspects, the invention provides a pharmaceutical composition including a MLT polypeptide or portion thereof, encoded by a mlt nucleic acid or an ortholog of the nucleic acid molecule, and a pharmaceutical excipient, that ameliorates a parasite infection in an animal.

In other related aspects, the invention provides a pharmaceutical composition including a mlt nucleic acid or an RNA mlt nucleic acid inhibitor (e.g., double-stranded RNA, antisense RNA, or siRNA), or portion thereof, and a pharmaceutical excipient, which ameliorates a parasite infection in an animal.

In another aspect, the invention provides a method of diagnosing an organism having a parasitic infection. The method involves contacting a sample from the organism with a mlt nucleic acid probe and detecting an increased level of a mlt nucleic acid in the sample relative to the level in a control sample not having a parasitic infection, thereby diagnosing the organism as having a parasitic infection.

In another aspect, the invention provides a method for diagnosing an organism having a parasitic infection. The method involves detecting an increased level of a MLT polypeptide in a sample from the organism relative to the level in a control sample not having a parasitic infection, thereby diagnosing the organism as having a parasite infection. In one embodiment, this method of detection is an immunological method involving an antibody against a MLT polypeptide.

In other related aspects, the invention provides a biocide including a biocide excipient and a mlt nucleic acid, or portion thereof, that disrupts Ecdysozoan molting by at least 10%, 20%, 30%, 40%, 50%, 60%, or even 70%, 80%, 90%, 95%, or 99%.

In other related aspects, the invention provides a biocide including a biocide excipient and an RNA mlt nucleic acid inhibitor (e.g., double-stranded RNA, antisense RNA, or siRNA), or portion thereof, that disrupts Ecdysozoan molting by at least 10%, 20%, 30%, 40%, 50%, 60%, or even 70%, 80%, 90%, 95%, or 99%.

In other related aspects, the invention provides a biocide including a biocide excipient and a MLT polypeptide, or portion thereof, or an ortholog of a MLT polypeptide that disrupts Ecdysozoan molting by at least 10%, 20%, 30%, 40%, 50%, 60%, or even 70%, 80%, 90%, 95%, or 99%.

In other aspects, the invention provides an insecticide including an insecticide excipient and a MLT polypeptide or portion thereof, encoded by a MLT nucleic acid, or ortholog, that disrupts insect molting by at least 10%, 20%, 30%, 40%, 50%, 60%, or even 70%, 80%, 90%, 95%, or 99%.

In other related aspects, the invention provides an insecticide including an insecticide excipient and a mlt nucleic acid, or portion thereof, or ortholog, and disrupts insect molting by at least 10%, 20%, 30%, 40%, 50%, 60%, or even 70%, 80%, 90%, 95%, or 99%.

In other related aspects, the invention provides an insecticide including an insecticide excipient and an RNA mlt nucleic acid inhibitor (e.g., double-stranded RNA, antisense RNA, or siRNA) that disrupts insect molting by at least 10%, 20%, 30%, 40%, 50%, 60%, or even 70%, 80%, 90%, 95%, or 99%.

In other aspects, the invention provides a nematicide including a nematicide excipient and an MLT polypeptide, or portion thereof, encoded by a mlt nucleic acid molecule, or ortholog.

In other related aspects, the invention provides a nematicide including a nematicide excipient and a mlt nucleic acid, or portion thereof, or ortholog, that disrupts nematode molting by at least 10%, 20%, 30%, 40%, 50%, 60%, or even 70%, 80%, 90%, 95%, or 99%.

In other related aspects, the invention provides a nematicide including a nematicide excipient and an RNA mlt nucleic acid inhibitor (e.g., double-stranded RNA, antisense RNA, or siRNA), that disrupts nematode molting by at least 10%, 20%, 30%, 40%, 50%, 60%, or even 70%, 80%, 90%, 95%, or 99%.

In another related aspect, the invention provides a transgenic organism (e.g., Ecdysozoan) expressing a mlt nucleic acid molecule or an RNA mlt nucleic acid inhibitor (e.g., double-stranded RNA, antisense RNA, or siRNA) at a level sufficient to disrupt molting in the progeny of an Ecdysozoan (e.g., a nematode, a parasitic nematode, or an insect) breeding with the transgenic organism relative to a control nematode, parasitic nematode, or insect not bred with the organism. In various embodiments, the mlt nucleic acid molecule or RNA mlt nucleic acid inhibitor is expressed under the control of a conditional promoter. In some applications, for the control of a population of Ecdysozoan pests, a transgenic organism expressing a mat nucleic acid molecule or an RNA mlt nucleic acid inhibitor, or portion thereof, under the control of a conditional promoter, for example, may be released into an area infested with an Ecdysozoan pest (e.g., a nematode or insect pest). The transgenic organism transmits the mlt nucleic acid transgene during mating with wild-type Ecdysozoan pests to disrupt molting in the progeny, and controls a population of Ecdysozoan pests.

In other related aspects, the invention provides a transgenic plant expressing a mlt nucleic acid or an RNA mlt nucleic acid inhibitor (e.g., double-stranded RNA, antisense RNA, or siRNA), or portion thereof, where a cell of the plant expresses the mlt nucleic acid or RNA mlt nucleic acid inhibitor at a level sufficient to disrupt molting in an Ecdysozoan (e.g., a nematode, a parasitic nematode, or an insect) that contacts (e.g., feeds on) the plant relative to a control nematode, parasitic nematode, or insect not contacted with the plant.

In other aspects, the invention provides a transgenic organism (e.g., insect or domestic mammal, such as a cow, sheep, goat, pig, or horse) expressing a mlt nucleic acid or an RNA mlt nucleic acid inhibitor (e.g., double-stranded RNA, antisense RNA, or siRNA), or portion thereof, at a level sufficient to disrupt molting in a nematode, a parasitic nematode, or an insect that contacts, (e.g., parasitizes or feeds on) the transgenic organism relative to a control nematode, parasitic nematode, or insect not contacted with the organism. Such transgenic organisms would be expected to be more resistant to parasitic nematode infection than control organisms not expressing a transgene. In preferred embodiments, the transgenic organism is an insect host organism (e.g., blackfly) capable of being infected with an Ecdysozoan parasite (e.g., nematode) that spends part of its life cycle as an insect parasite and part of its life cycle as a human parasite. Expression of the transgene in the transgenic host organism inhibits molting in the Ecdysozoan parasite, and is useful in controlling a human parasitic infection.

In preferred embodiments of the above aspects, a mlt nucleic acid is any one or all of the following B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK62.8, ZK270.1, ZK430.8, ZK686.3, ZK783.1, ZK970.4, C09F12.1, C09H10.2, C17H12.14, C37C3.2, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, ZK1014.1, H15N14.1, F26H9.6, Y63D3A.5, C56C10.3, ZK180.4, F57H12.1, C39F7.4, Y113G7A.3, R160.1, C02C6.1, E03H4.8, F59E10.3, K12H4.4, D1014.3, C13B9.3, F43D9.3, B0336.10, B0393.1, C04F12.4, C23G10.3, D1007.6, F28D1.7, F35H10.4, F37C12.11, F37C12.9, F40F11.1, F53A3.3, T01C3.6, T05F1.3, Y45F10D.12, or Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, F10C1.5, or a portion thereof, or an ortholog of any or all of these nucleic acids. In other embodiments, the mlt nucleic acid is a component of a secretory pathway (e.g. ZK1014.1, H15N14.1, F26H9.6, Y63D3A.5, C56C10.3, ZK180.4, F57H12.1, C39F7.4, Y113G7A.3, R160.1, C02C6.1, E03H4.8, F59E10.3, K12H4.4, D1014.3, C13B9.3, and F43D9.3). In other embodiments, the mlt nucleic acid is a protein that functions in protein synthesis and produces mlt defects in less than 5% of larvae (e.g. B0336.10, B0393.1, C04F12.4, C23G10.3, D1007.6, F28D1.7, F35H10.4, F37C12.11, F37C12.9, F40F11.1, F53A3.3, T01C3.6, T05F1.3, Y45F10D.12).

In preferred embodiments of any of the above aspects, a mlt ortholog is any or all of the following mlt nucleic acids: M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122, AY060893, AY058709, AA161577, CAAC01000031, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, 11744849, BG735807.

In other preferred embodiments of any of the above aspects, a Drosophila ortholog includes any or all of the following mlt nucleic acids: ref|NM_{—}079167, gb|M90806, ref|NM_{—}079419, ref|NM_{—}080092, gb|AY075331, ref|NM_{—}057698, ref|NM_{—}132335, ref|NM_{—}134871, gb|AAF51201, ref|NM_{—}136653, ref|NM_{—}057520, ref|NM_{—}080132, gb|AY094832, emb|AJ487018, ref|NM_{—}080072, emb|AJ011925, ref|NM_{—}078644, ref|NM_{—}132550, ref|NM_{—}079972, gb|AY089504, emb|X78577, gb|AY118647, gb|AY071265, ref|NM_{—}140652, ref|NM_{—}078577, emb|X58374, ref|NM_{—}134578, gb|AY058709, gb|AY060235, gb|AY052122, AY060893, gb|AY113364, ref|NM_{—}135238, ref|NM_{—}057621, ref|NM_{—}136498, ref|NM_{—}143476, ref|NM_{—}137449, gb|M16152, ref|NM_{—}057268, ref|NM_{—}139674, gb|L02793, gb|AY060635, gb|AC008339.

In other preferred embodiments of any of the previous aspect, the nucleic acid sequence is selected from those listed in Tables 1A, 1B, 4A-4D, or 7.

By “biocide” is meant any agent, compound, or molecule that slows, delays, inhibits, or arrests the growth, viability, molting, or reproduction of any Ecdysozoan by at least 5%, 10%, 20%, 30%, 40%, 50%, 60%, or even by as much as 70%, 80%, 90%, 95%, or 99%.

By “Ecdysozoan” is meant the clade of organisms that molt. Ecdysozoans include arthropods, tardigrades, onychophorans, nematodes, nematomorphs, kinorhynchs, loriciferans, and priapulids.

By “molting” is meant the shedding and synthesis of cuticle that occurs during the life cycle of an Ecdysozoan, such as a nematode or insect.

By “disrupts molting” is meant that the process of cuticle shedding is delayed, inhibited, slowed, or arrested. In some applications, the molting process is disrupted by larval arrest.

By “mlt nucleic acid” is meant a nucleic acid molecule, or an ortholog thereof, whose inactivation (e.g., by RNAi) results in a molting defect or larval arrest phenotype in an Ecdysozoan. RNAi of a mlt gene results in a Mlt phenotype or larval arrest phenotype in at least 1%, 3%, 5%, 10%, 20%, 30%, 40%, 50%, 60%, or even in 70%, 80%, 90%, 95%, or 99% of the larvae exposed to dsRNA-expressing bacteria.

By “RNA mlt nucleic acid inhibitor” is meant a double-stranded RNA, antisense RNA, or siRNA, or portion thereof, that when administered to an Ecdysozoan results in a molting defect or larval arrest phenotype. Typically, an RNA mlt nucleic acid inhibitor comprises at least a portion of a mlt nucleic acid molecule, or an ortholog thereof, or comprises at least a portion of the complementary strand of a mlt nucleic acid molecule. For example, a mlt nucleic acid molecule includes any or all of the nucleic acids listed in Tables 1A, 1B, 4A-4D, and 7.

By “MLT polypeptide” is meant any amino acid molecule encoded by a mlt nucleic acid. Typically, a MLT polypeptide functions in molting in an Ecdysozoan (e.g., nematode or insect).

By “parasite” is meant any multicellular organism that lives on or within the cells, tissues, or organs of a genetically distinct host organism.

By “parasitic nematode” is meant any nematode that lives on or within the cells, tissues, or organs of a genetically distinct host organism (e.g., plant or animal). For example, parasitic nematodes include, but are not limited to, any ascarid, filarid, or rhabditid (e.g., Onchocerca volvulus, Ancylostoma, Ascaris, Ascaris lumbricoides, Ascaris suum, Baylisascaris, Baylisascaris procyonis, Brugia malayi, Diroflaria, Diroflaria immitis, Dracunculus, Haemonchus contortus, Heterorhabditis bacteriophora, Loa loa, root-knot nematodes, such as Meloidogyne, M. arenaria, M. chitwoodi, M. graminocola, M. graminis, M. hapla, M. incognita, Necator, M. microtyla, and M. naasi, cyst nematodes (for example, Heterodera sp. such as H. schachtii, H. glycines, H. sacchari, H. oryzae, H. avenae, H. cajani, H. elachista, H. goettingiana, H. graminis, H. mediterranea, H. mothi, H. sorghi, and H. zeae, or, for example, Globodera sp. such as G. rostochiensis and G. pallida) root-attacking nematodes (for example, Rotylenchulus reniformis, Tylenchuylus semipenetrans, Pratylenchus brachyurus, Radopholus citrophilus, Radopholus similis, Xiphinema americanum, Xiphinema rivesi, Paratrichodorus minor, Heterorhabditis heliothidis, and Bursaphelenchus xylophilus), and above-ground nematodes (for example, Anguina funesta, Anguina tritici, Ditylenchus dipsaci, Ditylenchus myceliphagus, and Aphenlenchoides besseyi), Parastrongyloides trichosuri, Pristionchus pacificus, Steinernema, Strongyloides stercoralis, Strongyloides ratti, Toxocara canis, Trichinella spiralis, Trichuris muris or Wuchereria bancrofti).

By “nematicide” is meant any agent, compound, or molecule that slows, delays, inhibits, or arrests the growth, viability, molting, or reproduction of any nematode by at least 5%, 10%, 20%, 30%, 40%, 50%, 60%, or even by as much as 70%, 80%, 90%, 95%, or 99%.

By “insecticide” is meant any agent, compound, or molecule that slows, delays, inhibits, or arrests the growth, viability, molting, or reproduction of any insect by at least 5%, 10%, 20%, 30%, 40%, 50%, 60%, or even by as much as 70%, 80%, 90%, 95%, or 99%.

By “anti-parasitic” is meant any agent, compound, or molecule that ameliorates a parasitic infection in a host organism. In some applications, an anti-parasitic agent slows, delays, inhibits, or arrests the growth, viability, molting, or reproduction of a parasite in a host organism.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms.

Drosophila melanogaster orthologs of C. elegans mlt genes include, but are not limited to, ref|NM_{—}079167, gb|M90806, ref|NM_{—}079419, ref|NM_{—}080092, gb|AY075331, ret NM_{—}057698, ref|NM_{—}132335, ref|NM_{—}134871, gb|AAF51201, ref|NM_{—}136653, ref|NM_{—}057520, ref|NM_{—}080132, gb|AY094832, emb|AJ487018, ref|NM_{—}080072, emb|AJ01925, ref|NM_{—}078644, ref|NM_{—}132550, ref|NM_{—}079972, gb|AY089504, emb|X78577, gb|AY118647, gb|AY071265, ref|NM_{—}140652, ref|NM_{—}078577, emb|X58374, ref|NM_{—}134578, gb|AY058709, gb|AY060235, gb|AY052122, AY060893, gb|AY113364, ref|NM_{—}135238, ref|NM_{—}057621, ref|NM_{—}136498, ref|NM_{—}143476, ref|NM_{—}137449, gb|M16152, ref|NM_{—}057268, ref|NM_{—}139674, gb|L02793, gb|AY060635, and gb|AC008339.

Nematode orthologs of C. elegans mlt genes include, but are not limited to, BG310588 in Onchocerca volvulus (e⁻¹²¹); BE758466 in Brugia malayi (e⁻¹⁰⁴); BG2271612 in Strongyloides stercoralis (e⁻⁸⁴); BM346811 in Parastrongyloides trichosuri (e⁻⁸⁹); BG226227 in Strongyloides stercoralis (9e 24); BF169279 in Trichuris muris (4^{e-11}); BG893621 in Strongyloides ratti (2e⁻²⁰); BQ625515 in Meloidogyne incognita (3e⁻²⁵); BI746672 in Meloidogyne arenaria (6e⁻³¹); AA471404 in Brugia malayi (2e⁻⁶⁸); BE579677 in Strongyloides stercoralis (2e⁻⁵³); BI500192 in Pristionchus pacificus (2e⁻⁶⁹); BI782938 in Ascaris suum (9e⁻⁵²); BI073876 in Strongyloides ratti (1e⁻⁴¹); BF060055 in Haemonchus contortus (4e⁻¹⁸); AI723670 in Brugia malayi (8e⁻⁴⁰); BI746256 in Meloidogyne arenaria (3.⁰⁰e⁻¹⁵); BM882137 in Parastrongyloides trichosuri (6e⁻³³); BM277122 in Trichuris muris (6e⁻¹⁵); BM880769 in Meloidogyne incognita (3e⁻⁴¹); BI501765 in Meloidogyne arenaria; BE581131 in Strongyloides stercoralis (1e⁻³⁴); AI5399702 in Onchocerca volvulus (e⁻³⁸); BE5802318 in Strongyloides stercoralis (e⁻³⁵); BE2389166 in Meloidogyne incognita (e⁻¹⁷); BE580288 in Strongyloides stercoralis, AA161577 in Brugia malayi (e⁻³⁹); CAAC01000016 in C. briggsae; BI744615 in Meloidogyne javanica (4e-44); BG224680 Strongyloides stercoralis (4e⁻⁴⁴); AW114337 Pristionchus pacificus (e⁻⁴¹), BM281377 in Ascaris suum (2e⁻⁴¹); BU585500 in Ascaris lumbricoides, BG577863 in Trichuris muris (e⁻²⁴); BQ091075 in Strongyloides ratti (6e⁻¹⁴); AW257707 in Onchocerca volvulus; BF014893 in Strongyloides stercoralis (7e-³⁵); BQ613344 in Meloidogyne incognita (5e⁻⁴⁷); CAAC01000088 in C. Briggsae, BG735742 in Meloidogyne javanica (4e⁻¹⁴); CAAC01000028; AA110597 in Brugia malayi (3e⁻⁵⁶); BI863834 in Parastrongyloides trichosuri (3e⁻⁶⁹); AI987143 in Pristionchus pacificus (3e⁻⁶⁰); BI782814 in Ascaris suum; BI744849 in Meloidogyne javanica; and BG735807 in Meloidogyne javanica (6e⁻³⁸).

Of particular interest are orthologs of the following genes: B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5. Other mlt genes may be identified using the methods of the invention described herein.

By “portion” is meant a fragment of a protein or nucleic acid that is substantially identical to a reference protein or nucleic acid, and retains at least 50% or 75%, more preferably 80%, 90%, or 95%, or even 99% of the biological activity of the reference protein or nucleic acid using a molting assay as described herein.

By “isolated polynucleotide” is meant a nucleic acid (e.g., a DNA) that is free of the genes, which, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “polypeptide” is meant any chain of amino acids, regardless of length or post-translational modification (for example, glycosylation or phosphorylation).

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80%, and most preferably 90% or even 95% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide of the invention.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “specifically binds” is meant a compound or antibody which recognizes and binds a polypeptide of the invention, but which does not substantially recognize and bind other molecules in a sample, for example, a biological sample, which naturally includes a polypeptide of the invention.

By “derived from” is meant isolated from or having the sequence of a naturally occurring sequence (e.g., a cDNA, genomic DNA, synthetic, or combination thereof).

By “immunological assay” is meant an assay that relies on an immunological reaction, for example, antibody binding to an antigen. Examples of immunological assays include ELISAs, Western blots, immunoprecipitations, and other assays known to the skilled artisan.

By “anti-sense” is meant a nucleic acid sequence, regardless of length, that is complementary to the coding strand or mRNA of a nucleic acid sequence. In one embodiment, an antisense RNA is introduced to an individual cell, tissue, organ, or to a whole animals. Desirably the anti-sense nucleic acid is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The anti-sense nucleic acid may contain a modified backbone, for example, phosphorothioate, phosphorodithioate, or other modified backbones known in the art, or may contain non-natural internucleoside linkages.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The anti-sense nucleic acid may contain a modified backbone, for example, phosphorothioate, phosphorodithioate, or other modified backbones known in the art, or may contain non-natural internucleoside linkages.

By “siRNA” is meant a double stranded RNA that complements a region of an mRNA. Optimally, an siRNA is 21, 22, 23, or 24 nucleotides in length and has a 2 base overhang at its 3′ end. siRNAs can be introduced to an individual cell, tissue, organ, or to a whole animals. For example, they may be introduced systemically via the bloodstream. Such siRNAs are used to downregulate mRNA levels or promoter activity. Desirably, the siRNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages.

By “hybridize” is meant pair to form a double-stranded molecule between complementary polynucleotide sequences (e.g., genes listed in Tables 1A, 1B, 4A-4D, and 7), or portions thereof, under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “transgene” is meant any piece of DNA which is inserted by artifice into a cell and typically becomes part of the genome of the organism which develops from that cell. Such a transgene may include a gene that is partly or entirely heterologous (i.e., foreign) to the transgenic organism, or may represent a gene homologous to an endogenous gene of the organism. A transgene of the invention may encode a MLT polypeptide or an RNA mlt nucleic acid inhibitor.

By “transgenic” is meant any cell which includes a DNA sequence which is inserted by artifice into a cell and becomes part of the genome of the organism which develops from that cell, or part of a heritable extra chromosomal array. As used herein, transgenic organisms may be either transgenic vertebrates, such as domestic mammals (e.g., sheep, cow, goat, or horse), mice, or rats, transgenic invertebrates, such as insects or nematodes, or transgenic plants.

By “cell” is meant a single-cellular organism, cell from a multi-cellular organism, or it may be a cell contained in a multi-cellular organism.

By “differentially expressed” is meant a difference in the expression level of a nucleic acid. This difference may be either an increase or a decrease in expression, when compared to control conditions.

By “therapeutic compound” is meant a substance that affects the function of an organism. Such a compound may be, for example, an isolated naturally occurring, semi-synthetic, or synthetic agent. For example, a therapeutic compound may be a drug that targets a parasite infecting a host organism. A therapeutic compound may decrease, suppress, attenuate, diminish, arrest, or stabilize the development or progression of disease, disorder, or infection in a eukaryotic host organism.

The invention provides for compositions and methods useful for inhibiting molting in an Ecdysozoan (e.g., a parasitic nematode, nematode or insect). Other features and advantages of the invention will be apparent from the detailed description, and from the claims.

### BRIEF DESCRIPTION OF THE DRAWINGS

FIGS. 1A-1E are micrographs showing Mlt phenotypes associated with RNAi of mlt-24, mlt-18, mlt-12, and mlt-13 in nematodes visualized using Nomarski optics. FIGS. 1A and 1B are micrographs showing the Mlt phenotype of a mlt-24(RNAi) nematode. FIG. 1C is a micrograph showing the Mlt phenotype of a mlt-18(RNAi) nematode. FIG. 1D is a micrograph showing the Mlt phenotype of a mlt-12(RNAi) nematode. FIG. 1E is a micrograph showing the Mlt phenotype of a mlt-13(RNAi) nematode. Black arrows indicate where excess cuticle remains attached to the larvae.

FIGS. 2A-2D show that molting genes are expressed in a pulse before each molt. FIG. 2A is a series of micrographs showing fluorescence from mlt-12::gfp-pest early in L1, at the L1/L2 molt, and early in L2. The L2 larvae was fluorescent before molting. Black arrows indicate cuticle separated from the body. FIGS. 2B and 2C are graphs showing the percentage of worms that were fluorescent over time, on a scale normalized to the period between molts for each worm under observation. The bar at the top of the graph indicates the worm's developmental stage. FIG. 2B shows results for Ex[mlt-12::gfp-pest] (dashed line) or Ex[mlt-10::gfp-pest] (solid line) larvae scored for detectable fluorescence and for molting once per hour from late in the L1 stage until early adulthood. FIG. 2C shows cycling fluorescence in worms expressing mlt-13::gfp-pest (dashed line) or mlt-18::gfp-pest (solid line), observed in the hypodermis and seam cells. FIG. 2C shows Northern analysis of mlt-10 messenger RNA levels. Ribosomal RNA stained with ethidium-bromide provides a loading control.

FIGS. 3A-3H are micrographs showing GFP fluorescence associated with Pmlt-18::GFP-PEST and Pmlt-13::GFP-PEST expression in transgenic nematodes. FIGS. 3A, 3C, and 3E are micrographs showing GFP fluorescence in transgenic Pmlt-18::GFP-PEST expressing nematodes during early L1, L1/L2 molt, and early L2. FIGS. 3B, 3D, and 3F are micrographs of nematodes visualized using Nomarski optics. The black arrow in FIG. 2D indicates shedding of the cuticle at the L1/L2 molt. Worms were synchronized after hatching and monitored through larval development. FIGS. 3G and 3H are micrographs of nematodes showing GFP fluorescence in transgenic Pmlt-13::GFP-PEST expressing nematodes during early L2 and L1/L2 molt. The inset in FIGS. 3G and 3H is a micrograph of the transgenic nematode visualized using Nomarski optics.

FIG. 4A is a graph showing the percentage of animals that were fluorescent before a defective molt, normalized to the percentage of control larvae that were fluorescent before molting from the same stage. Ex[mlt-12::gfp-pest], indicated with black bars, or Ex[mlt-10::gfp-pest] larvae, indicated with gray bars, were fed bacteria expressing dsRNA for each gene indicated. “n” indicates the number of larvae observed. Pairwise chi-square tests indicated that the decreased fraction of fluorescent Ex[mlt-12::gfp-pest] larvae after RNAi of nhr-23 or acn-1, and of fluorescent Ex[mlt-10::gfp-pest] larvae after RNAi of nhr-23, acn-1, or mlt-12, relative to control animals, is significant, with p<0.001 in all 5 tests.

FIG. 4B is a graph that shows the percentage of late L4 larvae with detectable fluorescence, for selected gene inactivations. Ex[mlt-10::gfp-pest] larvae were fed bacteria expressing dsRNA for each gene indicated. Values represent the weighted average of two independent trials.

FIGS. 5A-5G are a series of micrographs showing expression of molting gene gfp fusion genes in worms. FIGS. 5A-C show expression from mlt-24::gfp-pest. FIG. 5A shows fluorescence in the hypodermis (arrow) and seam cells (arrowhead) of an L4 larvae. FIG. 5B shows fluorescence in the rectal gland. The solid line traces the tail of the worm, the dashed line outlines the intestine. FIG. 5C is a pair of micrographs showing fluorescence and Nomarski images of the vulva of a young adult. FIG. 5D-5F are micrographs showing expression of acn-1::gfp-pest in a worm. FIG. 5D shows fluorescence in the excretory gland, duct, and pore cells (Exc), and in the glial cells (G) of interlabial neurons of larvae (lateral view). FIG. 5E shows fluorescence in the excretory gland (GN) and duct cells. A solid line traces the worm, and a dashed line outlines the posterior bulb of the pharynx. FIG. 5F shows fluorescence in the hypodermis and seam cells of a late L1 larvae. FIG. 5G shows fluorescence from mlt-18::gfp-pest in the hypodermis (arrow) and seam cells (arrowhead) of a late L1 larvae. FIG. 5H shows fluorescence from mlt-13::gfp in the hypodermis and seam cells of a late L3 larvae. The seam cell fluorescence from mlt-24::gfp-pest was observed only near the L4/Adult molt, when the cells terminally differentiate and fuse, whereas seam-cell fluorescence from mlt-13::gfp-pest and milt-18::gfp-pest was observed most often near larval-to-larval molts, when the cells divide. The anterior of the worm is at the right in all panels.

### DESCRIPTION OF THE INVENTION

The post-embryonic development of C. elegans proceeds through four larval stages that are separated by periodic molts when the collagen-like cuticle that encases the worm's body is shed and synthesized anew. As reported in more detail below, genes important for molting in C. elegans were identified by the present inventors through a genome-wide screen using bacterial-mediated RNA-interference (RNAi) to reduce gene function. Molting (mlt) gene inactivation by RNAi caused larvae to become trapped in old cuticle while attempting to molt. Inactivation of these genes, their orthologs in Ecdysozoans, or their encoded proteins by genetic or chemical means is expected to block molting and larval development in virtually any Ecdysozoan (e.g., nematodes and insects).

Four classes of genes central to molting function have been identified. The first class includes mlt genes that function specifically in nematodes (e.g., C09G5.6, C17G1.6, C23F12.1, C34G6.6, F08C6.1, F09B12.1, F16B4.3, F18A1.3, F45G2.5, F49C12.2, F53B8.1, H04M03.4, H19M22.2, K07D8.1, M6.1, M88.6, T05C12.10, W01F3.3, W08F4.6, Y111B2A.14, ZK262.8, ZK270.1, and ZK430.8). The protein products of such genes are likely to function in the execution phase of nematode molting and represent attractive targets for the development of highly specific nematicides. The second class includes mlt genes conserved in insects and nematodes, but not present in humans or yeast (e.g., C01H6.5, F11C1.6, F52B11.3, and ZK686.3). Nematicides and insecticides targeting such mlt genes, or their orthologs in insects or parasitic nematodes, are likely to specifically disrupt molting processes common to Ecdysozoans, and given this specificity are unlikely to adversely effect human health. The third class includes mlt genes whose inactivation by RNA results in highly penetrant molt defects (e.g., those molt genes listed in Tables 1A and Table 1B). Tables 1A and 1B include genes not previously identified as being involved in molting (e.g., B0024.14, C09G5.6, C11H1.3, C23F12.1, B0272.5, C34G6.6, C37C3.3, C42D8.5, CD4.4, CD4.6, D1054.15, F08C6.1, F09B12.1, F16H9.2, F18A1.3, F20G4.1, F25B4.6, F33A8.1, F33C8.3, F38H4.9, F40G9.1, F41C3.4, F41H10.7, F45G2.5, F49C12.12, F52B11.3, F53B8.1, F54A5.1, F54C9.2, F57B9.2, H04M03.4, H19M22.1, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T14F9.1, T19B10.2, T23F2.1, T24H7.2, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK430.8, ZK686.3, ZK783.1, ZK970.4, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5) as well as genes not previously suggested as targets for insecticides or nematicides (e.g., C01H6.5, C17G1.6, C45B2.7, F11C1.6, F18C12.2, F29D11.1, F53G12.3, F56C11.1, K04F10.4, T05C12.10, T27F2.1, Y23H5A.7, and ZK270.1). A fourth class includes mlt genes involved in the neuroendocrine control of molting. Such genes are expected to be conserved between nematodes and insects (e.g., Drosophila). C. elegans neuronal control genes are often refractory to RNAi; thus, RNAi against neuroendocrine control genes is likely to effect molting in only a small percentage of larvae. Neuroendocrine control genes will likely be identified among mlt genes whose inactivation by RNA interference results in molting defects in less than 5% of larvae (e.g., C09F12.1, C09H10.2, C17H12.14, C37C3.2, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y38F2AL.3, Y41D4B.21, Y41D4B.5, Y41D4B.5, Y45F10B.5, Y55H10A.1, ZK1236.3, ZK265.5, ZK265.6, ZK652.1, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, and Y71A12B.1. Additional mlt genes may be identified using a nematode strain having enhanced susceptibility to RNAi.

These compositions and methods are described further below.

RNAi Library Screen

To systematically identify genes required for molting in C. elegans, a library of 16,757 bacterial clones was used. Each HT115(DE3) E. coli clone (Timmons et al., Gene 263:103-112, 2001) expressed a double-stranded RNA corresponding to a single open reading frame (ORF) predicted in the C. elegans genome (Fraser et al., Nature, 408:325-30, 2000). Approximately 85% of all ORFs predicted to be present in the genome of C. elegans were represented in this library. Approximately 2,000 additional clones, which are publicly available through the Vidal lab ORFeome project at Harvard University (Orfeome project, Harvard University website) were also screened. The genes listed in Table 1B were identified in this screen.

Briefly, the bacterial colonies from each plate of the library were inoculated into 96-well microtiter dishes containing 300 ul of LB with 50 ug/ml of ampicillin. The bacteria were then cultured for approximately sixteen hours at 30° C. 30 ul of each overnight culture was plated onto a single well of a 24-well plate containing Nematode Growth Medium (NGM)-agar, IPTG (8 mM final concentration), and carbenicillin (25 ug/ml).

Early L1 larvae from wild-type (N2) worms were isolated using standard techniques, and approximately twenty larvae were added to each well. The worms were then incubated in individual wells at 20° C. for two and a half days with one of the 16,757 bacterial clones serving as a food source. Nematodes in each well were examined for molting defects by visual inspection using a standard light microscope. These assays were carried out “blind” (i.e., the researcher examining the nematode's molting phenotype was unaware of the identity of the bacterial clone present in the well at the time the phenotype was scored). A molting defect was identified by the presence of larvae with unshed cuticle attached to their bodies (the Mlt phenotype). Molting defects were never observed in control larvae fed on bacteria transformed with an empty vector. The majority of control larvae grew into gravid adult nematodes and sired progeny during the time of observation. As a positive internal control for the efficacy of post-embryonic RNAi, wild-type N2 larvae were concurrently fed HT115(DE3) bacteria expressing dsRNA corresponding to a known mlt gene, lrp-1.

C. elegans genes required for molting are listed in Tables 1A, 1B, 4A-4D, 7, and 8. Open reading frames initially identified as causing a Mlt phenotype were verified by re-screening two additional times. The identity of the gene represented by each bacterial colony was verified by sequencing. This was accomplished by sequencing the insert in the plasmid DNA isolated from the bacterial clone using primers complementary to flanking sequence present in the vector L440 (Timmons et al., Nature 391:806-811, 1998).

To evaluate the dauer molt, hatchlings of the temperature-sensitive, dauer constitutive mutants daf-2(e1370) and daf-7(e1372) were fed bacterial clones expressing dsRNA for each molting gene and cultivated at restrictive temperature (25° C.) for 3 days, such that control animals all became dauers. Animals were then shifted to permissive temperature (15° C.) for 2 days, allowing control animals to molt to the L4 stage. Observation of L2d or dauer larvae with the Mlt phenotype, in either genetic background, indicated that a given gene inactivation disrupted the L2d/dauer or dauer/L4molt.

Nomenclature

C. elegans genes whose inactivation by RNAi caused a molting defect, or Mlt phenotype, are shown in Tables 1A, 1B, 4A-4D, 7 and 8. These genes are identified by a C. elegans gene name and by an open reading frame number. Genes not previously assigned a C. elegans gene name are identified herein as mlt-12 to mlt-93. Eleven genes identified in our screen had been previously identified as functioning in molting, but had not been previously identified as targets for a nematicide, insecticide, or other compound that inhibits molting. These genes include C01H6.5 (nhr-23), C45B2.7 (ptr-4), F11C1.6 (nhr-25), F18C12.2 (rme-8), F29D11.1 (lrp-1), F53G12.3, F56C11.1, K04F10.4 (bli-4), T05C12.10 (qhg-1), T27F2.1 (C. elegans Skip), and ZK270.1 (ptr-23). Orthologs of these genes were not previously identified. Some genes not previously identified as functioning in molting had been previously assigned a C. elegans gene name. In keeping with C. elegans nomenclature practices, genes previously assigned a C. elegans gene name have not been renamed.

Mlt Phenotypes

Post-embryonic RNAi against milt genes listed in Tables 1A and 1B produced molting-specific defects in 5-100% of larvae (Table 1A and Table 1B). The majority of these worms also exhibited a larval arrest phenotype. This list identifies target genes by C. elegans cosmid name and open reading frame number. Homology searches using the blast algorithm and information available at wormbase (www.wormbase.org), a central repository of data on C. elegans, were used to identify the function of encoded proteins. At least three mlt genes, mlt-24, mlt-25, and mlt-27, encode proteins predicted to function as secreted proteases. These proteases are likely to function in the process of cuticle release, or, possibly, in the processing of peptide molting hormones.

TABLE 1A
RNAi Produced Molt Defects in 5-100% of Exposed Larvae

| Gene | ORF | Accession No. | Function | Reference for Mlt phenotypee |
|---|---|---|---|---|
| mlt-19 | B0024.14 | ref\|NM_073255 | Pro-collagen | |
| nhr-23 | C01H6.5 | ref\|NM_059638 | nuclear hormone receptor | Kostrouchova et al., 1998¹ |
| | | | transcription factor | |
| bli-1 | C09G5.6 | ref\|NM_063910 | cuticle collagen | |
| | C11H1.3 | ref\|NM_077984 | | |
| mlt-24 | C17G1.6 | ref\|NM_077268 | Metalloprotease, secreted | Morita et al., 2002² |
| mlt-20 | C23F12.1 | ref\|NM_077180 | endothelial actin-binding protein | |
| | | | repeats | |
| mlt-21 | B0272.5 | same as above | endothelial actin-binding protein | |
| | | | repeats | |
| mlt-14 | C34G6.6 | ref\|NM_059305 | repetitive Cys motifs; 4 PAN | |
| | | | domains | |
| mlt-22 | C37C3.3 | | | |
| mlt-27 | C42D8.5 | ref\|NM_076466 | Angiotension converting | |
| | | | enzyme, metalloprotease | |
| ptr-4 | C45B2.7 | ref\|NM_076612 | sterol-sensing domain | Zugasti et al., 2002³ |
| mlt-23 | CD4.4 | ref\|NM_072073 | coiled coil | |
| mlt-28 | CD4.6 | pir\|T32525 | protease | |
| mlt-29 | D1054.15 | ref\|NM_073362 | G-protein beta WD-40 repeats | |
| | | | beta-transducin-like | |
| mlt-21 | C26C6.3 | NM_059708. | Astacin metalloprotease | |
| acn-1 | C42D8.5 | NM_076466 | Angiotension converting enzyme | |
| mlt-20 | F08C6.1 | ref\|NM_076885 | ADAM/reprolysin | |
| | | | metalloprotease, 12 of | |
| | | | Thrombospondin type I domain | |
| mlt-13 | F09B12.1 | ref\|NM_078111 | MAM domains, secreted | |
| nhr-25 | F11C1.6 | ref\|NM_077761 | nuclear hormone recptor | Gissendanner and Sluder, 2000⁴ |
| mlt-30 | F16H9.2 | ref\|NM_077722 | nuclear hormone receptor | |
| lir-1 | F18A1.3 | emb\|AJ130959 | Transcription factor like lin-26 | |
| rme-8 | F18C12.2 | gb\|AF372457 | endocytosis DNAJ domain | Zhang et al., 2001⁵ |
| mlt-31 | F20G4.1 | ref\|NM_059784 | | |
| mlt-32 | F25B4.6 | ref\|NM_072095 | hydroxymethlglutaryl-CoA | |
| | | | synthase | |
| lrp-1 | F29D11.1 | ref\|NM_059726 | LDL-receptor related | Yochem et al., 1999⁶ |
| let-858 | F33A8.1 | ref\|NM_063962 | | |
| mlt-33 | F33C8.3 | ref\|NM_078044 | tetraspanin | |
| mlt-34 | F38H4.9 | ref\|NM_069846 | Hs P2AB serine/threonine | |
| | | | phosphatase | |
| mlt-35 | F40G9.1 | ref\|NM_064775 | Ankryin repeats | |
| mlt-36 | F41C3.4 | ref\|NM_062446 | | |
| elo-5 | F41H10.7 | ref\|NP_500793 | GNS1/SUR4 family | |
| mlt-17 | F45G2.5 | ref\|NM_067371 | SS pancreatic trypsin inhibitor | |
| mlt-38 | F49C12.12 | ref\|NM_069234 | transmembrane protein | |
| mlt-15 | F52B11.3 | ref\|NP_502699 | 4 PAN domains. Secretory | |
| | | | protein | |
| mlt-39 | F53B8.1 | pir∥T22551 | Hu PLEC1 orthologue; plectin, | |
| | | | kakapo homolog | |
| mlt-40 | F53G12.3 | ref\|NM_058283 | NADPH oxidase | Fraser, 2000⁷ |
| mlt-41 | F54A5.1 | ref\|NM_058402 | | |
| stc-1 | F54C9.2 | ref\|NM_063407 | Heat shock 70 Kd protein | |
| | | | (HSP70) | |
| | F53G12.3 | | animal haem peroxidase; | |
| | | | gp91/phox1 | |
| DuOx | F56C11.1 | ref\|NM_058285 | NADPH oxidase; animal haem | Fraser, 2000⁷ |
| | | | peroxidase; gp91/phox1 | |
| mlt-42 | F57B9.2 | ref\|NM_066115 | Tx human 1 Proline Rich, 1 | |
| | | | Glycosylytransferase family 5 | |
| mlt-43 | H04M03.4 | ref\|NP_500884 | | |
| let-805 | H19M22.1 | ref\|NM_065198 | myotactin form A | |
| bli-4 | K04F10.4 | ref\|NM_059427 | subtilase protease | Thacker et al., 1995⁸ |
| mlt-44 | K05C4.1 | pir\|T23336 | proteasome subunit | |
| mlt-45 | K06B4.5 | ref\|NM_074499 | nuclear hormone receptor | |
| mlt-46 | K07C5.6 | ref\|NM_073260 | zinc finger | |
| mup-4 | K07D8.1 | ref\|NM_066244 | mup-4 ion-channel SEA | |
| | | | domains, Ca-binding EGF | |
| | | | domains | |
| lag-1 | K08B4.1 | ref\|NM_068515 | DNA-binding protein, IPT/TIG | |
| | | | domain | |
| mlt-47 | K09H9.6 | ref\|NM_058707 | homologueof Dm Peter Pan, | |
| | | | which is required for larval | |
| | | | growth | |
| mlt-48 | M03F4.7 | ref\|NM_076443 | calcium binding protein, EF-hand | |
| | | | family 13x | |
| mlt-49 | M03F8.3 | ref\|NM_072146 | crn HAT (Half-A-TPR) repeat | |
| | | | 10x, TPR repeat 3x | |
| mlt-50 | M162.6 | ref\|NM_075434 | | |
| ifc-2 | M6.1 | ref\|NM_075732 | intermediate filament protein A | |
| pan-1 | M88.6 | ref\|NM_065523 | lecuine-rich repeats | |
| ran-4 | R05D11.3 | ref\|NM_059921 | Nuclear import; Nuclear | |
| | | | Transport Factor 2 (NTF2) | |
| | | | homologue | |
| kin-2 | R07E4.6 | ref\|NM_076598 | | |
| mlt-52 | R11G11.1 | ref\|NM_070836 | nuclear homrone receptor | |
| mlt-53 | T01C3.1 | ref\|NM_074284 | WD domain, G-beta repeats x13 | |
| mlt-54 | T01H3.1 | ref\|NM_063258 | proteolipid protein PPA1 like | |
| | | | protein | |
| | Y41D4B.10 | NM_067707 | Delta-serrate ligand precursor | |
| qhg-1 | T05C12.10 | ref\|NM_063324 | hedgehog-like, hint module | Wang et al., 1999⁹ |
| mlt-55 | T14F9.1 | ref\|NM_076011 | ATPase subunit | |
| mlt-56 | T19B10.2 | ref\|NM_073447 | secretory protein | |
| mlt-57 | T23F2.1 | ref\|NM_076531 | glycosyltransferase | |
| mlt-58 | T24H7.2 | ref\|NM_062848 | Heat shock protein hsp70, | |
| | | | Cytochrome b/b6 | |
| Ce Skip | T27F2.1 | ref\|NM_073549 | Drosophila puff specific protein | Kostrouchova et al., 2002¹⁰ |
| | | | BX42 like | |
| | F10C1.5 | NM_062737 | DSX DNA binding domain | |
| mlt-18 | W01F3.3 | ref\|NM_075592 | multiple BPTI-like domains, | |
| | | | secretory protein | |
| mlt-12 | W08F4.6 | ref\|NM_061358 | novel secretory protein | |
| mlt-59 | W09B6.1 | ref\|NM_061521 | acetyl-CoA carboxylase | |
| ifa-2 | W10G6.3 | ref\|NM_078247 | intermediate filament protein | |
| pqn-80 | Y111B2A.14 | ref\|NM_067244 | prion-like | |
| mlt-60 | Y37D8A.10 | ref\|NM_067275 | transmembrane protein | |
| mlt-61 | Y38F2AL.3 | ref\|NM_067786 | ATPase | |
| mlt-62 | Y48B6A.3 | ref\|NM_067371 | 5′-3′ exonuclease domain; | |
| | | | eggshell protein | |
| unc-52 | ZC101.2 | ref\|NM_064645 | basement membrane | |
| | | | proteoglycan | |
| mlt-63 | ZK1073.1 | ref\|NM_078233 | | |
| mlt-64 | ZK1151.1 | ref\|NM_060597 | plectrin | |
| mlt-65 | ZK262.8 | ref\|NM_075208 | Myosin head (motor domain) | |
| ptr-23 | ZK270.1 | ref\|NM_061202 | sterol-sensing domain | Schulze et al., 2002¹¹ |
| mlt-11 | ZK430.8 | ref\|NM_062376 | animal haem peroxidase; ShTk | |
| | | | domain | |
| mlt-67 | ZK686.3 | ref\|NM_066290 | Ankryin repeat | |
| mlt-16 | ZK783.1 | ref\|NM_066269 | ECM microfibril component (Hs | |
| | | | FBN-1 homolog) | |
| mlt-68 | ZK970.4 | ref\|NM_063816 | H+-transporting ATPase | |

¹Kostrouchova et al., Proc. Natl. Acad. Sci. 99: 9554-9559, 2002
²Morita et al, EMBO 23: 1063-1073.
³Zugasti et al., 2002 European Worm Meeting
⁴Gissendanner et al, Dev. Biol, 221: 259-72, 2000
⁵Zhang et al., Mol. Biol. Cell, 12: 2011-21, 2001
⁶Yochem et al., Development, 126: 597-606
⁷Fraser et al., Nature, 408: 325-30, 2000
⁸Thacker et al., Genes Dev. 9: 956-71, 1995
⁹Wang et al., 1999, International Worm Meeting
¹⁰Kostrouchova et al., Proc. Natl. Acad. Sci. 98: 7360-5, 2001
¹¹Schulze et al., 2002 European Worm Meeting

TABLE 1B
Genes identified in RNAi screen of clones from Vidal Orfeome Project

| | | | |
|---|---|---|---|
| Predicted | Gene | | Brief Molecular I.D./ |
| Gene | name | Accession # | Domains |

High frequency of Mlt phenotype

| | | | |
|---|---|---|---|
| Y54E10BR.5 | | ref\|NM_058691 | Signal Peptidase |
| B0513.1 | gei-5 | ref\|NM_070273 | GEX-3 interacting protein |
| R06A4.9 | | ref\|NM_064584 | WD domain, G beta repeats, HMG1/Y DNA binding domain |
| Y105E8B.1 | lev-11 | ref\|NM_061138 | tropomyosin |
| Y47D3B.1 | | ref\|NM_067064 | DUF23 |
| Y54F10AL.2 | est-1 | ref\|NM_065164 | telomerase subunit |
| T17H7.3 | | ref\|NM_064848 | |
| H27M09.5 | | ref\|NM_059558 | novel |
| F45E10.2 | | ref\|NM_063970 | solute carrier family 22 member |
| F25H8.6 | | ref\|NM_069384 | DNA binding, BED zinc finger |
| K04A8.6 | | ref\|NM_072260 | F-box |
| ZC13.3 | | ref\|NM_075772 | MAM domain |
| T19A5.3 | | ref\|NM_072907 | novel |

low frequency of Mlt phenotype

| | | | |
|---|---|---|---|
| F32D8.6 | emo-1 | ref\|NM_073377 | Protein translocation - Sec61 ortholog |
| F53F4.3 | | ref\|NM_073966 | novel |
| F56C9.12 | | ref\|NR_001470 | novel |
| T25B9.10 | | ref\|NM_069598 | endo/exonuclease phosphatase family |
| ZK154.3 | mec-7 | ref\|NM_076912 | beta-tubulin |
| Y37D8A.19 | | ref\|NM_067286 | novel secreted protein |
| Y37D8A.21 | | ref\|NM_067285 | RNA binding, RNP domain |
| Y71F9AL.7 | | ref\|NM_058666 | novel transmembrane protein |
| Y51H1A.3 | | ref\|NM_064506 | NADH dehydrogenase 1 beta subcomplex 8 19 kDa like |
| W03F9.10 | | ref\|NM_070740 | DUF382, Proline rich, PSP, HMG-1 DNA binding |
| ZK945.2 | pas-7 | ref\|NM_063776 | proteosome alpha subunit |
| ZK637.4 | | ref\|NM_066563 | novel putative nuclear protein |
| C30F8.2 | | ref\|NM_059114 | H+ transporting ATPase C subunit |
| F32H2.9 | tba-6 | ref\|NM_060018 | tubulin alpha |
| Y87G2A.5 | vrs-2 | ref\|NM_060976 | cytoplasmic valyl tRNA syhtethase |
| Y53F4B.22 | arp-1 | ref\|NM_064707 | actin like |
| Y77E11A.13 | npp-20 | ref\|NM_067686 | nuclear core protein, related to essential transport protein SEC1 |
| C15H11.7 | pas-1 | ref\|NM_074170 | 26s proteosome subunit |
| Y113G7B.23 | psa-1 | ref\|NM_075505 | SWI/SNF complex chromatin remodeling |
| C53H9.1 | rpl-27 | ref\|NM_058504 | large ribosomal subunit 27 |
| W09C5.6 | rpl-31 | ref\|NM_060990 | large ribosomal subunit 31 |
| T24B8.1 | rpl-32 | ref\|NM_063533 | large ribosomal subunit 32 |
| Y71A12B.1 | rps-6 | ref\|NM_061034 | small ribosomal subunit S6 |

Cuticle Retention Phenotypes

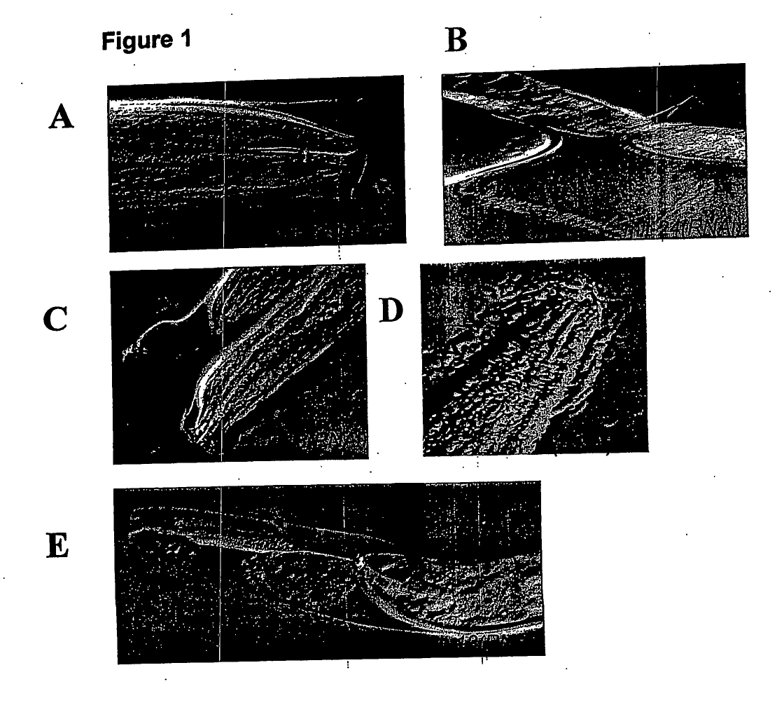

All Mlt larvae failed to fully shed their cuticles. For example, RNAi against mlt-12, mlt-13, mlt-18, and mlt-24 resulted in larvae partially encased in a sheath of unshed cuticle (FIGS. 1A-1E). The Mlt phenotype observed in these animals resembled the phenotype of lrp-1 (RNAi) nematodes. lrp-1 was previously shown to be required for molting (Yochem et al., Development, 126: 597-606, 1999).

Interestingly, specific differences were observed in cuticle retention among Mlt larvae. The tissue of mlt-13(RNAi) animals remained tethered to old cuticle expelled from the buccal cavity, suggesting a defect early in the execution of molting (FIG. 1E). In contrast, unc-52(RNAi) nematodes arrested with sheaths of cuticle extending from their posteriors, and appeared paralyzed except for small head movements. The phenotype of unc-52(RNAi) nematodes suggested a defect in the final stages of ecdysis. Undetached cuticle was observed around the most anterior portion of mlt-12(RNAi) animals (FIG. 1D). This anterior region corresponds to the location of the cells hyp2 through hyp6. Approximately 20% of mlt-24(RNAi) animals had cuticular sheaths wrapped around their mid-sections (FIGS. 1A and 1B). The discovery of phenotypic classes among Mlt larvae indicated that sets of mlt genes likely act together at specific steps of ecdysis, or that some mlt genes are required for apolysis of cuticle covering only one or two regions of the body. Further, most, if not all, genes uncovered appear essential for all four molts, since their inactivation produces molting-defective larvae at several developmental stages. The majority of gene inactivations also disrupted molts into, or out of, dauer, an alternative developmental stage that is adapted for survival in unfavorable conditions and resembles the infective form of parasitic nematodes. Generally, animals that failed to complete a molt also ceased to develop, but they would occasionally escape old cuticle after several hours, only to become trapped again at the next molt, as observed in qhg-1(RNAi) larvae.

Reproductive Phenotype

While the majority of Mlt larvae arrest development and die, possibly as a consequence of starvation, Mlt animals trapped in cuticle during the L4-to-adult transition occasionally produced a limited number of progeny. This was observed in qhg-1 (RNAi), nhr-23(RNAi), and mlt-13(RNAi) animals.

Phenotype Associated with Secretory Pathway Defects

RNAi against many genes known to function in the secretory pathway, such as the worm orthologs of the vesicle coat proteins SEC-23 and B-COP, disrupted molting (Table 2). Those secretory pathway components that gave a Mlt phenotype when inactivated by RNAi are listed in Table 2. The genes are listed by C. elegans cosmid name and open reading frame number. Homology searches using the blast algorithm and information available at wormbase (www.wormnbase.org), a central repository of data on C. elegans, were used to identify the function of encoded proteins.

TABLE 2
RNAi against Secretory Pathway Components Produced Molt Defects

| Gene | ORF | Molecular Function or Identity |
|---|---|---|
| nsf-1 | H15N14.1 | vesicular fusion; like human NSF |
| rab-5 | F26H9.6 | ras superfamily GTPase |
| tfg-1 | Y63D3A.5 | part of COPII complex; vesicle trafficking |
| snf-7 | C56C10.3 | vacuolar sorting |
| sar-1 | ZK180.4 | GTP-binding protein |
| arf-3 | F57H12.1 | \|GTP-binding protein |
| rab-1 | C39F7.4 | ras-family |
| sec-23 | Y113G7A.3 | COPII complex vesicular transport |
| dpy-23 | R160.1 | Clathrin adaptor complexes medium chain 7x |
| dyn-1 | C02C6.1 | dynamin family 8X |
| mlt-69 | E03H4.8 | beta coatomer-like |
| mlt-70 | F59E10.3 | Clathrin adaptor complex small chain |
| mlt-71 | K12H4.4 | signal peptidase |
| mlt-72 | D1014.3 | alpha-SNAP, NSF attachment protein |
| mlt-73 | C13B9.3 | clathrin adaptor |
| mlt-74 | F43D9.3 | sec1 family |
| | F41C3.4 | homolog of got-1 (GenBank Acc. No. |
| | | NM_062446) |
| | F38A1.8 | SRP-54 (GenBank Acc. No. NM_171254) |

Interestingly, the bodies of animals undergoing RNAi against secretory pathway genes tended to disintegrate over time, distinguishing them from other Mlt larvae. The isolation of sixteen secretory pathway genes in a screen for larvae unable to molt indicated that a functional secretory pathway is needed either to generate new cuticle or to export enzymes that allow larvae to break free of the old cuticle.

Larval Arrest Phenotypes

RNAi against genes shown in Table 3A produced molting defects in less than five percent of larvae, and also produced an early larval arrest phenotype (i.e., arrest in the L1 or L2 stage) in the majority of animals. RNAi against genes shown in Table 3B produced molting defects in 10% or less of larvae. This list identifies the target genes by C. elegans cosmid name and open reading frame number. Homology searches using the blast algorithm and information available at wormbase (www.wormbase.org), a central repository of data on C. elegans, were used to identify the function of encoded proteins.

TABLE 3A
RNAi Produced Molt Defects in Less than 5% of Exposed Larvae

| Gene | ORF | Molecular Function/Protein Domains |
|---|---|---|
| rpl-23 | B0336.10 | ribosomal protein |
| rps-0 | B0393.1 | ribosomal protein |
| rpl-14 | C04F12.4 | ribosomal protein L14 |
| rps-3 | C23G10.3 | ribosomal protein |
| rps-10 | D1007.6 | ribosomal protein |
| rps-23 | F28D1.7 | ribosomal protein |
| rpn-7 | F35H10.4 | ribosomal protein |
| rps-21 | F37C12.11 | ribosomal protein |
| rps-14 | F37C12.9 | ribosomal protein |
| rps-11 | F40F11.1 | ribosomal protein |
| rps-22 | F53A3.3 | ribosomal protein |
| rps-16 | T01C3.6 | ribosomal protein |
| rps-19 | T05F1.3 | ribosomal protein S19 |
| rpl-18 | Y45F10D.12 | ribosomal protein |
| mlt-75 | C09F12.1 | secretory protein |
| mlt-76 | C09H10.2 | Forkhead-associated (FHA) domain |
| mlt-77 | C17H12.14 | ATPase |
| mlt-78 | C37C3.2 | Domain found in IF2B/IF5 2x |
| mlt-79 | D2085.1 | |
| mog-5 | EEED8.5 | RNA helicase DEAD/DEAH box helicase |
| mig-10 | F10E9.7 | PH domain |
| mlt-80 | F19F10.9 | |
| mlt-81 | F28F8.5 | |
| mlt-82 | F32D1.2 | ATP synthase epsilion chain |
| vha-5 | F35H10.4 | H+ ion transport V-type ATPase 116 kDa subunit |
| | | family |
| mlt-83 | F41E7.1 | TM G-protein beta WD-40 repeats |
| mlt-84 | F42A8.1 | TGFB path |
| mlt-85 | F54B3.3 | AAA ATPase |
| mlt-86 | F55A3.3 | general chromatin factor |
| mlt-87 | F56F3.5 | Ribosomal protein S3A |
| mlt-88 | H06I04.4a | 4 ubiquitin domains, CH2 Zinc finger |
| mlt-89 | K06A4.6 | |
| mlt-90 | K10D6.1 | GABA receptor beta subunit |
| mlt-91 | R06A10.1 | |
| mlt-92 | T07D10.1 | transmembrane protein |
| lin-29 | Y17G7A.2 | lin-29 |
| mlt-93 | Y23H5A.7 | aminoacyl-tRNA synthetase |
| vha-11 | Y38F2AL.3 | ATPase |
| | Y41D4B.21 | |
| | Y41D4B.5 | ion channel protein |
| | Y45F10B.5 | |
| | Y55H10A.1 | Cadherin |
| | ZK1236.3 | |
| | ZK265.5 | |
| | ZK265.6 | G-protein coupled receptor |
| | ZK652.1 | small nuclear ribonucleoprotein |

TABLE 3B
Gene inactivations that cause molting defects in 10% or less of larvae

| Gene | ORF | Accession # | Molecular Identity |
|---|---|---|---|
| | B0348.1 | ref\|NM_070727 | nematode-specific protein family |
| clc-1 | C09F12.1 | ref\|NM_077446 | claudin-like |
| | C23F12.1 | ref\|NM_077179 | endothelial actin-binding protein repeats |
| | C37C3.2 | gb\|U64857 | domain found in IF2B/IF5 |
| | CD4.4 | ref\|NM_072073 | coiled 4-coil domain |
| pas-6 | CD4.6 | ref\|NM_072071 | proteosome subunit |
| cdc-5 | D1081.8 | ref\|NM_059902 | myb-like DNA binding domain |
| pyr-1 | D2085.1 | ref\|NM_063437 | glutamine-dependent carbamoyl-phosphate synthase |
| mog-5 | EEED8.5 | ref\|NM_062618 | RNA helicase DEAD/DEAH box helicase |
| | F19F10.9 | ref\|NM_072551 | SART-1 family |
| | F28F8.5 | ref\|NM_074471 | coiled 4-coil domain, nematode specific |
| vha-5 | F35H10.4 | ref\|NM_068998 | H+ trans. V-type ATPase |
| | F25B4.6 | ref\|NM_072095 | hydroxymethylglutaryl-coenzyme A synthase |
| clo-5 | F41H10.7 | ref\|NM_068392 | fatty acid elongation |
| | F42A8.1 | ref\|NM_063590 | signal sequence, nematode specific |
| rpn-7 | F49C12.8 | ref\|NM_069231 | proteasome regulatory particle |
| | F53G12.4 | ref\|NM_058282 | coiled 4-coil domain, nematode specific |
| | F54B3.3 | ref\|NM_063809 | AAA ATPase |
| | F55A3.3 | ref\|NM_060420 | metallopeptidase family M24 |
| stc-1 | F54C9.2 | ref\|NM_063407 | truncated HSP |
| | H04M03.4 | ref\|NM_068483 | novel |
| ubl-1 | H06104.4 | ref\|NM_171089 | 4 ubiquitin domains, CH2 Zinc linger |
| ceh-6 | K02B12.1 | ref\|NM_059903 | homeobox |
| | K06A4.6 | ref\|NM_073045 | zinc metalloprotease like |
| slu-7 | K07C5.6 | ref\|NM_073260 | splicing factor |
| lag-1 | K08B4.1 | ref\|NM_171350 | transcriptional regulator |
| | R06A10.1 | ref\|NM_05841 | ER membrane protein, nematode specific |
| kin-2 | R07E4.6 | ref\|NM_07659 | cAMP-dependent protein kinase |
| cbp-1 | R10E11.1 | ref\|NM_066760 | CBP/p300 homolog |
| | T06D8.6 | ref\|NM_064002 | cylochrome c cl home lyase |
| | T19B10.2 | ref\|NM_073447 | coiled coil domain, nematode specific |
| vha-4 | T01H3.1 | ref\|NM_063258 | vacuular proton ATPase, V0 proteolipid subunit C. |
| | T07D10.1 | ref\|NM_060791 | signal peptide, nematode specific |
| crs-1 | Y23H5A.7 | ref\|NM_058612 | cysteinyl tRNA Synthetase |
| vha-11 | Y38F2AL.3 | ref\|NM_067786 | vacuolar H+ ATPase |
| vha-3 | Y38F2AL.4 | ref\|NM_067787 | vacuolar H+ ATPase |
| | Y45F10B.5 | ref\|NM_070216 | transmembrane domains, nematode-specific |
| | Y55H10A.1 | ref\|NM_067931 | H+ lysosomal ATPase like |
| sca-1 | K11D9.2 | ref\|NM_066984 | Sarco-Endoplasmic Reticulum Calcium ATPase |
| | ZK1236.3 | ref\|NM_066460 | nematode specific |
| snr-5 | ZK652.1 | ref\|NM_066307 | small nuclear ribonuclear protein Sm F |
| rpl-23 | B0336.10 | ref\|NM_065830 | ribosomal protein |
| rps-0 | B0393.1 | ref\|NM_065577 | ribosomal protein |
| rpl-14 | C04F12.4 | ref\|NM_060175 | ribosomal protein L14 |
| rps-3 | C23G10.3 | ref\|NM_065948 | ribosomal protein |
| rps-10 | D1007.6 | ref\|NM_058997 | ribosomal protein |
| rps-23 | F28D1.7 | ref\|NM_069964 | ribosomal protein |
| rps-21 | F37C12.11 | ref\|NM_066178 | ribosomal protein |
| rps-14 | F37C12.9 | ref\|NM_066171 | ribosomal protein |
| rps-11 | F40F11.1 | ref\|NM_069785 | ribosomal protein |
| rps-22 | F53A3.3 | ref\|NM_065080 | ribosomal protein |
| rpl-15 | K11H12.2 | ref\|NM_066422 | ribosomal protein |
| rps-16 | T01C3.6 | ref\|NM_074289 | ribosomal protein |
| rps-19 | T05F1.3 | ref\|NM_060154 | ribosomal protein S19 |
| rpl-18 | Y45F10D.12 | ref\|NM_070254 | ribosomal protein |
| rps-1 | F56F3.5 | ref\|NM_065509 | ribosomal protein S3A |
| | C09H10.2 | ref\|NM_063974 | ribosomal protein L44 |
| | Y41D4B.5 | ref\|NM_067714 | ribosomal protein S28e |

The Mlt phenotype was observed after several days of exposure to dsRNA. Table 3A includes genes that encode ribosomal proteins that are likely to be required for larval growth and development, and are unlikely to be specifically required for molting. Table 3A also includes genes that are likely to function in neurons that regulate ecdysis. RNAi against neuroendocrine genes is expected to be relatively ineffective, given that neuronal genes are often refractory to RNAi. Nonetheless, such neural control genes are expected to be conserved among Ecdysozoans and therefore represent targets for the development of nematicides and insecticides. Neuronal mlt genes are inactivated in relatively few larvae exposed to dsRNA-expressing-bacteria.

Improved methods of RNAi are expected to identify additional mlt genes that function in the neuroendocrine regulation of molting. For example, the use of mutants that show enhanced RNAi, such as nematodes having a mutation in rrf-3 (Simmer et al., Curr Biol. 12: 1317, 2002) may increase the sensitivity of the RNAi-based screen for mlt genes. Nematodes having an rrf-3 mutation may be screened using the methods described herein to identify new mlt genes. RNAi clones that disrupt molting only in hypersensitive strains likely act in neuroendocrine signaling pathways common to all Ecdysozoans (e.g., flies and nematodes). Drugs that targeted such proteins would be expected to disrupt molting in most Ecdysozoans, while having no adverse side effects on humans.

Pleiotropic Phenotypes

Pleiotropic phenotypes were associated with RNAi against sixteen open reading frames identified in the Mlt screen (e.g., F56C11.1 (DuOx), F53G12.3, F55A3.3, F18A1.3 (lir-1), ZK430.8, F41C3.4, Y48B6A.3, K07D8.1 (mup-4), W01G6.3, F57B9.2, K08B4.1 (lag-1), F49C12.12, F38H4.9, F25B4.6, ZK262.8, M162.6, ZK1073.1).

Conservation of mlt Genes

Table 4A shows the conservation of a subset of mlt genes across phylogeny, identifying the RNAi target genes by C. elegans cosmid name and open reading frame number, and their orthologs in Drosophila melanogaster (Dm), Homo sapiens (Hs), and Saccharomyces cerevisiae (Sc) by Genbank accession number and blast score. DNA sequences corresponding to the mlt genes of interest were retrieved from the repositories of sequence information at the NCBI website (http://www.ncbi.nlm.nih.gov/) or at wormbase (www.wormbase.org). The DNA sequence was then used for standard translating blast [tBLASTN] searching using the NCBI website (http://www.ncbi.nlm.nih.gov/BLAST/). For each mlt gene, Table 4A identifies the Genebank accession number and blast score for the top blast hit from Drosophila melanogaster (Dm), Homo sapiens (Hs), and Saccharomyces cerevisiae (Sc). The DNA sequence corresponding to the top ortholog candidate produced by tblastn was retrieved from Genbank (http://www.ncbi.nlm.nih.gov/) and used for a BLASTx search of C. elegans proteins using the wormbase site (http://www.wormbase.org/db/searches/blast). In one preferred embodiment, conservation of the mlt gene in flies or humans was indicated when the BLASTx search produced the starting MLT protein as the top score. These most highly conserved sequences are shaded in deep color in Table 4A. All other related sequences are shaded with lighter color. These methods provided for the identification of orthologs of C. elegans mlt genes (Tables 1A, 1B, 4A-4D, 7 and 8) revealed by our RNAi analysis. An ortholog is a protein that is highly related to a reference sequence. One skilled in the art would expect an ortholog to functionally substitute for the reference sequence. Tables 4A and 7 list exemplary orthologs by Genbank accession number and blast score.

TABLE 4A
Conserved mlt Genes

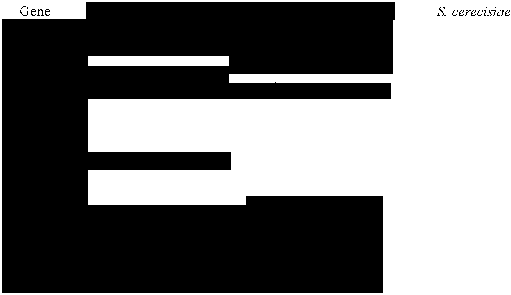

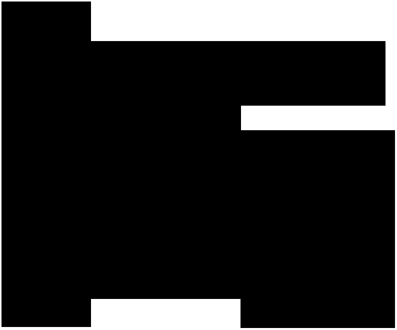

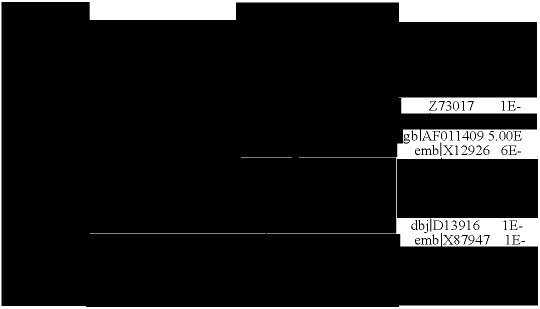

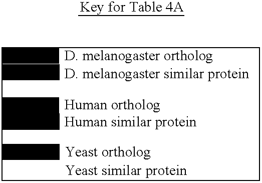

Table 4B lists C. elegans genes and Drosophila and human orthologs identified using a tblastn search.

TABLE 4B
Selected gene inactivations associated with molting defects

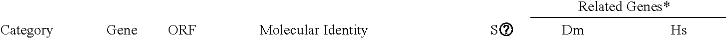

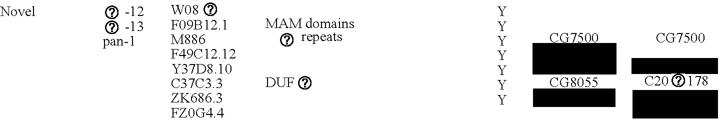

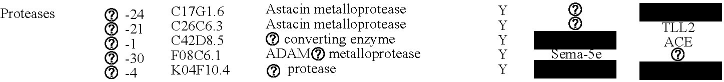

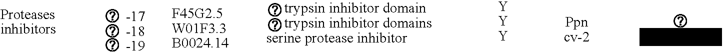

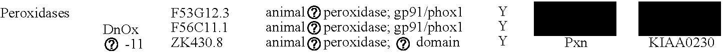

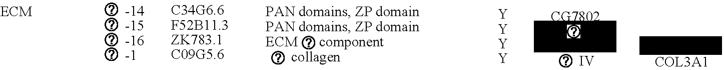

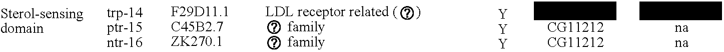

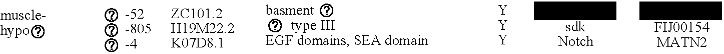

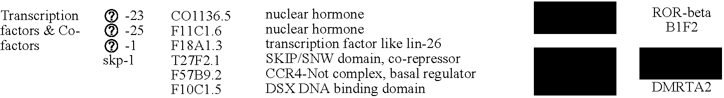

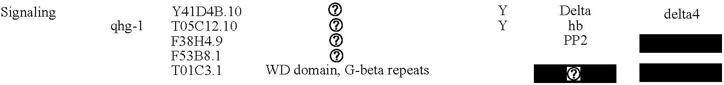

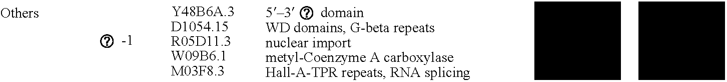

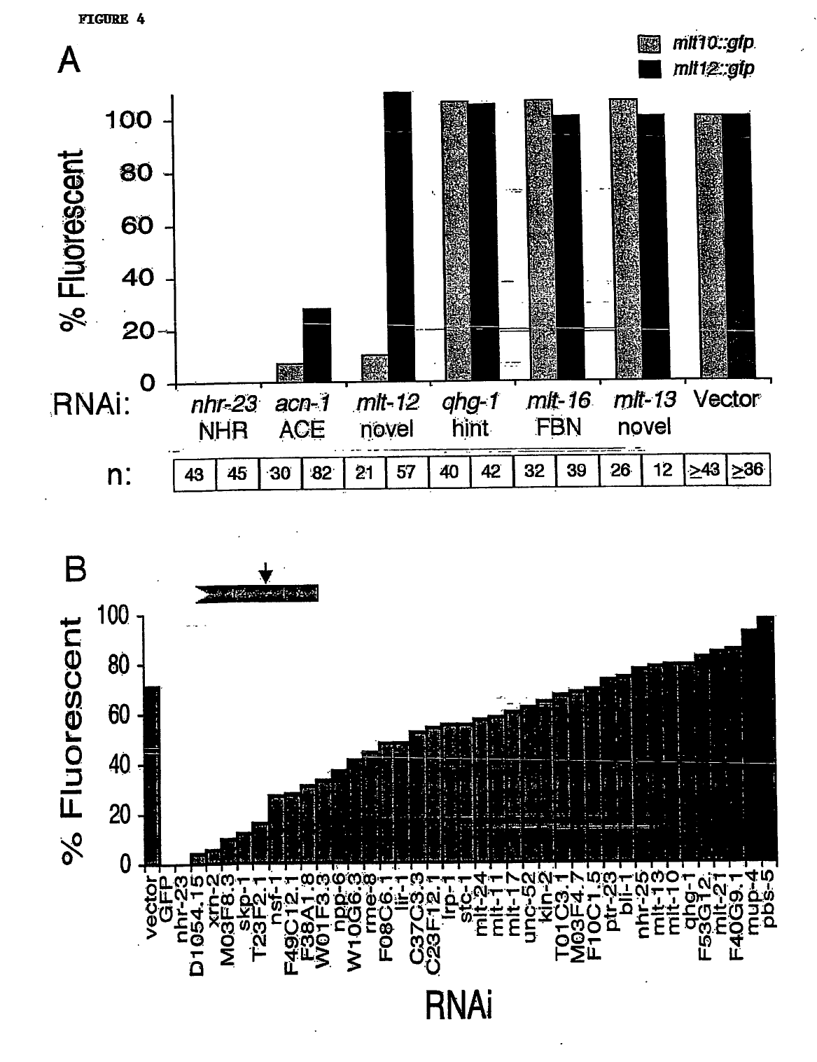

Figure 4B Legend: Top hits from tblastn searches of the human or fly genome using the predicted C. elegans
gene product. Dark shading indicates that a blastx search with the predicted human or fly protein uncovered the
corresponding C. elegans protein as the top-scoring match in C. elegans, identifying potential orthologs.
Y indicates the presence of a secretory signal peptide (SP) in the predicted gene product.

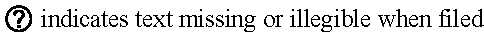

Table 4C identifies genes whose inactivation disrupts molting and related genes in other species.

TABLE 4C
C. elegans genes that disrupt molting and their coounterparts in other species

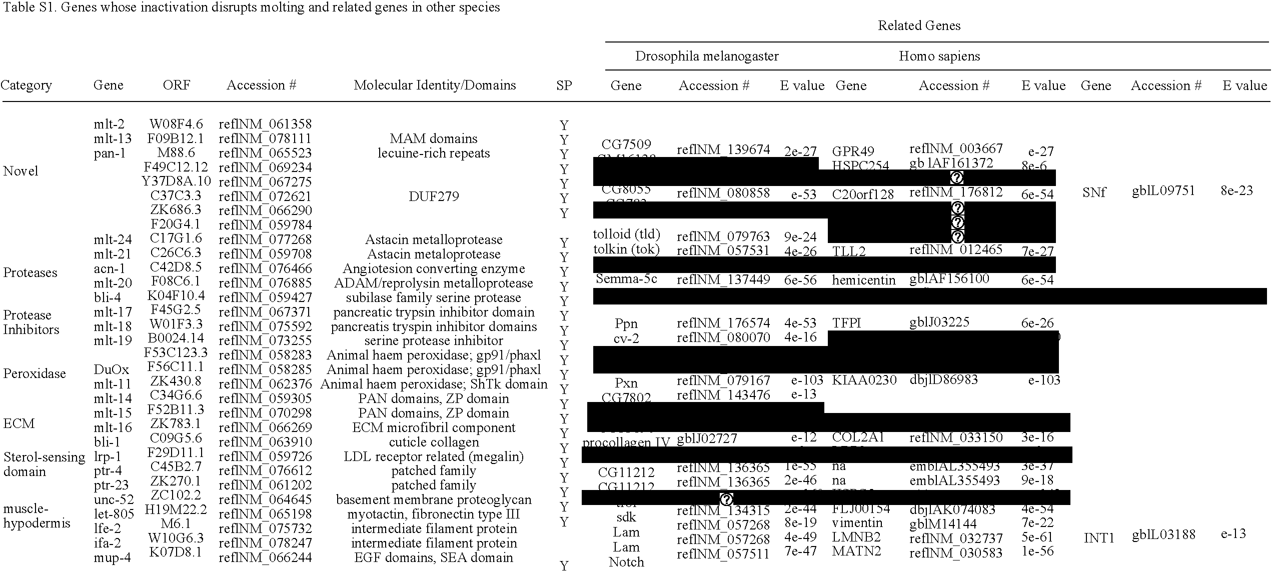

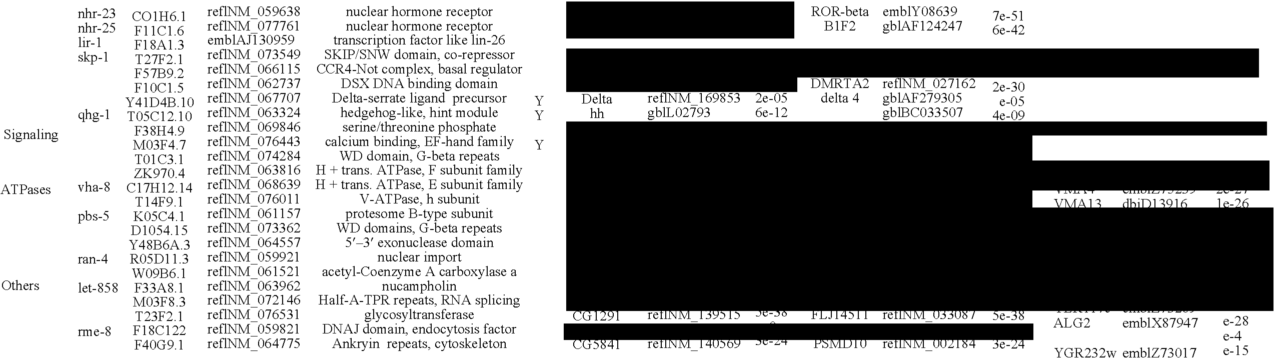

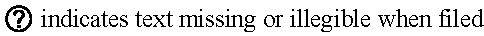

TABLE 4D
Table S2. Homologs of selected molting genes in parasitic nematode species

| | | Related Genes (1) | | | | | | | |
|---|---|---|---|---|---|---|---|---|---|
| | | | | | | Strongyloides | | | |
| | | Onchocerea volvulus | | Brugla maiayi | | stercoralis | | Strongyloides ratti | |
| Gene | ORF | Accession # | E value | Accession # | E value | Accession # | E value | Accession # | E value |
| nhr-23 | C01H6.5 | | | dbj\|AU000440 | | | | | |
| bli-1 | C09G5.6 | gb\|BF482033 | 4E−19 | gb\|AI066836 | 3E−22 | gb\|BG226349 | 7E−25 | | |
| mlt-24 | C17G1.6 | | | gb\|AA471557 | 5E−17 | gb\|BG224501 | 9E−35 | gb\|BI741990 | 3E−39 |
| mlt-21 | C26C6.3 | | | | | gb\|BE224326 | 6E−32 | gb\|BI323632 | 6e−29 |
| mlt-14 | C34G6.6 | | | gb\|AA471404 | 2E−68 | gb\|BE579677 | 2E−53 | gb\|BI073876 | 1E−41 |
| acn-1 | C42D8.5 | | | | | | | | |
| | CD4.4 | | | | | | | | |
| | D1054.15 | gb\|BE202350 | 9E−49 | | | | | | |
| mlt-20 | F08C6.1 | | | | | gb\|BE581131 | 2E−34 | | |
| mlt-13 | F09B12.1 | gb\|AI665735 | 4E−10 | | | gb\|BG226227 | 1E−23 | | |
| nhr-25 | F11C1.6 | | | dbj\|AU000440 | | gb\|BE581104 | 1E−27 | | |
| lrp-1 | F29D11.r | | | gb\|AW055802 | 3E−38 | | | gb\|BG893946 | 3E−27 |
| | F38H4.9 | | | | | | | | |
| | F40G9.1 | | | | | | | | |
| mlt-17 | F45G2.5 | | | | | | | | |
| | F49C12.12 | | | | | gb\|BE029934 | 1E−15 | | |
| mlt-15 | F52B11.3 | | | gb\|AA661399 | 4E−48 | gb\|BE580180 | 1E−73 | gb\|BG893830 | 7E−80 |
| | F53G12.3 | | | gb\|AA161577 | 1E−39 | | | | |
| stc-1 | F54C9.2 | | | | | gb\|BG226148 | 1E−44 | | |
| DuOX | F56C11.1 | | | gb\|AA161577 | 1E−39 | | | | |
| | F57B9.2 | | | | | | | | |
| | H04M03.4 | gb\|AA294602 | 5E−15 | gb\|AI770981 | 2E−27 | gb\|BG227443 | 2E−75 | gb\|BI397280 | 1E−61 |
| | H19M22.2 | | | | | | | | |
| bli-4 | KD4F10.4 | | | | | gb\|BE028912 | 7E−24 | gb\|BQ091197 | 2E−18 |
| mup-4 | K07D8.1 | | | gb\|AI783143 | 1E−66 | gb\|BE223322 | 2E−23 | gb\|BI450575 | 3E−38 |
| | M03F4.7 | | | gb\|BM889340 | 1E−78 | gb\|BG226767 | 4E−85 | gb\|BI450741 | 2E−98 |
| ifc-2 | M6.1 | gb\|AA917260 | 2E−19 | gb\|AW675831 | 8E−19 | gb\|BF014961 | 3E−28 | gb\|BI742464 | 8E−19 |
| pan-1 | M88.6 | | | | | | | | |
| | T01C3.1 | | | | | | | | |
| qhg-1 | T05C12.10 | gb\|AW257707 | 1E−22 | | | gb\|BF014893 | 1E−34 | | |
| | T14F9.1 | | | | | gb\|BG226359 | 5E−70 | | |
| | T23F2.1 | | | | | gb\|BE579591 | 7E−75 | | |
| skp-1 | T27F2.1 | | | | | | | | |
| mlt-18 | W01F3.3 | | | | | gb\|BE580288 | 3E−20 | gb\|BG893620 | 2E−19 |
| mlt-12 | W08F4.6 | gb\|BG310588 | E−121 | gb\|BE753466 | E−104 | gb\|BG227161 | 2E−84 | | |
| | W09B6.1 | | | | | gb\|BE580061 | 8E−43 | gb\|BQ091288 | 7E−29 |
| ifa-2 | W10G6.3 | gb\|BF199444 | 2E−62 | gb\|AW675831 | 2E−75 | gb\|BE224367 | 7E−43 | gb\|BI742464 | 8E−50 |
| | Y37D8A.10 | | | | | gb\|BE029374 | 1E−37 | | |
| | Y48B6A.3 | | | | | | | gb\|BI324097 | 6E−40 |
| unc-52 | ZC101.2 | gb\|AW980135 | 4E−62 | gb\|AI723671 | 5E−60 | gb\|BG227295 | 1E−49 | gb\|BI323571 | 3E−44 |
| ptr-23 | ZK270.1 | gb\|BE202282 | 2E−11 | gb\|AW257677 | 3E−43 | gb\|BE579648 | 5E−54 | | |
| mlt-11 | ZK430.8 | | | gb\|AI723670 | 8E−40 | gb\|BG227360 | 5E−72 | gb\|BI073673 | 5E−31 |
| | ZK686.3 | | | gb\|AA842318 | 8E−22 | gb\|BE581316 | 2E−48 | | |
| | | Related Genes (1) | | | | | | | |
| | | Ancylostoma | | | | | | | |
| | | ceylanicum | | Aocylostoma caninum | | Necator americanus | | Ascaris sum | |
| Gene | ORF | Accession # | E value | Accession # | E value | Accession # | E value | Accession # | E value |
| ahr-23 | C01H6.5 | | | | | | | gb\|BM281749 | 2E−39 |
| blt-1 | C09G5.6 | | | | | | | gb\|AW165858 | 1E−26 |
| mlt-24 | C17G1.6 | | | gb\|BQ667369 | 3E−21 | gb\|BU089288 | 2E−29 | gb\|BQ835552 | 8E−41 |
| mlt-21 | C26C6.3 | gb\|CB276958 | 2E−36 | | | gb\|BU088646 | 2E−33 | gb\|BU965942 | 8E−38 |
| mlt-14 | C34G6.6 | | | | | | | gb\|BI782938 | 9E−52 |
| ocn-1 | C42D8.5 | | | | | | | | |
| | CD4.4 | | | | | | | | |
| | D1054.15 | | | gb\|BG232752 | 4E−77 | gb\|BU088714 | 1E−108 | | |
| mlt-20 | F08C6.1 | | | | | | | | |
| mlt-13 | F09B12.1 | | | | | | | | |
| nhr-25 | F11C1.6 | | | | | | | gb\|BM280724 | 6E−20 |
| trp-1 | F29D11.r | | | | | | | | |
| | F38H4.9 | | | | | gb\|BU666328 | 1e−118 | gb\|BI1782814 | 8E−89 |
| | F40G9.1 | | | gb\|CA341524 | 3E−37 | gb\|BG467849 | 3E−13 | gb\|BI594288 | 8E−29 |
| mlt-17 | F45G2.5 | | | | | | | | |
| | F49C12.12 | gb\|BQ274691 | 1E−34 | gb\|BF250630 | 1E−22 | gb\|BU089096 | 9E−37 | | |
| mlt-15 | F52B11.3 | | | | | | | | |
| | F53G12.3 | | | gb\|AW735074 | 6E−64 | | | | |
| src-1 | F54C9.2 | | | | | | | | |
| DuOX | F56C11.1 | | | gb\|AW735074 | 6E−64 | | | | |
| | F57B9.2 | | | gb\|BU780997 | 6E−53 | | | | |
| | H04M03.4 | | | | | | | | |
| | H19M22.2 | | | gb\|BF250605 | | | | | |
| blt-4 | KD4F10.4 | | | gb\|BQ666394 | 2E−24 | gb\|BU087198 | 4E−14 | | |
| mup-4 | K07D8.1 | | | | | | | | |
| | M03F4.7 | | | gb\|BQ666411 | 1E−111 | | | gb\|BM319475 | 1E−103 |
| ifc-2 | M6.1 | | | | | | | gb\|BM280603 | 1E−28 |
| pan-1 | M88.6 | | | | | | | | |
| | T01C3.1 | | | | | | | | |
| qhg-1 | T05C12.10 | | | | | | | | |
| | T14F9.1 | | | gb\|BM130242 | 4E−72 | gb\|BU086612 | 5E−65 | gb\|BI594547 | 4E−67 |
| | T23F2.1 | | | | | | | | |
| skp-1 | T27F2.1 | | | | | gb\|BU087096 | 1E−22 | | |
| mlp-18 | W01F3.3 | | | gb\|BM077795 | 2E−19 | gb\|BU666009 | 1E−21 | | |
| mlp-12 | W08F4.6 | | | | | | | | |
| | W09B6.1 | | | gb\|BQ125044 | 2E−61 | gb\|BU666204 | 2E−15 | gb\|BI782124 | 2E−47 |
| ifa-2 | W10G6.3 | | | | | | | gb\|BM280603 | 1E−84 |
| | Y37D8A.10 | gb\|BQ288481 | 2E−59 | | | gb\|BU666155 | 9E−27 | | |
| | Y48B6A.3 | | | | | | | | |
| unc-52 | ZC101.2 | | | gb\|BE352403 | 4E−19 | | | gb\|BI782862 | 1E−13 |
| ptr-23 | ZK270.1 | | | gb\|BM130388 | 9E−98 | gb\|BU086563 | | gb\|BM033843 | 7E−20 |
| mlp-11 | ZK430.8 | | | | | | | | |
| | ZK686.3 | | | | | gb\|BG467473 | 6E−22 | | |
| | | Related Genes (1) | | | | | | Related Genes | |
| | | Haemonchus contortus | | Dirofilaria immlus | | Trichurls muris | | Trichinella spiralis | |
| Gene | ORF | Accession # | E value | Accession # | E value | Accession # | E value | Accession # | value |
| ahr-23 | C01H6.5 | | | | | | | gb\|BG353339 | 3E−29 |
| blt-1 | C09G5.6 | | | | | | | | |
| mlt-24 | C17G1.6 | | | | | | | gb\|BQ738378 | 2E−17 |
| mlt-21 | C26C6.3 | | | | | | | | |
| mlt-14 | C34G6.6 | gb\|BF060055 | 4E−18 | | | gb\|BG577864 | 4E−12 | | |
| ocn-1 | C42D8.5 | | | | | gb\|BM277122 | 6E−15 | gb\|BG520845 | 1E−15 |
| | CD4.4 | | | | | | | gb\|BG519941 | 6E−31 |
| | D1054.15 | | | | | | | gb\|BG520170 | 2E−26 |
| mlt-20 | F08C6.1 | | | | | | | | |
| mlt-13 | F09B12.1 | | | | | gb\|BF169279 | 5E−11 | | |
| nhr-25 | F11C1.6 | | | | | | | | |
| trp-1 | F29D11.r | | | | | | | | |
| | F38H4.9 | | | | | | | | |
| | F40G9.1 | | | | | | | | |
| mlt-17 | F45G2.5 | | | | | | | | |
| | F49C12.12 | | | | | gb\|BM174586 | 2E−19 | gb\|BQ543136 | 4E−17 |
| mlt-15 | F52B11.3 | | | | | | | | |
| | F53G12.3 | | | | | | | | |
| src-1 | F54C9.2 | | | | | | | | |
| DuOX | F56C11.1 | | | | | | | | |
| | F57B9.2 | | | | | | | | |
| | H04M03.4 | | | | | | | | |
| | H19M22.2 | | | | | | | | |
| blt-4 | KD4F10.4 | gb\|CA033722 | 1E−95 | | | | | gb\|BQ693113 | 1E−51 |
| mup-4 | K07D8.1 | | | | | | | | |
| | M03F4.7 | gb\|CA033609 | 1E−62 | | | | | | |
| ifc-2 | M6.1 | gb\|BF060126 | 4E−25 | | | gb\|BM174670 | 8E−32 | gb\|BG353660 | 5E−26 |
| pan-1 | M88.6 | | | | | | | | |
| | T01C3.1 | | | | | | | | |
| qhg-1 | T05C12.10 | | | | | | | | |
| | T14F9.1 | | | | | | | | |
| | T23F2.1 | | | | | | | | |
| skp-1 | T27F2.1 | | | | | | | | |
| mlp-18 | W01F3.3 | gb\|BF422862 | 9E−18 | | | gb\|BM174557 | 9E−21 | gb\|BQ692168 | 2E−8 |
| mlp-12 | W08F4.6 | | | | | | | | |
| | W09B6.1 | | | | | | | | |
| ifa-2 | W10G6.3 | gb\|BF060126 | 5E−57 | gb\|BQ455787 | 1E−35 | gb\|BF049882 | 2E−69 | gb\|BG353660 | 6E−68 |
| | Y37D8A.10 | gb\|BI595303 | 3E−68 | | | | | | |
| | Y48B6A.3 | | | | | | | | |
| unc-52 | ZC101.2 | gb\|BE496755 | 3E−99 | gb\|BQ454813 | 5E−58 | | | | |
| ptr-23 | ZK270.1 | | | | | | | | |
| mlp-11 | ZK430.8 | | | | | | | gb\|BG353679 | 3E−28 |
| | ZK686.3 | gb\|BF423018 | 9E−74 | | | | | | |
| | | Related Genes | | | | | | | |
| | | Toxocara canis | | Globodera pallida | | Globodera rostochiensis | | Meloidogyne arenaria | |
| Gene | ORF | Accession # | E value | Accession # | E value | Accession # | E value | Accession # | E value |
| ahr-23 | C01H6.5 | | | | | | | | |
| blt-1 | C09G5.6 | | | gb\|BM415129 | 1E−20 | | | | |
| mlt-24 | C17G1.6 | | | | | | | gb\|BI747415 | 2E−17 |
| mlt-21 | C26C6.3 | | | | | gb\|BM343299 | | gb\|BI747765 | 2E−25 |
| mlt-14 | C34G6.6 | | | | | | | gb\|BI745765 | 4E−10 |
| ocn-1 | C42D8.5 | | | | | | | gb\|BI501765 | 4E−41 |
| | CD4.4 | | | | | | | | |
| | D1054.15 | | | gb\|BM415102 | 4E−56 | | | gb\|BI863068 | 2E−87 |
| mlt-20 | F08C6.1 | | | | | | | | |
| mlt-13 | F09B12.1 | | | | | | | | |
| nhr-25 | F11C1.6 | | | | | gb\|BM354985 | 2E−17 | | |
| trp-1 | F29D11.r | | | gb\|BM415763 | E−119 | gb\|AW506417 | 2E−86 | | |
| | F38H4.9 | | | | | | | | |
| | F40G9.1 | | | | | | | | |
| mlt-17 | F45G2.5 | | | | | | | | |
| | F49C12.12 | | | | | gb\|AW506351 | 2E−36 | | |
| mlt-15 | F52B11.3 | | | | | | | | |
| | F53G12.3 | | | | | | | | |
| src-1 | F54C9.2 | | | | | | | | |
| DuOX | F56C11.1 | | | | | | | | |
| | F57B9.2 | | | | | | | | |
| | H04M03.4 | | | | | | | gb\|BI745690 | 1E−61 |
| | H19M22.2 | | | | | gb\|BM343207 | | | |
| blt-4 | KD4F10.4 | | | | | gb\|AW506559 | 8E−34 | | |
| mup-4 | K07D8.1 | | | | | | | | |
| | M03F4.7 | gb\|BM966480 | 9E−90 | gb\|BM415425 | 1E−66 | | | | |
| ifc-2 | M6.1 | gb\|BM965806 | 4E−16 | | | | | | |
| pan-1 | M88.6 | | | | | | | gb\|BI746256 | 3E−15 |
| | T01C3.1 | | | | | | | | |
| qhg-1 | T05C12.10 | | | | | | | | |
| | T14F9.1 | | | gb\|BM415082 | 2E−60 | gb\|BM345905 | 2E−73 | | |
| | T23F2.1 | | | | | | | | |
| skp-1 | T27F2.1 | | | | | | | | |
| mlp-18 | W01F3.3 | | | | | | | gb\|BI746672 | 6E−31 |
| mlp-12 | W08F4.6 | | | | | | | | |
| | W09B6.1 | gb\|BM966530 | 1E−37 | | | | | | |
| ifa-2 | W10G6.3 | gb\|BM965806 | 1E−52 | | | gb\|BM344699 | 3E−78 | gb\|BI747934 | 8E−53 |
| | Y37D8A.10 | | | | | | | gb\|BI747379 | 4E−30 |
| | Y48B6A.3 | | | | | gb\|BM345416 | 3E−13 | | |
| unc-52 | ZC101.2 | gb\|BM965583 | 9E−59 | | | gb\|AW506417 | 1E−14 | | |
| ptr-23 | ZK270.1 | | | | | gb\|BM344825 | 7E−18 | gb\|BI746878 | 2E−12 |
| mlp-11 | ZK430.8 | | | | | | | | |
| | ZK686.3 | | | gb\|AW505639 | 1E−32 | | | | |
| | | Related Genes | | | | | | | |
| | | Meloidogyne incognita | | Meloidogyne javanica | | Meloidogyne hapta | | Heterodera glycines | |
| Gene | ORF | Accession # | E value | Accession # | E value | Accession # | E value | Accession # | E value |
| ahr-23 | C01H6.5 | | | | | | | | |
| blt-1 | C09G5.6 | | | | | | | | |
| mlt-24 | C17G1.6 | | | | | | | | |
| mlt-21 | C26C6.3 | | | | | | | | |
| mlt-14 | C34G6.6 | | | gb\|BI744615 | 4E−44 | | | | |
| ocn-1 | C42D8.5 | gb\|BM881559 | 8E−41 | gb\|BG735807 | 6E−38 | gb\|BM902335 | 9E−26 | | |
| | CD4.4 | | | | | | | | |
| | D1054.15 | | | | | | | | |
| mlt-20 | F08C6.1 | | | | | | | | |
| mlt-13 | F09B12.1 | | | | | | | | |
| nhr-25 | F11C1.6 | | | | | | | | |
| trp-1 | F29D11.r | | | | | gb\|BM901359 | 2E−43 | | |
| | F38H4.9 | | | gb\|BI744849 | 4E−79 | | | | |
| | F40G9.1 | | | | | | | | |
| mlt-17 | F45G2.5 | | | | | | | | |
| | F49C12.12 | | | gb\|BI745272 | 3E−12 | gb\|BU094732 | 2E−30 | gb\|BF013515 | 2E−36 |
| mlt-15 | F52B11.3 | | | | | gb\|BM952243 | 9E−71 | | |
| | F53G12.3 | | | | | | | | |
| src-1 | F54C9.2 | | | | | | | | |
| DuOX | F56C11.1 | | | | | | | | |
| | F57B9.2 | gb\|BM881751 | 3E−36 | | | | | | |
| | H04M03.4 | | | | | gb\|BM902109 | 2E−56 | | |
| | H19M22.2 | | | | | | | | |
| blt-4 | KD4F10.4 | gb\|BM880593 | 9E−14 | | | gb\|BM901742 | 2E−20 | | |
| mup-4 | K07D8.1 | gb\|BE238861 | 8E−38 | | | | | | |
| | M03F4.7 | | | | | gb\|BM900690 | 2E−83 | | |
| ifc-2 | M6.1 | gb\|BQ613722 | 8E−25 | | | gb\|BQ836630 | | | |
| pan-1 | M88.6 | | | gb\|BG735742 | 5E−14 | | | | |
| | T01C3.1 | | | | | | | | |
| qhg-1 | T05C12.10 | gb\|BQ613344 | 7E−47 | | | | | | |
| | T14F9.1 | | | gb\|BG735889 | 5E−65 | | | gb\|BF014612 | 2E−54 |
| | T23F2.1 | gb\|BM880892 | 6E−65 | gb\|BI744669 | 3E−52 | gb\|BM883631 | 2E−57 | | |
| skp-1 | T27F2.1 | gb\|BM881774 | 8E−22 | gb\|BI142900 | 3E−44 | gb\|BM900937 | 1E−33 | | |
| mlp-18 | W01F3.3 | gb\|BM882536 | 6E−30 | gb\|BI745590 | 9E−18 | gb\|BM902581 | 5E−19 | | |
| mlp-12 | W08F4.6 | | | | | | | | |
| | W09B6.1 | | | | | | | | |
| ifa-2 | W10G6.3 | gb\|BQ613497 | 1E−68 | | | gb\|BM901834 | 6E−66 | | |
| | Y37D8A.10 | gb\|BM882772 | 4E−24 | | | | | gb\|BI396794 | 1E−29 |
| | Y48B6A.3 | | | | | | | | |
| unc-52 | ZC101.2 | gb\|BQ613494 | 3E−21 | | | gb\|BM901402 | 2E−44 | | |
| ptr-23 | ZK270.1 | gb\|BE340858 | 6E−14 | | | gb\|BQ090105 | 2E−17 | | |
| mlp-11 | ZK430.8 | | | | | gb\|BM883419 | 1E−36 | gb\|BI396718 | 1E−27 |
| | ZK686.3 | | | | | | | | |

| | | Related Genes | | | | | |
|---|---|---|---|---|---|---|---|
| | | | | Parastrongyloides | | | |
| | | Pristionchus pacificus | | trichosurl | | Ostertagia ostertagi | |
| Gene | ORF | Accession # | E value | Accession # | E value | Accession # | E value |
| ahr-23 | C01H6.5 | | | | | | |
| blt-1 | C09G5.6 | | | | | gb\|BG734092 | 1E−22 |
| mlt-24 | C17G1.6 | gb\|BI500840 | 2E−23 | gb\|BI451087 | 2E−34 | gb\|BG733933 | 6E−20 |
| mlt-21 | C26C6.3 | | | gb\|BI451087 | 5E−35 | gb\|BG734159 | 9E−26 |
| mlt-14 | C34G6.6 | gb\|BI500192 | 2E−69 | | | | |
| ocn-1 | C42D8.5 | gb\|AW114662 | 3E−39 | gb\|BI451241 | 6E−33 | | |
| | CD4.4 | gb\|AW097092 | 8E−24 | | | | |
| | D1054.15 | gb\|AW115214 | 3E−29 | | | | |
| mlt-20 | F08C6.1 | | | | | | |
| mlt-13 | F09B12.1 | | | | | | |
| nhr-25 | F11C1.6 | | | | | | |
| trp-1 | F29D11.r | | | | | | |
| | F38H4.9 | | | gb\|BI863834 | 2E−69 | gb\|BQ097609 | 1E−104 |
| | F40G9.1 | | | | | | |
| mlt-17 | F45G2.5 | | | | | | |
| | F49C12.12 | | | gb\|BM513019 | 5E−30 | | |
| mlt-15 | F52B11.3 | | | | | | |
| | F53G12.3 | | | | | | |
| src-1 | F54C9.2 | gb\|AW052295 | 1E−55 | | | | |
| DuOX | F56C11.1 | | | | | | |
| | F57B9.2 | | | | | | |
| | H04M03.4 | | | | | | |
| | H19M22.2 | | | | | | |
| blt-4 | KD4F10.4 | | | gb\|BI451155 | 2E−63 | gb\|BQ099039 | 5E−18 |
| mup-4 | K07D8.1 | | | | | | |
| | M03F4.7 | gb\|AI986802 | 2E−52 | | | gb\|BM396658 | 3E−79 |
| ifc-2 | M6.1 | | | | | gb\|BQ099825 | 7E−20 |
| pan-1 | M88.6 | | | | | | |
| | T01C3.1 | | | | | | |
| qhg-1 | T05C12.10 | | | | | | |
| | T14F9.1 | | | gb\|BM513291 | 5E−69 | | |
| | T23F2.1 | gb\|BI703617 | 4E−13 | | | | |
| skp-1 | T27F2.1 | | | | | | |
| mlp-18 | W01F3.3 | | | | | | |
| mlp-12 | W08F4.6 | | | gb\|BM346811 | 6E−89 | | |
| | W09B6.1 | | | | | | |
| ifa-2 | W10G6.3 | | | gb\|BI322222 | 1E−43 | gb\|BM896621 | 6E−77 |
| | Y37D8A.10 | gb\|AW052236 | 7E−51 | gb\|BI744051 | 3E−29 | | |
| | Y48B6A.3 | | | | | gb\|BQ457533 | 6E−52 |
| unc-52 | ZC101.2 | | | gb\|BM513799 | 1E−14 | | |
| ptr-23 | ZK270.1 | gb\|AA193996 | 1E−62 | gb\|BI863807 | 4E−31 | | |
| mlp-11 | ZK430.8 | | | | | | |
| | ZK686.3 | gb\|AW097184 | 9E−71 | | | | |

(1) Top hits from tblastn searches with the predicted C. elegans gene product versus translated cDNAs isolated from the indicated species.

mlt-26, which encodes the worm ortholog of fibrilin-1, is conserved in humans. The human gene is associated with Marfan syndrome. MLT-14 and MLT-15 are homologous to NompA, a component of specialized extracellular matrix (ECM) in flies (Chung et al., Neuron 29:415-28, 2001). Putative modification enzymes include MLT-24 and MLT-21, tolloid family metalloproteases that might direct cuticle assembly by processing procollagens or other ECM proteins, just as tolloid family members regulate vertebrate ECM formation, in part, by cleaving procollagen C-propeptides (Unsold et al. JBC 277:5596-602, 2002; Rattenholl et al., JBC 277:26372-8, 2002). MLT-17 and MLT-18 likely inhibit extracelullar proteases, since both proteins contain domains similar to BPTI, and a comparable ECM protein of D. melanogaster inhibits metalloproteinases in vitro (Kramerova et al., Dev 127:5475-85, 2000). Of three peroxidases essential for molting, one, DuOx, probably crosslinks cuticle collagens (Edens et al., J. Cell Biol 154:879-91, 2001). Together, these enzymes likely regulate the spatial and temporal dynamics of epithelial remodeling during molting, and regulation of the corresponding genes may therefore ensure the orderly synthesis and breakdown of cuticle.

Neuroendocrine pathways regulate molting in arthropods, and likely also operate in nematodes. In insects, pulses of the steroid hormone 20-hydroxyecdysone trigger molting and metamorphosis, and the neuropeptide PTTH stimulates ecdysone synthesis in the prothoracic glands (Gilbert et al., Ann. Rev. Entomol. 47:883-916, 2002). The peptide hormone ETH drives behavioral routines essential for ecdysis (Park et al., Dev. 129:493-503, 2002; Zitnan et al., Science 271: 88-91, 1996), and the neuropeptide eclosion hormone (EH) triggers ETH secretion from epitracheal glands, in part. Environmental and 4 physiologic cues modulate secretion of PTTH, suggesting extensive neural input to the neuroendocrine secretions that govern molting (Gilbert et al., Ann. Rev. Entomol. 47:883-916, 2002).

In C. elegans, the requirement for two orphan nuclear hormone receptors, NHR-23 and NHR-25, orthologous, respectively, to the ecdysone-responsive gene products DHR3 and Ftfz-F1 of Drosophila melanogaster (Kostrouchova Dev. 125:1617-26, 1998; Gissendanner et al., Dev Biol 221:259-72, 2000), implicates an endocrine trigger for molting, possibly derived from steroids. Consistently, molting requires cholesterol, the biosynthetic precursor of all steroid hormones (Yochem et al. Dev. 126:597-606, 1999). Further, molting of the nematode Aphelenchus avenae requires a diffusible signal from the anterior of the worm (Davies et al., Int. J. Parasitol 24:649-55, 1994), pointing to an endocrine cue. Ecdysone itself, however, is unlikely to serve as a nematode molting hormone, because orthologs of the ecdysone receptor components ECR and USP have not been identified in the fully-sequenced genome of C. elegans (Sluder et al., Trends Genet 17:206-13, 2001), and because ecdysteroids have not been detected in any free-living nematode (Chitwood, Crit Rev Biochm Mol Biol 34:273-84, 1999). Several genes uncovered in our screen encode signaling molecules and transcription factors that might transduce endocrine signals for molting between neurons and epithelial cells (Table 1A and Table 1B), such as QHG-1 (quahog), a protein with a C-terminal Hint domain like that found in hedgehog (Aspock et al., Gen. Res. 9:909-23, 1999), as well NHR-23 and NHR-25, both synthesized in epithelial cells (Kostrouchova et al., Dev 125:1617-26, 1998; Gissendanner, Dev Biol 221:259-72, 2000). The mlt-12 or Y41D4B.10 genes might specify intercellular signals regulating molting, since the corresponding proteins contain secretory signal sequences, but lack transmembrane domains or motifs characteristic of ECM proteins. Moreover, dibasic sites in MLT-12 suggest proteolytic processing, while Y41D4B.10p resembles a delta/serrate ligand. ACN-1 is also predicted to function in the endocrine phase of molting, as the protein is 28% identical to human angiotensin converting enzyme (ACE), the peptide protease that cleaves angiotensin 1 to 5 angiotensin II. ACN-1 is unlikely to catalyze proteolysis, because the active-sites residues of ACE are not conserved in the predicted ACN-1 protein. Nevertheless, ACN-1 could regulate production of a peptide molting hormone.

Twenty-three of the mlt genes identified herein (e.g., C09G5.6, C17G1.6, C23F12.1, C34G6.6, F08C6.1, F09B12.1, F16B4.3, F18A1.3, F45G2.5, F49C12.2, F53B8.1, H04M03.4, H19M22.2, K07D8.1, M6.1, M88.6, T05C12.10, W01F3.3, W08F4.6, Y111B2A.14, ZK262.8, ZK270.1, and ZK430.8) appear unique to nematodes since sequence orthologs of the corresponding proteins were not identified in D. melanogaster or H. sapiens, but were readily identified among the predicted products of cDNAs derived from parasitic nematode species that infect mammals and insects. For mlt-12, thirty-two different cDNAs (Table 7) isolated from a library of molting O. volvulus larvae, the parasite associated with African River Blindness, were found to be orthologous. Whereas many cDNAs matching mlt-12 (e⁻¹²¹) were found in a library from molting O. volvulus (Table 4C), a similar gene was not found in the fly or human genomes. Identifying genes essential for C. elegans molting enables the development of safe and effective nematicides that, for example, target gene products conserved only in nematodes. One attractive target is MLT-12, because the mlt-12 gene is conserved and highly expressed at the molt in a parasitic nematode.

Molting proteases, like MLT-24, also represent attractive targets for the development of small molecule antagonists, given the success of drug development on protease targets for high blood pressure and HIV (Cvetkovic et al, 63:769-802, 2003). Moreover, pesticides that target molecular components of molting shared between arthropods and nematodes, such as the ECM proteins MLT-14 and MLT-15, are expected to harm only Ecdysozoans, and therefore be much less toxic to humans than current insecticides.

The methods of the invention are useful for treating or preventing an O. volvulus parasitic infection by inhibiting O. volvulus mlt-12. In one embodiment, an RNA O. volvulus mlt-12 nucleic acid inhibitor is administered to an infected person or to a person at risk of infection, for example, a person living in an area in which O. volvulus is endemic. This administration inhibits molting in O. volvulus, interrupts the life cycle of the causitive agent of African River Blindness, and treats or prevents an O. volvulus infection. Because there is no mlt-12 human homologue, administration of a chemical compound or RNA nucleic acid inhibitor of mlt-12 would be expected to produce few, if any, adverse human side effects.

Several of the mlt genes identified herein and presented in Table 4A were found in insects and nematodes, but not in yeast, suggesting that their protein products are good candidates to function in molting in all Ecdysozoans. In particular, mlt-15, which corresponds to F52B11.3, and ZK686.3 have orthologs in Drosophila, but homologous genes were not identified in other metazoans or yeast. Genes present in Ecdysozoans (e.g., Drosophila, C. elegans and other nematodes), but missing or divergent in non-molting organisms (e.g., chordate clade members, such as vertebrates), likely function in molt neuroregulatory pathways. Given that Ecdysozoans are distant from humans and are the only animals that molt, it is likely that mlt genes that are present only in Ecdysozoans can be inhibited with drugs or siRNAs that will not have adverse side effects in humans.

Regulation of mlt Gene Expression

To determine if the newly-identified mlt genes are periodically or continually expressed during larval development, gene fusions were generated in which GFP was expressed under the control of the mlt-12, mlt-13, mlt-18, mlt-10, mlt-24, and acn-1 promoters. To shorten the half-life of the GFP fusion proteins to approximately one hour in vivo, a PEST sequence driving rapid protein degradation (Loetscher et al., J. Biol. Chem. 266:11213-20) was added to the end of the GFP open reading frame. The fusion genes were each microinjected into temperature-sensitive pha-1(e2123) mutant animals along with a pha-1(+) rescuing construct. Table 5 lists strains used in this study.

TABLE 5
Strains Used in mlt GFP PEST Expression

| Strain | Genotype | Source | Reference |
|---|---|---|---|
| N2 | wild-type | CGC | |
| GE24 | pha-1(e2123) III | CGC | Granato et al., |
| | | | 1994 |
| NL2099 | rrf-3(pk1426) II | CGC | |
| GR1348 | pha-1(e2123) mgEx646[Pₘₗₜ₋₁₀:: | this study | |
| | GFP-PEST pha-1⁺] | | |
| GR1349 | pha-1(e2123) mgEx647[Pₘₗₜ₋₁₂:: | this study | |
| | GFP-PEST pha-1⁺] | | |
| GR1350 | pha-1(e2123) mgEx648[Pₘₗₜ₋₁₃:: | this study | |
| | GFP-PEST pha-1⁺] | | |
| GR1351 | pha-1(e2123) mgEx649[Pₘₗₜ₋₁₈:: | this study | |
| | GFP-PEST pha-1⁺] | | |
| GR1368 | pha-1(e2123) mgEx656 [mlt- | this study | |
| | 24::gfp-pest pha-1] | | |
| GR1367 | pha-1(e2123) mgEx654 [acn- | this study | |
| | 1::gfp-pest pha-1] | | |
| GR1348 | pha-1(e2123) mgEx657 [mlt- | this study | |
| | 10::gfp-pest pha-1] | | |
| GR1387 | pha-1(e2123) mgEx659 [mlt- | this study | |
| | 13::gfp pha-1] | | |

Table 6 lists the primers used to construct the mlt GFP-PEST fusion genes.

TABLE 6
mlt Gene Primers
Table 56. Primers for construction of GFP fusion genes

| Gene | Primer U1 | Primer U2 |
|---|---|---|
| mlt-12 | 5′ TAAATTTTGGAGGGTCTCGGC 3′ | 5′ GGAAAAACGACACGACTATGG 3′ |
| mlt-13 | 5′ TTAATTGCCGCGCAAAATGCG 3′ | 5′ ATGCGACGAAATCACTACTCGG 3′ |
| mlt-18 | 5′ GCGATGGAGTACCACTTGGCGATTTTTGG 3′ | 5′ GCTAGAAATGGGTGAAATCGGTCTTCCGG 3′ |
| acn-1 | 5′ ACCGTGATTGGACTGTTTTCAGTGCACC 3′ | 5′ ACCGTGATTGGACTGTTTTCAGTGCACC 3′ |
| mlt-24 | 5′ GCTTTGAACCCGCAGACACTAAGATTGG 3′ | 5′ TGAACTGACGAAACTGGGAGGATAACCG 3′ |
| mlt-10 | 5′ GTTAGCCTTCCAACCTGAATAGAGAACAGG 3′ | 5′ GTTAGCCTTCCAACCTGAATAGAGAACAGG 3′ |
| Gene | Primer FU‡ | Primer F1 |
| mlt-12 | 5′ TTTAAAATCAAATTTCTCAGGTAATG-R1 3′ | 5′ R2-CATTACCTGAGAAATTTGATTTTAAA 3′ |
| mlt-13 | 5′ TATCCGACCACACTACCATCAGAATG-R1 3′ | 5′ R2-CATTCTGATGGTAGTGTGGTCGGATA 3′ |
| mlt-18 | 5′ AATTCCTATCAGTTGTCGGGTAATG-R1 3′ | 5′ R2-CATTACCCGACAACTGATAGGAATT 3′ |
| acn-1 | 5′ TTATTTATAGTTGTTTTTCAGATG-R1 3′ | 5′ R2-CATCTGAAAAACAACTATAAATAA 3′ |
| mlt-24 | 5′ TCTTGATGTTCTATTTTGCAGAATG-R1 3′ | 5′ R2-CATTCTGCAAAATAGAACATCAAGA 3′ |
| mlt-10 | 5′ GTAATAAATTTTGGCAATAAATCATG-R1 3′ | 5′ R2-CATGATTTATTGCCAAAATTTATTAC 3′ |

‡R1 refers to the sequence 5′ CGGGATTGGCCAAAGGACCCAAAG 3′
R2 refers to the sequence complementary to R1

For each reporter, genomic DNA isolated from N2 worms was amplified using primers A1 (SEQ ID NOs:1-3) and FL (SEQ ID NOs:10-12), while DNA from pAF207 was amplified using primers FU (SEQ ID NOs:7-9) and CAW31 (5′ GCCGCATAGTTAAGCCAGCC 3′ (SEQ ID NO:13), (Wolkow et al., Science 290: 147-50, 2000), using high-fidelity Taq. The EXPAND LONG TEMPLATE PCR SYSTEM (Roche Molecular Biochemicals), a kit containing PCR reagents, was used for all reactions.

The two PCR products were annealed and the resulting polynucleotide amplified using primers A2 (SEQ ID NO:4-6) and CAW32 (5′ CCGCTTACAGACAAGCTGTGACCG 3′) (SEQ ID NO:16). To add the PEST sequence to the C-terminus of GFP, nucleotides 1399-1524 of pd1EGFP-N1 (Invitrogen) were inserted into pPD95_{—}81 provided by A. Fire) between the last coding codon and the stop codon of GFP. This generated vector pAF207. The reporter constructs fpAF15, fpAF9, and fpAF12 correspond, respectively, to Pmlt-12::GFP-PEST, Pmlt-13::GFP-PEST, and Pmlt-18::GFP-PEST. In Table 6, R₁ refers to the DNA sequence: 5′ CGGGATTGGCCAAAGGACCCAAAG 3′(SEQ ID NO:14) and R₂ refers to the DNA sequence 5′ CTTTGGGTCCTTTGGCCAATCCCG 3′ (SEQ ID NO:15). To generate the extrachromosomal arrays mg647, mg648, and mg649, respectively, fpAF15, fpAF9, and fpAF12 were purified by gel electrophoresis and then microinjected into pha-1(e 2123) mutant animals along with the pha-1⁺ plasmid pBX at 3 ng/ul (Granato et al., Nucleic Acids Res., 22: 1762-3, 1994) and pBS DNA bringing the final DNA concentration to 100 ng/ul. Transgenic lines were recovered as described (Granato et al., Nucleic Acids Res., 22: 1762-3, 1994).

A fusion gene between mlt-13 and standard gfp was constructed using pPD95_{—}81 as the PCR template. PCR reactions were performed under conditions described previously (Fraser et al., Nature 408:325-30, 2000). To generate the extrachromosomal arrays mgEx647, mgEx648, mgEx649, mgEx656, mgEx654, mgEx657, and mg659, the PCR products corresponding to, respectively, mlt-12::gfp-pest, mlt-13::gfp-pest, mlt-18::gfp-pest, mlt-24::gfp-pest, acn-1::gfp-pest, mlt-10::gfp-pest, and mlt-13::gfp, each at 10 ng/ul, were microinjected into temperature-sensitive pha-1 (e2123) mutant animals along with the pha-1(+) plasmid pBX (6) at 3 ng/ul and pBS DNA at 87 ng/ul, allowing for the recovery and cultivation of worm populations in which virtually all animals maintained the fusion genes, because only pha-1(+) transgenic embryos survive at 25° C. (Kamath et al., Nature 421:231-7, 2003). To verify that GFP-PEST molecules are degraded by the proteosome, we found that RNAi of the proteosome subunit gene pbs-5 sustained fluorescence from mlt-10::gfp in larvae arrested for 2 days.

Use of the pha-1 (e2123) genetic background allowed for the cultivation of worm populations in which virtually all animals expressed the extrachromosal array, because only transgenic animals expressing pha-1(+) survive embryonic development at 25° C. (Granato et al., Nucleic Acids Res., 22: 1762-3, 1994). Temporal oscillations in gene expression were observed as changes in GFP-fluorescence over the period of a single molting cycle. Worms were visualized by Nomarski optics using standard techniques, and fluorescence was quantified using OPENLAB software (Improvision Inc. Lexington, Mass.).

Monitoring mlt::gfp Fusion Gene Expression

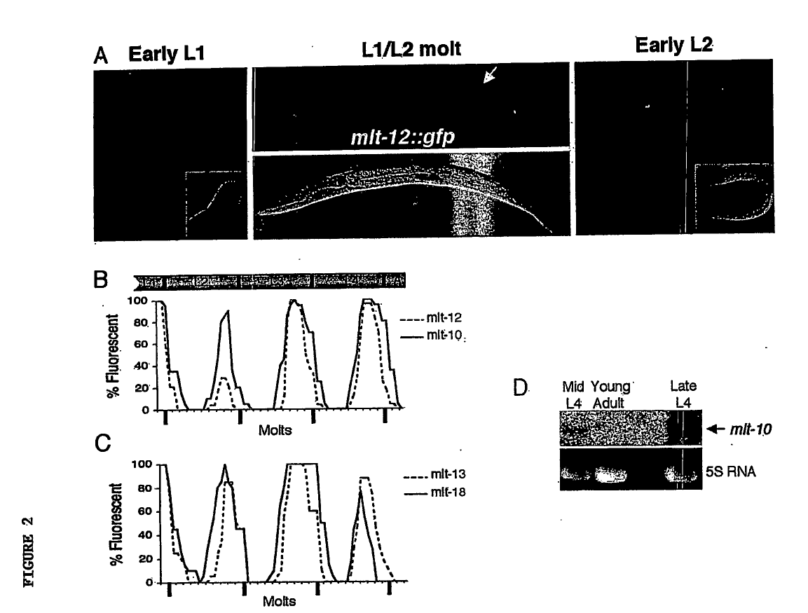

To monitor temporal expression of the mlt gene gfp fusion genes, synchronized L1 hatchlings of GR1348, GR1349, GR1350, or GR1351 (Table 5) were plated on NGM with E. coli strain OP50 as a food source and incubated at 25° C. Fluorescent larvae were selected 14 hours later to ensure the use of non-mosaic, highly synchronous animals. Larvae were scored once every hour for detectable fluorescence, using a Zeiss Stemi-SV6 microscope, and for molting, indicated by shedding of the cuticle. Each animal was transferred to a new plate after each molt. In FIG. 2, we report the percentage of animals that were fluorescent over time, on a scale normalized to the period between molts for each worm under observation. As an example, a larvae that molted from L1 to L2 at noon, molted from L2 to L3 at 8 PM, was fluorescent at 7 p.m. and 8 p.m., and was not fluorescent at 6 p.m. or 9 p.m. would be recorded as fluorescent from time 1.75 to time 2.0, or, from 87.5 to 100% of the L2 stage. Calculations of the average duration of fluorescence, with the 95% confidence interval, include observations from larvae during the L2, L3, and L4 stages. Because many of the extrachromosomal arrays were associated with some larval lethality, only larvae that completed all four molts were included in the final analysis. A total of 24 larvae were analyzyed for mlt-12::gfp; 20, for the other reporters.

Fluorescence from all six gfp fusion genes was observed in epithelial cells that secrete cuticle, in larvae and, in some cases, late embryos. All six reporters were expressed in the hypodermis and, for mlt-13, mlt-18, mlt-24, and acn-1, also in the lateral seam cells, which are essential for molting and morphogenesis of the cuticle. FIGS. 2A-2D show that a pulse of fluorescence was observed in the hypodermis prior to each of the four molts, for all six gfp fusion genes. Fluorescence from mlt-12::gfp was first detected approximately 3 hours before the L1/L2 molt, which occurred roughly 17 hours after starved hatchlings were fed and cultivated at 25° C. The intensity of fluorescence increased until lethargus, a brief period when larvae cease moving or feeding before molting, and then decreased rapidly, such that fluorescence was barely detectable 2 hours after the molt (FIG. 2A). Monitoring individual Ex[mlt-12::gfp] larvae over the course of development, fluorescence was observed starting at 65±2% and ending at 90±2% of the way through each larval stage (FIG. 2B).

Cultivation of worms at 15° C. delayed the first appearance of fluorescence in L1 larvae, and the first molt, by approximately 15 hours, and also expanded the period between peaks in fluorescence and between molts to the same extent Similarly, the pulse of hypodermal expression for the mlt-13 or mlt-10 reporters began, respectively, 64±3% or 63±2% of the way through each larval stage. Hypodermal fluorescence from mlt-18::gfp was detected earlier, from 51±2% to 72±3% of each stage, suggesting that MLT-18 antiprotease synthesized midway through a larval stage might repress proteases that are post-translationally activated at ecdysis. Fluorescence from mlt-13::gfp and mlt-18::gfp in seam cells also cycled in phase with molting, but often preceded and persisted longer than fluorescence in the hypodermis (FIG. 2C).

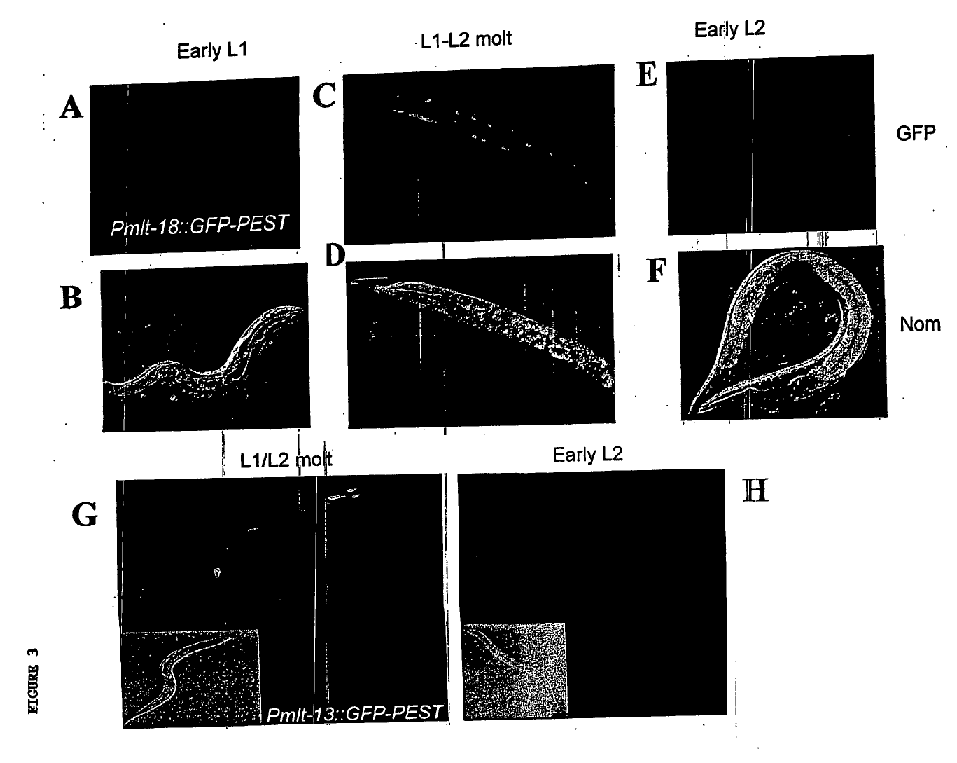

FIGS. 3A-3H show that fluorescence associated with Pmlt18::GFP-PEST was detectable in the hypodermis during late intermolt and intensified until ecydsis. After ecydsis, fluorescence dissipated rapidly and did not increase until the onset of the next molt. Fluorescence associated with Pmlt-13::GFP-PEST was observed in the anterior cells of the hypodermis during lethargus and molting, and in the seam cells when they underwent division, close to the time of lethargus (FIGS. 3G and 3H). Fluorescence associated with Pmlt-12::GFP-PEST was observed in the hypodermis shortly before each of the four molts. The ability of the mlt-12, mlt-13, and mlt-18 promoters to drive cyclic GFP expression in synchrony with the molting cycle identifies these genes as components of a periodic gene expression program required for molting. Moreover, the expression, timing, and pattern of mlt-12 in hypodermis and of mlt-13 and mlt-18 in both hypodermis and seam cells is consistent with a role for these genes in ecdysis, given that hypodermal cells secrete cuticle and seam cells are required for molting.

Northern Analysis

To verify that cycling fluorescence from a gfp-pest fusion gene reflects dynamic temporal regulation of gene expression, we examined the level of one milt gene message by northern analysis. The abundance of mlt-10 mRNA in late L4 larvae exceeded that of mid L4 larvae by a factor of 6, and mlt-10 mRNA was barely detectable in young adults (FIG. 2D), consistent with the observation that fluorescence from mlt-10::gfppest peaks late in each larval stage.

For northern analysis, RNA from extracts of mid L4, late L4, and young adult animals was resolved and hybridized with a mlt-10 probe, corresponding to base pair 5070 to 6997 of cosmid C09E8 (GenBank Accession No: AF077529) (Lee et al., Science 300:644-647, 2003). Message levels were quantified using Imagequant software and a phosphorimager.

To order gene expression cascades, synchronized hatchlings of GR1348 and GR1349 were fed bacteria expressing dsRNA for each gene of interest, or, as a comtrol, fed isogenic bacteria not expressing dsRNA for a worm gene. After incubation for no more than 15 hours at 25° C., single, fluorescent larvae were transferred to 24 well RNAi plates seeded with the appropriate bacteria. For each developmental stage, larvae were observed over a 6 to 9 hour time period starting when control larvae first became fluorescent, and scored every 2 to 3 hours for detectable fluorescence and for the Mlt phenotype. In FIGS. 4A and 4B, we report the percentage of animals that were fluorescent prior to a defective molt, normalized to the fraction of control larvae that were fluorescent before molting from the same stage. Note that RNAi of mlt-12 or acn-1 prevented completion of the L2/L3 molt, whereas RNAi of qhg-1, mlt-16, or mlt-13 interfered most often with the L3/L4 or L4/A molts. RNAi of nhr-23 prevented completion of the L2/L3 molt in most Ex[mlt-12::gfp] larvae, but interfered with the L3/L4 or L4/A molts in Ex[mlt-10::gfp] larvae. Fluorescence was observed in 95% (n=56), 100% (n=43), or 94% (n=48) of control Ex[mlt-10::gfp] larvae during, respectively, the L2, L3, or L4 stage. Fluorescence was observed in 74% (n=57) or 70% (n=36) of L2 stage Ex[mlt-12::gfp] larvae, and in 90% (n=49) of L4 stage Ex[mlt-12::gfp] larvae.

To screen the full set of molting gene inactivations, approximately 20 synchronized hatchlings of GR1348 were fed each bacterial clone in 24 well format, in two trials. The percent of larvae with detectable fluorescence was scored 1 to 3 hours before the L2/L3, L3/L4, and L4/A molts, when the majority of control GR1348 larvae were fluorescent.

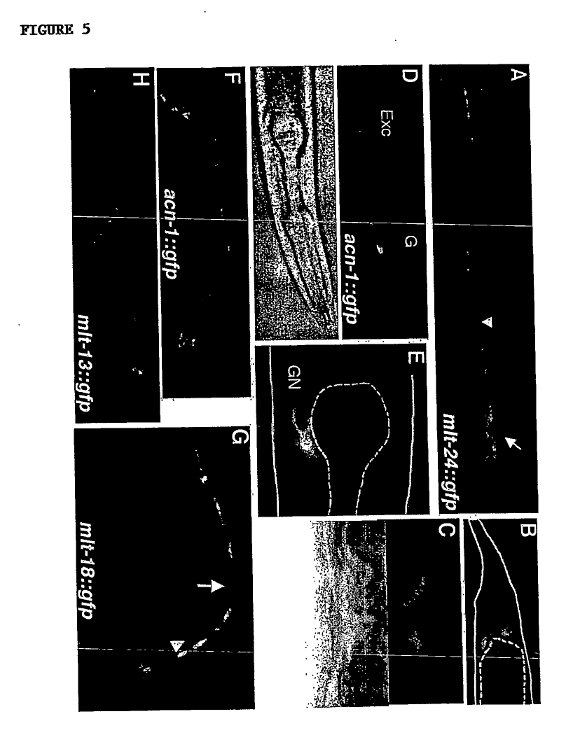

Fluorescence from particular gfp fusion genes was also observed in specialized epithelia including the rectal gland, rectal epithelia, the excretory duct and pore cells, and vulval precursors (FIG. 5). Interestingly, the acn-1 fusion gene was also expressed in the excretory gland cell of larvae (FIG. 5). This gland cell may release or receive endocrine signals regulating molting (Chitwood, Crit Rev Biochm Mol Biol 34:273-84, 1999), and ACN-1 produced in the gland could regulate such an endocrine output. RNAi of acn-1 likely reduces expression in the gland cell, since RNAi of gfp reduces fluorescence from acn-1::gfp in the entire excretory system. Fluorescence from mlt-12::GFP was also observed in a single posterior neuron that remains to be identified.

Taken together, the spatio-temporal expression pattern off fusion genes suggests that mlt-10, mlt-12, mlt-13, mlt-24, milt-18, and acn-1 are expressed transiently before molting in epithelial cells that synthesize cuticle, and thus define a periodic gene expression program essential for molting. The upstream regulators driving mlt gene expression might also control collagen and nuclear hormone receptor genes whose expression oscillates over the molting cycle (Johnstone et al., EMBO J. 15:3633-9, 1996).

Newly-identified mlt genes may be organized into genetic pathways using epistasis analysis. One strategy for organizing the newly-identified mlt genes into genetic pathways is to examine the expression of the Pmlt-GFP-PEST reporter genes in larvae undergoing RNAi against each of the newly-identified mlt genes.

The nuclear hormone receptor gene, nhr-23, was inactivated by RNAi (as described above) in Ex[Pmlt-12::GFP-PEST] larvae. GFP fluorescence was then detected by fluorescence microscopy at the time of the L3/L4 or L4/adult molt. Fluorescence associated with Pmlt-12::GFP-PEST was often not detectable in Mlt nematodes newly trapped in cuticle. In contrast, fluorescence associated with Pmlt-12::GFP-PEST was detected in Mlt nematodes undergoing RNAi against lrp-1, rme-8, mlt-24, or mlt-26. Control larvae, which were Non-Mlt larvae fed bacteria transformed with an empty vector, also displayed Pmlt-12::GFP-PEST fluorescence.

This observation, that nhr-23(RNAi) larvae carrying mlt-12::gfp or mlt-10::gfp failed to become fluorescent prior to their unsuccessful molt (FIG. 4A), suggested that the nuclear hormone receptor NHR-23, synthesized in epithelial cells (Kostrouchova Dev. 125:1617-26, 1998), initiates the pulse of mlt gene expression late in each larval stage, thereby provoking an epithelial response to an endocrine cue for molting. Consistently, inactivation of nhr-23 diminished hypodermal fluorescence from mlt-24::gfp and mlt-18::gfp. Signaling via NHR-23 may coordinate collagen production with the synthesis of MLT proteins that direct cuticle assembly, since nhr-23 also drives expression of the cuticle collagen gene dpy-7 (Kostrouchova et al., PNAS 98:7360-5, 2001). Moreover, MLT-12 likely functions downstream of NHR-23 in a regulatory cascade, since inactivation of mlt-12 also abrogates expression of mlt-10::gfp, but not of mlt-12::gfp (FIG. 3A). MLT-12 secreted from the hypodermis could serve as an autocrine signal for molting, but could also signal to muscle cells, or provide feedback to neurons.

The majority of acn-1(RNAi) larvae also failed to express either mlt-12::gfp or mlt-10::gfp before an unsuccessful molt (FIG. 4A), consistent with the view that ACN-1 synthesized in the hypodermis or excretory gland functions in the endocrine phase of molting. In contrast, after inactivation of the hedgehog-like gene qhg-1, the fibrillin homolog mlt-16, or the novel gene mlt-13, as many larvae expressed the fusion genes as did control larvae molting from the same developmental stage, suggesting that these genes function downstream of, or in parallel to, induction of mlt-10, in the execution phase of molting.

To order the action of additional molting genes, we monitored fluorescence from mlt-10::gfp in 58 gene inactivations. Populations of Ex[mlt-10::gfp] larvae fed each dsRNA were observed late in the L2, L3, and L4 stages. Inactivation of five genes abrogated expression of mlt-10::gfp in 85% or more of larvae during one stage, and blocked development shortly thereafter (FIG. 4B). The five genes, Y41D4B.10, W09B6.1, D1054.15, M03F8.3, and Y48B6A.3, likely function upstream of mlt-10, and encode, respectively, a secretory protein resembling delta/serrate ligands, acetyl-Coenzyme A carboxylase, homologs of the RNA splicing factors PLRG-1 (Ajuh et al., JBC 276:42370-81, 2001), or CRN (Chung et al. RNA 5:1042-54, 1999; Chung et al., Biochim Biophys Acta 1576: 287-97, 2002), and an exoribonuclease 54% identical to human XRN2 (Zhang et al., Genomics 59:252-4, 1999). Since microRNAs regulate developmental transitions in C. elegans (Reinhart et al., Nature 403:901-6, 2000), one intriguing possibility is that the product of Y48B6A.3 negatively regulates the abundance of one or more microRNAs whose target genes drive the L4-to-Adult molt. Among Ex[mlt-10::gfp] larvae fed 34 other dsRNAs, an equal or greater fraction became fluorescent as control larvae of the same stage (FIG. 4B). Molting-defective, fluorescent larvae were observed upon inactivation of mlt-24, F45G2.5, ZK430.8, unc-52, W10G6.3, kin-2, bli-1, and DuOx, strongly suggesting that the genes function downstream, or in parallel, to mlt-10 expression.

By analogy with arthropods, we expect that neuroendocrine cues initiate molting in C. elegans, ultimately stimulating epithelial cells to synthesize a new cuticle and release the old one. Together, gene annotations, expression patterns, and ordering experiments suggested that our screen identified several endocrine regulators of molting, including MLT-12, ACN-1, and NHR-23, as well as enzymes and ECM components essential for remodeling the exoskeleton.

Similar epistatic analyses are expected to place many, if not all, of the new mlt genes into genetic pathways characterized by early steps associated with neuroendocrine signaling or later steps promoting escape from the old cuticle.

Ecdysozoan Orthologs

DNA sequences corresponding to mlt genes of interest were retrieved from the repositories of sequence information at either the NCBI website (http://www.ncbi.nlm.nih.gov/) or wormbase (www.wormbase.org). The DNA sequence was then used for standard translating blast [tBLASTN] searching using the NCBI website (http://www.ncbi.nlm.nih.gov/BLASTA. The DNA sequence corresponding to the top ortholog candidate produced by tblastn was retrieved from Genbank (http://www.ncbi.nlm.nih.gov/) and used for a BLASTx search of C. elegans proteins using the wormbase site (http://www.wormbase.org/db/searches/blast). These methods provide for the identification of orthologs of C. elegans mlt genes (Tables 1A, 1B, 4A-4D, and 7) revealed by our RNAi analysis. An ortholog is a protein that is highly related to a reference sequence. One skilled in the art would expect an ortholog to functionally substitute for the reference sequence. Tables 4A-4D and 7 list exemplary orthologs by Genbank accession number.

C. elegans gene: M6.1

| | | Assession | |
|---|---|---|---|
| Species | EST ID | Number | E value |
| Ascaris suum | ki02g09.y1 | gb\|BM280603 | 1e−28 |
| Ascaris suum | kk52b05.y1 | gb\|BQ382546 | 1e−26 |
| Ascaris suum | As_L3_09B01_SKPL | gb\|BI594018 | 1e−25 |
| Ascaris suum | kj92f03.y1 | gb\|BM965152 | 3e−24 |
| Ascaris suum | As_nc_10C07_SKPL | gb\|BI594311 | 1e−22 |
| Ascaris suum | ki08f11.y1 | gb\|BM281039 | 2e−18 |
| Brugia malayi | SWYD25CAU14E02SK | gb\|AW675831 | 8e−19 |
| Brugia malayi | SWYACAL08E03SK | gb\|BE758356 | 5e−18 |
| Haemonchus | Hc_d11_11E10_SKPL | gb\|BF060126 | 4e−25 |
| contortus | | | |
| Haemonchus | Hc_d11_18E03_SKPL | gb\|BF422872 | 2e−20 |
| contortus | | | |
| Haemonchus | Hc_d11_09G03_SKPL | gb\|BF059991 | 1e−16 |
| contortus | | | |
| Meloidogyne | rd19e10.y1 | gb\|BQ613722 | 8e−25 |
| incognita | | | |
| Meloidogyne | rd02c03.y1 | gb\|BQ613170 | 1e−24 |
| incognita | | | |
| Meloidogyne | rd08a12.y1 | gb\|BQ613497 | 2e−24 |
| incognita | | | |
| Meloidogyne hapla | rf48d08.y1 | gb\|BQ836630 | 1e−21 |
| Onchocerca | SWOv3MCAM52D01SK | gb\|BF824665 | 4e−16 |
| volvulus | | | |
| Onchocerca | SWOvL3CAN13E07 | gb\|AA917260 | 2e−19 |
| volvulus | | | |
| Ostertagia ostertagi | ph69a09.y1 | gb\|BQ099825 | 7e−20 |
| Strongyloides ratti | kt51c06.y4 | gb\|BI742464 | 8e−19 |
| Strongyloides | kq58d04.y1 | gb\|BF014961 | 3e−28 |
| stercoralis | | | |
| Strongyloides | kq25d02.y1 | gb\|BE579290 | 7e−20 |
| stercoralis | | | |
| Strongyloides | kq31d11.y1 | gb\|BE579614 | 1e−20 |
| stercoralis | | | |
| Strongyloides | kq07e05.y1 | gb\|BG227475 | 1e−19 |
| stercoralis | | | |
| Strongyloides | kq38a11.y1 | gb\|BE580177 | 3e−19 |
| stercoralis | | | |
| Toxocara canis | ko17e01.y1 | gb\|BM965806 | 4e−16 |
| Trichinella spiralis | ps41c08.y1 | gb\|BG353660 | 5e−26 |
| Trichinella spiralis | ps21c11.y4 | gb\|BG732010 | 3e−20 |
| Trichuris muris | Tm_ad_32C10_SKPL | gb\|BM174670 | 8e−32 |

C. elegans gene: ZC101.2

| Species | EST ID | Assession Number | E value |
|---|---|---|---|
| Anopheles gambiae | 17000659084026 | gb\|BM601480 | 6e−31 |
| Anopheles gambiae | 17000687479592 | gb\|BM596670 | 3e−20 |
| Anopheles gambiae | 17000687506857 | gb\|BM598004 | 2e−15 |
| Anopheles gambiae | 17000687368906 | gb\|BM588620 | 4e−15 |
| Anopheles gambiae | 17000687134459 | gb\|BM612519 | 5e−15 |
| Anopheles gambiae | 17000687565373 | gb\|BM637990 | 2e−13 |
| Aedes aegypti | AEMTBL28 | gb\|AI618963 | 5e−23 |
| Ancylostoma caninum | pb38e07.y1 | gb\|BQ666249 | 5e−13 |
| Ascaris suum | kh43d01.y1 | gb\|BI782862 | 1e−13 |
| Bombyx mori | AV399222 | dbj\|AV399222 | 2e−21 |
| Brugia malayi | BSBmL3SZ44P24SK | gb\|AI723671 | 5e−60 |
| Brugia malayi | SWL4CAK11D03SK | gb\|AW600207 | 9e−53 |
| Brugia malayi | SWYD25CAU01B01SK | gb\|AW179566 | 2e−45 |
| Brugia malayi | MB3D6V3B03T3 | gb\|AA661133 | 2e−27 |
| Brugia malayi | SWYD25CAU13H12SK | gb\|AW676004 | 4e−25 |
| Dirofilaria immitis | ke10h02.y1 | gb\|BQ454813 | 5e−58 |
| Dirofilaria immitis | ke15g10.y1 | gb\|BQ454884 | 2e−14 |
| Globodera rostochiensis | GE1828 | gb\|AW506417 | 1e−14 |
| Haemonchus contortus | Hc_d11_08E04_SKPL | gb\|BE496755 | 3e−99 |
| Ancylostoma caninum | pa32g09.y1 | gb\|BE352403 | 4e−19 |
| Meloidogyne hapla | rc29b02.y1 | gb\|BM901402 | 2e−44 |
| Meloidogyne hapla | rc45d03.y1 | gb\|BM901130 | 3e−40 |
| Meloidogyne hapla | rc47g03.y1 | gb\|BM901696 | 3e−39 |
| Meloidogyne incognita | rd08a06.y1 | gb\|BQ613494 | 3e−21 |
| Meloidogyne incognita | MD0294 | gb\|BE217664 | 2e−15 |
| Onchocerca volvulus | SWOvL2CAS04B06SK | gb\|AW980135 | 4e−62 |
| Onchocerca volvulus | SWOvL3CAN52A02SK | gb\|AI132759 | 7e−43 |
| Onchocerca volvulus | SWOv3MCAM25F01SK | gb\|AI581466 | 4e−14 |
| Onchocerca volvulus | SWOvL3CAN18G05 | gb\|AI096109 | 4e−44 |
| Onchocerca volvulus | SWOv3MCA770SK | gb\|AA294548 | 7e−36 |
| Parastrongyloides trichosuri | kx99e03.y2 | gb\|BM513799 | 1e−14 |
| Strongyloides ratti | kt72h10.y1 | gb\|BI323571 | 3e−44 |
| Strongyloides ratti | kt75c08.y3 | gb\|BI502464 | 2e−39 |
| Strongyloides ratti | kt20f09.y1 | gb\|BG894201 | 1e−36 |
| Strongyloides ratti | kt33h03.y1 | gb\|BI073703 | 2e−21 |
| Strongyloides ratti | kt27e05.y3 | gb\|BI450558 | 9e−17 |
| Strongyloides stercoralis | kq04h11.y1 | gb\|BG227295 | 1e−49 |
| Strongyloides stercoralis | kq42h08.y1 | gb\|BE581152 | 1e−49 |
| Strongyloides stercoralis | kq26e04.y1 | gb\|BE579360 | 2e−32 |
| Toxocara canis | ko14a04.y1 | gb\|BM965583 | 9e−59 |

C. elegans gene: D1054.15

| | | Assession | |
|---|---|---|---|
| Species | EST ID | Number | E value |
| Anopheles gambiae | 17000687163725 | gb\|BM577379 | 3e−71 |
| Anopheles gambiae | 17000687054314 | gb\|BM600555 | 1e−37 |
| Anopheles gambiae | 17000687477449 | gb\|BM595864 | 6e−25 |
| Amblyomma | EST575203 | gb\|BM292661 | 6e−33 |
| variegatum | | | |
| Ancylostoma | pa80h05.y1 | gb\|BG232752 | 4e−77 |
| caninum | | | |
| Meloidogyne | rm16b05.y1 | gb\|BI863068 | 2e−87 |
| arenaria | | | |
| Globodera pallida | OP20173 | gb\|BM415102 | 4e−56 |
| Necator americanus | Na_L3_47E12_SAC | gb\|BU088714 | e−108 |
| Onchocerca | SWOvMfCAR07F05SK | gb\|BE202350 | 9e−49 |
| volvulus | | | |
| Pristionchus | rs62h03.y1 | gb\|AW115214 | 3e−29 |
| pacificus | | | |
| Trichinella spiralis | ps30a03.y2 | gb\|BG520170 | 2e−28 |

C. elegans gene: Y37D8A.10

| Species | EST ID | Assession Number | E value |
|---|---|---|---|
| Anopheles gambiae | 17000687279294 | gb\|BM583815 | 2e−24 |
| Anopheles gambiae | 17000687137751 | gb\|BM612986 | 1e−22 |
| Anopheles gambiae | 17000687067307 | gb\|BM601081 | 8e−22 |
| Bombyx mori | AV402441 | dbj\|AV402441 | 4e−28 |
| Haemonchus contortus | Hc_L3_04D09_SKPL | gb\|BI595303 | 3e−68 |
| Heterodera glycines | ro61h02.y3 | gb\|BI396794 | 1e−29 |
| Heterodera glycines | ro73d01.y1 | gb\|BI749054 | 4e−27 |
| Meloidogyne arenaria | rm39d08.y1 | gb\|BI747379 | 4e−30 |
| Meloidogyne arenaria | rm23g05.y1 | gb\|BI746177 | 3e−26 |
| Meloidogyne incognita | rb29e05.y1 | gb\|BM882772 | 4e−24 |
| Meloidogyne incognita | ra93b11.y1 | gb\|BM774415 | 2e−20 |
| Necator americanus | Na_L3_32G04_SAC | gb\|BU666155 | 9e−27 |
| Necator americanus | Na_L3_27B01_SAC | gb\|BU088007 | 3e−26 |
| Parastrongyloides trichosuri | kx55a12.y1 | gb\|BI744051 | 3e−29 |
| Pristionchus pacificus | rs33b11.y1 | gb\|AW052236 | 7e−51 |
| Strongyloides stercoralis | kp31d05.y1 | gb\|BE029374 | 1e−37 |
| Ancylostoma ceylanicum | pj18d12.y1 | gb\|BQ288481 | 2e−59 |
| Ancylostoma ceylanicum | pj18b06.y1 | gb\|BQ288451 | 4e−57 |
| Ancylostoma ceylanicum | pj18f04.y1 | gb\|BQ288495 | 4e−57 |
| Ancylostoma ceylanicum | pj19a07.y1 | gb\|BQ288871 | 4e−57 |
| Ancylostoma ceylanicum | pj19e09.y1 | gb\|BQ288915 | 4e−57 |
| Ancylostoma ceylanicum | pj19f07.y1 | gb\|BQ288924 | 4e−57 |
| Ancylostoma ceylanicum | pj19g09.y1 | gb\|BQ288934 | 4e−57 |
| Ancylostoma ceylanicum | pj20b03.y1 | gb\|BQ289634 | 4e−57 |
| Ancylostoma ceylanicum | pj21b01.y1 | gb\|BQ289718 | 4e−57 |
| Ancylostoma ceylanicum | pj21d08.y1 | gb\|BQ289743 | 4e−57 |
| Ancylostoma ceylanicum | pj21e04.y1 | gb\|BQ289749 | 4e−57 |
| Ancylostoma ceylanicum | pj22a07.y1 | gb\|BQ289455 | 4e−57 |
| Ancylostoma ceylanicum | pj22b06.y1 | gb\|BQ289464 | 4e−57 |
| Ancylostoma ceylanicum | pj22h05.y1 | gb\|BQ289530 | 4e−57 |
| Ancylostoma ceylanicum | pj23a11.y1 | gb\|BQ289548 | 4e−57 |
| Ancylostoma ceylanicum | pj23d03.y1 | gb\|BQ289565 | 4e−57 |
| Ancylostoma ceylanicum | pj24d12.y1 | gb\|BQ289067 | 4e−57 |
| Ancylostoma ceylanicum | pj24e03.y1 | gb\|BQ289070 | 2e−57 |
| Ancylostoma ceylanicum | pj24g03.y1 | gb\|BQ289088 | 2e−57 |
| Ancylostoma ceylanicum | pj25b04.y1 | gb\|BQ288958 | 4e−57 |
| Ancylostoma ceylanicum | pj25c06.y1 | gb\|BQ288971 | 4e−57 |
| Ancylostoma ceylanicum | pj26c09.y1 | gb\|BQ289134 | 3e−57 |
| Ancylostoma ceylanicum | pj28d09.y1 | gb\|BQ289322 | 4e−57 |
| Ancylostoma ceylanicum | pj28e11.y1 | gb\|BQ289334 | 4e−57 |
| Ancylostoma ceylanicum | pj28f04.y1 | gb\|BQ289338 | 2e−57 |
| Ancylostoma ceylanicum | pj28f07.y1 | gb\|BQ289341 | 2e−57 |
| Ancylostoma ceylanicum | pj28h06.y1 | gb\|BQ289361 | 2e−57 |
| Ancylostoma ceylanicum | pj29c04.y1 | gb\|BQ289391 | 4e−57 |
| Ancylostoma ceylanicum | pj29d03.y1 | gb\|BQ289401 | 4e−57 |
| Ancylostoma ceylanicum | pj30e06.y1 | gb\|BQ288830 | 4e−57 |
| Ancylostoma ceylanicum | pj30g06.y1 | gb\|BQ288847 | 4e−57 |
| Ancylostoma ceylanicum | pj30h03.y1 | gb\|BQ288855 | 4e−57 |
| Ancylostoma ceylanicum | pj31a06.y1 | gb\|BQ288703 | 4e−57 |
| Ancylostoma ceylanicum | pj31c11.y1 | gb\|BQ288727 | 2e−57 |
| Ancylostoma ceylanicum | pj31d01.y1 | gb\|BQ288729 | 2e−57 |
| Ancylostoma ceylanicum | pj31d04.y1 | gb\|BQ288732 | 4e−57 |
| Ancylostoma ceylanicum | pj33d04.y1 | gb\|BQ288645 | 2e−57 |
| Ancylostoma ceylanicum | pj33d09.y1 | gb\|BQ288650 | 4e−57 |
| Ancylostoma ceylanicum | pj33g10.y1 | gb\|BQ288684 | 4e−57 |
| Ancylostoma ceylanicum | pj33h04.y1 | gb\|BQ288689 | 4e−57 |
| Ancylostoma ceylanicum | pj34a03.y1 | gb\|BQ274663 | 2e−57 |
| Ancylostoma ceylanicum | pj34d06.y1 | gb\|BQ274700 | 4e−57 |
| Ancylostoma ceylanicum | pj35e01.y1 | gb\|BQ274789 | 2e−57 |
| Ancylostoma ceylanicum | pj35e12.y1 | gb\|BQ274800 | 4e−57 |
| Ancylostoma ceylanicum | pj35f05.y1 | gb\|BQ274803 | 2e−57 |
| Ancylostoma ceylanicum | pj36c11.y1 | gb\|BQ275536 | 4e−57 |
| Ancylostoma ceylanicum | pj36e10.y1 | gb\|BQ275566 | 4e−57 |
| Ancylostoma ceylanicum | pj38a12.y1 | gb\|BQ274837 | 4e−57 |
| Ancylostoma ceylanicum | pj38b02.y1 | gb\|BQ274838 | 4e−57 |
| Ancylostoma ceylanicum | pj38g07.y1 | gb\|BQ274896 | 4e−57 |
| Ancylostoma ceylanicum | pj38g12.y1 | gb\|BQ274900 | 4e−57 |
| Ancylostoma ceylanicum | pj39f02.y1 | gb\|BQ274962 | 4e−57 |
| Ancylostoma ceylanicum | pj39g08.y1 | gb\|BQ274977 | 4e−57 |
| Ancylostoma ceylanicum | pj39h11.y1 | gb\|BQ274990 | 4e−57 |
| Ancylostoma ceylanicum | pj40b05.y1 | gb\|BQ275007 | 4e−57 |
| Ancylostoma ceylanicum | pj40b06.y1 | gb\|BQ275008 | 4e−57 |
| Ancylostoma ceylanicum | pj40b11.y1 | gb\|BQ275012 | 4e−57 |
| Ancylostoma ceylanicum | pj41e03.y1 | gb\|BQ275122 | 2e−57 |
| Ancylostoma ceylanicum | pj41e07.y1 | gb\|BQ275126 | 4e−57 |
| Ancylostoma ceylanicum | pj41f02.y1 | gb\|BQ275133 | 4e−57 |
| Ancylostoma ceylanicum | pj42b02.y1 | gb\|BQ275176 | 4e−57 |
| Ancylostoma ceylanicum | pj42b12.y1 | gb\|BQ275185 | 4e−57 |
| Ancylostoma ceylanicum | pj42c11.y1 | gb\|BQ275195 | 4e−57 |
| Ancylostoma ceylanicum | pj42e03.y1 | gb\|BQ275208 | 4e−57 |
| Ancylostoma ceylanicum | pj42g08.y1 | gb\|BQ275233 | 2e−57 |
| Ancylostoma ceylanicum | pj43a09.y1 | gb\|BQ275256 | 4e−57 |
| Ancylostoma ceylanicum | pj43b04.y1 | gb\|BQ275262 | 4e−57 |
| Ancylostoma ceylanicum | pj43d07.y1 | gb\|BQ275287 | 4e−57 |
| Ancylostoma ceylanicum | pj43e04.y1 | gb\|BQ275295 | 4e−57 |
| Ancylostoma ceylanicum | pj45c09.y1 | gb\|BQ275446 | 4e−57 |
| Ancylostoma ceylanicum | pj45c12.y1 | gb\|BQ275449 | 4e−57 |
| Ancylostoma ceylanicum | pj46f09.y1 | gb\|BQ275735 | 4e−57 |
| Ancylostoma ceylanicum | pj46f12.y1 | gb\|BQ275738 | 4e−57 |
| Ancylostoma ceylanicum | pj47g06.y1 | gb\|BQ275825 | 5e−57 |
| Ancylostoma ceylanicum | pj48a03.y1 | gb\|BQ275842 | 4e−57 |
| Ancylostoma ceylanicum | pj48a11.y1 | gb\|BQ275850 | 4e−57 |
| Ancylostoma ceylanicum | pj48b09.y1 | gb\|BQ275860 | 4e−57 |
| Ancylostoma ceylanicum | pj48e12.y1 | gb\|BQ275895 | 4e−57 |
| Ancylostoma ceylanicum | pj50g03.y1 | gb\|BQ276059 | 2e−57 |
| Ancylostoma ceylanicum | pj50g07.y1 | gb\|BQ276063 | 4e−57 |
| Ancylostoma ceylanicum | pj51b04.y1 | gb\|BQ276091 | 4e−57 |
| Ancylostoma ceylanicum | pj51g04.y1 | gb\|BQ276145 | 2e−57 |
| Ancylostoma ceylanicum | pj53c01.y1 | gb\|BQ288078 | 4e−57 |
| Ancylostoma ceylanicum | pj54d07.y1 | gb\|BQ288160 | 4e−57 |
| Ancylostoma ceylanicum | pj56c04.y1 | gb\|BQ288297 | 4e−57 |
| Ancylostoma ceylanicum | pj56f08.y1 | gb\|BQ288327 | 4e−57 |
| Ancylostoma ceylanicum | pj57c06.y1 | gb\|BQ288376 | 2e−57 |
| Ancylostoma ceylanicum | pj57g03.y1 | gb\|BQ288415 | 4e−57 |
| Ancylostoma ceylanicum | pj19c08.y1 | gb\|BQ288893 | 7e−56 |
| Ancylostoma ceylanicum | pj19g02.y1 | gb\|BQ288927 | 4e−56 |
| Ancylostoma ceylanicum | pj20f10.y1 | gb\|BQ289683 | 4e−56 |
| Ancylostoma ceylanicum | pj21f06.y1 | gb\|BQ289758 | 2e−56 |
| Ancylostoma ceylanicum | pj22b10.y1 | gb\|BQ289468 | 4e−56 |
| Ancylostoma ceylanicum | pj23e11.y1 | gb\|BQ289589 | 1e−56 |
| Ancylostoma ceylanicum | pj24g11.y1 | gb\|BQ289093 | 5e−56 |
| Ancylostoma ceylanicum | pj24h06.y1 | gb\|BQ289100 | 1e−56 |
| Ancylostoma ceylanicum | pj25c04.y1 | gb\|BQ288969 | 2e−56 |
| Ancylostoma ceylanicum | pj26a09.y1 | gb\|BQ289111 | 1e−56 |
| Ancylostoma ceylanicum | pj27d08.y1 | gb\|BQ289233 | 2e−56 |
| Ancylostoma ceylanicum | pj27f06.y1 | gb\|BQ289254 | 9e−56 |
| Ancylostoma ceylanicum | pj28b02.y1 | gb\|BQ289292 | 5e−56 |
| Ancylostoma ceylanicum | pj28e06.y1 | gb\|BQ289330 | 9e−56 |
| Ancylostoma ceylanicum | pj28h04.y1 | gb\|BQ289359 | 5e−56 |
| Ancylostoma ceylanicum | pj30f04.y1 | gb\|BQ288837 | 2e−56 |
| Ancylostoma ceylanicum | pj31a12.y1 | gb\|BQ288708 | 4e−56 |
| Ancylostoma ceylanicum | pj31h01.y1 | gb\|BQ288774 | 4e−56 |
| Ancylostoma ceylanicum | pj32a08.y1 | gb\|BQ288531 | 2e−56 |
| Ancylostoma ceylanicum | pj32g07.y1 | gb\|BQ288598 | 2e−56 |
| Ancylostoma ceylanicum | pj33a10.y1 | gb\|BQ288621 | 4e−56 |
| Ancylostoma ceylanicum | pj34g06.y1 | gb\|BQ274732 | 2e−56 |
| Ancylostoma ceylanicum | pj34g09.y1 | gb\|BQ274734 | 1e−56 |
| Ancylostoma ceylanicum | pj35d10.y1 | gb\|BQ274788 | 9e−56 |
| Ancylostoma ceylanicum | pj35e05.y1 | gb\|BQ274793 | 2e−56 |
| Ancylostoma ceylanicum | pj35g08.y1 | gb\|BQ274814 | 2e−56 |
| Ancylostoma ceylanicum | pj37e02.y1 | gb\|BQ275637 | 2e−56 |
| Ancylostoma ceylanicum | pj38d11.y1 | gb\|BQ274868 | 7e−56 |
| Ancylostoma ceylanicum | pj38e07.y1 | gb\|BQ274876 | 9e−56 |
| Ancylostoma ceylanicum | pj38f07.y1 | gb\|BQ274885 | 9e−56 |
| Ancylostoma ceylanicum | pj39e10.y1 | gb\|BQ274958 | 9e−56 |
| Ancylostoma ceylanicum | pj39f12.y1 | gb\|BQ274970 | 2e−56 |
| Ancylostoma ceylanicum | pj40e07.y1 | gb\|BQ275043 | 2e−56 |
| Ancylostoma ceylanicum | pj40f11.y1 | gb\|BQ275056 | 4e−56 |
| Ancylostoma ceylanicum | pj40h07.y1 | gb\|BQ275074 | 7e−56 |
| Ancylostoma ceylanicum | pj41e06.y1 | gb\|BQ275125 | 2e−56 |
| Ancylostoma ceylanicum | pj43a07.y1 | gb\|BQ275254 | 2e−56 |
| Ancylostoma ceylanicum | pj44d05.y1 | gb\|BQ275371 | 7e−56 |
| Ancylostoma ceylanicum | pj46b08.y1 | gb\|BQ275695 | 2e−56 |
| Ancylostoma ceylanicum | pj46h04.y1 | gb\|BQ275751 | 4e−56 |
| Ancylostoma ceylanicum | pj47e05.y1 | gb\|BQ275803 | 5e−56 |
| Ancylostoma ceylanicum | pj47g03.y1 | gb\|BQ275822 | 9e−56 |
| Ancylostoma ceylanicum | pj48e07.y1 | gb\|BQ275890 | 9e−56 |
| Ancylostoma ceylanicum | pj48h05.y1 | gb\|BQ275920 | 2e−56 |
| Ancylostoma ceylanicum | pj51c10.y1 | gb\|BQ276107 | 1e−56 |
| Ancylostoma ceylanicum | pj51d07.y1 | gb\|BQ276116 | 2e−56 |
| Ancylostoma ceylanicum | pj51f12.y1 | gb\|BQ276141 | 4e−56 |
| Ancylostoma ceylanicum | pj53b08.y1 | gb\|BQ288073 | 4e−56 |
| Ancylostoma ceylanicum | pj53b11.y1 | gb\|BQ288076 | 4e−56 |
| Ancylostoma ceylanicum | pj53d09.y1 | gb\|BQ288094 | 9e−56 |
| Ancylostoma ceylanicum | pj55d09.y1 | gb\|BQ288231 | 1e−56 |
| Ancylostoma ceylanicum | pj56a11.y1 | gb\|BQ288274 | 3e−56 |
| Ancylostoma ceylanicum | pj47g05.y1 | gb\|BQ275824 | 2e−55 |
| Ancylostoma ceylanicum | pj49g12.y1 | gb\|BQ275986 | 8e−55 |
| Ancylostoma ceylanicum | pj50e07.y1 | gb\|BQ276042 | 1e−55 |
| Ancylostoma ceylanicum | pj21d03.y1 | gb\|BQ289738 | 3e−53 |
| Ancylostoma ceylanicum | pj24c02.y1 | gb\|BQ289052 | 3e−53 |
| Ancylostoma ceylanicum | pj23h05.y1 | gb\|BQ289614 | 2e−52 |
| Ancylostoma ceylanicum | pj28a01.y1 | gb\|BQ289280 | 2e−52 |
| Ancylostoma ceylanicum | pj36d11.y1 | gb\|BQ275548 | 2e−52 |
| Ancylostoma ceylanicum | pj38f01.y1 | gb\|BQ274881 | 3e−51 |
| Ancylostoma ceylanicum | pj50f12.y1 | gb\|BQ276056 | 1e−50 |
| Ancylostoma ceylanicum | pj43h10.y1 | gb\|BQ275332 | 2e−48 |
| Ancylostoma ceylanicum | pj20g08.y1 | gb\|BQ289691 | 8e−44 |
| Ancylostoma ceylanicum | pj45g02.y1 | gb\|BQ275483 | 2e−24 |

C. elegans gene: W01F3.3

| | | Assession | |
|---|---|---|---|
| Species | EST ID | Number | E value |
| Anopheles gambiae | 17000687309881 | gb\|BM642414 | 9e−24 |
| Ancylostoma | pb20b05.y1 | gb\|BM077795 | 2e−19 |
| caninum | | | |
| Haemonchus | Hc_d11_18C12_SKPL | gb\|BF422862 | 9e−18 |
| contortus | | | |
| Caenorhabditis | pk19f02.r1 | gb\|R04105 | 2e−33 |
| briggsae | | | |
| Meloidogyne | rm30c06.y1 | gb\|BI746672 | 6e−31 |
| arenaria | | | |
| Meloidogyne | rb02g12.y1 | gb\|BM882536 | 6e−30 |
| incognita | | | |
| Meloidogyne | rd16d07.y1 | gb\|BQ625515 | 3e−25 |
| incognita | | | |
| Meloidogyne | ra89g12.y1 | gb\|BM774133 | 1e−10 |
| incognita | | | |
| Meloidogyne hapla | rc37e10.y1 | gb\|BM902581 | 5e−19 |
| Meloidogyne hapla | rc54a09.y2 | gb\|BQ089876 | 4e−08 |
| Meloidogyne | rk82f08.y3 | gb\|BI745590 | 9e−18 |
| javanica | | | |
| Necator americanus | Na_L3_31A05_SA | gb\|BU666009 | 1e−21 |
| Strongyloides ratti | kt12a05.y2 | gb\|BG893620 | 2e−19 |
| Strongyloides ratti | kt12a06.y2 | gb\|BG893621 | 2e−19 |
| Strongyloides ratti | kt36a12.y1 | gb\|BI073867 | 2e−19 |
| Strongyloides ratti | kt32b02.y1 | gb\|BI073544 | 4e−19 |
| Strongyloides ratti | kt15d05.y1 | gb\|BG893793 | 5e−18 |
| Strongyloides | kq39d09.y1 | gb\|BE580288 | 3e−20 |
| stercoralis | | | |
| Strongyloides | kq19h11.y1 | gb\|BG226301 | 9e−18 |
| stercoralis | | | |
| Trichuris muris | Tm_ad_30H04_SKPL | gb\|BM174557 | 9e−21 |
| Trichinella spiralis | pt03g01.y1 | gb\|BQ692168 | 2e−08 |

C. elegans gene: T24H7.2

| Species | EST ID | Assession Number | E value |
|---|---|---|---|
| Anopheles gambiae | 17000687490394 | gb\|BM597171 | 8e−26 |
| Bombyx mori | AU003373 | dbj\|AU003373 | 8e−34 |
| Bombyx mori | AU000515 | dbj\|AU000515 | 4e−33 |
| Bombyx mori | AU000521 | dbj\|AU000521 | 1e−33 |
| Bombyx mori | AU003962 | dbj\|AU003962 | 9e−32 |
| Bombyx mori | AV405756 | dbj\|AV405756 | 5e−30 |
| Bombyx mori | AU003842 | dbj\|AU003842 | 6e−29 |
| Bombyx mori | AU003119 | dbj\|AU003119 | 2e−28 |
| Bombyx mori | AU002974 | dbj\|AU002974 | 9e−27 |
| Bombyx mori | AU004644 | dbj\|AU004644 | 9e−27 |
| Bombyx mori | AV406070 | dbj\|AV406070 | 1e−27 |
| Bombyx mori | AV401482 | dbj\|AV401482 | 2e−26 |
| Bombyx mori | AV398101 | dbj\|AV398101 | 2e−25 |
| Bombyx mori | AU003732 | dbj\|AU003732 | 6e−25 |
| Bombyx mori | AU004041 | dbj\|AU004041 | 2e−25 |
| Bombyx mori | AU005109 | dbj\|AU005109 | 1e−25 |
| Bombyx mori | AU000006 | dbj\|AU000006 | 7e−24 |
| Bombyx mori | AU004834 | dbj\|AU004834 | 2e−24 |
| Bombyx mori | AU002644 | dbj\|AU002644 | 2e−23 |
| Bombyx mori | AU002841 | dbj\|AU002841 | 2e−23 |
| Bombyx mori | AU004017 | dbj\|AU004017 | 2e−23 |
| Bombyx mori | AU004636 | dbj\|AU004636 | 1e−23 |
| Bombyx mori | AV404009 | dbj\|AV404009 | 1e−23 |
| Helicoverpa armigera | DH03D07 | gb\|BU038682 | 1e−28 |
| Helicoverpa armigera | DH03C12 | gb\|BU038678 | 3e−26 |
| Meloidogyne incognita | rd23c04.y1 | gb\|BQ548270 | 1e−30 |
| Parastrongyloides | kx97e04.y2 | gb\|BM513653 | 1e−40 |
| trichosuri | | | |
| Parastrongyloides | kx91g06.y1 | gb\|BM513534 | 4e−40 |
| trichosuri | | | |
| Parastrongyloides | kx97e04.y1 | gb\|BM514195 | 4e−40 |
| trichosuri | | | |
| Parastrongyloides | kx91h03.y1 | gb\|BM513542 | 2e−39 |
| trichosuri | | | |
| Parastrongyloides | kx94f07.y1 | gb\|BM514994 | 1e−37 |
| trichosuri | | | |
| Parastrongyloides | kx88e07.y1 | gb\|BM513356 | 6e−37 |
| trichosuri | | | |
| Parastrongyloides | kx94a01.y1 | gb\|BM514944 | 2e−28 |
| trichosuri | | | |
| Strongyloides | kp73b04.y1 | gb\|BE223128 | 3e−32 |
| stercoralis | | | |

C. elegans gene: C23F12.1

| | | Assession | |
|---|---|---|---|
| Species | EST ID | Number | E value |
| Anopheles gambiae | 17000687479257 | gb\|BM596487 | 4e−28 |
| Anopheles gambiae | 17000687145608 | gb\|BM614899 | 1e−13 |
| Anopheles gambiae | 17000687373180 | gb\|BM651037 | 2e−12 |
| Anopheles gambiae | 17000687310164 | gb\|BM586066 | 3e−10 |
| Meloidogyne hapla | rc36h09.y1 | gb\|BM902526 | 2e−24 |
| Meloidogyne | MD0572 | gb\|BE238916 | 6e−17 |
| incognita | | | |
| Meloidogyne hapla | rc35f02.y1 | gb\|BM902409 | 1e−16 |
| Onchocerca | SWOvAFCAP28B08SK | gb\|AI539970 | 2e−38 |
| volvulus | | | |
| Strongyloides | kq38g03.y1 | gb\|BE580231 | 8e−35 |
| stercoralis | | | |
| Strongyloides | kq18a07.y1 | gb\|BG226155 | 4e−32 |
| stercoralis | | | |

C. elegans gene: M03F4.7

| | | Assession | |
|---|---|---|---|
| Species | EST ID | Number | E value |
| Anopheles gambiae | 17000687321631 | gb\|BM587777 | 6e−30 |
| Anopheles gambiae | 17000687087727 | gb\|BM609137 | 1e−28 |
| Ancylostoma | pb41c07.y1 | gb\|BQ666411 | e−111 |
| caninum | | | |
| Ancylostoma | pb46d07.y1 | gb\|BQ666710 | e−110 |
| caninum | | | |
| Ancylostoma | pb55f03.y1 | gb\|BQ667584 | e−108 |
| caninum | | | |
| Ancylostoma | pb44g02.y1 | gb\|BQ666619 | e−107 |
| caninum | | | |
| Ancylostoma | pb56d04.y1 | gb\|BQ667626 | e−107 |
| caninum | | | |
| Ancylostoma | pb40e01.y1 | gb\|BQ666362 | e−104 |
| caninum | | | |
| Ancylostoma | pb41f01.y1 | gb\|BQ666431 | e−104 |
| caninum | | | |
| Ancylostoma | pb07g02.y1 | gb\|BI744344 | 2e−99 |
| caninum | | | |
| Ancylostoma | pb61h06.y1 | gb\|BQ667467 | 3e−92 |
| caninum | | | |
| Ancylostoma | pb24a04.y1 | gb\|BM129955 | 7e−87 |
| caninum | | | |
| Ancylostoma | pb34e05.y1 | gb\|BQ125307 | 3e−83 |
| caninum | | | |
| Ancylostoma | pb27c03.y1 | gb\|BM130151 | 5e−80 |
| caninum | | | |
| Ancylostoma | pb36b05.y1 | gb\|BQ666117 | 3e−70 |
| caninum | | | |
| Anopheles gambiae | 4A3B-AAC-F-10-F | emb\|AJ283528 | 6e−37 |
| Ascaris suum | ki65c01.y1 | gb\|BM319475 | e−103 |
| Ascaris suum | ki31h12.y1 | gb\|BM284247 | 3e−99 |
| Ascaris suum | ki05h11.y1 | gb\|BM280852 | 8e−93 |
| Ascaris suum | kk76f12.y1 | gb\|BU966016 | 1e−92 |
| Ascaris suum | kk07a07.y1 | gb\|BQ095577 | 2e−80 |
| Ascaris suum | ki29d01.y1 | gb\|BM284064 | 8e−67 |
| Brugia malayi | kb09b01.y1 | gb\|BM889340 | 1e−78 |
| Brugia malayi | kb21h09.y1 | gb\|BU781519 | 4e−78 |
| Brugia malayi | kb05d10.y1 | gb\|BM889092 | 2e−77 |
| Brugia malayi | kb08c11.y1 | gb\|BM889289 | 4e−76 |
| Brugia malayi | kb09a09.y1 | gb\|BM889336 | 4e−75 |
| Brugia malayi | kb35a07.y1 | gb\|BU917823 | 1e−72 |
| Brugia malayi | kb33g08.y1 | gb\|BU917746 | 4e−43 |
| Brugia malayi | SWYD25CAU08A09SK | gb\|AW257642 | 2e−33 |
| Haemonchus | Hc_d11_05C04_SAC | gb\|BF059828 | 8e−56 |
| contortus | | | |
| Haemonchus | pw13a11.y1 | gb\|CA033609 | 1e−62 |
| contortus | | | |
| Meloidogyne hapla | rc40d09.y1 | gb\|BM900690 | 2e−83 |
| Meloidogyne hapla | rc29a06.y1 | gb\|BM901456 | 1e−74 |
| Globodera pallida | OP20499 | gb\|BM415425 | 1e−66 |
| Onchocerca | SWOvAMCAQ03E09SK | gb\|AI095964 | 6e−40 |
| volvulus | | | |
| Ostertagia ostertagi | ph54b09.y1 | gb\|BM896658 | 3e−79 |
| Ostertagia ostertagi | ph54b06.y1 | gb\|BM896656 | 4e−77 |
| Ostertagia ostertagi | ph50g05.y1 | gb\|BM896993 | 1e−66 |
| Pristionchus | rs17c06.y1 | gb\|AI986802 | 2e−52 |
| pacificus | | | |
| Pristionchus | rs36d12.y1 | gb\|AW052520 | 3e−48 |
| pacificus | | | |
| Strongyloides ratti | kt77b06.y2 | gb\|BI450741 | 2e−98 |
| Strongyloides ratti | kt25b12.y3 | gb\|BI450405 | 8e−83 |
| Strongyloides ratti | kt77b06.y1 | gb\|BI142485 | 1e−76 |
| Strongyloides | kp90f12.y1 | gb\|BG226555 | 1e−79 |
| stercoralis | | | |
| Strongyloides | kp93c08.y1 | gb\|BG226767 | 4e−85 |
| stercoralis | | | |
| Strongyloides | kq44d02.y1 | gb\|BE581256 | 5e−85 |
| stercoralis | | | |
| Strongyloides | kq32e11.y1 | gb\|BE579808 | 3e−58 |
| stercoralis | | | |
| Toxocara canis | ko07h06.y1 | gb\|BM966480 | 9e−90 |
| Toxocara canis | ko09d02.y1 | gb\|BM966578 | 8e−90 |
| Toxocara canis | ko29c01.y1 | gb\|BQ089597 | 7e−81 |

C. elegans gene: K04F10.4

| | | Assession | |
|---|---|---|---|
| Species | EST ID | Number | E value |
| Anopheles gambiae | 17000687506656 | gb\|BM633120 | 1e−24 |
| Anopheles gambiae | 17000687507484 | gb\|BM633599 | 6e−23 |
| Ancylostoma caninum | pb41a04.y1 | gb\|BQ666394 | 2e−24 |
| Ancylostoma caninum | pb45d03.y1 | gb\|BQ666654 | 2e−24 |
| Ancylostoma caninum | pb55h11.y1 | gb\|BQ667604 | 2e−24 |
| Ancylostoma caninum | pb57d02.y1 | gb\|BQ667675 | 4e−22 |
| Ancylostoma caninum | pb56c12.y1 | gb\|BQ667624 | 2e−21 |
| Ancylostoma caninum | pb06d11.y1 | gb\|BI744250 | 4e−20 |
| Ancylostoma caninum | pb06e07.y1 | gb\|BI744258 | 5e−20 |
| Ancylostoma caninum | pb62e01.y1 | gb\|BQ667504 | 1e−17 |
| Ancylostoma caninum | pb51a04.y1 | gb\|BQ667006 | 2e−15 |
| Apis mellifera | BB170002B20B06.5 | gb\|BI503119 | 5e−27 |
| Globodera | GE2051 | gb\|AW506559 | 8e−34 |
| rostochiensis | | | |
| Haemonchus contortus | pw14h05.y1 | gb\|CA033722 | 1e−95 |
| Meloidogyne hapla | rc48c03.y1 | gb\|BM901742 | 2e−20 |
| Meloidogyne hapla | rf27a01.y1 | gb\|BQ837484 | 1e−20 |
| Meloidogyne hapla | rc47e08.y1 | gb\|BM901678 | 2e−19 |
| Meloidogyne hapla | rf69b12.y1 | gb\|BU094482 | 7e−14 |
| Meloidogyne incognita | rb16a10.y1 | gb\|BM880593 | 9e−14 |
| Meloidogyne incognita | ra87a11.y1 | gb\|BM773890 | 1e−13 |
| Necator americanus | Na_L3_17G04_SAC | gb\|BU087198 | 4e−14 |
| Ostertagia ostertagi | ph25b11.y2 | gb\|BQ099039 | 5e−18 |
| Ostertagia ostertagi | ph25d06.y2 | gb\|BQ099057 | 3e−13 |
| Parastrongyloides | kx11d08.y3 | gb\|BI451155 | 2e−63 |
| trichosuri | | | |
| Parastrongyloides | kx09d05.y3 | gb\|BI322885 | 2e−58 |
| trichosuri | | | |
| Parastrongyloides | kx14f11.y3 | gb\|BI322659 | 9e−54 |
| trichosuri | | | |
| Parastrongyloides | kx13e05.y3 | gb\|BI322554 | 8e−50 |
| trichosuri | | | |
| Parastrongyloides | kx37f06.y1 | gb\|BI743006 | 3e−37 |
| trichosuri | | | |
| Parastrongyloides | kx35g09.y1 | gb\|BI742844 | 4e−35 |
| trichosuri | | | |
| Parastrongyloides | kx38c05.y1 | gb\|BI743068 | 2e−12 |
| trichosuri | | | |
| Strongyloides ratti | ku14a12.y1 | gb\|BQ091197 | 2e−18 |
| Strongyloides | kp21e05.y1 | gb\|BE028912 | 7e−24 |
| stercoralis | | | |
| Strongyloides | kp31f09.y1 | gb\|BE029399 | 4e−24 |
| stercoralis | | | |
| Strongyloides | kp25f12.y1 | gb\|BE029166 | 2e−22 |
| stercoralis | | | |
| Strongyloides | kp72e12.y1 | gb\|BG225849 | 1e−19 |
| stercoralis | | | |
| Strongyloides | kp41h12.y1 | gb\|BE030358 | 7e−16 |
| stercoralis | | | |
| Strongyloides | kp70g06.y1 | gb\|BG225690 | 7e−16 |
| stercoralis | | | |
| Strongyloides | kp68c10.y1 | gb\|BG225473 | 3e−15 |
| stercoralis | | | |
| Strongyloides | kp74h04.y1 | gb\|BE223285 | 2e−14 |
| stercoralis | | | |
| Strongyloides | kp40c03.y1 | gb\|BE030223 | 1e−13 |
| stercoralis | | | |
| Strongyloides | kp40g11.y1 | gb\|BE030270 | 2e−12 |
| stercoralis | | | |
| Strongyloides | kq43e03.y1 | gb\|BE581195 | 2e−73 |
| stercoralis | | | |
| Strongyloides | kq11c12.y1 | gb\|BG227598 | 5e−42 |
| stercoralis | | | |
| Strongyloides | kp96f07.y1 | gb\|BG227075 | 2e−41 |
| stercoralis | | | |
| Strongyloides | kq35e07.y1 | gb\|BE579996 | 3e−18 |
| stercoralis | | | |
| Trichinella spiralis | pt11b03.y1 | gb\|BQ693113 | 1e−51 |
| Trichinella spiralis | pt15a05.y1 | gb\|BQ692444 | 7e−27 |
| Trichinella spiralis | ps89g02.y1 | gb\|BQ541838 | 4e−19 |
| Trichinella spiralis | pt08f06.y1 | gb\|BQ692908 | 3e−18 |
| Trichinella spiralis | pt10c09.y1 | gb\|BQ693042 | 5e−18 |
| Trichinella spiralis | pt02e07.y1 | gb\|BQ692074 | 1e−15 |

C. elegans gene: F41C3.4

| | | Assession | |
|---|---|---|---|
| Species | EST ID | Number | E value |
| Anopheles gambiae | 17000687149117 | gb\|BM616703 | 2e−27 |
| Anopheles gambiae | 17000687069029 | gb\|BM602087 | 3e−22 |
| Anopheles gambiae | 17000687370128 | gb\|BM649918 | 5e−16 |
| Anopheles gambiae | 17000687307553 | gb\|BM585633 | 9e−16 |
| Anopheles gambiae | AL692646 | emb\|AL692646 | 7e−11 |
| Ancylostoma caninum | pj14f02.y1 | gb\|BM131161 | 3e−52 |
| Brugia malayi | MBAFCX8E03T3 | gb\|AA509202 | 2e−11 |
| Haemonchus contortus | pw09h01.y1 | gb\|CA034321 | 8e−46 |
| Haemonchus contortus | pw04h10.y1 | gb\|CA033875 | 2e−45 |
| Haemonchus contortus | pw06g03.y1 | gb\|CA034012 | 2e−45 |
| Haemonchus contortus | pw11c02.y1 | gb\|CA033489 | 2e−45 |
| Haemonchus contortus | pw11f07.y1 | gb\|CA033516 | 2e−45 |
| Haemonchus contortus | pw13f10.y1 | gb\|CA033653 | 2e−45 |
| Haemonchus contortus | pw16e06.y1 | gb\|CA033344 | 2e−45 |
| Haemonchus contortus | pw07e03.y1 | gb\|CA034184 | 3e−44 |
| Haemonchus contortus | pw11b07.y1 | gb\|CA033483 | 3e−43 |
| Haemonchus contortus | pw14c04.y1 | gb\|CA033687 | 9e−37 |
| Haemonchus contortus | pw11a08.y1 | gb\|CA033477 | 1e−22 |
| Meloidogyne arenaria | rm17b07.y1 | gb\|BI745692 | 1e−32 |
| Strongyloides ratti | kt15c03.y1 | gb\|BG893781 | 8e−20 |

C. elegans gene: F49C12.12

| Species | EST ID | Assession Number | E value |
|---|---|---|---|
| Anopheles gambiae | 17000687157397 | gb\|BM617424 | 5e−13 |
| Anopheles gambiae | 17000659084146 | gb\|BM603802 | 7e−13 |
| Anopheles gambiae | 17000687163115 | gb\|BM576950 | 7e−13 |
| Anopheles gambiae | 17000687275479 | gb\|BM582159 | 7e−13 |
| Anopheles gambiae | 17000687478936 | gb\|BM623580 | 7e−13 |
| Anopheles gambiae | 17000687493042 | gb\|BM625373 | 7e−13 |
| Ancylostoma caninum | pb02e11.y1 | gb\|BF250630 | 1e−22 |
| Ancylostoma caninum | pa80g12.y1 | gb\|BG232750 | 1e−13 |
| Ancylostoma ceylanicum | pj34c09.y1 | gb\|BQ274691 | 1e−34 |
| Ancylostoma ceylanicum | pj26b10.y1 | gb\|BQ289124 | 1e−33 |
| Ancylostoma ceylanicum | pj47a06.y1 | gb\|BQ275763 | 4e−33 |
| Ancylostoma ceylanicum | pj53e04.y1 | gb\|BQ288100 | 6e−33 |
| Ancylostoma ceylanicum | pj55c11.y1 | gb\|BQ288222 | 2e−33 |
| Bombyx mori | AU004305 | dbj\|AU004305 | 9e−13 |
| Bombyx mori | AV404505 | dbj\|AV404505 | 1e−12 |
| Globodera rostochiensis | GE1768 | gb\|AW506351 | 2e−36 |
| Heterodera glycines | ro14f12.y1 | gb\|BF013515 | 2e−36 |
| Manduca sexta | EST1141 | gb\|BF047044 | 6e−12 |
| Meloidogyne hapla | rf52c12.y2 | gb\|BU094732 | 2e−30 |
| Meloidogyne javanica | rk98d03.y1 | gb\|BI745272 | 3e−12 |
| Necator americanus | Na_L3_52B05_SAC | gb\|BU089096 | 9e−37 |
| Necator americanus | Na_L3_13A10_SAC | gb\|BU086831 | 1e−36 |
| Ostertagia ostertagi | ph82h03.y1 | gb\|BQ457787 | 1e−05 |
| Parastrongyloides trichosuri | kx83e06.y1 | gb\|BM513019 | 5e−30 |
| Parastrongyloides trichosuri | kx83a12.y1 | gb\|BM512987 | 9e−19 |
| Strongyloides stercoralis | kp36g11.y1 | gb\|BE029934 | 1e−15 |
| Trichinella spiralis | ps85c06.y1 | gb\|BQ543136 | 4e−17 |
| Trichinella spiralis | ps01f05.y1 | gb\|BG232803 | 2e−14 |
| Trichuris muris | Tm_ad_31C04_SKPL | gb\|BM174586 | 2e−19 |

C. elegans gene: C01H6.5

| | | Assession | |
|---|---|---|---|
| Species | EST ID | Number | E value |
| Anopheles gambiae | 17000687115955 | gb\|BM611525 | 1e−18 |
| Anopheles gambiae | 17000687438370 | gb\|BM618330 | 2e−18 |
| Ascaris suum | ki20a12.y1 | gb\|BM281749 | 2e−39 |
| Ascaris suum | ki04c07.y1 | gb\|BM280724 | 3e−21 |
| Ascaris suum | kj40b03.y1 | gb\|BM568658 | 3e−21 |
| Apis mellifera | BB160005B10B06.5 | gb\|BI511357 | 1e−50 |
| Apis mellifera | BB160003A10G01.5 | gb\|BI510638 | 2e−23 |
| Apis mellifera | BB160016A20C12.5 | gb\|BI514819 | 1e−22 |
| Apis mellifera | BB160017A10C06.5 | gb\|BI514984 | 2e−18 |
| Bombyx mori | AU000440 | dbj\|AU000440 | 6e−27 |
| Bombyx mori | AV398791 | dbj\|AV398791 | 7e−19 |
| Trichinella spiralis | ps26g10.y1 | gb\|BG353339 | 3e−29 |

C. elegans gene: F57B9.2

| | | Assession | |
|---|---|---|---|
| Species | EST ID | Number | E value |
| Anopheles gambiae | 17000687476900 | gb\|BM622947 | 7e−41 |
| Anopheles gambiae | 4A3A-AAY-A-12-R | emb\|AJ282447 | 2e−25 |
| Ancylostoma caninum | pj60d02.y3 | gb\|BU780997 | 6e−53 |
| Amblyomma variegatum | EST577652 | gb\|BM291118 | 2e−51 |
| Meloidogyne incognita | rb13e12.y1 | gb\|BM881751 | 3e−36 |

C. elegans gene: C09G5.6

| | | Assession | |
|---|---|---|---|
| Species | EST ID | Number | E value |
| Ascaris suum | MBAsBWA298M13R | gb\|AW165858 | 1e−26 |
| Ascaris suum | ki01e01.y1 | gb\|BM280488 | 3e−26 |
| Ascaris | Al_am_43C11_T3 | gb\|BU586933 | 6e−25 |
| lumbricoides | | | |
| Ascaris suum | MBAsBWA064M13R | gb\|AW165746 | 6e−25 |
| Ascaris suum | MBAsBWA101M13R | gb\|AW165662 | 6e−25 |
| Ascaris suum | As_bw_11D06_M13R | gb\|BG733657 | 7e−25 |
| Ascaris suum | kh96f09.y1 | gb\|BM285267 | 5e−24 |
| Ascaris suum | kh93f02.y1 | gb\|BM285005 | 3e−23 |
| Ascaris suum | kh94c02.y1 | gb\|BM285056 | 3e−22 |
| Ascaris suum | kh98c07.y1 | gb\|BM284719 | 2e−21 |
| Ascaris suum | As_bw_11D11_M13R | gb\|BG733660 | 1e−21 |
| Ascaris suum | MBAsBWA069M13R | gb\|AW165751 | 6e−20 |
| Ascaris suum | MBAsBWA079M13R | gb\|AW165757 | 1e−20 |
| Ascaris suum | ki03c09.y1 | gb\|BM280644 | 1e−20 |
| Ascaris suum | ki10h03.y1 | gb\|BM281210 | 1e−20 |
| Ascaris | Al_am_36G05_T3 | gb\|BU586727 | 4e−19 |
| lumbricoides | | | |
| Ascaris suum | MBAsBWA115M13R | gb\|AW165673 | 3e−19 |
| Ascaris | Al_am_06E07_T3 | gb\|BU585487 | 2e−18 |
| lumbricoides | | | |
| Ascaris suum | MBAsBWA108M13R | gb\|AW165669 | 9e−18 |
| Ascaris suum | ki07g08.y1 | gb\|BM280986 | 9e−18 |
| Ascaris suum | As_nc_11A05_SKPL | gb\|BI594341 | 1e−17 |
| Brugia malayi | SWBmL3SDI01B01SK | gb\|AI066836 | 3e−22 |
| Brugia malayi | SWBmL3SBH08A07SK | gb\|AA933446 | 5e−21 |
| Brugia malayi | SWYD25CAU13E10SK | gb\|AW675970 | 1e−21 |
| Brugia malayi | SWYD25CAU07H07SK | gb\|AW225415 | 3e−18 |
| Brugia malayi | SWYD25CAU08E01SK | gb\|AW257678 | 4e−18 |
| Brugia malayi | SWAMCAC16G06SK | gb\|AI083297 | 2e−18 |
| Brugia malayi | MBAFCX3C05T3 | gb\|AA471504 | 3e−18 |
| Globodera pallida | OP20201 | gb\|BM415129 | 1e−20 |
| Onchocerca | SWOv3MCAM47A04SK | gb\|BF482033 | 4e−19 |
| volvulus | | | |
| Onchocerca | SWOv3MCAM54F12SK | gb\|BF942751 | 1e−18 |
| volvulus | | | |
| Onchocerca | SWOvAFCAP48F12SK | gb\|BF114585 | 2e−18 |
| volvulus | | | |
| Onchocerca | SWOv3MCAM54B04SK | gb\|BF918253 | 5e−18 |
| volvulus | | | |
| Onchocerca | SWOv3MCAM49B01SK | gb\|BF599258 | 7e−18 |
| volvulus | | | |
| Onchocerca | SWOv3MCAM56A04SK | gb\|BG310491 | 7e−18 |
| volvulus | | | |
| Onchocerca | SWOv3MCAM55E02SK | gb\|BG310586 | 1e−17 |
| volvulus | | | |
| Onchocerca | SWOv3MCAM58G11SK | gb\|BF718930 | 2e−17 |
| volvulus | | | |
| Onchocerca | SWOvL2CAS04B05SK | gb\|AW980134 | 3e−18 |
| volvulus | | | |
| Onchocerca | SWOvL2CAS12F11SK | gb\|BE552486 | 2e−17 |
| volvulus | | | |
| Onchocerca | SWOvL3CAN29D10SK | gb\|AI511508 | 2e−17 |
| volvulus | | | |
| Onchocerca | SWOv3MCA1795SK | gb\|AA618829 | 4e−18 |
| volvulus | | | |
| Onchocerca | SWOv3MCA1241SK | gb\|AI111204 | 7e−18 |
| volvulus | | | |
| Ostertagia ostertagi | Oo_L4_01H05_SKPL | gb\|BG734092 | 1e−22 |
| Ostertagia ostertagi | Oo_L4_02F08_SKPL | gb\|BG734148 | 2e−20 |
| Ostertagia ostertagi | Oo_L4_02F06_SKPL | gb\|BG734146 | 1e−19 |
| Ostertagia ostertagi | Oo_L4_02C04_SKPL | gb\|BG734117 | 8e−19 |
| Ostertagia ostertagi | Oo_L4_03D09_SKPL | gb\|BG891779 | 1e−18 |
| Strongyloides | kq20e08.y1 | gb\|BG226349 | 7e−25 |
| stercoralis | | | |
| Strongyloides | kq60b02.y1 | gb\|BF015009 | 7e−25 |
| stercoralis | | | |
| Strongyloides | kp95e12.y1 | gb\|BG227018 | 1e−17 |
| stercoralis | | | |
| Strongyloides | kq38g09.y1 | gb\|BE580236 | 1e−17 |
| stercoralis | | | |

C. elegans gene: F38A1.8

| | | Assession | |
|---|---|---|---|
| Species | EST ID | Number | E value |
| Anopheles gambiae | 17000668812767 | gb\|BM631762 | 3e−17 |
| Anopheles gambiae | 17000687134447 | gb\|BM612513 | 2e−14 |
| Anopheles gambiae | 17000687443762 | gb\|BM593736 | 1e−12 |
| Anopheles gambiae | 17000687151121 | gb\|BM617129 | 5e−11 |
| Anopheles gambiae | 17000687509165 | gb\|BM634491 | 4e−10 |
| Ancylostoma caninum | pb34d04.y1 | gb\|BQ125296 | 6e−24 |
| Anopheles gambiae | 4A3A-AAY-A-03-F | emb\|AJ280813 | 6e−29 |
| Amblyomma | EST577711 | gb\|BM291177 | 6e−26 |
| variegatum | | | |
| Amblyomma | EST575079 | gb\|BM292537 | 1e−21 |
| variegatum | | | |
| Apis mellifera | BB160006B10D05.5 | gb\|BI511656 | 3e−25 |
| Apis mellifera | BB160008B20D07.5 | gb\|BI512333 | 6e−15 |
| Bombyx mori | AV400988 | dbj\|AV400988 | 1e−21 |
| Meloidogyne javanica | rk65c03.y1 | gb\|BG736990 | 9e−17 |
| Parastrongyloides | kx31d03.y1 | gb\|BI501368 | 6e−31 |
| trichosuri | | | |
| Strongyloides | kq13c10.y1 | gb\|BG227780 | 1e−24 |
| stercoralis | | | |
| Zeldia punctata | rp11b10.y1 | gb\|AW773519 | 4e−22 |

C. elegans gene: F54C9.2

| Species | EST ID | Assession Number | E value |
|---|---|---|---|
| Amblyomma variegatum | EST577517 | gb\|BM290983 | 7e−35 |
| Amblyomma variegatum | EST577724 | gb\|BM291190 | 2e−35 |
| Bombyx mori | AU002973 | dbj\|AU002973 | 9e−39 |
| Bombyx mori | AU003385 | dbj\|AU003385 | 7e−39 |
| Bombyx mori | AV405994 | dbj\|AV405994 | 3e−39 |
| Bombyx mori | AV401902 | dbj\|AV401902 | 7e−38 |
| Bombyx mori | AV398157 | dbj\|AV398157 | 9e−38 |
| Bombyx mori | AU000006 | dbj\|AU000006 | 2e−38 |
| Bombyx mori | AV401963 | dbj\|AV401963 | 2e−37 |
| Bombyx mori | AV402885 | dbj\|AV402885 | 7e−37 |
| Bombyx mori | AU004017 | dbj\|AU004017 | 8e−37 |
| Bombyx mori | AU006113 | dbj\|AU006113 | 4e−37 |
| Bombyx mori | AU000664 | dbj\|AU000664 | 5e−36 |
| Bombyx mori | AU003373 | dbj\|AU003373 | 1e−36 |
| Bombyx mori | AU003442 | dbj\|AU003442 | 5e−36 |
| Bombyx mori | AU003705 | dbj\|AU003705 | 2e−36 |
| Bombyx mori | AU003286 | dbj\|AU003286 | 6e−35 |
| Bombyx mori | AU004420 | dbj\|AU004420 | 6e−35 |
| Bombyx mori | AU004716 | dbj\|AU004716 | 7e−35 |
| Bombyx mori | AU006399 | dbj\|AU006399 | 5e−35 |
| Bombyx mori | AV404137 | dbj\|AV404137 | 7e−35 |
| Bombyx mori | AV405329 | dbj\|AV405329 | 3e−35 |
| Bombyx mori | AV401750 | dbj\|AV401750 | 2e−34 |
| Bombyx mori | AV398101 | dbj\|AV398101 | 5e−34 |
| Bombyx mori | AU000646 | dbj\|AU000646 | 2e−34 |
| Bombyx mori | AU003356 | dbj\|AU003356 | 2e−34 |
| Bombyx mori | AU003364 | dbj\|AU003364 | 2e−34 |
| Bombyx mori | AU003396 | dbj\|AU003396 | 3e−34 |
| Bombyx mori | AU003686 | dbj\|AU003686 | 6e−34 |
| Bombyx mori | AU003777 | dbj\|AU003777 | 1e−34 |
| Bombyx mori | AU004205 | dbj\|AU004205 | 5e−34 |
| Bombyx mori | AU004626 | dbj\|AU004626 | 2e−34 |
| Bombyx mori | AU004827 | dbj\|AU004827 | 8e−34 |
| Bombyx mori | AV404445 | dbj\|AV404445 | 3e−34 |
| Bombyx mori | AV405771 | dbj\|AV405771 | 8e−34 |
| Bombyx mori | AV405924 | dbj\|AV405924 | 2e−34 |
| Bombyx mori | AV406118 | dbj\|AV406118 | 4e−34 |
| Bombyx mori | AV398235 | dbj\|AV398235 | 2e−33 |
| Bombyx mori | AV398367 | dbj\|AV398367 | 2e−33 |
| Bombyx mori | AV398398 | dbj\|AV398398 | 2e−33 |
| Bombyx mori | AU000243 | dbj\|AU000243 | 2e−33 |
| Bombyx mori | AU002763 | dbj\|AU002763 | 2e−33 |
| Bombyx mori | AU003119 | dbj\|AU003119 | 1e−33 |
| Bombyx mori | AU003402 | dbj\|AU003402 | 2e−33 |
| Bombyx mori | AU003811 | dbj\|AU003811 | 2e−33 |
| Bombyx mori | AU004599 | dbj\|AU004599 | 2e−33 |
| Bombyx mori | AU004708 | dbj\|AU004708 | 1e−33 |
| Bombyx mori | AV404361 | dbj\|AV404361 | 2e−33 |
| Bombyx mori | AV406241 | dbj\|AV406241 | 3e−33 |
| Pristionchus pacificus | rs33h02.y1 | gb\|AW052295 | 1e−55 |
| Strongyloides stercoralis | kq09h07.y1 | gb\|BG226148 | 1e−44 |

C. elegans gene: F08C6.1

| Species | EST ID | Assession Number | E value |
|---|---|---|---|
| Strongyloides stercoralis | kq42f07.y1 | gb\|BE581131 | 2e−34 |

C. elegans gene: H04M03.4

| | | Assession | |
|---|---|---|---|
| Species | EST ID | Number | E value |
| Brugia malayi | SWAMCAC31A02SK | gb\|AI770981 | 2e−27 |
| Brugia malayi | SWAMCA827SK | gb\|AA007720 | 8e−21 |
| Meloidogyne arenaria | rm17b03.y1 | gb\|BI745690 | 1e−61 |
| Meloidogyne hapla | rc32c07.y1 | gb\|BM902109 | 2e−56 |
| Meloidogyne hapla | rc62e03.y1 | gb\|BQ090180 | 9e−31 |
| Meloidogyne hapla | rc51b07.y2 | gb\|BQ089651 | 9e−12 |
| Onchocerca volvulus | SWOv3MCA840SK | gb\|AA294602 | 5e−15 |
| Onchocerca volvulus | SWOv3MCA233SK | gb\|AA294264 | 2e−11 |
| Strongyloides ratti | kt23d09.y3 | gb\|BI397280 | 1e−61 |
| Strongyloides ratti | kt17d09.y1 | gb\|BG894044 | 1e−57 |
| Strongyloides ratti | kt09f02.y1 | gb\|BG894269 | 3e−35 |
| Strongyloides ratti | kt14e07.y1 | gb\|BG893462 | 3e−35 |
| Strongyloides | kq07b03.y1 | gb\|BG227443 | 2e−75 |
| stercoralis | | | |
| Strongyloides | kq36f05.y1 | gb\|BE580066 | 1e−55 |
| stercoralis | | | |

C. elegans gene: Y48B6A.3

| Species | EST ID | Assession Number | E value |
|---|---|---|---|
| Ostertagia ostertagi | ph79d04.y1 | gb\|BQ457535 | 6e−52 |
| Globodera rostochiensis | rr63d03.y1 | gb\|BM345416 | 3e−13 |
| Strongyloides ratti | kt53e08.y3 | gb\|BI324097 | 6e−40 |

C. elegans gene: T27F2.1

| | | Assession | |
|---|---|---|---|
| Species | EST ID | Number | E value |
| Anopheles gambiae | 17000687243104 | gb\|BM580695 | 1e−37 |
| Anopheles gambiae | 17000687309019 | gb\|BM641931 | 1e−36 |
| Apis mellifera | BB160009A10E09.5 | gb\|BI512416 | 2e−38 |
| Meloidogyne hapla | rc43c01.y1 | gb\|BM900937 | 1e−33 |
| Meloidogyne incognita | rb13h02.y1 | gb\|BM881774 | 8e−22 |
| Necator americanus | Na_L3_16E01_SAC | gb\|BU087096 | 1.4e−22 |

C. elegans gene: T14F9.1

| Species | EST ID | Assession Number | E value |
|---|---|---|---|
| Anopheles gambiae | 17000687110468 | gb\|BM610289 | 4e−75 |
| Anopheles gambiae | 17000687367798 | gb\|BM648864 | 1e−62 |
| Anopheles gambiae | 17000687565365 | gb\|BM637983 | 2e−58 |
| Anopheles gambiae | 17000687324034 | gb\|BM647561 | 2e−57 |
| Anopheles gambiae | 17000687560412 | gb\|BM635758 | 2e−56 |
| Anopheles gambiae | 17000687163827 | gb\|BM577458 | 2e−55 |
| Anopheles gambiae | 17000687119262 | gb\|BM611925 | 2e−54 |
| Anopheles gambiae | 17000668915702 | gb\|BM596992 | 8e−48 |
| Anopheles gambiae | 17000687377484 | gb\|BM653132 | 2e−45 |
| Anopheles gambiae | 17000687164768 | gb\|BM578365 | 1e−42 |
| Anopheles gambiae | 17000687499422 | gb\|BM629010 | 5e−41 |
| Anopheles gambiae | 17000687368814 | gb\|BM649439 | 2e−39 |
| Anopheles gambiae | 17000687496339 | gb\|BM597467 | 3e−38 |
| Anopheles gambiae | 17000687243041 | gb\|BM580645 | 6e−38 |
| Anopheles gambiae | 17000687384459 | gb\|BM590932 | 6e−38 |
| Ancylostoma caninum | pb28e07.y1 | gb\|BM130242 | 4e−72 |
| Ascaris suum | As_nc_16B02_SKPL | gb\|BI594547 | 4e−67 |
| Apis mellifera | BB170001B10D01.5 | gb\|BI504920 | 2e−39 |
| Bombyx mori | AU003538 | dbj\|AU003538 | 3e−80 |
| Bombyx mori | AU002118 | dbj\|AU002118 | 1e−44 |
| Bombyx mori | AU006312 | dbj\|AU006312 | 5e−36 |
| Globodera rostochiensis | rr09e06.y1 | gb\|BM345905 | 2e−73 |
| Heterodera glycines | ro25h04.y1 | gb\|BF014612 | 2e−54 |
| Heterodera glycines | ro28a10.y1 | gb\|BF014776 | 7e−54 |
| Meloidogyne javanica | rk48d04.y1 | gb\|BG735889 | 5e−65 |
| Globodera pallida | OP20152 | gb\|BM415082 | 2e−60 |
| Necator americanus | Na_L3_10D12_SAC | gb\|BU086612 | 5e−65 |
| Parastrongyloides trichosuri | kx75e08.y1 | gb\|BM513291 | 5e−69 |
| Strongyloides stercoralis | kq20f07.y1 | gb\|BG226359 | 5e−70 |
| Strongyloides stercoralis | kq22d06.y1 | gb\|BE579107 | 2e−56 |

C. elegans gene: C34G6.6

| | | Assession | |
|---|---|---|---|
| Species | EST ID | Number | E value |
| Ascaris suum | kh44c05.y1 | gb\|BI782938 | 9e−52 |
| Brugia malayi | MBAFCX2B06T3 | gb\|AA471404 | 2e−68 |
| Brugia malayi | SWAMCAC32C03SK | gb\|AI795199 | 4e−63 |
| Haemonchus | Hc_d11_10F03_SKPL | gb\|BF060055 | 4e−18 |
| contortus | | | |
| Meloidogyne | rk89c03.y1 | gb\|BI744615 | 4e−44 |
| javanica | | | |
| Meloidogyne | rm18b11.y1 | gb\|BI745765 | 4e−10 |
| arenaria | | | |
| Pristionchus | rs76h10.y1 | gb\|BI500192 | 2e−69 |
| pacificus | | | |
| Strongyloides ratti | kt36b11.y1 | gb\|BI073876 | 1e−41 |
| Strongyloides ratti | kt37a09.y1 | gb\|BI073944 | 2e−41 |
| Strongyloides ratti | kt70c11.y1 | gb\|BI323373 | 1e−36 |
| Strongyloides ratti | kt62e08.y1 | gb\|BI323179 | 2e−36 |
| Strongyloides | kq30c01.y1 | gb\|BE579677 | 2e−53 |
| stercoralis | | | |
| Strongyloides | kq41b02.y1 | gb\|BE580410 | 1e−47 |
| stercoralis | | | |
| Strongyloides | kq33h12.y1 | gb\|BE579888 | 4e−22 |
| stercoralis | | | |
| Strongyloides | kq63d06.y1 | gb\|BF015363 | 7e−20 |
| stercoralis | | | |
| Strongyloides | kq05d11.y1 | gb\|BG227329 | 3e−11 |
| stercoralis | | | |
| Trichuris muris | Tm_ad_12H10_SKPL | gb\|BG577864 | 4e−12 |

C. elegans gene: T01H3.1

| Species | EST ID | Assession Number | E value |
|---|---|---|---|
| Anopheles gambiae | 17000687162874 | gb\|BM576761 | 2e−49 |
| Anopheles gambiae | 17000687307464 | gb\|BM585573 | 2e−49 |
| Anopheles gambiae | 17000659020522 | gb\|BM599204 | 1e−48 |
| Anopheles gambiae | 17000687372976 | gb\|BM589399 | 1e−48 |
| Anopheles gambiae | 17000687556220 | gb\|BM634907 | 1e−48 |
| Anopheles gambiae | 17000687310364 | gb\|BM642665 | 5e−48 |
| Anopheles gambiae | 17000687389290 | gb\|BM656815 | 5e−48 |
| Anopheles gambiae | 17000687496331 | gb\|BM597462 | 2e−47 |
| Anopheles gambiae | 17000687284475 | gb\|BM640730 | 2e−46 |
| Anopheles gambiae | 17000659202014 | gb\|BM618205 | 5e−45 |
| Anopheles gambiae | 17000687151325 | gb\|BM617284 | 3e−44 |
| Anopheles gambiae | 17000687308708 | gb\|BM641772 | 6e−44 |
| Anopheles gambiae | 17000687276191 | gb\|BM582703 | 3e−43 |
| Anopheles gambiae | 17000687042988 | gb\|BM599367 | 2e−42 |
| Anopheles gambiae | 17000687118079 | gb\|BM611782 | 2e−42 |
| Anopheles gambiae | 17000687108061 | gb\|BM609935 | 2e−40 |
| Anopheles gambiae | 17000687130255 | gb\|BM612274 | 4e−39 |
| Anopheles gambiae | 17000687322687 | gb\|BM646650 | 6e−39 |
| Anopheles gambiae | 17000687569900 | gb\|BM639302 | 4e−37 |
| Anopheles gambiae | 17000687383534 | gb\|BM654480 | 7e−37 |
| Anopheles gambiae | 17000687566347 | gb\|BM638292 | 7e−37 |
| Anopheles gambiae | 17000687145897 | gb\|BM615037 | 6e−36 |
| Anopheles gambiae | 17000687437980 | gb\|BM618257 | 2e−33 |
| Anopheles gambiae | 17000687498198 | gb\|BM628172 | 6e−30 |
| Anopheles gambiae | 17000687147324 | gb\|BM615629 | 3e−26 |
| Amblyomma variegatum | EST574690 | gb\|BM292148 | 3e−49 |
| Apis mellifera | BB170024A10E06.5 | gb\|BI509938 | 7e−48 |
| Apis mellifera | BB160011B10C08.5 | gb\|BI513199 | 2e−33 |
| Bombyx mori | AU005205 | dbj\|AU005205 | 2e−33 |
| Globodera rostochiensis | GE1711 | gb\|AW506310 | 4e−67 |
| Haemonchus contortus | Hc_ad_15H10_SKPL | gb\|BM39010 | 2e−76 |
| Ancylostoma caninum | pa18g12.y1 | gb\|AW627173 | 8e−25 |
| Heterodera glycines | ro10e01.y1 | gb\|BF013645 | 4e−46 |
| Heterodera glycines | ro84c04.y1 | gb\|BI748962 | 1e−20 |
| Zeldia punctata | rp06a10.y1 | gb\|AW773378 | 3e−60 |
| Zeldia punctata | rp01e11.y1 | gb\|AW783702 | 3e−52 |
| Manduca sexta | EST968 | gb\|BF046871 | 2e−35 |
| Meloidogyne javanica | rk60d11.y1 | gb\|BG736647 | 3e−39 |
| Meloidogyne javanica | rk74g05.y1 | gb\|BI142836 | 1e−39 |
| Necator americanus | Na_L3_37A02_SAC | gb\|BU666330 | 8e−33 |
| Strongyloides ratti | kt49e06.y4 | gb\|BI502419 | 6e−38 |
| Strongyloides stercoralis | kq25f06.y1 | gb\|BE579307 | 3e−29 |
| Trichinella spiralis | ps31h12.y2 | gb\|BG438616 | 1e−50 |
| Trichinella spiralis | pt25f08.y1 | gb\|BQ737954 | 1e−50 |

C. elegans gene: F38H4.9

| Species | EST ID | Assession Number | E value |
|---|---|---|---|
| Anopheles gambiae | 17000687077251 | gb\|BM605757 | e−110 |
| Anopheles gambiae | 17000687112957 | gb\|BM610732 | 2e−99 |
| Anopheles gambiae | 17000687498699 | gb\|BM628538 | 3e−98 |
| Anopheles gambiae | 17000687491478 | gb\|BM624539 | 1e−95 |
| Anopheles gambiae | 17000687162264 | gb\|BM576307 | 2e−93 |
| Anopheles gambiae | 17000687237697 | gb\|BM579205 | 9e−91 |
| Anopheles gambiae | 17000687494575 | gb\|BM626183 | 1e−90 |
| Anopheles gambiae | 17000687373656 | gb\|BM651182 | 3e−87 |
| Anopheles gambiae | 17000687387542 | gb\|BM656160 | 3e−87 |
| Anopheles gambiae | 17000687439479 | gb\|BM618770 | 3e−87 |
| Anopheles gambiae | 17000687138537 | gb\|BM613259 | 9e−83 |
| Anopheles gambiae | 17000687386006 | gb\|BM591368 | 2e−77 |
| Anopheles gambiae | 17000687075820 | gb\|BM605128 | 3e−77 |
| Anopheles gambiae | 17000687444639 | gb\|BM594603 | 2e−75 |
| Anopheles gambiae | 17000687311718 | gb\|BM643158 | 9e−75 |
| Ascaris suum | kh42g04.y1 | gb\|BI782814 | 8e−89 |
| Ascaris suum | ki30c03.y1 | gb\|BM284127 | 9e−80 |
| Ascaris suum | kj60c12.y1 | gb\|BM569375 | 3e−55 |
| Amblyomma variegatum | EST576450 | gb\|BM289916 | 2e−74 |
| Apis mellifera | BB170030B20B04.5 | gb\|BI507201 | 7e−80 |
| Bombyx mori | AU000600 | dbj\|AU000600 | 3e−91 |
| Bombyx mori | AU000644 | dbj\|AU000644 | 3e−91 |
| Meloidogyne javanica | rk93b04.y1 | gb\|BI744849 | 4e−79 |
| Necator americanus | Na_L3_35H12_SAC | gb\|BU666328 | e−118 |
| Necator americanus | Na_L3_16C05_SAC | gb\|BU087079 | 1e−99 |
| Necator americanus | Na_L3_17H12_SAC | gb\|BU087214 | 1e−37 |
| Necator americanus | Na_L3_51B04_SAC | gb\|BU089013 | 5e−20 |
| Ostertagia ostertagi | ph05a12.y2 | gb\|BQ097609 | e−104 |
| Ostertagia ostertagi | ph08g10.y2 | gb\|BQ097814 | 2e−99 |
| Parastrongyloides trichosuri | kx48h12.y1 | gb\|BI863834 | 2e−69 |

C. elegans gene: K09H9.6

| | | Assession | |
|---|---|---|---|
| Species | EST ID | Number | E value |
| Anopheles gambiae | 17000687438560 | gb\|BM592590 | 1e−26 |
| Anopheles gambiae | 17000687439428 | gb\|BM618729 | 1e−25 |
| Brugia malayi | SWAMCAC19C09SK | gb\|AI083314 | 8e−27 |
| Sarcoptes scabiei | ESSU0232 | gb\|BG817810 | 4e−27 |
| Strongyloides | kp98c02.y1 | gb\|BG227182 | 2e−28 |
| stercoralis | | | |
| Trichinella spiralis | ps51c09.y1 | gb\|BG520770 | 4e−21 |

C. elegans gene: F54A5.1

| | | Assession | |
|---|---|---|---|
| Species | EST ID | Number | E value |
| Parastrongyloides trichosuri | kx21a02.y1 | gb\|BI451197 | 4e−40 |

C. elegans gene: F33A8.1

| | | Assession | |
|---|---|---|---|
| Species | EST ID | Number | E value |
| Bombyx mori | AV405747 | dbj\|AV405747 | 6e−58 |
| Meloidogyne | MD0049 | gb\|BE191668 | 3e−36 |
| incognita | | | |

C. elegans gene: ZK686.3

| | | Assession | |
|---|---|---|---|
| Species | EST ID | Number | E value |
| Anopheles gambiae | 17000687101940 | gb\|BM609414 | 1e−46 |
| Anopheles gambiae | 17000687160079 | gb\|BM617864 | 3e−44 |
| Anopheles gambiae | 17000687499984 | gb\|BM629333 | 7e−40 |
| Anopheles gambiae | 17000687320084 | gb\|BM586921 | 6e−39 |
| Anopheles gambiae | 17000687564993 | gb\|BM637706 | 2e−38 |
| Anopheles gambiae | 17000687385741 | gb\|BM655170 | 1e−37 |
| Anopheles gambiae | 17000687441179 | gb\|BM593025 | 1e−35 |
| Anopheles gambiae | 17000687087920 | gb\|BM609287 | 2e−32 |
| Anopheles gambiae | 17000687113255 | gb\|BM610801 | 1e−29 |
| Anopheles gambiae | 17000668938573 | gb\|BM636391 | 9e−21 |
| Anopheles gambiae | 4A3A-AAO-F-10-R | emb\|AJ282089 | 5e−25 |
| Anopheles gambiae | 4A3A-ABC-G-08-R | emb\|AJ282843 | 2e−19 |
| Apis mellifera | EST242 | gb\|BE844497 | 1e−25 |
| Apis mellifera | EST241 | gb\|BE844496 | 2e−10 |
| Amblyomma | EST575352 | gb\|BM292810 | 7e−52 |
| variegatum | | | |
| Amblyomma | EST574536 | gb\|BM291994 | 3e−51 |
| variegatum | | | |
| Apis mellifera | BB160010B10H06.5 | gb\|BI512874 | 1e−34 |
| Bombyx mori | AU004344 | dbj\|AU004344 | 1e−52 |
| Brugia malayi | MBAFCW6H10T3 | gb\|AA842318 | 8e−22 |
| Brugia malayi | SWMFCA462SK | gb\|AA022342 | 1e−21 |
| Globodera pallida | pal201 | gb\|AW505639 | 1e−32 |
| Haemonchus | Hc_d11_21A04_SKPL | gb\|BF423018 | 9e−74 |
| contortus | | | |
| Haemonchus | Hc_d11_13F09_SKPL | gb\|BF060296 | 4e−36 |
| contortus | | | |
| Caenorhabditis | pk41g11.s1 | gb\|R05170 | 8e−33 |
| briggsae | | | |
| Necator americanus | Na_L3_04C08_SAC | gb\|BG467473 | 6e−22 |
| Pristionchus | rs40g12.y1 | gb\|AW097184 | 9e−71 |
| pacificus | | | |
| Pristionchus | rs30c07.y1 | gb\|AI989236 | 4e−29 |
| pacificus | | | |
| Strongyloides | kq49c03.y1 | gb\|BE581316 | 2e−48 |
| stercoralis | | | |
| Strongyloides | kq08h12.y1 | gb\|BG226083 | 3e−47 |
| stercoralis | | | |
| Strongyloides | kq23b07.y1 | gb\|BE579155 | 4e−27 |
| stercoralis | | | |

C. elegans gene: F09B12.1

| | | Assession | |
|---|---|---|---|
| Species | EST ID | Number | E value |
| Onchocerca | SWOv3MCAM23F06SK | gb\|AI665735 | 4e−10 |
| volvulus | | | |
| Strongyloides | kq19a02.y1 | gb\|BG226227 | 1e−23 |
| stercoralis | | | |
| Strongyloides | kq43f01.y1 | gb\|BE581202 | 1e−13 |
| stercoralis | | | |
| Trichuris muris | Tm_ad_03C11_SKPL | gb\|BF169279 | 5e−11 |

C. elegans gene: K07D8.1

| | | Assession | |
|---|---|---|---|
| Species | EST ID | Number | E value |
| Anopheles gambiae | 17000687507565 | gb\|BM633656 | 3e−15 |
| Brugia malayi | BSBmL3SZ15A23SK | gb\|AI783143 | 1e−66 |
| Meloidogyne | MD0517 | gb\|BE238861 | 8e−38 |
| incognita | | | |
| Strongyloides ratti | kt27g02.y3 | gb\|BI450575 | 3e−38 |
| Strongyloides ratti | kt88d03.y1 | gb\|BI502339 | 6e−33 |
| Strongyloides | kp75c05.y1 | gb\|BE223322 | 2e−23 |
| stercoralis | | | |

C. elegans gene: ZK1073.1

| | | Assession | |
|---|---|---|---|
| Species | EST ID | Number | E value |
| Anopheles gambiae | 17000687445431 | gb\|BM620760 | 3e−29 |
| Anopheles gambiae | 17000687311462 | gb\|BM642979 | 3e−27 |
| Anopheles gambiae | 17000687069233 | gb\|BM602173 | 8e−27 |
| Anopheles gambiae | 17000687085881 | gb\|BM608010 | 8e−17 |
| Anopheles gambiae | 17000687277442 | gb\|BM583063 | 2e−15 |
| Anopheles gambiae | 17000668639510 | gb\|BM629367 | 1e−13 |
| Anopheles gambiae | 17000687379911 | gb\|BM654068 | 2e−13 |
| Amblyomma variegatum | EST577485 | gb\|BM290951 | 9e−23 |
| Amblyomma variegatum | EST575426 | gb\|BM292884 | 8e−19 |
| Amblyomma variegatum | EST576458 | gb\|BM289924 | 2e−19 |
| Amblyomma variegatum | EST574248 | gb\|BM291706 | 2e−18 |
| Amblyomma variegatum | EST574565 | gb\|BM292023 | 2e−18 |
| Amblyomma variegatum | EST575109 | gb\|BM292567 | 2e−18 |
| Amblyomma variegatum | EST575360 | gb\|BM292818 | 1e−18 |
| Amblyomma variegatum | EST575673 | gb\|BM293144 | 2e−18 |
| Amblyomma variegatum | EST576512 | gb\|BM289978 | 5e−18 |
| Amblyomma variegatum | EST576929 | gb\|BM290395 | 4e−18 |
| Amblyomma variegatum | EST577334 | gb\|BM290800 | 2e−18 |
| Amblyomma variegatum | EST576568 | gb\|BM290034 | 2e−17 |
| Amblyomma variegatum | EST576853 | gb\|BM290319 | 9e−15 |
| Bombyx mori | AV400999 | dbj\|AV400999 | 1e−20 |
| Bombyx mori | AV400998 | dbj\|AV400998 | 4e−15 |
| Globodera rostochiensis | rr26f04.y1 | gb\|BM355559 | 1e−50 |
| Globodera rostochiensis | rr08g01.y1 | gb\|BM345835 | 3e−35 |
| Ancylostoma caninum | pa49f11.y1 | gb\|AW735249 | 6e−46 |
| Heterodera glycines | ro77a08.y1 | gb\|BI749346 | 4e−37 |
| Heterodera glycines | ro60f02.y3 | gb\|BI396703 | 4e−26 |
| Heterodera glycines | ro76c03.y1 | gb\|BI749286 | 2e−25 |
| Heterodera glycines | ro57a04.y4 | gb\|BI451623 | 6e−16 |
| Heterodera glycines | ro75g12.y1 | gb\|BI749253 | 1e−16 |
| Meloidogyne incognita | rd12e01.y1 | gb\|BQ548499 | 7e−73 |
| Meloidogyne arenaria | rm15c02.y1 | gb\|BI863000 | 3e−15 |
| Ostertagia ostertagi | ph39b03.y1 | gb\|BM897271 | 9e−34 |
| Parastrongyloides | kx43h07.y1 | gb\|BI743414 | 1e−31 |
| trichosuri | | | |
| Pristionchus pacificus | rs88f09.y1 | gb\|BM320361 | 9e−92 |
| Pristionchus pacificus | rt04c04.y2 | gb\|BM566361 | 1e−23 |
| Strongyloides ratti | kt66c09.y1 | gb\|BI323694 | 2e−57 |
| Strongyloides stercoralis | kp87b07.y1 | gb\|BE223687 | 1e−36 |
| Strongyloides stercoralis | kq04g11.y1 | gb\|BG227286 | 8e−59 |
| Trichinella spiralis | pt34f08.y1 | gb\|BQ693400 | 3e−52 |
| Trichinella spiralis | pt41e05.y1 | gb\|BQ739201 | 4e−42 |
| Trichinella spiralis | ps06g08.y1 | gb\|BG302307 | 3e−34 |

C. elegans gene: CD4.4

| Species | EST ID | Assession Number | E value |
|---|---|---|---|
| Bombyx mori | AU003753 | dbj\|AU003753 | 2e−06 |
| Pratylenchus penetrans | pz11e06.y1 | gb\|BQ626542 | 2e−19 |
| Pratylenchus penetrans | pz21d10.y1 | gb\|BQ580851 | 2e−19 |
| Pratylenchus penetrans | pz28a06.y1 | gb\|BQ626857 | 1e−06 |
| Pristionchus pacificus | rs39f03.y1 | gb\|AW097092 | 8e−24 |
| Pristionchus pacificus | rs53g02.y1 | gb\|AW114710 | 5e−18 |
| Pristionchus pacificus | rs37f03.y1 | gb\|AW052618 | 2e−14 |
| Trichinella spiralis | ps05d02.y2 | gb\|BG519941 | 6e−11 |
| Trichinella spiralis | ps05d02.y3 | gb\|BG521059 | 3e−11 |

C. elegans gene: F11C1.6

| | | Assession | |
|---|---|---|---|
| Species | EST ID | Number | E value |
| Anopheles gambiae | 17000687115955 | gb\|BM611525 | 3e−41 |
| Ascaris suum | ki04c07.y1 | gb\|BM280724 | 6e−20 |
| Ascaris suum | kj40b03.y1 | gb\|BM568658 | 6e−20 |
| Ascaris suum | ki20a12.y1 | gb\|BM281749 | 5e−18 |
| Ascaris suum | kk53a06.y1 | gb\|BQ382607 | 4e−18 |
| Ascaris suum | kh20b07.y1 | gb\|BI783431 | 1e−17 |
| Ascaris suum | kk28e12.y1 | gb\|BQ381181 | 1e−17 |
| Ascaris suum | kk34c05.y1 | gb\|BQ381563 | 1e−17 |
| Ascaris suum | kk36a10.y1 | gb\|BQ382856 | 1e−17 |
| Ascaris suum | kk40g10.y1 | gb\|BQ383122 | 1e−17 |
| Ascaris suum | kk58c01.y1 | gb\|BQ383209 | 1e−17 |
| Apis mellifera | BB160003A10G01.5 | gb\|BI510638 | 4e−22 |
| Apis mellifera | BB160005B10B06.5 | gb\|BI511357 | 7e−19 |
| Apis mellifera | BB160017A10C06.5 | gb\|BI514984 | 4e−18 |
| Apis mellifera | BB160016A20C12.5 | gb\|BI514819 | 1e−17 |
| Bombyx mori | AU000440 | dbj\|AU000440 | 3e−17 |
| Globodera | rr19d05.y1 | gb\|BM354985 | 2e−17 |
| rostochiensis | | | |
| Strongyloides | kq42c09.y1 | gb\|BE581104 | 1e−27 |
| stercoralis | | | |

C. elegans gene: F16B4.3

| | | Assession | |
|---|---|---|---|
| Species | EST ID | Number | E value |
| Pristionchus | rs06b03.r1 | gb\|AA191781 | 2e−13 |
| pacificus | | | |

C. elegans gene: Y38F2AL.3

| | | Assession | |
|---|---|---|---|
| Species | EST ID | Number | E value |
| Anopheles gambiae | 17000687310422 | gb\|BM642705 | 2e−46 |
| Anopheles gambiae | 17000687111489 | gb\|BM610534 | 1e−41 |
| Anopheles gambiae | 17000687374739 | gb\|BM651831 | 2e−36 |
| Anopheles gambiae | 17000687444802 | gb\|BM594743 | 2e−35 |
| Anopheles gambiae | 17000687564429 | gb\|BM637448 | 1e−29 |
| Bombyx mori | AU005959 | dbj\|AU005959 | 6e−64 |
| Brugia malayi | BSBmMFSZ08G14SK | gb\|AI007333 | 2e−79 |
| Globodera | rr24f03.y1 | gb\|BM355406 | 1e−69 |
| rostochiensis | | | |
| Caenorhabditis | pk05f06.s1 | gb\|R03292 | 2e−32 |
| briggsae | | | |
| Meloidogyne arenaria | rm27a09.y1 | gb\|BI746435 | 2e−80 |
| Parastrongyloides | kx20h12.y3 | gb\|BI322419 | 3e−44 |
| trichosuri | | | |
| Pristionchus pacificus | rs80f06.y1 | gb\|BI500714 | 1e−82 |
| Strongyloides ratti | kt66a07.y1 | gb\|BI323674 | 5e−47 |
| Strongyloides ratti | kt46f02.y3 | gb\|BI323910 | 7e−39 |
| Strongyloides | kq10b05.y1 | gb\|BG227519 | 1e−69 |
| stercoralis | | | |
| Strongyloides | kq50d07.y1 | gb\|BE581674 | 8e−64 |
| stercoralis | | | |
| Strongyloides | kq41h12.y1 | gb\|BE580542 | 4e−42 |
| stercoralis | | | |
| Strongyloides | kp45f12.y1 | gb\|BG224376 | 2e−29 |
| stercoralis | | | |
| Trichinella spiralis | pt40c10.y1 | gb\|BQ739097 | 2e−65 |
| Trichinella spiralis | ps03d04.y3 | gb\|BG520983 | 1e−49 |
| Trichinella spiralis | pt07a03.y1 | gb\|BQ692776 | 2e−45 |
| Trichinella spiralis | ps03d04.y1 | gb\|BG302151 | 5e−37 |
| Trichinella spiralis | ps12g06.y1 | gb\|BG322017 | 2e−35 |

C. elegans gene: W09B6.1

| | | Assession | |
|---|---|---|---|
| Species | EST ID | Number | E value |
| Anopheles gambiae | 17000687083533 | gb\|BM606557 | 2e−48 |
| Anopheles gambiae | 17000687439275 | gb\|BM618618 | 2e−46 |
| Anopheles gambiae | 17000687321659 | gb\|BM587799 | 2e−43 |
| Anopheles gambiae | 17000687086088 | gb\|BM608168 | 5e−42 |
| Anopheles gambiae | 17000687315677 | gb\|BM645103 | 2e−38 |
| Anopheles gambiae | 17000687236701 | gb\|BM579048 | 3e−33 |
| Anopheles gambiae | 17000659179265 | gb\|BM610911 | 5e−30 |
| Ancylostoma caninum | pb31a01.y1 | gb\|BQ125044 | 2e−61 |
| Ascaris suum | kh05g07.y1 | gb\|BI782124 | 2e−47 |
| Ascaris suum | kh06f09.y1 | gb\|BI782194 | 4e−47 |
| Ascaris suum | kk05h08.y1 | gb\|BQ095491 | 4e−43 |
| Ascaris suum | kh01h12.y1 | gb\|BI781835 | 7e−42 |
| Necator americanus | Na_L3_34C04_SAC | gb\|BU666204 | 2.e−15 |
| Strongyloides ratti | ku15c12.y1 | gb\|BQ091288 | 7e−29 |
| Strongyloides | kq36e08.y1 | gb\|BE580061 | 8e−43 |
| stercoralis | | | |
| Strongyloides | kq60a06.y1 | gb\|BF015002 | 4e−38 |
| stercoralis | | | |
| Strongyloides | kq52b10.y1 | gb\|BE581778 | 9e−31 |
| stercoralis | | | |
| Toxocara canis | ko08f10.y1 | gb\|BM966530 | 1e−37 |
| Toxocara canis | ko24c07.y1 | gb\|BQ089283 | 1e−37 |

C. elegans gene: T19B10.2

| | | Assession | |
|---|---|---|---|
| Species | EST ID | Number | E value |
| Brugia malayi | SW3D9CA428SK | gb\|AA585672 | 1e−63 |
| Haemonchus | Hc_d11_28B01_SKPL | gb\|BF423321 | 6e−55 |
| contortus | | | |
| Onchocerca | SWOv3MCAM47D12SK | gb\|BF482074 | 2e−48 |
| volvulus | | | |
| Onchocerca | SWOvAFCAP42G02SK | gb\|AW600024 | 8e−57 |
| volvulus | | | |
| Onchocerca | SWOvAFCAP37H10SK | gb\|AW562321 | 2e−50 |
| volvulus | | | |
| Onchocerca | SWOv3MCAM04C01SK | gb\|AI053004 | 3e−40 |
| volvulus | | | |
| Onchocerca | SWOv3MCAM33B12SK | gb\|AW288189 | 5e−24 |
| volvulus | | | |
| Pristionchus | rs17d11.y1 | gb\|AI986817 | 2e−61 |
| pacificus | | | |
| Strongyloides ratti | kt46c03.y3 | gb\|BI323886 | 2e−57 |
| Strongyloides ratti | kt76d02.y3 | gb\|BI502537 | 2e−57 |
| Strongyloides ratti | kt15h05.y1 | gb\|BG893826 | 3e−49 |
| Strongyloides ratti | kt64a08.y1 | gb\|BI323577 | 9e−12 |
| Strongyloides | kq07e10.y1 | gb\|BG227479 | 3e−67 |
| stercoralis | | | |
| Strongyloides | kp97f11.y1 | gb\|BG227146 | 1e−59 |
| stercoralis | | | |
| Strongyloides | kq43c11.y1 | gb\|BE581183 | 1e−57 |
| stercoralis | | | |
| Strongyloides | kq51c07.y1 | gb\|BE581720 | 1e−36 |
| stercoralis | | | |
| Trichinella spiralis | ps16e05.y2 | gb\|BG520446 | 2e−12 |

C. elegans gene: F40G9.1

| Species | EST ID | Assession Number | E value |
|---|---|---|---|
| Ancylostoma | pk22g03.x1 | gb\|CA341524 | 3e−37 |
| caninum | | | |
| Ascaris suum | As_nc_09H02_SKPL | gb\|BI594288 | 8e−29 |
| Apis mellifera | BB160019B20G12.5 | gb\|BI515617 | 3e−10 |
| Necator | Na_L3_03F10_SAC | gb\|BG467849 | 3e−13 |
| americanus | | | |

C. elegans gene: M88.6

| | | Assession | |
|---|---|---|---|
| Species | EST ID | Number | E value |
| Anopheles gambiae | 17000659338584 | gb\|BM622660 | 1e−16 |
| Anopheles gambiae | 17000687147208 | gb\|BM615535 | 5e−16 |
| Anopheles gambiae | 17000687312380 | gb\|BM643448 | 3e−14 |
| Anopheles gambiae | 17000687503711 | gb\|BM632047 | 2e−12 |
| Anopheles gambiae | 17000687317758 | gb\|BM646000 | 1e−11 |
| Anopheles gambiae | 17000687566305 | gb\|BM638257 | 1e−11 |
| Anopheles gambiae | 17000687507490 | gb\|BM633604 | 1e−10 |
| Anopheles gambiae | 17000687446031 | gb\|BM621183 | 2e−10 |
| Anopheles gambiae | 17000687490365 | gb\|BM597156 | 3e−10 |
| Anopheles gambiae | 17000687556043 | gb\|BM634822 | 7e−10 |
| Anopheles gambiae | 17000687507788 | gb\|BM633679 | 9e−10 |
| Apis mellifera | BB170030A10B11.5 | gb\|BI505904 | 2e−12 |
| Apis mellifera | BB170016A20D05.5 | gb\|BI510550 | 5e−08 |
| Bombyx mori | AV403012 | dbj\|AV403012 | 4e−11 |
| Bombyx mori | AV400933 | dbj\|AV400933 | 3e−10 |
| Meloidogyne arenaria | rm24h02.y1 | gb\|BI746256 | 3e−15 |
| Meloidogyne javanica | rk43d08.y1 | gb\|BG735742 | 5e−14 |
| Meloidogyne javanica | rk97f05.y1 | gb\|BI745212 | 2e−12 |

C. elegans gene: CD4.6

| Species | EST ID | Assession Number | E value |
|---|---|---|---|
| Anopheles gambiae | 17000687371664 | gb\|BM650501 | 6e−67 |
| Anopheles gambiae | 17000687322329 | gb\|BM588180 | 3e−62 |
| Anopheles gambiae | 17000687071573 | gb\|BM602678 | 3e−60 |
| Anopheles gambiae | 17000687068376 | gb\|BM601670 | 7e−60 |
| Anopheles gambiae | 17000687313631 | gb\|BM644198 | 2e−56 |
| Anopheles gambiae | 17000687439860 | gb\|BM619034 | 2e−56 |
| Anopheles gambiae | 17000687277359 | gb\|BM583000 | 2e−55 |
| Anopheles gambiae | 17000687619748 | gb\|BM598398 | 5e−47 |
| Artemia franciscana | ar10-065 | gb\|BQ605277 | 1e−63 |
| Amblyomma | EST576373 | gb\|BM289839 | 2e−69 |
| variegatum | | | |
| Bombyx mori | AV398746 | dbj\|AV398746 | 1e−61 |
| Globodera | rr26d03.y1 | gb\|BM355545 | 2e−67 |
| rostochiensis | | | |
| Heterodera glycines | ro21g02.y1 | gb\|BF014394 | 2e−68 |
| Meloidogyne javanica | rk14h04.y1 | gb\|BE578613 | 4e−61 |
| Meloidogyne arenaria | rm38a11.y1 | gb\|BI747271 | 8e−53 |
| Meloidogyne hapla | rc34d01.y1 | gb\|BM902290 | 6e−51 |
| Meloidogyne javanica | rk70b10.y1 | gb\|BI143067 | 1e−45 |
| Globodera pallida | OP20486 | gb\|BM415412 | 3e−72 |
| Pristionchus pacificus | rs39e05.y1 | gb\|AW097083 | 2e−76 |
| Strongyloides ratti | kt29h05.y1 | gb\|BI073353 | 6e−69 |
| Trichinella spiralis | pt02c08.y1 | gb\|BQ692053 | 3e−63 |
| Trichinella spiralis | ps98a12.y1 | gb\|BQ542423 | 2e−47 |

C. elegans gene: F52B11.3

| | | Assession | |
|---|---|---|---|
| Species | EST ID | Number | E value |
| Brugia malayi | MBAFCW3E10T3 | gb\|AA661399 | 4e−48 |
| Meloidogyne hapla | rc57a01.y1 | gb\|BM952243 | 9e−71 |
| Meloidogyne hapla | rc58e09.y1 | gb\|BQ090007 | 4e−56 |
| Meloidogyne hapla | rc26c02.y1 | gb\|BM901200 | 4e−06 |
| Meloidogyne arenaria | rm36a05.y1 | gb\|BI747105 | 7e−06 |
| Meloidogyne arenaria | rm03h03.y1 | gb\|BI501693 | 7e−06 |
| Strongyloides ratti | kt15h10.y1 | gb\|BG893830 | 7e−80 |
| Strongyloides stercoralis | kq38b02.y1 | gb\|BE580180 | 1e−73 |
| Strongyloides stercoralis | kq61e08.y1 | gb\|BF015258 | 2e−72 |
| Strongyloides stercoralis | kq24d08.y1 | gb\|BE579237 | 1e−53 |
| Strongyloides stercoralis | kq42f04.y1 | gb\|BE581128 | 1e−43 |
| Strongyloides stercoralis | kp96c07.y1 | gb\|BG227056 | 8e−37 |

C. elegans gene: F41H10.7

| Species | EST ID | Assession Number | E value |
|---|---|---|---|
| Anopheles gambiae | 17000687316343 | gb\|BM645334 | 5e−20 |
| Ancylostoma caninum | pb13c07.y1 | gb\|BM077653 | 2e−40 |
| Ancylostoma caninum | pj06b02.y1 | gb\|BM130528 | 2e−40 |
| Ancylostoma caninum | pj13h01.y1 | gb\|BM131118 | 5e−40 |
| Ancylostoma caninum | pj01g12.y1 | gb\|BI704649 | 3e−34 |
| Ancylostoma caninum | pj05b02.y1 | gb\|BM130446 | 4e−25 |
| Ascaris suum | kk23a03.y2 | gb\|BQ381206 | 1e−48 |
| Ascaris suum | kk23a11.y2 | gb\|BQ381214 | 6e−47 |
| Ascaris suum | kj50f05.y1 | gb\|BM515548 | 7e−35 |
| Ascaris suum | As_adfo_05B04_T3 | gb\|CA303612 | 7e−35 |
| Ascaris suum | ki05a11.y1 | gb\|BM280791 | 4e−34 |
| Ascaris suum | kj52c04.y1 | gb\|BM515678 | 3e−34 |
| Ascaris suum | As_adfg_09B10_T3 | gb\|BU605554 | 1e−34 |
| Ascaris suum | ki44g05.y1 | gb\|BM283072 | 2e−33 |
| Ascaris suum | kj49f11.y1 | gb\|BM515471 | 4e−33 |
| Ascaris suum | kj96b03.y1 | gb\|BQ095112 | 4e−33 |
| Ascaris suum | kj96c04.y1 | gb\|BQ095124 | 1e−33 |
| Ascaris suum | As_adfo_07A06_T3 | gb\|CA303746 | 1e−32 |
| Ascaris suum | kh96b06.y1 | gb\|BM285220 | 2e−30 |
| Ascaris suum | ki71h03.y1 | gb\|BM319371 | 2e−15 |
| Brugia malayi | SWMFCA2329SK | gb\|AA545829 | 9e−40 |
| Brugia malayi | SWBmL3SBH11E05SK | gb\|AI079048 | 3e−28 |
| Brugia malayi | SWMFCA2496SK | gb\|AA563533 | 3e−26 |
| Globodera rostochiensis | rr17f12.y1 | gb\|BM354846 | 2e−53 |
| Haemonchus contortus | pw07g07.y1 | gb\|CA034206 | 2e−34 |
| Haemonchus contortus | pw07g09.y1 | gb\|CA034208 | 2e−34 |
| Haemonchus contortus | pw14b10.y1 | gb\|CA033681 | 2e−33 |
| Haemonchus contortus | pw10e09.y1 | gb\|CA034357 | 1e−29 |
| Meloidogyne javanica | rk65e12.y1 | gb\|BG737014 | 5e−29 |
| Meloidogyne javanica | rk98g01.y1 | gb\|BI745300 | 1e−28 |
| Meloidogyne javanica | rk74d11.y1 | gb\|BI142820 | 1e−22 |
| Meloidogyne hapla | rf67e06.y1 | gb\|BU094358 | 6e−20 |
| Meloidogyne hapla | rc35e08.y1 | gb\|BM902404 | 2e−12 |
| Meloidogyne hapla | rf86e02.y2 | gb\|BU095464 | 2e−12 |
| Meloidogyne incognita | rb10d11.y1 | gb\|BM881480 | 6e−10 |
| Globodera pallida | OP20484 | gb\|BM415410 | 2e−28 |
| Necator americanus | Na_L3_24H05_SAC | gb\|BU087819 | 2e−14 |
| Onchocerca volvulus | SWOv3MCAM49H12SK | gb\|BF599190 | 2e−61 |
| Onchocerca volvulus | SWOvL3CAN71H05SK | gb\|BF154352 | 8e−25 |
| Onchocerca volvulus | SWOvAFCAP15C07SK | gb\|AI308680 | 2e−49 |
| Onchocerca volvulus | SWOvAFCAP27H07SK | gb\|AI539947 | 1e−22 |
| Onchocerca volvulus | SWOv3MCAM36B09SK | gb\|AW308544 | 9e−17 |
| Onchocerca volvulus | SWOv3MCA1157SK | gb\|AI045995 | 6e−38 |
| Parastrongyloides trichosuri | kx61d04.y1 | gb\|BM356240 | 6e−20 |
| Parastrongyloides trichosuri | kx68g05.y1 | gb\|BM346265 | 1e−19 |
| Parastrongyloides trichosuri | kx77d07.y1 | gb\|BM512626 | 1e−19 |
| Parastrongyloides trichosuri | kx81e05.y1 | gb\|BM512881 | 1e−19 |
| Parastrongyloides trichosuri | kx98d10.y2 | gb\|BM513719 | 1e−16 |
| Parastrongyloides trichosuri | ky01b10.y1 | gb\|BM514338 | 1e−16 |
| Parastrongyloides trichosuri | ky01b10.y3 | gb\|BQ274049 | 1e−16 |
| Parastrongyloides trichosuri | kx76b10.y1 | gb\|BM812691 | 7e−11 |
| Pristionchus pacificus | rs36h03.y1 | gb\|AW052554 | 3e−30 |
| Pristionchus pacificus | rs32b04.y1 | gb\|AW114333 | 2e−27 |
| Pristionchus pacificus | rs08h02.r1 | gb\|AA191857 | 5e−16 |
| Strongyloides ratti | kt24a11.y3 | gb\|BI397325 | 7e−11 |

C. elegans gene: ZK783.1

| | | Assession | |
|---|---|---|---|
| Species | EST ID | Number | E value |
| Anopheles gambiae | 17000687050491 | gb\|BM599848 | 7e−17 |
| Anopheles gambiae | 17000687313297 | gb\|BM643943 | 1e−16 |
| Apis mellifera | BB170008A10D11.5 | gb\|BI507755 | 2e−17 |
| Bombyx mori | AV403913 | dbj\|AV403913 | 1e−18 |
| Bombyx mori | AV405815 | dbj\|AV405815 | 9e−17 |

C. elegans gene: W10G6.3

| Species | EST ID | Assession Number | E value |
|---|---|---|---|
| Ascaris suum | ki02g09.y1 | gb\|BM280603 | 1e−84 |
| Ascaris suum | kh29h03.y1 | gb\|BI784031 | 3e−74 |
| Ascaris suum | kj92f03.y1 | gb\|BM965152 | 1e−72 |
| Ascaris lumbricoides | Al_am_44B09_T3 | gb\|BU586964 | 3e−67 |
| Ascaris suum | ki08f11.y1 | gb\|BM281039 | 1e−65 |
| Ascaris suum | kh97g02.y1 | gb\|BM284670 | 5e−63 |
| Ascaris suum | kh67d02.y1 | gb\|BM033773 | 1e−62 |
| Ascaris suum | kk52b05.y1 | gb\|BQ382546 | 9e−62 |
| Ascaris suum | As_L3_09B01_SKPL | gb\|BI594018 | 3e−61 |
| Ascaris suum | kh07f03.y1 | gb\|BI782261 | 6e−60 |
| Ascaris suum | kk55f09.y1 | gb\|BQ382765 | 2e−60 |
| Ascaris suum | As_nc_20E04_SKPL | gb\|BI594703 | 7e−56 |
| Ascaris lumbricoides | Al_am_39H04_T3 | gb\|BU586869 | 6e−48 |
| Ascaris suum | kh23d05.y1 | gb\|BI784404 | 1e−47 |
| Ascaris suum | As_nc_10C07_SKPL | gb\|BI594311 | 8e−47 |
| Ascaris suum | As_adfo_18A08_T3 | gb\|CA304479 | 7e−46 |
| Ascaris suum | MBAsBWA018M13R | gb\|AW165649 | 1e−44 |
| Ascaris suum | kj97h10.y1 | gb\|BQ095255 | 2e−44 |
| Ascaris suum | kj91c09.y1 | gb\|BM965046 | 2e−42 |
| Ascaris suum | As_nc_07A04_SKPL | gb\|BI594184 | 6e−39 |
| Ascaris suum | kj10g11.y1 | gb\|BM567150 | 4e−37 |
| Ascaris suum | As_nc_17F06_SKPL | gb\|BI594620 | 3e−37 |
| Ascaris suum | ki07a10.y1 | gb\|BM280930 | 4e−34 |
| Ascaris lumbricoides | Al_am_19E10_T3 | gb\|BU585851 | 2e−29 |
| Ascaris suum | kj16d10.y1 | gb\|BM567546 | 1e−29 |
| Brugia malayi | SWYD25CAU14E02SK | gb\|AW675831 | 2e−75 |
| Brugia malayi | SWMFCA1385SK | gb\|AA231989 | 8e−50 |
| Brugia malayi | MB3D6V8E10T3 | gb\|AA841889 | 1e−49 |
| Brugia malayi | SWYACAL08E03SK | gb\|BE758356 | 6e−45 |
| Brugia malayi | SW3ICA2430SK | gb\|AA255390 | 3e−42 |
| Brugia malayi | MB3D6V8A06T3 | gb\|AA841843 | 1e−35 |
| Brugia malayi | KJBmL3SZ4B22SK | gb\|AI944353 | 2e−30 |
| Brugia malayi | RRAMCA1524SK | gb\|AA430804 | 2e−29 |
| Dirofilaria immitis | ke22g11.y1 | gb\|BQ455787 | 1e−35 |
| Globodera rostochiensis | rr58b08.y1 | gb\|BM344699 | 3e−78 |
| Globodera rostochiensis | rr65c04.y1 | gb\|BM345560 | 5e−75 |
| Globodera rostochiensis | rr30c09.y1 | gb\|BM355843 | 2e−59 |
| Globodera rostochiensis | rr30a02.y1 | gb\|BM355821 | 3e−52 |
| Haemonchus contortus | Hc_d11_11E10_SKPL | gb\|BF060126 | 5e−57 |
| Haemonchus contortus | Hc_d11_18E03_SKPL | gb\|BF422872 | 1e−56 |
| Litomosoides sigmodontis | JALsL3C179SAC | gb\|AW152844 | 1e−74 |
| Meloidogyne incognita | rd08a12.y1 | gb\|BQ613497 | 1e−68 |
| Meloidogyne incognita | rd19e10.y1 | gb\|BQ613722 | 1e−68 |
| Meloidogyne hapla | rc49c01.y1 | gb\|BM901834 | 6e−66 |
| Meloidogyne hapla | rc26d08.y1 | gb\|BM901218 | 9e−65 |
| Meloidogyne hapla | rc37g03.y1 | gb\|BM902598 | 6e−64 |
| Meloidogyne incognita | rd02c03.y1 | gb\|BQ613170 | 3e−64 |
| Meloidogyne hapla | rc42h03.y1 | gb\|BM900907 | 3e−62 |
| Meloidogyne hapla | rf48d08.y1 | gb\|BQ836630 | 4e−62 |
| Meloidogyne arenaria | rm47f07.y1 | gb\|BI747934 | 8e−53 |
| Meloidogyne arenaria | rm28c11.y1 | gb\|BI746528 | 1e−48 |
| Meloidogyne javanica | rk75h03.y1 | gb\|BI142900 | 3e−44 |
| Onchocerca volvulus | SWOvAFCAP49B12SK | gb\|BF199444 | 2e−62 |
| Onchocerca volvulus | SWOv3MCAM52D01SK | gb\|BF824665 | 1e−58 |
| Onchocerca volvulus | SWOv3MCAM51A02SK | gb\|BF727562 | 4e−58 |
| Onchocerca volvulus | SWOvL2CAS06B03SK | gb\|AW980259 | 3e−77 |
| Onchocerca volvulus | SWOvAFCAP02E12SK | gb\|AI077021 | 7e−73 |
| Onchocerca volvulus | SWOv3MCAM26G09SK | gb\|AI670483 | 5e−59 |
| Onchocerca volvulus | SWOvL2CAS03E05SK | gb\|AI444905 | 9e−50 |
| Onchocerca volvulus | SWOv3MCAM07B07SK | gb\|AI317899 | 7e−46 |
| Onchocerca volvulus | SWOvAFCB315SK | gb\|AI815264 | 2e−82 |
| Onchocerca volvulus | SWOvL3CAN13E07 | gb\|AA917260 | 2e−51 |
| Onchocerca volvulus | SWOv3MCA822SK | gb\|AA294585 | 2e−51 |
| Ostertagia ostertagi | ph53g02.y1 | gb\|BM896621 | 6e−77 |
| Ostertagia ostertagi | ph69a09.y1 | gb\|BQ099825 | 3e−43 |
| Parastrongyloides trichosuri | kx18a11.y3 | gb\|BI322222 | 1e−43 |
| Strongyloides ratti | kt51c06.y4 | gb\|BI742464 | 8e−50 |
| Strongyloides stercoralis | kp60g10.y1 | gb\|BE224367 | 7e−43 |
| Strongyloides stercoralis | kp89h11.y1 | gb\|BG226499 | 5e−35 |
| Strongyloides stercoralis | kq58d04.y1 | gb\|BF014961 | 2e−66 |
| Strongyloides stercoralis | kq16d05.y1 | gb\|BG227868 | 9e−59 |
| Strongyloides stercoralis | kq25d02.y1 | gb\|BE579290 | 2e−52 |
| Strongyloides stercoralis | kq07e05.y1 | gb\|BG227475 | 3e−50 |
| Strongyloides stercoralis | kq38a11.y1 | gb\|BE580177 | 5e−50 |
| Strongyloides stercoralis | kq01b02.y1 | gb\|BG226921 | 4e−46 |
| Strongyloides stercoralis | kq43f12.y1 | gb\|BE581211 | 3e−45 |
| Strongyloides stercoralis | kq59e08.y1 | gb\|BF014970 | 8e−41 |
| Strongyloides stercoralis | kq31d11.y1 | gb\|BE579614 | 4e−34 |
| Strongyloides stercoralis | kq17c02.y1 | gb\|BG227920 | 3e−30 |
| Toxocara canis | ko17e01.y1 | gb\|BM965806 | 1e−52 |
| Trichinella spiralis | ps41c08.y1 | gb\|BG353660 | 6e−68 |
| Trichinella spiralis | ps21c11.y4 | gb\|BG732010 | 2e−66 |
| Trichuris muris | Tm_ad_02F09_SKPL | gb\|BF049882 | 2e−69 |
| Trichuris muris | Tm_ad_32C10_SKPL | gb\|BM174670 | 3e−69 |
| Trichuris muris | Tm_ad_34B05_SKPL | gb\|BM174819 | 3e−41 |
| Trichuris muris | Tm_ad_28B10_SKPL | gb\|BM174335 | 2e−38 |
| Trichuris muris | Tm_ad_41B01_SKPL | gb\|BM1277502 | 4e−34 |
| Trichuris muris | Tm_ad_30G11_SKPL | gb\|BM174554 | 4e−30 |

C. elegans gene: C17G1.6

| Species | EST ID | Assession Number | E value |
|---|---|---|---|
| Ancylostoma caninum | pb60d09.y1 | gb\|BQ667369 | 3e−21 |
| Ancylostoma caninum | pj59h02.y3 | gb\|BU780981 | 3e−17 |
| Ascaris suum | kk63b06.y1 | gb\|BQ835552 | 8e−41 |
| Ascaris suum | kk75f04.y1 | gb\|BU965942 | 5e−41 |
| Ascaris suum | kk67a07.y1 | gb\|BQ835133 | 5e−39 |
| Ascaris suum | kk81d05.y1 | gb\|BU966321 | 5e−39 |
| Ascaris suum | kk82h03.y1 | gb\|BU966423 | 1e−22 |
| Bombyx mori | AU002182 | dbj\|AU002182 | 2e−18 |
| Brugia malayi | MBAFCX3H02T3 | gb\|AA471557 | 5e−17 |
| Meloidogyne arenaria | rm39h08.y1 | gb\|BI747415 | 2e−17 |
| Meloidogyne arenaria | rm44a01.y1 | gb\|BI747765 | 4e−16 |
| Necator americanus | Na_L3_54E05_SAC | gb\|BU089288 | 2e−29 |
| Necator americanus | Na_L3_46G04_SAC | gb\|BU088646 | 7e−27 |
| Necator americanus | Na_L3_33B12_SAC | gb\|BU088268 | 3e−26 |
| Necator americanus | Na_L3_42D01_SAC | gb\|BU666771 | 2e−25 |
| Necator americanus | Na_L4_01D08_SAC | gb\|BG467914 | 2e−24 |
| Necator americanus | Na_L3_43E08_SAC | gb\|BU666872 | 5e−20 |
| Necator americanus | Na_L3_28E03_SAC | gb\|BU088135 | 1e−19 |
| Necator americanus | Na_L3_18C11_SAC | gb\|BU087246 | 3e−18 |
| Necator americanus | Na_L3_23G12_SAC | gb\|BU087729 | 1e−16 |
| Ostertagia ostertagi | Oo_ad_01F02_LambdaGT11FO | gb\|BG733933 | 6e−20 |
| Ostertagia ostertagi | ph69g06.y1 | gb\|BQ099886 | 8e−18 |
| Ostertagia ostertagi | ph37c02.y1 | gb\|BM897683 | 1e−17 |
| Ostertagia ostertagi | ph43h10.y1 | gb\|BM897848 | 1e−17 |
| Ostertagia ostertagi | ph47c08.y1 | gb\|BM896734 | 4e−17 |
| Ostertagia ostertagi | ph38g05.y1 | gb\|BM897764 | 2e−16 |
| Ostertagia ostertagi | ph44f10.y1 | gb\|BM897904 | 1e−15 |
| Parastrongyloides trichosuri | kx10f03.y3 | gb\|BI451087 | 2e−34 |
| Parastrongyloides trichosuri | kx16e12.y3 | gb\|BI322818 | 6e−29 |
| Parastrongyloides trichosuri | kx13e07.y3 | gb\|BI322556 | 9e−22 |
| Parastrongyloides trichosuri | kx16d11.y3 | gb\|BI322807 | 1e−18 |
| Parastrongyloides trichosuri | kx34b01.y1 | gb\|BI742691 | 1e−18 |
| Parastrongyloides trichosuri | kx27f02.y1 | gb\|BI501067 | 1e−17 |
| Parastrongyloides trichosuri | kx35c07.y1 | gb\|BI742807 | 1e−17 |
| Parastrongyloides trichosuri | kx42f06.y1 | gb\|BI743922 | 1e−15 |
| Parastrongyloides trichosuri | kx26a10.y1 | gb\|BI500947 | 4e−15 |
| Pristionchus pacificus | rs82e09.y1 | gb\|BI500840 | 2e−23 |
| Pristionchus pacificus | rt09b02.y1 | gb\|BQ087806 | 7e−19 |
| Pristionchus pacificus | rs73h01.y1 | gb\|BI500514 | 1e−15 |
| Pristionchus pacificus | rs36b09.y1 | gb\|AW052495 | 5e−20 |
| Strongyloides ratti | kt84a03.y1 | gb\|BI741990 | 3e−39 |
| Strongyloides ratti | kt65c12.y1 | gb\|BI323632 | 3e−21 |
| Strongyloides ratti | kt22h07.y1 | gb\|BG894012 | 8e−18 |
| Strongyloides ratti | ku07c07.y1 | gb\|BM879025 | 6e−16 |
| Strongyloides stercoralis | kp47c05.y1 | gb\|BG224501 | 9e−35 |
| Strongyloides stercoralis | kp25g06.y1 | gb\|BE029170 | 3e−33 |
| Strongyloides stercoralis | kp60b07.y1 | gb\|BE224326 | 5e−33 |
| Strongyloides stercoralis | kp60f02.y1 | gb\|BE224353 | 8e−28 |
| Strongyloides stercoralis | kp04a10.y1 | gb\|AW496628 | 7e−25 |
| Strongyloides stercoralis | kp05g07.y1 | gb\|AW496678 | 5e−25 |
| Strongyloides stercoralis | kp24d04.y1 | gb\|BE029064 | 1e−25 |
| Strongyloides stercoralis | kp36h12.y1 | gb\|BE029947 | 3e−25 |
| Strongyloides stercoralis | kp54h12.y1 | gb\|BE224535 | 2e−25 |
| Strongyloides stercoralis | kp85d05.y1 | gb\|BE223897 | 9e−25 |
| Strongyloides stercoralis | kp50g09.y1 | gb\|BG224805 | 3e−25 |
| Strongyloides stercoralis | kp48d11.y1 | gb\|BG224592 | 4e−25 |
| Strongyloides stercoralis | kp40f09.y1 | gb\|BE030258 | 6e−24 |
| Strongyloides stercoralis | kp23b01.y1 | gb\|BE028980 | 8e−23 |
| Strongyloides stercoralis | kp53g03.y1 | gb\|BE224009 | 8e−23 |
| Strongyloides stercoralis | kp73g06.y1 | gb\|BE223185 | 5e−23 |
| Strongyloides stercoralis | kp84c04.y2 | gb\|BE581022 | 2e−23 |
| Strongyloides stercoralis | kp45a04.y1 | gb\|BG224325 | 8e−23 |
| Strongyloides stercoralis | kp26g12.y1 | gb\|BE029255 | 4e−22 |
| Strongyloides stercoralis | kp78d11.y2 | gb\|BE579761 | 2e−22 |
| Strongyloides stercoralis | kp35h02.y1 | gb\|BE029861 | 2e−21 |
| Strongyloides stercoralis | kp09g10.y1 | gb\|AW587924 | 4e−20 |
| Strongyloides stercoralis | kp26f12.y1 | gb\|BE029245 | 4e−20 |
| Strongyloides stercoralis | kp54d06.y1 | gb\|BE224068 | 2e−20 |
| Strongyloides stercoralis | kp85b01.y1 | gb\|BE223873 | 1e−20 |
| Strongyloides stercoralis | kp49d06.y1 | gb\|BG224673 | 2e−20 |
| Strongyloides stercoralis | kp58g05.y1 | gb\|BE224258 | 2e−19 |
| Strongyloides stercoralis | kp44a03.y1 | gb\|BG225948 | 1e−19 |
| Strongyloides stercoralis | kp66h07.y1 | gb\|BG225405 | 7e−19 |
| Strongyloides stercoralis | kp29d05.y1 | gb\|BE029604 | 6e−18 |
| Strongyloides stercoralis | kp39g02.y1 | gb\|BE030188 | 3e−18 |
| Strongyloides stercoralis | kp41f04.y1 | gb\|BE030329 | 2e−18 |
| Strongyloides stercoralis | kp55e05.y1 | gb\|BE224547 | 5e−18 |
| Strongyloides stercoralis | TNSSFH0001 | gb\|N21795 | 3e−18 |
| Strongyloides stercoralis | kp29g02.y1 | gb\|BE029626 | 7e−17 |
| Strongyloides stercoralis | kp63g05.y1 | gb\|BE224503 | 7e−17 |
| Strongyloides stercoralis | kp80b11.y2 | gb\|BE580669 | 7e−17 |
| Strongyloides stercoralis | kp86b04.y1 | gb\|BE223626 | 9e−17 |
| Strongyloides stercoralis | kp65e09.y1 | gb\|BG225224 | 4e−17 |
| Strongyloides stercoralis | kp25d11.y1 | gb\|BE029146 | 6e−16 |
| Strongyloides stercoralis | kp34d10.y1 | gb\|BE029758 | 2e−16 |
| Strongyloides stercoralis | kp34e03.y1 | gb\|BE029762 | 3e−16 |
| Strongyloides stercoralis | kp37b04.y1 | gb\|BE029959 | 2e−16 |
| Strongyloides stercoralis | kp57h08.y1 | gb\|BE224605 | 6e−16 |
| Strongyloides stercoralis | kp58e10.y1 | gb\|BE224244 | 6e−16 |
| Strongyloides stercoralis | kp60e04.y1 | gb\|BE224345 | 7e−16 |
| Strongyloides stercoralis | kp61b01.y1 | gb\|BG225005 | 2e−16 |
| Strongyloides stercoralis | kp48f04.y1 | gb\|BG224608 | 3e−16 |
| Strongyloides stercoralis | kp72b08.y1 | gb\|BG225809 | 3e−16 |
| Strongyloides stercoralis | kp03g10.y1 | gb\|AW496617 | 3e−15 |
| Strongyloides stercoralis | kp22h08.y1 | gb\|BE028968 | 1e−15 |
| Strongyloides stercoralis | kp71d04.y1 | gb\|BG225739 | 1e−15 |
| Strongyloides stercoralis | kp71c12.y1 | gb\|BG225735 | 3e−15 |
| Strongyloides stercoralis | kp61e11.y1 | gb\|BG225048 | 4e−15 |
| Strongyloides stercoralis | kq08h10.y1 | gb\|BG226082 | 3e−29 |
| Trichinella spiralis | pt31e04.y1 | gb\|BQ738378 | 2e−17 |

C. elegans gene: T05C12.10

| | | Assession | |
|---|---|---|---|
| Species | EST ID | Number | E value |
| Meloidogyne | rd30d09.y1 | gb\|BQ613344 | 7e−47 |
| incognita | | | |
| Meloidogyne | rb25a03.y1 | gb\|BM882030 | 3e−17 |
| incognita | | | |
| Onchocerca | SWOv3MCAM30H08SK | gb\|AW257707 | 1e−22 |
| volvulus | | | |
| Onchocerca | SWOv3MCAM21D03SK | gb\|AI444860 | 2e−12 |
| volvulus | | | |
| Strongyloides | kq58h02.y1 | gb\|BF014893 | 1e−34 |
| stercoralis | | | |
| Strongyloides | kq19a09.y1 | gb\|BG226231 | 2e−24 |
| stercoralis | | | |
| Strongyloides | kq23h04.y1 | gb\|BE579200 | 3e−22 |
| stercoralis | | | |

C. elegans gene: R05D11.3

| Species | EST ID | Assession Number | E value |
|---|---|---|---|
| Anopheles gambiae | 17000687042995 | gb\|BM599374 | 2e−28 |
| Anopheles gambiae | 17000687072218 | gb\|BM603030 | 2e−28 |
| Anopheles gambiae | 17000687443914 | gb\|BM593861 | 2e−28 |
| Anopheles gambiae | 17000687314705 | gb\|BM586411 | 2e−27 |
| Anopheles gambiae | 17000687162185 | gb\|BM576239 | 1e−26 |
| Anopheles gambiae | 17000687478579 | gb\|BM623382 | 3e−23 |
| Anopheles gambiae | 17000687489887 | gb\|BM623861 | 3e−23 |
| Ascaris lumbricoides | Al_am_06F12_T3 | gb\|BU585500 | 4e−44 |
| Ascaris suum | ki56a07.y1 | gb\|BM281377 | 3e−41 |
| Ascaris suum | MBAsBWA194M13R | gb\|AW165779 | 6e−38 |
| Ascaris lumbricoides | Al_am_08A06_T3 | gb\|BU585565 | 4e−32 |
| Ascaris suum | kj45g01.y2 | gb\|BM517341 | 1e−31 |
| Ascaris suum | ki47g06.y1 | gb\|BM283275 | 4e−29 |
| Ascaris lumbricoides | Al_am_28D07_T3 | gb\|BU586336 | 1e−22 |
| Apis mellifera | BB160006B20G02.5 | gb\|BI511717 | 7e−29 |
| Apis mellifera | BB160015A20D12.5 | gb\|BI514405 | 7e−29 |
| Apis mellifera | BB170018A10D07.5 | gb\|BI509477 | 7e−29 |
| Bombyx mori | AU004592 | dbj\|AU004592 | 2e−27 |
| Bombyx mori | AU006081 | dbj\|AU006081 | 6e−27 |
| Bombyx mori | AV406293 | dbj\|AV406293 | 6e−27 |
| Bombyx mori | AV404938 | dbj\|AV404938 | 5e−24 |
| Globodera rostochiensis | rr58f12.y1 | gb\|BM344746 | 3e−41 |
| Heterodera glycines | ro22c04.y1 | gb\|BF014168 | 4e−39 |
| Heterodera glycines | ro27a03.y1 | gb\|BF014695 | 1e−34 |
| Meloidogyne hapla | rc70d01.y1 | gb\|BQ125588 | 8e−38 |
| Ostertagia ostertagi | ph86a03.y1 | gb\|BQ625869 | 2e−39 |
| Pristionchus pacificus | rs32b08.y1 | gb\|AW114337 | 6e−41 |
| Strongyloides ratti | ku24e01.y1 | gb\|BQ091075 | 7e−44 |
| Strongyloides stercoralis | kp53g06.y1 | gb\|BE224012 | 4e−44 |
| Strongyloides stercoralis | kp46d05.y1 | gb\|BG224431 | 4e−44 |
| Strongyloides stercoralis | kp49e01.y1 | gb\|BG224680 | 4e−44 |
| Strongyloides stercoralis | kp26a06.y1 | gb\|BE029191 | 3e−43 |
| Trichuris muris | Tm_ad_12H09_SKPL | gb\|BG577863 | 2e−24 |

C. elegans gene: C42D8.5

| Species | EST ID | Assession Number | E value |
|---|---|---|---|
| Anopheles gambiae | 17000687312136 | gb\|BM586309 | 1e−24 |
| Anopheles gambiae | 17000687442312 | gb\|BM593385 | 1e−20 |
| Anopheles gambiae | 17000687147222 | gb\|BM615549 | 3e−20 |
| Anopheles gambiae | 17000687284463 | gb\|BM640720 | 6e−20 |
| Anopheles gambiae | 17000687388771 | gb\|BM591666 | 6e−20 |
| Anopheles gambiae | 17000687076415 | gb\|BM605493 | 1e−18 |
| Anopheles gambiae | 17000687306814 | gb\|BM641014 | 1e−17 |
| Anopheles gambiae | 17000687321320 | gb\|BM587522 | 1e−16 |
| Anopheles gambiae | 17000687140860 | gb\|BM614061 | 5e−14 |
| Anopheles gambiae | 17000687383033 | gb\|BM590327 | 9e−13 |
| Anopheles gambiae | 17000668455074 | gb\|BM592015 | 8e−11 |
| Anopheles gambiae | 17000687446260 | gb\|BM595069 | 6e−06 |
| Anopheles gambiae | 17000687498456 | gb\|BM628360 | 2e−05 |
| Anopheles gambiae | 17000687317872 | gb\|BM646088 | 3e−04 |
| Amblyomma variegatum | EST576720 | gb\|BM290186 | 1e−23 |
| Apis mellifera | BB170014A10G05.5 | gb\|BI509028 | 9e−26 |
| Apis mellifera | BB170005B10F11.5 | gb\|BI504652 | 4e−13 |
| Apis mellifera | BB170029A10E09.5 | gb\|BI509998 | 8e−11 |
| Bombyx mori | AU004618 | dbj\|AU004618 | 2e−17 |
| Bombyx mori | AU005275 | dbj\|AU005275 | 2e−11 |
| Bombyx mori | AU004718 | dbj\|AU004718 | 4e−06 |
| Manduca sexta | EST816 | gb\|BE015590 | 3e−20 |
| Meloidogyne arenaria | rm04g06.y1 | gb\|BI501765 | 4e−41 |
| Meloidogyne incognita | rb11d01.y1 | gb\|BM881559 | 8e−41 |
| Meloidogyne incognita | rb18a12.y1 | gb\|BM880769 | 3e−41 |
| Meloidogyne incognita | rb26b04.y1 | gb\|BM882125 | 7e−40 |
| Meloidogyne javanica | rk44e09.y1 | gb\|BG735807 | 6e−38 |
| Meloidogyne arenaria | rm47b10.y1 | gb\|BI747899 | 2e−35 |
| Meloidogyne incognita | rb26c05.y1 | gb\|BM882137 | 2e−35 |
| Meloidogyne hapla | rc34h03.y1 | gb\|BM902335 | 9e−26 |
| Parastrongyloides trichosuri | kx21e10.y1 | gb\|BI451241 | 6e−33 |
| Pristionchus pacificus | rs54d09.y1 | gb\|AW114662 | 3e−39 |
| Trichinella spiralis | ps52g05.y1 | gb\|BG520845 | 1e−15 |
| Trichuris muris | Tm_ad_35E08_SKPL | gb\|BM277122 | 6e−15 |
| Trichuris muris | Tm_ad_31D02_SKPL | gb\|BM174595 | 9e−13 |

C. elegans gene: ZK430.8

| | | Assession | |
|---|---|---|---|
| Species | EST ID | Number | E value |
| Anopheles gambiae | 17000687491429 | gb\|BM624505 | 3e−25 |
| Anopheles gambiae | 17000687503479 | gb\|BM597766 | 9e−23 |
| Anopheles gambiae | 17000687144729 | gb\|BM614748 | 2e−22 |
| Anopheles gambiae | 17000659431849 | gb\|BM584934 | 2e−21 |
| Anopheles gambiae | 17000687475690 | gb\|BM595494 | 8e−21 |
| Anopheles gambiae | 17000687085569 | gb\|BM607770 | 2e−20 |
| Anopheles gambiae | 17000687385695 | gb\|BM655137 | 4e−19 |
| Aedes aegypti | AEMTAN84 | gb\|AI650118 | 1e−22 |
| Aedes aegypti | AEMTBE10 | gb\|AI657546 | 2e−21 |
| Amblyomma | EST576420 | gb\|BM289886 | 2e−47 |
| variegatum | | | |
| Amblyomma | EST576491 | gb\|BM289957 | 1e−45 |
| variegatum | | | |
| Bombyx mori | AU005825 | dbj\|AU005825 | 1e−24 |
| Brugia malayi | BSBmL3SZ44P22SK | gb\|AI723670 | 8e−40 |
| Brugia malayi | SWAMCAC30E11SK | gb\|AI784735 | 3e−26 |
| Brugia malayi | SWAMCA791SK | gb\|W69058 | 2e−19 |
| Heterodera glycines | ro60g11.y3 | gb\|BI396718 | 1e−27 |
| Meloidogyne hapla | rc06f11.y1 | gb\|BM883419 | 1e−36 |
| Strongyloides ratti | kt33e05.y1 | gb\|BI073673 | 5e−31 |
| Strongyloides | kq05g08.y1 | gb\|BG227360 | 5e−72 |
| stercoralis | | | |
| Trichinella spiralis | ps41e07.y1 | gb\|BG353679 | 3e−28 |

C. elegans gene: W08F4.6

| Species | EST ID | Assession Number | E value |
|---|---|---|---|
| Brugia malayi | SWYACAL11B04SK | gb\|BE758466 | e−104 |
| Brugia malayi | SWYD25CAU09E12SK | gb\|AW352455 | 2e−93 |
| Brugia malayi | SWYACAL10F05SK | gb\|BE758438 | 1e−86 |
| Brugia malayi | SWMFCA2071SK | gb\|AA480716 | 1e−77 |
| Brugia malayi | SWMFCA2926SK | gb\|AA598365 | 1e−77 |
| Brugia malayi | SWMFCA2164SK | gb\|AA283595 | 9e−59 |
| Brugia malayi | SWAMCA1093SK | gb\|AA032101 | 1e−51 |
| Brugia malayi | RRAMCA1520SK | gb\|AA430774 | 2e−44 |
| Brugia malayi | RRAMCA2132SK | gb\|AI574633 | 6e−05 |
| Onchocerca volvulus | SWOv3MCAM55E04SK | gb\|BG310588 | e−121 |
| Onchocerca volvulus | SWOvAFCAP46H10SK | gb\|BE949537 | 4e−97 |
| Onchocerca volvulus | SWOv3MCAM54B10SK | gb\|BF918270 | 1e−84 |
| Onchocerca volvulus | SWOv3MCAM53A07SK | gb\|BF824723 | 1e−74 |
| Onchocerca volvulus | SWOv3MCAM51C05SK | gb\|BF727588 | 3e−74 |
| Onchocerca volvulus | SWOv3MCAM52D06SK | gb\|BF824670 | 4e−64 |
| Onchocerca volvulus | SWOv3MCAM51F03SK | gb\|BF727618 | 2e−53 |
| Onchocerca volvulus | SWOv3MCAM61A06SK | gb\|BG809067 | 7e−52 |
| Onchocerca volvulus | SWOvAFCAP49D03SK | gb\|BF199456 | 9e−46 |
| Onchocerca volvulus | SWOvAFCAP48B10SK | gb\|BF064382 | 3e−39 |
| Onchocerca volvulus | SWOvAFCAP35C02SK | gb\|AW562139 | 1e−93 |
| Onchocerca volvulus | SWOv3MCAM28D06SK | gb\|AI692125 | 5e−91 |
| Onchocerca volvulus | SWOvMfCAR10H04SK | gb\|AW874896 | 1e−87 |
| Onchocerca volvulus | SWOv3MCAM38A10SK | gb\|AW313047 | 2e−75 |
| Onchocerca volvulus | SWOvAFCAP34D11SK | gb\|AW562114 | 1e−73 |
| Onchocerca volvulus | SWOv3MCAM12D08SK | gb\|AI322100 | 3e−72 |
| Onchocerca volvulus | SWOv3MCAM38E03SK | gb\|AW313086 | 6e−71 |
| Onchocerca volvulus | SWOv3MCAM26F11SK | gb\|AI670476 | 5e−63 |
| Onchocerca volvulus | SWOvAFCAP16F04SK | gb\|AI318006 | 1e−51 |
| Onchocerca volvulus | SWOvAFCAP28E11SK | gb\|AI540006 | 1e−50 |
| Onchocerca volvulus | SWOvAFCAP35E04SK | gb\|AW562163 | 1e−50 |
| Onchocerca volvulus | SWOv3MCAM37F09SK | gb\|AW313003 | 2e−50 |
| Onchocerca volvulus | SWOv3MCAM23B03SK | gb\|AI603814 | 5e−39 |
| Onchocerca volvulus | SWOv3MCAM37E10SK | gb\|AW312994 | 5e−35 |
| Onchocerca volvulus | SWOvMfCAR04C04SK | gb\|AI381166 | 9e−32 |
| Onchocerca volvulus | SWOvAFCAP15A02SK | gb\|AI771077 | 5e−31 |
| Onchocerca volvulus | SWOvAFCAP25C08SK | gb\|AI368292 | 4e−22 |
| Onchocerca volvulus | SWOv3MCA1962SK | gb\|AA618916 | 4e−97 |
| Onchocerca volvulus | SWOv3MCA705SK | gb\|AA294494 | 6e−61 |
| Onchocerca volvulus | SWOv3MCA1335SK | gb\|AA293981 | 3e−56 |
| Onchocerca volvulus | SWOv3MCA107SK | gb\|AA293944 | 4e−53 |
| Onchocerca volvulus | SWOv3MCA1898SK | gb\|AA618908 | 1e−20 |
| Parastrongyloides trichosuri | kx60h05.y1 | gb\|BM346811 | 6e−89 |
| Parastrongyloides trichosuri | kx72f10.y1 | gb\|BM513102 | 5e−55 |
| Parastrongyloides trichosuri | kx76e12.y1 | gb\|BM812715 | 4e−47 |
| Parastrongyloides trichosuri | kx23a05.y1 | gb\|BI451341 | 2e−31 |
| Strongyloides stercoralis | kp97h03.y1 | gb\|BG227161 | 2e−84 |
| Strongyloides stercoralis | kq65c07.y1 | gb\|BF015176 | 3e−66 |
| Strongyloides stercoralis | kq38c10.y1 | gb\|BE580196 | 4e−65 |

C. elegans gene: C11H1.3

| | | Assession | |
|---|---|---|---|
| Species | EST ID | Number | E value |
| Anopheles gambiae | 17000687504402 | gb\|BM632519 | 8e−14 |
| Ascaris suum | kj59a10.y1 | gb\|BM569296 | 2e−11 |
| Brugia malayi | BSBmL3SZ15J6SK | gb\|AI783191 | 7e−37 |
| Brugia malayi | MBAFCW6C02T3 | gb\|AA842256 | 7e−19 |
| Brugia malayi | BSBmL3SZ45E24SK | gb\|AI723685 | 4e−10 |
| Trichinella spiralis | ps40b05.y1 | gb\|BG353953 | 5e−11 |
| Trichuris muris | Tm_ad_08A04_SKPL | gb\|BG577585 | 1e−19 |

C. elegans gene: T23F2.1

| Species | EST ID | Assession Number | E value |
|---|---|---|---|
| Meloidogyne incognita | rb19d10.y1 | gb\|BM880892 | 6e−65 |
| Meloidogyne hapla | rc09a08.y1 | gb\|BM883631 | 1e−57 |
| Meloidogyne hapla | rc19d06.y1 | gb\|BM884107 | 5e−57 |
| Meloidogyne hapla | rc80b07.y1 | gb\|BQ627436 | 2e−57 |
| Meloidogyne javanica | rk89h09.y1 | gb\|BI744669 | 3e−52 |
| Ostertagia ostertagi | ph80a06.y1 | gb\|BQ457577 | 5e−36 |
| Pristionchus pacificus | rs74f08.y1 | gb\|BI703617 | 4e−13 |
| Pristionchus pacificus | rs73e09.y1 | gb\|BI703595 | 2e−10 |
| Strongyloides stercoralis | kq31b05.y1 | gb\|BE579591 | 7e−75 |
| Strongyloides stercoralis | kp96a08.y1 | gb\|BG227048 | 1e−22 |

C. elegans gene: R07E4.6

| | | Assession | |
|---|---|---|---|
| Species | EST ID | Number | E value |
| Anopheles gambiae | 17000687445042 | gb\|BM594887 | 3e−70 |
| Amblyomma variegatum | EST574524 | gb\|BM291982 | 9e−68 |
| Globodera rostochiensis | rr28f03.y1 | gb\|BM355711 | 3e−63 |
| Meloidogyne hapla | rf47g07.y1 | gb\|BQ836585 | 9e−76 |
| Meloidogyne hapla | rf44f11.y1 | gb\|BQ836331 | 4e−73 |
| Meloidogyne arenaria | rm39a02.y1 | gb\|BI747341 | 5e−66 |
| Meloidogyne hapla | rf37e06.y1 | gb\|BQ837060 | 4e−66 |
| Meloidogyne hapla | rf45g11.y1 | gb\|BQ836426 | 4e−66 |
| Meloidogyne hapla | rf50g04.y2 | gb\|BU094140 | 4e−66 |
| Meloidogyne hapla | rf53g05.y2 | gb\|BU094227 | 4e−66 |
| Meloidogyne hapla | rf58d10.y1 | gb\|BQ835832 | 4e−66 |
| Meloidogyne hapla | rf67f06.y1 | gb\|BU094368 | 4e−66 |
| Meloidogyne arenaria | rm02d02.y1 | gb\|BI501589 | 1e−65 |
| Meloidogyne arenaria | rm02f05.y1 | gb\|BI501609 | 1e−65 |
| Meloidogyne arenaria | rm02g08.y1 | gb\|BI501619 | 2e−65 |
| Meloidogyne arenaria | rm02h06.y1 | gb\|BI501626 | 3e−65 |
| Meloidogyne arenaria | rm03b06.y1 | gb\|BI501643 | 1e−65 |
| Meloidogyne arenaria | rm03f11.y1 | gb\|BI501681 | 1e−65 |
| Meloidogyne arenaria | rm04b02.y1 | gb\|BI501716 | 1e−65 |
| Meloidogyne arenaria | rm04b09.y1 | gb\|BI501721 | 9e−65 |
| Meloidogyne arenaria | rm04c09.y1 | gb\|BI501729 | 4e−65 |
| Meloidogyne arenaria | rm04c12.y1 | gb\|BI501732 | 1e−65 |
| Meloidogyne arenaria | rm04g02.y1 | gb\|BI501762 | 1e−65 |
| Meloidogyne arenaria | rm04g05.y1 | gb\|BI501764 | 1e−65 |
| Meloidogyne arenaria | rm05d02.y1 | gb\|BI501806 | 2e−65 |
| Meloidogyne arenaria | rm06f06.y1 | gb\|BI501906 | 3e−65 |
| Meloidogyne arenaria | rm07a04.y1 | gb\|BI501933 | 3e−65 |
| Meloidogyne arenaria | rm07a08.y1 | gb\|BI501937 | 9e−65 |
| Meloidogyne arenaria | rm07f06.y1 | gb\|BI501985 | 3e−65 |
| Meloidogyne arenaria | rm13b12.y1 | gb\|BI862855 | 1e−65 |
| Meloidogyne arenaria | rm14g07.y1 | gb\|BI862971 | 1e−65 |
| Meloidogyne arenaria | rm16g11.y1 | gb\|BI863129 | 1e−65 |
| Meloidogyne arenaria | rm17c08.y1 | gb\|BI745704 | 1e−65 |
| Meloidogyne arenaria | rm18d04.y1 | gb\|BI745781 | 1e−65 |
| Meloidogyne arenaria | rm18e04.y1 | gb\|BI745792 | 9e−65 |
| Meloidogyne arenaria | rm19f11.y1 | gb\|BI745875 | 3e−65 |
| Meloidogyne arenaria | rm21a07.y1 | gb\|BI745964 | 1e−65 |
| Meloidogyne arenaria | rm21d04.y1 | gb\|BI745991 | 1e−65 |
| Meloidogyne arenaria | rm23a02.y1 | gb\|BI746123 | 1e−65 |
| Meloidogyne arenaria | rm23c11.y1 | gb\|BI746144 | 1e−65 |
| Meloidogyne arenaria | rm26a06.y1 | gb\|BI746349 | 3e−65 |
| Meloidogyne arenaria | rm28c08.y1 | gb\|BI746525 | 3e−65 |
| Meloidogyne arenaria | rm29g05.y1 | gb\|BI746637 | 1e−65 |
| Meloidogyne arenaria | rm30f07.y1 | gb\|BI746703 | 3e−65 |
| Meloidogyne arenaria | rm31d04.y1 | gb\|BI746759 | 1e−65 |
| Meloidogyne arenaria | rm31f01.y1 | gb\|BI746774 | 1e−65 |
| Meloidogyne arenaria | rm32a08.y1 | gb\|BI746802 | 1e−65 |
| Meloidogyne arenaria | rm33c02.y1 | gb\|BI746890 | 1e−65 |
| Meloidogyne arenaria | rm35a06.y1 | gb\|BI747032 | 3e−65 |
| Meloidogyne arenaria | rm35d09.y1 | gb\|BI747063 | 1e−65 |
| Meloidogyne arenaria | rm37e04.y1 | gb\|BI747228 | 9e−65 |
| Meloidogyne arenaria | rm39h04.y1 | gb\|BI747413 | 1e−65 |
| Meloidogyne arenaria | rm40d11.y1 | gb\|BI747460 | 1e−65 |
| Meloidogyne arenaria | rm40f08.y1 | gb\|BI747479 | 3e−65 |
| Meloidogyne arenaria | rm41c05.y1 | gb\|BI747526 | 1e−65 |
| Meloidogyne arenaria | rm45b05.y1 | gb\|BI747647 | 7e−65 |
| Meloidogyne arenaria | rm45b12.y1 | gb\|BI747653 | 5e−65 |
| Meloidogyne arenaria | rm45e12.y1 | gb\|BI747681 | 1e−65 |
| Meloidogyne arenaria | rm45h12.y1 | gb\|BI747711 | 1e−65 |
| Meloidogyne arenaria | rm46g06.y1 | gb\|BI747868 | 9e−65 |
| Meloidogyne arenaria | rm47a11.y1 | gb\|BI747891 | 4e−65 |
| Meloidogyne arenaria | rm47c05.y1 | gb\|BI747905 | 1e−65 |
| Meloidogyne arenaria | rm47c09.y1 | gb\|BI747909 | 1e−65 |
| Meloidogyne arenaria | rm47d03.y1 | gb\|BI747914 | 1e−65 |
| Meloidogyne hapla | rf26f11.y1 | gb\|BQ837462 | 2e−65 |
| Meloidogyne incognita | ra84f09.y1 | gb\|BM773674 | 9e−65 |
| Meloidogyne incognita | ra84f12.y1 | gb\|BM773677 | 1e−65 |
| Meloidogyne incognita | ra92d12.y1 | gb\|BM774355 | 1e−65 |
| Meloidogyne incognita | ra93c08.y1 | gb\|BM774423 | 4e−65 |
| Meloidogyne incognita | ra95b12.y1 | gb\|BM774573 | 1e−65 |
| Meloidogyne incognita | ra96h04.y1 | gb\|BM774720 | 1e−65 |
| Meloidogyne incognita | ra96h10.y1 | gb\|BM774726 | 9e−65 |
| Meloidogyne incognita | ra99c02.y1 | gb\|BM882309 | 1e−65 |
| Meloidogyne incognita | ra99d03.y1 | gb\|BM882321 | 1e−65 |
| Meloidogyne incognita | ra99h05.y1 | gb\|BM882366 | 1e−65 |
| Meloidogyne incognita | rb02h10.y1 | gb\|BM882545 | 9e−65 |
| Meloidogyne incognita | rb03a01.y1 | gb\|BM882548 | 1e−65 |
| Meloidogyne incognita | rb06c07.y1 | gb\|BM881126 | 7e−65 |
| Meloidogyne incognita | rb08d08.y1 | gb\|BM881299 | 1e−65 |
| Meloidogyne incognita | rb09d03.y1 | gb\|BM881380 | 9e−65 |
| Meloidogyne incognita | rb11b10.y1 | gb\|BM881544 | 1e−65 |
| Meloidogyne incognita | rb11f12.y1 | gb\|BM881592 | 1e−65 |
| Meloidogyne incognita | rb12g03.y1 | gb\|BM881679 | 1e−65 |
| Meloidogyne incognita | rb16b01.y1 | gb\|BM880596 | 1e−65 |
| Meloidogyne incognita | rb19e08.y1 | gb\|BM880901 | 1e−65 |
| Meloidogyne incognita | rb20d04.y1 | gb\|BM880267 | 1e−65 |
| Meloidogyne incognita | rb23a12.y1 | gb\|BM880504 | 4e−65 |
| Meloidogyne incognita | rb23g01.y1 | gb\|BM880561 | 9e−65 |
| Meloidogyne incognita | rb24b04.y1 | gb\|BM881952 | 4e−65 |
| Meloidogyne incognita | rb28d01.y1 | gb\|BM882671 | 1e−65 |
| Meloidogyne javanica | rk45d01.y1 | gb\|BG735927 | 4e−65 |
| Meloidogyne javanica | rk49a03.y1 | gb\|BG736042 | 5e−65 |
| Meloidogyne javanica | rk49b09.y1 | gb\|BG736055 | 1e−65 |
| Meloidogyne javanica | rk53a04.y1 | gb\|BG736196 | 4e−65 |
| Meloidogyne javanica | rk53c10.y1 | gb\|BG736217 | 4e−65 |
| Meloidogyne javanica | rk54h02.y1 | gb\|BG736324 | 4e−65 |
| Meloidogyne javanica | rk57b01.y1 | gb\|BG736436 | 4e−65 |
| Meloidogyne javanica | rk58f10.y1 | gb\|BG736536 | 4e−65 |
| Meloidogyne javanica | rk66b08.y1 | gb\|BG737056 | 4e−65 |
| Meloidogyne javanica | rk89g01.y1 | gb\|BI744652 | 5e−65 |
| Meloidogyne arenaria | rm18g05.y1 | gb\|BI745808 | 3e−64 |
| Meloidogyne arenaria | rm24a02.y1 | gb\|BI746195 | 1e−64 |
| Meloidogyne arenaria | rm26h04.y1 | gb\|BI746420 | 2e−64 |
| Meloidogyne arenaria | rm27c07.y1 | gb\|BI746449 | 2e−64 |
| Meloidogyne arenaria | rm37e08.y1 | gb\|BI747232 | 3e−64 |
| Meloidogyne arenaria | rm40h05.y1 | gb\|BI747498 | 2e−64 |
| Meloidogyne arenaria | rm42g03.y1 | gb\|BI747618 | 3e−64 |
| Meloidogyne incognita | ra83d08.y1 | gb\|BM773565 | 3e−64 |
| Meloidogyne incognita | ra89e11.y1 | gb\|BM774108 | 1e−64 |
| Meloidogyne incognita | ra90b04.y1 | gb\|BM774157 | 5e−64 |
| Meloidogyne incognita | rb08b07.y1 | gb\|BM881275 | 5e−64 |
| Meloidogyne incognita | rb12c06.y1 | gb\|BM881640 | 8e−64 |
| Meloidogyne incognita | rb13f04.y1 | gb\|BM881755 | 5e−64 |
| Meloidogyne incognita | rb14a12.y1 | gb\|BM881795 | 2e−64 |
| Meloidogyne incognita | rb15b06.y1 | gb\|BM881885 | 3e−64 |
| Meloidogyne incognita | rb20f11.y1 | gb\|BM880296 | 5e−64 |
| Meloidogyne incognita | rb30a09.y1 | gb\|BM882822 | 2e−64 |
| Meloidogyne incognita | rb30e01.y1 | gb\|BM882861 | 1e−64 |
| Meloidogyne javanica | rk45f07.y1 | gb\|BG735952 | 1e−64 |
| Meloidogyne javanica | rk53d07.y1 | gb\|BG736223 | 5e−64 |
| Meloidogyne javanica | rk57h07.y1 | gb\|BG736497 | 1e−64 |
| Meloidogyne javanica | rk60a03.y1 | gb\|BG736616 | 5e−64 |
| Meloidogyne javanica | rk60e11.y1 | gb\|BG736654 | 5e−64 |
| Meloidogyne javanica | rk62d09.y1 | gb\|BG736793 | 5e−64 |
| Meloidogyne javanica | rk64h06.y1 | gb\|BG736964 | 5e−64 |
| Meloidogyne javanica | rl01a06.y1 | gb\|BI863144 | 5e−64 |
| Meloidogyne arenaria | rm05c01.y1 | gb\|BI501797 | 2e−63 |
| Meloidogyne arenaria | rm34e11.y1 | gb\|BI746992 | 1e−63 |
| Meloidogyne hapla | rc05b08.y1 | gb\|BM883283 | 2e−63 |
| Meloidogyne hapla | rc08c12.y1 | gb\|BM883572 | 2e−63 |
| Meloidogyne hapla | rc32b09.y1 | gb\|BM902099 | 2e−63 |
| Meloidogyne hapla | rc42b02.y1 | gb\|BM900837 | 1e−63 |
| Meloidogyne hapla | rc44g03.y1 | gb\|BM901068 | 1e−63 |
| Meloidogyne hapla | rc47g08.y1 | gb\|BM901701 | 2e−63 |
| Meloidogyne hapla | rc48e03.y1 | gb\|BM901766 | 1e−63 |
| Meloidogyne hapla | rc49g09.y1 | gb\|BM901884 | 2e−63 |
| Meloidogyne incognita | ra84b02.y1 | gb\|BM773624 | 5e−63 |
| Meloidogyne incognita | ra84b07.y1 | gb\|BM773629 | 5e−63 |
| Meloidogyne incognita | ra84g09.y1 | gb\|BM773686 | 4e−63 |
| Meloidogyne incognita | ra85b02.y1 | gb\|BM773712 | 5e−63 |
| Meloidogyne incognita | ra85f05.y1 | gb\|BM773761 | 5e−63 |
| Meloidogyne incognita | ra86d03.y1 | gb\|BM773827 | 5e−63 |
| Meloidogyne incognita | ra86g11.y1 | gb\|BM773868 | 1e−63 |
| Meloidogyne incognita | ra87b02.y1 | gb\|BM773892 | 2e−63 |
| Meloidogyne incognita | ra88b11.y1 | gb\|BM773988 | 5e−63 |
| Meloidogyne incognita | ra88h01.y1 | gb\|BM774046 | 5e−63 |
| Meloidogyne incognita | ra88h12.y1 | gb\|BM774056 | 5e−63 |
| Meloidogyne incognita | ra91a06.y1 | gb\|BM774236 | 5e−63 |
| Meloidogyne incognita | ra92c01.y1 | gb\|BM774335 | 5e−63 |
| Meloidogyne incognita | ra92e09.y1 | gb\|BM774363 | 5e−63 |
| Meloidogyne incognita | ra93f06.y1 | gb\|BM774450 | 1e−63 |
| Meloidogyne incognita | ra94h05.y1 | gb\|BM774549 | 5e−63 |
| Meloidogyne incognita | ra95g06.y1 | gb\|BM774622 | 3e−63 |
| Meloidogyne incognita | ra95h06.y1 | gb\|BM774634 | 5e−63 |
| Meloidogyne incognita | ra96g11.y1 | gb\|BM774715 | 5e−63 |
| Meloidogyne incognita | ra97d11.y1 | gb\|BM774770 | 2e−63 |
| Meloidogyne incognita | ra97f05.y1 | gb\|BM774785 | 5e−63 |
| Meloidogyne incognita | ra97g12.y1 | gb\|BM774803 | 4e−63 |
| Meloidogyne incognita | ra98c09.y1 | gb\|BM774842 | 5e−63 |
| Meloidogyne incognita | ra98e05.y1 | gb\|BM774861 | 5e−63 |
| Meloidogyne incognita | ra99f05.y1 | gb\|BM882343 | 5e−63 |
| Meloidogyne incognita | rb01c12.y1 | gb\|BM882405 | 5e−63 |
| Meloidogyne incognita | rb01h09.y1 | gb\|BM882460 | 5e−63 |
| Meloidogyne incognita | rb02c02.y1 | gb\|BM882484 | 5e−63 |
| Meloidogyne incognita | rb05e03.y1 | gb\|BM881054 | 5e−63 |
| Meloidogyne incognita | rb06a03.y1 | gb\|BM881099 | 2e−63 |
| Meloidogyne incognita | rb06d12.y1 | gb\|BM881142 | 5e−63 |
| Meloidogyne incognita | rb07c08.y1 | gb\|BM881207 | 5e−63 |
| Meloidogyne incognita | rb07g10.y1 | gb\|BM881252 | 5e−63 |
| Meloidogyne incognita | rb08c10.y1 | gb\|BM881289 | 5e−63 |
| Meloidogyne incognita | rb08h09.y1 | gb\|BM881343 | 5e−63 |
| Meloidogyne incognita | rb09f08.y1 | gb\|BM881408 | 5e−63 |
| Meloidogyne incognita | rb09g04.y1 | gb\|BM881416 | 5e−63 |
| Meloidogyne incognita | rb11a06.y1 | gb\|BM881529 | 5e−63 |
| Meloidogyne incognita | rb12h03.y1 | gb\|BM881691 | 5e−63 |
| Meloidogyne incognita | rb12h10.y1 | gb\|BM881697 | 5e−63 |
| Meloidogyne incognita | rb14g02.y1 | gb\|BM881850 | 2e−63 |
| Meloidogyne incognita | rb14g04.y1 | gb\|BM881852 | 4e−63 |
| Meloidogyne incognita | rb14g06.y1 | gb\|BM881854 | 1e−63 |
| Meloidogyne incognita | rb15a03.y1 | gb\|BM881872 | 5e−63 |
| Meloidogyne incognita | rb16b05.y1 | gb\|BM880600 | 5e−63 |
| Meloidogyne incognita | rb16h07.y1 | gb\|BM880663 | 2e−63 |
| Meloidogyne incognita | rb17b06.y1 | gb\|BM880685 | 5e−63 |
| Meloidogyne incognita | rb18d06.y1 | gb\|BM880798 | 5e−63 |
| Meloidogyne incognita | rb22e08.y1 | gb\|BM880457 | 5e−63 |
| Meloidogyne incognita | rb24f01.y1 | gb\|BM881994 | 5e−63 |
| Meloidogyne incognita | rb25f11.y1 | gb\|BM882090 | 3e−63 |
| Meloidogyne incognita | rb25g03.y1 | gb\|BM882094 | 5e−63 |
| Meloidogyne incognita | rb26d05.y1 | gb\|BM882147 | 2e−63 |
| Meloidogyne incognita | rb26g11.y1 | gb\|BM882186 | 2e−63 |
| Meloidogyne incognita | rb27a05.y1 | gb\|BM882203 | 5e−63 |
| Meloidogyne incognita | rb29f01.y1 | gb\|BM882780 | 1e−63 |
| Meloidogyne incognita | rb30a01.y1 | gb\|BM882816 | 5e−63 |
| Meloidogyne incognita | rb30d12.y1 | gb\|BM882860 | 3e−63 |
| Meloidogyne incognita | rb31h02.y1 | gb\|BM882986 | 5e−63 |
| Meloidogyne javanica | rk43a07.y1 | gb\|BG735712 | 4e−63 |
| Meloidogyne javanica | rk43e09.y1 | gb\|BG735752 | 2e−63 |
| Meloidogyne javanica | rk43e12.y1 | gb\|BG735755 | 2e−63 |
| Meloidogyne javanica | rk53e04.y1 | gb\|BG736231 | 2e−63 |
| Meloidogyne javanica | rk53h02.y1 | gb\|BG736256 | 2e−63 |
| Meloidogyne javanica | rk60b04.y1 | gb\|BG736624 | 2e−63 |
| Meloidogyne javanica | rk62f12.y1 | gb\|BG736812 | 1e−63 |
| Meloidogyne javanica | rk63c05.y1 | gb\|BG736846 | 2e−63 |
| Meloidogyne javanica | rk65e08.y1 | gb\|BG737010 | 1e−63 |
| Meloidogyne javanica | rk65g02.y1 | gb\|BG737025 | 3e−63 |
| Meloidogyne javanica | rk66f07.y1 | gb\|BG737097 | 5e−63 |
| Meloidogyne javanica | rk66g09.y1 | gb\|BG737108 | 2e−63 |
| Meloidogyne javanica | rk66g10.y1 | gb\|BG737109 | 1e−63 |
| Meloidogyne javanica | rk72b09.y1 | gb\|BI143215 | 3e−63 |
| Meloidogyne javanica | rk81d09.y3 | gb\|BI745501 | 5e−63 |
| Meloidogyne javanica | rk81d10.y3 | gb\|BI745502 | 2e−63 |
| Meloidogyne javanica | rk81f12.y3 | gb\|BI745518 | 5e−63 |
| Meloidogyne javanica | rk81g01.y3 | gb\|BI745519 | 5e−63 |
| Meloidogyne javanica | rk89g05.y1 | gb\|BI744656 | 5e−63 |
| Meloidogyne javanica | rk90d08.y1 | gb\|BI744549 | 5e−63 |
| Meloidogyne javanica | rk90g10.y1 | gb\|BI744581 | 5e−63 |
| Meloidogyne javanica | rk90g11.y1 | gb\|BI744582 | 2e−63 |
| Meloidogyne javanica | rk91b12.y1 | gb\|BI744693 | 5e−63 |
| Meloidogyne javanica | rk92a03.y1 | gb\|BI744754 | 3e−63 |
| Meloidogyne javanica | rk97e03.y1 | gb\|BI745201 | 5e−63 |
| Meloidogyne javanica | rk99c07.y1 | gb\|BI745347 | 5e−63 |
| Meloidogyne javanica | rk99h08.y1 | gb\|BI745392 | 2e−63 |
| Meloidogyne javanica | rl02d04.y1 | gb\|BI863247 | 2e−63 |
| Meloidogyne javanica | rl05d03.y1 | gb\|BI863458 | 5e−63 |
| Strongyloides ratti | ku14g06.y1 | gb\|BQ091242 | 2e−65 |
| Strongyloides stercoralis | kp53h07.y1 | gb\|BE224025 | 5e−84 |
| Strongyloides stercoralis | kq04b03.y1 | gb\|BG227238 | 2e−76 |
| Strongyloides stercoralis | kq18e12.y1 | gb\|BG226203 | 4e−66 |

C. elegans gene: F25B4.6

| | | Assession | |
|---|---|---|---|
| Species | EST ID | Number | E value |
| Anopheles gambiae | 17000687438069 | gb\|BM592421 | 4e−44 |
| Apis mellifera | BB170031B10F03.5 | gb\|BI505742 | 1e−45 |
| Bombyx mori bra | AV400509 | dbj\|AV400509 | 4e−30 |
| Necator americanus | Na_L3_09H09_SAC | gb\|BU086573 | 4e−54 |
| Strongyloides ratti | kt71f08.y1 | gb\|BI323469 | 1e−24 |

C. elegans gene: C45B2.7

| | | Assession | E |
|---|---|---|---|
| Species | EST ID | Number | value |
| Anopheles gambiae | 17000687494627 | gb\|BM626221 | 5e−16 |
| Ancylostoma caninum | pb30d08.y1 | gb\|BM130388 | 3e−16 |
| Ancylostoma caninum | pb44h12.y1 | gb\|BQ666635 | 9e−15 |
| Ancylostoma caninum | pb09e11.y1 | gb\|BI744487 | 1e−13 |
| Ancylostoma caninum | pb31h06.y1 | gb\|BQ125114 | 1e−13 |
| Ancylostoma caninum | pb31h07.y1 | gb\|BQ125115 | 1e−13 |
| Ancylostoma caninum | pb07d02.y1 | gb\|BI744318 | 5e−12 |
| Ancylostoma caninum | pb30b09.y1 | gb\|BM130369 | 9e−12 |
| Ancylostoma caninum | pb02b07.y1 | gb\|BF250603 | 4e−10 |
| Ascaris suum | kh68b11.y1 | gb\|BM033843 | 1e−18 |
| Ascaris suum | kk20b06.y1 | gb\|BQ096501 | 5e−13 |
| Ascaris suum | kk27h06.y1 | gb\|BQ381130 | 4e−12 |
| Brugia malayi | SWYD25CAU08D12SK | gb\|AW257677 | 1e−14 |
| Brugia malayi | kb34c04.y1 | gb\|BU917772 | 3e−11 |
| Manduca sexta | EST292 | gb\|AI187503 | 3e−17 |
| Meloidogyne arenaria | rm35b03.y1 | gb\|BI747039 | 1e−54 |
| Meloidogyne javanica | rk99c03.y1 | gb\|BI745344 | 3e−31 |
| Meloidogyne hapla | rc59a10.y1 | gb\|BM952341 | 4e−12 |
| Meloidogyne arenaria | rm32d04.y1 | gb\|BI746830 | 1e−10 |
| Onchocerca volvulus | SWOvAMCAQ10E05SK | gb\|BE202282 | 1e−15 |
| Ostertagia ostertagi | Oo_ad_02F04_LambdaGT11FO | gb\|BG734000 | 4e−13 |
| Parastrongyloides | kx48f05.y1 | gb\|BI863807 | 3e−11 |
| trichosuri | | | |
| Strongyloides stercoralis | kq39f03.y1 | gb\|BE580303 | 3e−21 |

C. elegans gene: C37C3.3

| | | Assession | |
|---|---|---|---|
| Species | EST ID | Number | E value |
| Aedes aegypti | EST | gb\|BM144106 | 2e−11 |
| Anopheles gambiae | 17000687367709 | gb\|BM648797 | 1e−43 |
| Anopheles gambiae | 17000687384243 | gb\|BM590770 | 1e−41 |
| Anopheles gambiae | 17000687447857 | gb\|BM621866 | 1e−41 |
| Ancylostoma | pj99f09.y1 | gb\|CA033302 | 4e−11 |
| caninum | | | |
| Amblyomma | EST577974 | gb\|BM291440 | 6e−45 |
| variegatum | | | |
| Bombyx mori | AU000259 | dbj\|AU000259 | 3e−50 |
| Bombyx mori | AV401044 | dbj\|AV401044 | 2e−42 |
| Bombyx mori | AU006392 | dbj\|AU006392 | 1e−40 |
| Haemonchus | Hc_d11_25E08_SKPL | gb\|BF423278 | 4e−47 |
| contortus | | | |
| Ancylostoma | pa46g09.y1 | gb\|AW735046 | 5e−27 |
| caninum | | | |
| Ancylostoma | pb03g12.y1 | gb\|BF250735 | 8e−23 |
| caninum | | | |
| Zeldia punctata | rp11c10.y1 | gb\|AW773524 | 1e−46 |
| Meloidogyne | rk17h04.y1 | gb\|BE578050 | 2e−38 |
| javanica | | | |
| Meloidogyne | rk52a07.y1 | gb\|BG736156 | 2e−24 |
| javanica | | | |
| Meloidogyne | rk66e08.y1 | gb\|BG737087 | 1e−24 |
| javanica | | | |
| Necator americanus | Na_ad_01F02_SAC | gb\|BG734490 | 2e−17 |
| Pristionchus | rt01d05.y2 | gb\|BM812517 | 2e−58 |
| pacificus | | | |
| Pristionchus | rt01d05.y1 | gb\|BM565711 | 3e−53 |
| pacificus | | | |
| Pristionchus | rs26a01.y1 | gb\|AI988844 | 6e−18 |
| pacificus | | | |
| Strongyloides | kp41g07.y1 | gb\|BE030342 | 7e−51 |
| stercoralis | | | |
| Strongyloides | kp18h06.y1 | gb\|AW588105 | 2e−39 |
| stercoralis | | | |
| Trichinella spiralis | pt34g08.y1 | gb\|BQ693409 | 1e−52 |
| Trichinella spiralis | ps21a08.y4 | gb\|BG731987 | 1e−50 |
| Trichinella spiralis | ps31d12.y1 | gb\|BG353562 | 3e−39 |
| Trichinella spiralis | pt13a05.y1 | gb\|BQ693271 | 9e−35 |
| Trichinella spiralis | ps31d12.y2 | gb\|BG438577 | 2e−29 |
| Trichuris muris | Tm_ad_29E03_SKPL | gb\|BM174441 | 2e−35 |
| Trichuris muris | Tm_ad_08B07_SKPL | gb\|BG577593 | 4e−35 |

C. elegans gene: F45G2.5

| | | Assession | |
|---|---|---|---|
| Species | EST ID | Number | E value |
| Ostertagia | ph79d04.y1 | gb\|BQ457535 | 6e−52 |
| ostertagi | | | |

C. elegans gene: K08B4.1

| | | Assession | |
|---|---|---|---|
| Species | EST ID | Number | E value |
| Brugia malayi | BSBmMFSZ22D12SK | gb\|AW013739 | 2e−59 |
| Brugia malayi | kb06e04.y1 | gb\|BM889162 | 7e−21 |
| Heterodera glycines | ro82c01.y1 | gb\|BI748790 | 5e−21 |
| Trichuris muris | Tm_ad_03F11_SKPL | gb\|BF169284 | 3e−15 |

C. elegans gene: ZK970.4

| Species | EST ID | Assession Number | E value |
|---|---|---|---|
| Caenorhabditis briggsae | | gb\|AC084593 | 1e−27 |
| Manduca sexta | | emb\|X67130 | 3e−33 |
| Anopheles gambiae | | emb\|Z69979 | 7e−31 |

C. elegans gene: H19M22.1

| Species | EST ID | Assession Number | E value |
|---|---|---|---|
| Globodera rostochiensis | rr35f05.y2 | gb\|BM343207 | 2e−13 |
| Ancylostoma caninum | pb02b10.y1 | gb\|BF250605 | 3e−17 |

C. elegans gene: ZK270.1

| | | Assession | E |
|---|---|---|---|
| Species | EST ID | Number | value |
| Anopheles gambiae | 17000687494627 | gb\|BM626221 | 1e−16 |
| Ancylostoma caninum | pb30d08.y1 | gb\|BM130388 | 9e−98 |
| Ancylostoma caninum | pb44h12.y1 | gb\|BQ666635 | 2e−90 |
| Ancylostoma caninum | pb31h06.y1 | gb\|BQ125114 | 6e−86 |
| Ancylostoma caninum | pb09e11.y1 | gb\|BI744487 | 5e−84 |
| Ancylostoma caninum | pb31h07.y1 | gb\|BQ125115 | 3e−84 |
| Ancylostoma caninum | pb07d02.y1 | gb\|BI744318 | 1e−79 |
| Ancylostoma caninum | pb30b09.y1 | gb\|BM130369 | 1e−78 |
| Ancylostoma caninum | pb29b03.y1 | gb\|BM130286 | 3e−75 |
| Ancylostoma caninum | pb57c05.y1 | gb\|BQ667670 | 4e−52 |
| Ancylostoma caninum | pb57d12.y1 | gb\|BQ667681 | 4e−52 |
| Ancylostoma caninum | pb46a01.y1 | gb\|BQ666692 | 2e−50 |
| Ancylostoma caninum | pb41h05.y1 | gb\|BQ666447 | 6e−11 |
| Ascaris suum | kh68b11.y1 | gb\|BM033843 | 7e−20 |
| Ascaris suum | kk20b06.y1 | gb\|BQ096501 | 6e−16 |
| Ascaris suum | kk27h06.y1 | gb\|BQ381130 | 1e−14 |
| Ascaris suum | kh95h02.y1 | gb\|BM285196 | 3e−12 |
| Brugia malayi | SWYD25CAU08D12SK | gb\|AW257677 | 3e−43 |
| Brugia malayi | kb13a04.y1 | gb\|BU781174 | 5e−19 |
| Globodera rostochiensis | rr59g08.y1 | gb\|BM344825 | 7e−18 |
| Ancylostoma caninum | pb02b07.y1 | gb\|BF250603 | 1e−13 |
| Litomosoides | JALsL3C008SAC | gb\|AW152689 | 3e−16 |
| sigmodontis | | | |
| Manduca sexta | EST292 | gb\|AI187503 | 1e−17 |
| Meloidogyne hapla | rc61f09.y1 | gb\|BQ090105 | 2e−17 |
| Meloidogyne hapla | rc59a10.y1 | gb\|BM952341 | 8e−16 |
| Meloidogyne incognita | MD0882 | gb\|BE240858 | 6e−14 |
| Meloidogyne arenaria | rm33a11.y1 | gb\|BI746878 | 2e−12 |
| Meloidogyne hapla | rc55a06.y1 | gb\|BM952077 | 5e−12 |
| Meloidogyne hapla | rc34h08.y1 | gb\|BM902339 | 6e−11 |
| Meloidogyne javanica | rk57a05.y1 | gb\|BG736428 | 3e−11 |
| Meloidogyne javanica | rk79a05.y1 | gb\|BI324434 | 6e−11 |
| Necator americanus | Na_L3_09G07_SAC | gb\|BU086563 | 3e−62 |
| Necator americanus | Na_L3_36B10_SAC | gb\|BU088351 | 2e−42 |
| Necator americanus | Na_L3_12F04_SAC | gb\|BU086791 | 1e−12 |
| Onchocerca volvulus | SWOvAMCAQ10E05SK | gb\|BE202282 | 2e−11 |
| Ostertagia ostertagi | Oo_ad_02F04_LambdaGT11FO | gb\|BG734000 | 2e−13 |
| Parastrongyloides | kx48f05.y1 | gb\|BI863807 | 4e−31 |
| trichosuri | | | |
| Parastrongyloides | kx46c04.y1 | gb\|BI863606 | 8e−27 |
| trichosuri | | | |
| Pristionchus pacificus | rs10e10.r1 | gb\|AA193996 | 1e−62 |
| Strongyloides stercoralis | kq31g12.y1 | gb\|BE579648 | 5e−45 |
| Strongyloides stercoralis | kq39f03.y1 | gb\|BE580303 | 9e−15 |

For example, the C. elegans gene mlt-12, which corresponds to open reading frame W08F4.6, has exemplary orthologs in parasitic nematodes including BG310588 in Onchocerca volvulus (e⁻¹²¹); BE758466 in Brugia malayi (e104); BG2271612 in Strongyloides stercoralis (e⁻⁸⁴); and BM3468116 in Parastrongyloides trichosuri (e⁻⁸⁹). The C. elegans gene mlt-13, which corresponds to open reading frame F09B12.1, has exemplary orthologs in parasitic nematodes including BG226227 in Strongyloides stercoralis (9e⁻²⁴) and BF169279 in Trichuris muris (4e⁻¹¹). The C. elegans gene mlt-18, which corresponds to open reading frame W01F3.3, has exemplary orthologs in parastic nematodes including BG893621 in Strongyloides ratti (2e⁻²⁰); BQ625515 in Meloidogyne incognita (3e⁻²⁵); and BI746672 in Meloidogyne arenaria (6e⁻³¹). The C. elegans gene mlt-14, which corresponds to open reading frame C34G6.6, has exemplary orthologs in parastic nematodes including AA471404 in Brugia malayi (2e⁻⁶⁸); BE579677 in Strongyloides stercoralis (2e⁻⁵³); BI500192 in Pristionchus pacificus (2e⁻⁶⁹); BI782938 in Ascaris suum (9e⁻⁵²); BI073876 in Strongyloides ratti (1e⁻⁴¹); and BF060055 in Haemonchus contortus (4e⁻¹⁸). The C. elegans open reading frame ZK430.8 has an exemplary ortholog, AI723670, in Brugia malayi (8e⁻⁴⁰). The C. elegans gene pan-1, which corresponds to open reading frame M88.6 has exemplary orthologs in parastic nematodes including BI746256 in Meloidogyne arenaria (3.⁰⁰e⁻¹⁵). The C. elegans gene mlt-27, which corresponds to open reading frame C42D8.5 has exemplary orthologs in parastic nematodes including BM882137 in Parastrongyloides trichosuri (6e⁻³³); BM277122 in Trichuris muris (6e⁻¹⁵); BM880769 in Meloidogyne incognita (3e⁻⁴¹); and BI501765 in Meloidogyne arenaria. The C. elegans gene mlt-25 has exemplary orthologs in parasitic nematodes including BE581131 in Strongyloides stercoralis (1e⁻³⁴). The C. elegans open reading frame C23F12.1 has exemplary orthologs in parasitic nematodes including AI5399702 in Onchocerca volvulus (e6⁻³⁸); BE5802318 in Strongyloides stercoralis (e⁻³⁵); BE2389166 in Meloidogyne incognita (e6⁻¹⁷); BI501765 in Meloidogyne arenaria; BE581131 in Strongyloides stercoralis (1e⁻³⁴); AI5399702 in Onchocerca volvulus (e⁻³⁸); BE5802318 in Strongyloides stercoratis (e⁻³⁵); BE2389166 in Meloidogyne incognita (e⁻¹⁷); BE580288 in Strongyloides stercoralis; AA161577 in Brugia malayi (e⁻³⁹); CAAC01000016 in C. briggsae; BI744615 in Meloidogyne javanica (4e-44); BG224680 Strongyloides stercoralis (4e⁻⁴⁴); AW114337 Pristionchus pacificus (e⁻⁴¹), BM281377 in Ascaris suum (2e⁻⁴¹); BU585500 in Ascaris lumbricoides; BG577863 in Trichuris muris (e⁻²⁴); BQ091075 in Strongyloides ratti (6e⁻¹⁴); AW257707 in Onchocerca volvulus; BF014893 in Strongyloides stercoralis (7e-³⁵); BQ613344 in Meloidogyne incognita (5e⁻⁴⁷); CAAC01000088 in C. Briggsae, BG735742 in Meloidogyne javanica (4e⁻¹⁴); CAAC01000028; AA110597 in Brugia malayi (3e⁻⁵⁶); BI863834 in Parastrongyloides trichosuri (3e⁻⁶⁹); AI987143 in Pristionchus pacificus (3e⁻⁵⁶); BI782814 in Ascaris suum; BI744849 in Meloidogyne javanica; and BG735807 in Meloidogyne javanica (6e⁻³⁸).

RNA Interference

RNAi is a form of post-transcriptional gene silencing initiated by the introduction of double-stranded RNA (dsRNA) or antisense RNA. In C. elegans many expressed genes are subject to inactivation by RNAi (Fire et al., Nature 391:806-11, 1998; Fraser et al., Nature 408:325-30, 2000). RNAi may be accomplished by growing C. elegans on plates of E. coli expressing double stranded RNA. The nematodes feed on RNA-expressing bacteria, and this feeding is sufficient to cause the inactivation of specific target genes (Fraser et al., Nature 408:325-30, 2000; Kamath et al., Genome Biol 2, 2001). A double stranded RNA corresponding to one of the mlt genes described herein (e.g., one of those listed in Tables 1A, 1B, 4A-4D, and 7) is used to specifically silence mlt gene expression.

siRNA

Short twenty-one to twenty-five nucleotide double-stranded RNAs are effective at down-regulating gene expression in nematodes (Zamore et al., Cell 101: 25-33) and in mammalian tissue culture cell lines (Elbashir et al., Nature 411:494-498, 2001, hereby incorporated by reference). The further therapeutic effectiveness of this approach in mammals was demonstrated in vivo by McCaffrey et al. (Nature 418:38-39. 2002). The nucleic acid sequence of an Ecdysozoan gene ortholog can be used to design small interfering RNAs (siRNAs) that will inactivate mlt genes that have the specific 21 to 25 nucleotide RNA sequences used. siRNAs may be used, for example, as therapeutics to treat a parasitic nematode infection, as nematicides, or as insecticides.

Given the sequence of a mlt gene, siRNAs may be designed to inactivate that gene. For example, for a gene that consists of 2000 nucleotides, 1,978 different twenty-two nucleotide oligomers could be designed; this assumes that each oligomer has a two base pair 3′ overhang, and that each siRNA is one nucleotide residue from the neighboring siRNA. To inactivate a gene, only a few of these twenty-two nucleotide oligomers would be needed; approximately one dozen siRNAs, spaced across the 2,000 nucleotide gene, would likely be sufficient to significantly reduce target gene activity in an Ecdysozoan. Such siRNAs, for example, could be administered directly to an affected tissue, or administered systemically. C. elegans is used to identify siRNAs that cause a Mlt phenotype or larval arrest.

siRNAs that target nucleic acid sequences conserved among mlt genes would be expected to inactivate the corresponding gene in any species having that sequence. Although the protein sequences of mat genes are well conserved among widely divergent nematodes, for example, the nucleic acid sequences encoding them are not likely to exhibit the same level of conservation due to the degeneracy of the genetic code, which allows for wobble position substitutions. Thus, many siRNAs are expected to inactivate mRNAs only in specific target species. An siRNA designed to target a divergent region of O. volvulus mlt-12, for example, would be unlikely to affect other species.

Druggable Targets

The genomic survey described herein has identified a number of enzymes with small molecule substrates that function in molting. The Ecdysozoan orthologs of these worm genes represent targets, in this case for the disruption of molting, which would traditionally be selected for development of small molecule drugs. The orthologs of C. elegans genes listed in Tables 1A, 1B, 4A-4D, and 7, for example, are novel candidates for the development of nematicides, insecticides, and therapeutics for the treatment of parasitic infections.

Proteases are a particularly promising target for anti-parasitic development since large protease inhibitor libraries presently exist (the legacy of the development of ACE inhibitors, more recently HIV protease inhibitors, and undoubtedly CED-3 like cysteine protease inhibitors) and may be screened to identify inhibitors. The chemical backbone of drugs designed against a class of proteases, such as a cysteine protease, may be used as a starting point for developing and designing drug targets against other members within that class of enzymes. In one embodiment, a candidate compound that inhibits a protease could be identified using standard methods to monitor protease biological activity, for example, substrate proteolysis. A decrease in substrate proteolysis in the presence of the candidate compound, as compared to substrate proteolysis in the absence of the candidate compound, identifies that candidate compound as useful in the methods of the invention. In fact, it is reasonable to expect the substrate of that protease to be present in the lists of mlt genes provided herein, for example, in Tables 1A, 1B, 4A-4D, and 7. Protease/substrate pairs are identified by contacting recombinant proteases with recombinant candidate substrates and detecting substrate degradation or cleavage using an immunological assay, for example.

Isolation of Additional mlt Genes

Based on the nucleotide and amino acid sequences described herein, the isolation and identification of additional coding sequences of genes that function in molting is made possible using standard strategies and techniques that are well known in the art.

In one example, MLT polypeptides disclosed herein (e.g., those encoded by genes listed in Tables 1A, 1B, 4A-4D, and 7) are used to search a database, as described herein.

In another example, any organism that molts can serve as the nucleic acid source for the molecular cloning of such a gene, and these sequences are identified as ones encoding a protein exhibiting structures, properties, or activities associated with molt regulation disclosed herein (e.g., those listed in Tables 1A, 1B, 4A-4D, and 7).

In one particular example of such an isolation technique, any one of the nucleotide sequences described herein (e.g., those listed in Tables 1A, 1B, 4A-4D, and 7) may be used, together with conventional methods of nucleic acid hybridization screening. Such hybridization techniques and screening procedures are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. In one particular example, all or part of a mlt nucleic acid sequences listed in Tables 1A, 1B, 4A-4D, and 7 may be used as a probe to screen a recombinant DNA library for genes having sequence identity to a mlt gene. Hybridizing sequences are detected by plaque or colony hybridization according to standard methods.

Alternatively, using all or a portion of the nucleic acid sequence listed in Tables 1A, 1B, 4A-4D, and 7, one may readily design gene- or nucleic acid sequence-specific oligonucleotide probes, including degenerate oligonucleotide probes (i.e., a mixture of all possible coding sequences for a given amino acid sequence). These oligonucleotides may be based upon the sequence of either DNA strand and any appropriate portion of the nucleic acids, or nucleic acid sequences listed in Tables 1A, 1B, 4A-4D, and 7. General methods for designing and preparing such probes are provided, for example, in Ausubel et al. (supra), and Berger and Kimmel, (Guide to Molecular Cloning Techniques, 1987, Academic Press, New York). These oligonucleotides are useful for mlt gene isolation or for the isolation of virtually any gene listed in Tables 1A, 1B, 4A-4D, and 7, either through their use as probes capable of hybridizing to a mlt gene, or as complementary sequences or as primers for various amplification techniques, for example, polymerase chain reaction (PCR) cloning strategies. If desired, a combination of different, detectably-labeled oligonucleotide probes may be used for the screening of a recombinant DNA library. Such libraries are prepared according to methods well known in the art, for example, as described in Ausubel et al. (supra), or they may be obtained from commercial sources.

As discussed above, sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., (Proc. Natl. Acad. Sci. USA 85:8998, 1988).

Partial sequences, e.g., sequence tags, are also useful as hybridization probes for identifying full-length sequences, as well as for screening databases for identifying previously unidentified related virulence genes.

In general, the invention includes any nucleic acid sequence which may be isolated as described herein or which is readily isolated by homology screening or PCR amplification using any of the nucleic acid sequences disclosed herein (e.g., those listed in Tables 1A, 1B, 4A-4D, and 7).

It will be appreciated by those skilled in the art that, as a result of the degeneracy of the genetic code, a multitude of polynucleotide sequences encoding mlt genes, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring mlt genes (e.g., genes listed in Tables 1A, 1B, 4A-4D, and 7), and all such variations are to be considered as being specifically disclosed.

Although nucleotide sequences which mlt genes (e.g., genes listed in Tables 1A, 1B, 4A-4D, and 7), or their variants are preferably capable of hybridizing to the nucleotide sequence of the naturally occurring mlt genes (e.g., genes listed in Tables 1A, 1B, 4A-4D, and 7) under appropriately selected conditions of stringency, it may be advantageous to produce nucleotide sequences encoding mlt genes (e.g., genes listed in Tables 1A, 1B, 4A-4D, and 7), or their derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding mlt genes (e.g., genes listed in Tables 1A, 1B, 4A-4D, and 7) and their derivatives without altering the encoded amino acid sequences include the production of RNA transcripts having more desirable properties, such as a greater half-life, than transcripts produced from the naturally occurring sequence.

The invention also encompasses production of DNA sequences that encode mlt genes (e.g., genes listed in Tables 1A, 1B, 4A-4D, and 7), or fragments thereof generated entirely by synthetic chemistry. After production, the synthetic sequence may be inserted into any of the many available expression vectors and cell systems using reagents well known in the art. Moreover, synthetic chemistry may be used to introduce mutations into a sequence encoding any mlt gene (e.g., genes listed in Tables 1A, 1B, 4A-4D, and 7), or any fragment thereof.

Also encompassed by the invention are polynucleotide sequences that are capable of hybridizing to any mlt polynucleotide sequences (e.g., genes listed in Tables 1A, 1B, 4A-4D, and 7), and fragments thereof under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

Methods for DNA sequencing are well known in the art and may be used to practice any of the embodiments of the invention. The resulting sequences are analyzed using a variety of algorithms which are well known in the art. (See, e.g., Ausubel, F. M. (1997) Short Protocols in Molecular Biology, John Wiley & Sons, New York N.Y., unit 7.7)

In Silico Methods for the Isolation of Additional mlt Genes

In addition to these experimental approaches for the identification of additional mlt genes, mlt genes are also identified in silico using routine methods known to one skilled in the art and described herein. Such methods include searching genomic and EST databases for orthologs of C. elegans mlt genes, for example, mlt genes shown in Tables 1A, 1B, 4A-4D, and 7. Thus, as new genome sequences become available for insect pests (e.g., the new mosquito genome sequence) or parasitic nematodes, the nucleic acid or protein sequence of any one of the mlt genes listed in Tables 1A, 1B, 4A-4D, and 7, as well as mlt genes identified according to the methods of the invention (e.g., those that are identified in an enhanced mlt screens using C. elegans mutants with an increased susceptibility to RNAi) may be used to identify mlt orthologs. New mlt genes, for example, those mlt genes that function in the nervous system may be used in blastn, blastp, and tblastn comparisons to seek orthologs in new and existing genome databases. Just as degenerate oligonucleotide probes can be used in PCR and hybridization experiments, virtual probes (e.g., those degenerate nucleic acid sequences encoding a MLT polypeptide) may be used to query genome and EST databases for orthologs. In this way, orthologs of additional mlt genes will emerge.

Significantly, genomes that lack one or more mlt orthologs will also be identified using these methods. Such analyses will allow for the identification of mlt genes that are conserved, for example, only in nematodes. This will allow the development of highly specific nematicides. The identification of mlt genes that are conserved only among Ecdysozoans, and that are not present in vertebrates will allow the development of highly specific insecticides and nematicides unlikely to cause adverse side effects in vertebrates.

Polypeptide Expression

In general, MLT polypeptides of the invention may be produced by transformation of a suitable host cell with all or part of a mlt nucleic acid molecule (e.g., nucleic acids listed in Tables 1A, 1B, 4A-4D, and 7) or a fragment thereof in a suitable expression vehicle.

The MLT protein may be produced in a prokaryotic host, for example, E. coli, or in a eukaryotic host, for example, Saccharomyces cerevisiae, mammalian cells (for example, COS 1 or NIH 3T3 cells), or any of a number of plant cells or whole plant including, without limitation, algae, tree species, ornamental species, temperate fruit species, tropical fruit species, vegetable species, legume species, crucifer species, monocots, dicots, or in any plant of commercial or agricultural significance. Particular examples of suitable plant hosts include, but are not limited to, conifers, petunia, tomato, potato, pepper, tobacco, Arabidopsis, lettuce, sunflower, oilseed rape, flax, cotton, sugarbeet, celery, soybean, alfalfa, Medicago, lotus, Vigna, cucumber, carrot, eggplant, cauliflower, horseradish, morning glory, poplar, walnut, apple, grape, asparagus, cassava, rice, maize, millet, onion, barley, orchard grass, oat, rye, and wheat.

Such cells are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.). Descriptions and sources of useful host cells are also found in Vasil I. K., Cell Culture and Somatic Cell Genetics of Plants, Vol I, II, III Laboratory Procedures and Their Applications Academic Press, New York, 1984; Dixon, R. A., Plant Cell Culture—A Practical Approach, IRL Press, Oxford University, 1985; Green et al., Plant Tissue and Cell Culture, Academic Press, New York, 1987; and Gasser and Fraley, Science 244:1293, 1989.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). Also included in the invention are polypeptides which are modified in ways which do not abolish their biological activity (assayed, for example as described herein). Such changes may include certain mutations, deletions, insertions, or post-translational modifications, or may involve the inclusion of any of the polypeptides of the invention as one component of a larger fusion protein.

The invention farther includes analogs of any naturally occurring polypeptide of the invention. Analogs can differ from the naturally occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events). The aforementioned general techniques of polypeptide expression and purification can also be used to produce and isolate useful peptide fragments or analogs (described herein).

For eukaryotic expression, the method of transformation or transfection and the choice of vehicle for expression of the MLT polypeptide will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, K., Proc. Natl. Acad. Sci., U.S.A. 87:1228, 1990; Potrykas, I., Annu. Rev. Plant Physiol. Plant Mol. Biology 42:205, 1991; and BioRad (Hercules, Calif.) Technical Bulletin #1687 (Biolistic Particle Delivery Systems). Expression vehicles may be chosen from those provided, e.g., in Cloning Vectors: A Laboratory Manual (P. H. Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

Construction of Plant Transgenes

Transgenic plants containing a mlt transgene encoding a mlt polypeptide or containing a transgene encoding an RNA mlt nucleic acid inhibitor (e.g., dsRNA, siRNA, or antisense RNA) are useful for inhibiting molting in a Ecdysozoan contacting, feeding on, or parasitizing the plant. A transgenic plant, or population of such plants, expressing at least one mlt transgene (e.g., a MLT polypeptide or mlt nucleic acid inhibitor) would be expected to have increased resistance to Ecdysozoan (e.g., insect or nematode) damage or infestation. This is particularly desirable, given that Ecdysozoans can act as vectors for various plant diseases.

When designing an RNA mlt nucleic acid inhibitor for use in a transgenic plant, the specificity of the inhibitor must be considered. This is of particular importance when designing inhibitors that will induce plant immunity to Ecdysozoan (e.g., insect or nematode) infestation. In one particular example, the parasitic nematode, Heterodera schachti, is a beet parasite that expresses a mlt-14 ortholog. Expression of a Heterodera schachtii-specific RNA mlt-14 nucleic acid inhibitor in transgenic beets would be expected to disrupt molting and inhibit only in H. schactii, or closely related sister species, but would not be expected to affect other nematodes, insects, or vertebrates. The methods of the invention provide for highly specific nematicides and insecticides that minimize the ecological consequences of pesticide use. In most preferred embodiments, RNA mlt nucleic acid inhibitors target mlt genes conserved only in nematodes, and RNA mlt nucleic acid inhibitors are designed to target highly divergent regions of mlt genes.

For other applications an RNA mlt nucleic acid inhibitor that affects a wide range of Ecdysozoan pests is useful. Such RNA mlt nucleic acid inhibitors are designed to target well conserved regions of a mlt gene. These RNA mlt nucleic acid inhibitors are particularly useful, for example, when crop damage is caused by a wide range of nematode or insect pests. As new genome sequences become available, the design of ever more selective RNA mlt nucleic acid inhibitors and chemical compounds that target particular mlt gene regions will become a simple matter of comparative genomics.

In the case of insecticide development, even though the discovery of insect mlt genes is predicated on the conservation of mlt protein sequences between insects and nematodes, it is expected that the nucleic acid sequence of the orthologous mlt genes may not be well conserved. Thus, dsRNA, for example, an RNA mlt-14 nucleic acid inhibitor target just one particular pest. For other applications, it may be advantageous to target a particular region of a mlt gene that is well conserved among most insects. An RNA mlt nucleic acid inhibitor against a highly conserved region of a mlt gene would be useful, for example, in treating an area for a wide range of insect pests. As new genome sequences emerge, selection of compounds and nucleic acids that target particular mlt gene regions will become a simple matter of comparative genomics.

In one preferred embodiment, a mlt nucleic acid or RNA mlt nucleic acid inhibitor (e.g., double-stranded RNA, siRNA, or antisense RNA) is expressed by a stably-transfected plant cell line, a transiently-transfected plant cell line, or by a transgenic plant. A number of vectors suitable for stable or extrachromosomal transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra).

Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired mlt nucleic acid sequence is obtained as described above, it may be manipulated in a variety of ways known in the art. For example, where the sequence involves non-coding flanking regions, the flanking regions may be subjected to mutagenesis.

A mlt DNA sequence of the invention may, if desired, be combined with other DNA sequences in a variety of ways. A mlt DNA sequence of the invention may be employed with all or part of the gene sequences normally associated with a mlt protein. In its component parts, a DNA sequence encoding an MLT protein is combined in a DNA construct having a transcription initiation control region capable of promoting transcription and translation in a host cell.

In general, the constructs will involve regulatory regions functional in plants which provide for modified production of MLT protein as discussed herein. The open reading frame coding for the MLT protein or functional fragment thereof will be joined at its 5′ end to a transcription initiation regulatory region. Numerous transcription initiation regions are available which provide for constitutive or inducible regulation.

For applications where developmental, cell, tissue, hormonal, or environmental expression is desired, appropriate 5′ upstream non-coding regions are obtained from other genes, for example, from genes regulated during meristem development, seed development, embryo development, or leaf development.

Regulatory transcript termination regions may also be provided in DNA constructs of this invention as well. Transcript termination regions may be provided by the DNA sequence encoding a MLT protein or any convenient transcription termination region derived from a different gene source. The transcript termination region will contain preferably at least 1-3 kb of sequence 3′ to the structural gene from which the termination region is derived. Plant expression constructs having a mlt gene as the DNA sequence of interest for expression (in either the sense or antisense orientation) may be employed with a wide variety of plant life, particularly plant life involved in the production of storage reserves (for example, those involving carbon and nitrogen metabolism). Such genetically-engineered plants are useful for a variety of industrial and agricultural applications. Importantly, this invention is applicable to dicotyledons and monocotyledons, and will be readily applicable to any new or improved transformation or regeneration method.

The expression constructs include at least one promoter operably linked to at least one mlt gene (e.g., encoding a MLT polypeptide or RNA mlt nucleic acid inhibitor). An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters. Examples of plant expression constructs using these promoters are found in Fraley et al., U.S. Pat. No. 5,352,605. In most tissues of transgenic plants, the CaMV 35S promoter is a strong promoter (see, e.g., Odell et al., Nature 313:810, 1985). The CaMV promoter is also highly active in monocots (see, e.g., Dekeyser et al., Plant Cell 2:591, 1990; Terada and Shimamoto, Mol. Gen. Genet. 220:389, 1990). Moreover, activity of this promoter can be further increased (i.e., between 2-10 fold) by duplication of the CaMV 35S promoter (see e.g., Kay et al., Science 236:1299, 1987; Ow et al., Proc. Natl. Acad. Sci., U.S.A. 84:4870, 1987; and Fang et al., Plant Cell 1:141, 1989, and McPherson and Kay, U.S. Pat. No. 5,378,142).

Other useful plant promoters include, without limitation, the nopaline synthase (NOS) promoter (An et al., Plant Physiol. 88:547, 1988 and Rodgers and Fraley, U.S. Pat. No. 5,034,322), the octopine synthase promoter (Fromm et al., Plant Cell 1:977, 1989), figwort mosiac virus (FMV) promoter (Rodgers, U.S. Pat. No. 5,378,619), and the rice actin promoter (Wu and McElroy, W091/09948).

Exemplary monocot promoters include, without limitation, commelina yellow mottle virus promoter, sugar cane badna virus promoter, rice tungro bacilliform virus promoter, maize streak virus element, and wheat dwarf virus promoter.

For certain applications, it may be desirable to produce the MLT gene product in an appropriate tissue, at an appropriate level, or at an appropriate developmental time. For this purpose, there are an assortment of gene promoters, each with its own distinct characteristics embodied in its regulatory sequences, shown to be regulated in response to inducible signals such as the environment, hormones, and/or developmental cues. These include, without limitation, gene promoters that are responsible for heat-regulated gene expression (see, e.g., Callis et al., Plant Physiol. 88:965, 1988; Takahashi and Komeda, Mol. Gen. Genet. 219:365, 1989; and Takahashi et al. Plant J. 2:751, 1992), light-regulated gene expression (e.g., the pea rbcS-3A described by Kuhlemeier et al., Plant Cell 1:471, 1989; the maize rbcS promoter described by Schäffner and Sheen, Plant Cell 3:997, 1991; the chlorophyll a/b-binding protein gene found in pea described by Simpson et al., EMBO J. 4:2723, 1985; the Arabssu promoter; or the rice rbs promoter), hormone-regulated gene expression (for example, the abscisic acid (ABA) responsive sequences from the Em gene of wheat described by Marcotte et al., Plant Cell 1:969, 1989; the ABA-inducible HVA1 and HVA22, and rd29A promoters described for barley and Arabidopsis by Straub et al., Plant Cell 6:617, 1994 and Shen et al., Plant Cell 7:295, 1995; and wound-induced gene expression (for example, of wunI described by Siebertz et al., Plant Cell 1:961, 1989), organ-specific gene expression (for example, of the tuber-specific storage protein gene described by Roshal et al., EMBO J. 6:1155, 1987; the 23-kDa zein gene from maize described by Schernthaner et al., EMBO J. 7:1249, 1988; or the French bean β-phaseolin gene described by Bustos et al., Plant Cell 1:839, 1989), or pathogen-inducible promoters (for example, PR-1, prp-1, or -1,3 glucanase promoters, the fungal-inducible wirla promoter of wheat, and the nematode-inducible promoters, TobRB7-5A and Hmg-1, of tobacco and parsley, respectively).

Plant expression vectors may also optionally include RNA processing signals, e.g., introns, which have been shown to be important for efficient RNA synthesis and accumulation (Callis et al., Genes and Dev. 1:1183, 1987). The location of the RNA splice sequences can dramatically influence the level of transgene expression in plants. In view of this fact, an intron may be positioned upstream or downstream of an MLT polypeptide-encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84:744, 1987; An et al., Plant Cell 1:115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Frankfirt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

In addition, if desired, the plant expression construct may contain a modified or fully-synthetic structural mlt coding sequence that has been changed to enhance the performance of the gene in plants. Methods for constructing such a modified or synthetic gene are described in Fischoff and Perlak, U.S. Pat. No. 5,500,365.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller In: Genetic Engineering, vol 6, P W J Rigby, ed, London, Academic Press, 1987; and Lichtenstein, C. P., and Draper, J,. In: DNA Cloning, Vol II, D. M. Glover, ed, Oxford, IRI Press, 1985)), (2) the particle delivery system (see, e.g., Gordon-Kamm et al., Plant Cell 2:603 (1990); or BioRad Technical Bulletin 1687, supra), (3) microinjection protocols (see, e.g., Green et al., supra), (4) polyethylene glycol (PEG) procedures (see, e.g., Draper et al., Plant Cell Physiol. 23:451, 1982; or e.g., Zhang and Wu, Theor. Appl. Genet. 76:835, 1988), (5) liposome-mediated DNA uptake (see, e.g., Freeman et al., Plant Cell Physiol. 25:1353, 1984), (6) electroporation protocols (see, e.g., Gelvin et al., supra; Dekeyser et al., supra; Fromm et al., Nature 319:791, 1986; Sheen Plant Cell 2:1027, 1990; or Jang and Sheen Plant Cell 6:1665, 1994), and (7) the vortexing method (see, e.g., Kindle supra). The method of transformation is not critical to the invention. Any method which provides for efficient transformation may be employed. As newer methods are available to transform crops or other host cells, they may be directly applied. Suitable plants for use in the practice of the invention include, but are not limited to, sugar cane, wheat, rice, maize, sugar beet, potato, barley, manioc, sweet potato, soybean, sorghum, cassava, banana, grape, oats, tomato, millet, coconut, orange, rye, cabbage, apple, watermelon, canola, cotton, carrot, garlic, onion, pepper, strawberry, yam, peanut, onion, bean, pea, mango, citrus plants, walnuts, and sunflower.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, transfer and expression of transgenes in plant cells are now routine for one skilled in the art, and have become major tools to carry put gene expression studies in plants and to produce improved plant varieties of agricultural or commercial interest.

Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant; such techniques are described, e.g., in Vasil supra; Green et al., supra; Weissbach and Weissbach, supra; and Gelvin et al., supra.

In one particular example, a cloned MLT polypeptide expression construct under the control of the 35S CaMV promoter and the nopaline synthase terminator and carrying a selectable marker (for example, kanamycin resistance) is transformed into Agrobacterium. Transformation of leaf discs, with vector-containing Agrobacterium is carried out as described by Horsch et al. (Science 227:1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al. supra; Gelvin et al. supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed for transgenic plants expressing RNA mlt nucleic acid inhibitors and mlt nucleic acids encoding a MLT polypeptide. Such techniques include PCR amplification assays using oligonucleotide primers designed to amplify only transgene RNA templates and solution hybridization assays using transgene-specific probes (see, e.g., Ausubel et al., supra). Those RNA-positive plants that encode a MLT protein are then analyzed for protein expression by Western immunoblot analysis using MLT specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

Ectopic expression of one or more mlt genes or RNA mlt nucleic acid inhibitors is useful for the production of transgenic plants that disrupt molting in an Ecdysozoan (e.g., an insect or nematode) and have an increased level of resistance to insect or nematode infestation.

Transgenic Plants Expressing a mlt Transgene Disrupt Molting in an Insect or a Nematode

As discussed above, plasmid constructs designed for the expression of mlt nucleic acids or RNA mlt nucleic acid inhibitors (e.g., double-stranded RNA, siRNA, or antisense RNA) are useful, for example, for inhibiting molting in an Ecdysozoan (e.g., a parasitic insect or nematode) in contact with a transgenic plant transformed with at least one mlt nucleic acid or RNA mlt nucleic acid inhibitor. mlt nucleic acids that are isolated from an Ecdysozoan may be engineered for expression in a plant. The mlt nucleic acid may be expressed in its entirety, a portion of the mlt nucleic acid may be expressed, or an RNA mlt nucleic acid inhibitor comprising a mlt nucleic acid, or comprising the complementary strand of a mlt nucleic acid, may be expressed. The portion (e.g., 10%, 20%, 30%, 40%, 50%, 60%, 70%, 80%, 90%, or even 95%) of the full length nucleic acid may be selected to maximize specificity and minimize the effect of the nucleic acid expression on, for example, beneficial insects or nematodes. To disrupt molting in an Ecdysozoan, it is important to express an MLT protein or RNA mlt nucleic acid inhibitor at an effective level. Evaluation of the level of insect or nematode protection conferred to a plant by ectopic expression of a mlt nucleic acid or RNA mlt nucleic acid inhibitor is determined according to conventional methods and assays.

In one embodiment, constitutive ectopic expression of a mlt nucleic acid or RNA mlt nucleic acid inhibitor is generated by transforming a plant with a plant expression vector containing a nucleic acid sequence encoding an MLT polypeptide or RNA mlt nucleic acid inhibitor (e.g., double stranded RNA, antisense RNA, or siRNA). This expression vector is then used to transform a plant according to standard methods known to the skilled artisan and described in Fischhoff et al. (U.S. Pat. No. 5,500,365).

To assess resistance to nematodes or insects, transformed plants and appropriate control plants not expressing a transgene are grown to maturity, and a harmful insect or nematode is introduced to the plant under controlled conditions (for example, standard levels of temperature, humidity, and/or soil conditions). After a period of incubation sufficient to allow the growth and reproduction of a harmful insect or nematode on a control plant, nematodes or insects on transgenic and control plants are evaluated for their level of growth, viability, or reproduction according to conventional experimental methods. In one embodiment, the number of insects or nematodes and their progeny is recorded every twenty-four hours for seven days, fourteen days, twenty-one days, or twenty-eight days after inoculation. From these data, the effectiveness of transgene expression is determined. Transformed plants expressing a mlt nucleic acid or RNA mlt nucleic acid inhibitor that inhibits the growth, viability, or reproduction of a harmful insect or nematode relative to control plants are taken as being useful in the invention.

In another embodiment, plant damage in response to infestation with a harmful insect or parasitic nematode is evaluated according to standard methods. The level of insect or nematode damage in a plant expressing a mlt nucleic acid or RNA mlt nucleic acid inhibitor relative to a control plant not transformed with a mlt nucleic acid or RNA mlt nucleic acid inhibitor are compared. Transformed plants expressing a mlt nucleic acid or RNA mlt nucleic acid inhibitor that protects the plant from insect or nematode infestation, relative to a control plant not expressing a mlt nucleic acid or RNA mlt nucleic acid inhibitor, are taken as being useful in the invention.

Plants expressing a mlt nucleic acid or RNA mlt nucleic acid inhibitor (e.g., a mlt double-stranded RNA, antisense RNA or siRNA) are less vulnerable to insects, nematodes, and pest-transmitted diseases. The invention further provides for increased production efficiency, as well as for improvements in quality and yield of crop plants and ornamentals. Thus, the invention contributes to the production of high quality and high yield agricultural products, for example, fruits, ornamentals, vegetables, cereals and field crops having reduced spots, blemishes, and blotches that are caused by insects or nematodes; agricultural products with increased shelf-life and reduced handling costs; and high quality and yield crops for agricultural (for example, cereal and field crops), industrial (for example, oilseeds), and commercial (for example, fiber crops) purposes. Furthermore, because the invention reduces the necessity for chemical protection against plant pathogens, the invention benefits the environment where the crops are grown. Genetically-improved seeds and other plant products that are produced using plants expressing the nucleic acids described herein also render farming possible in areas previously unsuitable for agricultural production.

Production of Transgenic Domestic Mammals

Domesticated mammals (such as a cow, sheep, goat, pig, horse, dog, or cat) expressing a mlt nucleic acid or an RNA mlt nucleic acid inhibitor (e.g., double-stranded RNA, antisense RNA, or siRNA) are useful for inhibiting molting in an Ecdysozoan contacting (e.g., feeding on or parasitizing) the mammal. Such transgenic mammals will be resistant to Ecdysozoan parasites and will be useful in controlling insect or parasite infestation, or the spread of diseases transmitted by Ecdysozoan vectors. Methods for generating a transgenic mammal are known to the skilled artisan, and are described, for example, in WO 02/51997 and WO 02/070648. Transgenic mammals may be produced using standard methods for nuclear transfer, embryonic activation, embryo culture, and embryo transfer. Traditional methods for generating such mammals are described by Cibelli et al. (Science 1998: 280:1256-1258).

Production of Transgenic Ecdysozoans

Some human parasites spend a part of their life cycle parasitizing an insect host. Methods of the invention are useful in controlling such parasites. Transgenic insect hosts (e.g., Drosophila) expressing a mlt nucleic acid or an RNA mlt nucleic acid inhibitor (e.g., double-stranded RNA, antisense RNA, or siRNA) are useful for inhibiting molting in an Ecdysozoan (e.g., nematode) parasitizing the insect host. Such transgenic insects will be useful in controlling parasite infestation, or the spread of diseases transmitted by Ecdysozoan vectors.

In one embodiment, an insect (e.g., a black fly) is transformed with an RNA mlt nucleic acid inhibitor. Expression of the RNA mlt nucleic acid inhibitor kills parasitic nematode larvae (e.g., Onchocerca volvulus) within the insect host.

In another embodiment, transgenic Ecdysozoans (e.g., insects or nematodes) expressing a mlt nucleic acid or an RNA mlt nucleic acid inhibitor (e.g., double-stranded RNA, antisense RNA, or siRNA) are useful for inhibiting molting in an Ecdysozoan contacting (e.g., breeding with) the insect. A transgenic Ecdysozoan is bred to a naturally occurring Ecdysozoan to inhibit molting in the progeny and control an Ecdysozoan pest population. Methods for generating transgenic insects and nematodes are known to the skilled artisan, and are described, for example, by Kassis et al., (PNAS 89:1919-1923, 1992) and Chalfie et al., (Science 263:802-5, 1994).

Antibodies

The polypeptides disclosed herein or variants thereof or cells expressing them can be used as an immunogen to produce antibodies immunospecific for such polypeptides. “Antibodies” as used herein include monoclonal and polyclonal antibodies, chimeric, single chain, simianized antibodies and humanized antibodies, as well as Fab fragments, including the products of an Fab immunolglobulin expression library.

To generate antibodies, a coding sequence for a polypeptide of the invention may be expressed as a C-terminal fusion with glutathione S-transferase (GST) (Smith et al., Gene 67:31, 1988). The fusion protein is purified on glutathione-Sepharose beads, eluted with glutathione, cleaved with thrombin (at the engineered cleavage site), and purified to the degree necessary for immunization of rabbits. Primary immunizations are carried out with Freund's complete adjuvant and subsequent immunizations with Freund's incomplete adjuvant. Antibody titres are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specific recognition by Western blot or immunoprecipitation analysis (by the methods described in Ausubel et al., supra). Antibodies which specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al., Nature Biotech 14:309, 1996).

Preferably, antibodies of the invention are produced using fragments of the polypeptides disclosed herein which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Diagnostics

In another embodiment, antibodies which specifically bind any of the polypeptides described herein may be used for the diagnosis of a parasite infection, or a parasite-related disease. A variety of protocols for measuring such polypeptides, including immunological methods (such as ELISAs and RIAs) and FACS, are known in the art and provide a basis for diagnosing a parasite infection or a parasite-related disease.

In another aspect, hybridization with PCR probes which are capable of detecting polynucleotide sequences, including mlt genomic sequences, mlt open reading frames, or closely related molecules may be used to identify nucleic acid sequences which encode a MLT gene product. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences mlt genes (e.g., genes listed in Tables 1A, 1B, 4A-4D, and 7), allelic variants, or related sequences. Hybridization techniques may be used to identify mutations in mlt genes or may be used to monitor expression levels of these genes (for example, by Northern analysis, (Ausubel et al., supra).

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, and to develop and monitor the activities of therapeutic agents. Microarrays may be prepared, used, and analyzed using methods known in the art. (See, e.g., Brennan et al., U.S. Pat. No. 5,474,796; Schena et al., Proc. Natl. Acad. Sci. 93:10614, 1996; Baldeschweiler et al., PCT application WO95/251116, 1995; Shalon, D. et al., PCT application WO95/35505, 1995; Heller et al., Proc. Natl. Acad. Sci. 94:2150, 1997; and Heller et al., U.S. Pat. No. 5,605,662.)

Screening Assays

As discussed above, the identified mlt genes (e.g., genes listed in Tables 1A, 1B, 4A-4D, and 7) function in Ecdysozoan molting. Based on this discovery, screening assays were developed to identify compounds that inhibit the action of a MLT polypeptide or the expression of a mlt nucleic acid sequence. The method of screening may involve high-throughput techniques. In addition, these screening techniques may be carried out in cultured cells or in animals (such as nematodes).

Any number of methods are available for carrying out such screening assays. In one working example, candidate compounds are added at varying concentrations to the culture medium of cultured cells or nematodes expressing one of the nucleic acid sequences of the invention. Gene expression is then measured, for example, by standard Northern blot analysis (Ausubel et al., supra) or RT-PCR, using any appropriate fragment prepared from the nucleic acid molecule as a hybridization probe. The level of gene expression in the presence of the candidate compound is compared to the level measured in a control culture medium lacking the candidate molecule. A compound which promotes a decrease in the expression of a mlt gene (e.g., a gene listed in Tables 1A, 1B, 4A-4D, and 7) or functional equivalent is considered useful in the invention; such a molecule may be used, for example, as a nematicide, insecticide, or therapeutic to treat a parasitic-nematode infection. Such cultured cells include nematode cells (for example, C. elegans cells), mammalian, or insect cells.

In another working example, the effect of candidate compounds may be measured at the level of polypeptide production using the same general approach and standard immunological techniques, such as Western blotting or immunoprecipitation with an antibody specific for a MLT polypeptide encoded by a mlt gene (e.g., genes listed in Tables 1A, 1B, 4A-4D, and 7). For example, immunoassays may be used to detect or monitor the expression of at least one of the polypeptides of the invention in an organism. Polyclonal or monoclonal antibodies (produced as described above) which are capable of binding to such a polypeptide may be used in any standard immunoassay format (e.g., ELISA, Western blot, or RIA assay) to measure the level of the polypeptide. A compound that promotes a decrease in the expression of the polypeptide is considered particularly useful. Again, such a molecule may be used, for example, as a nematicide, insecticide, or therapeutic to delay, ameliorate, or treat a parasitic nematode infection.

In yet another working example, candidate compounds may be screened for those which specifically bind to and antagonize a MLT polypeptide encoded by a mlt gene (e.g., genes listed in Tables 1A, 1B, 4A-4D, and 7). The efficacy of such a candidate compound is dependent upon its ability to interact with a MLT polypeptide or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate molting may be assayed by any standard assay (e.g., those described herein).

In one particular working example, a candidate compound that binds to a MLT polypeptide, i.e., a polypeptide encoded by a mlt gene (e.g., a gene listed in Tables 1A, 1B, 4A-4D, and 7) may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the MLT polypeptide is identified on the basis of its ability to bind to the MLT polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to disrupt molting (e.g., as described herein). Compounds isolated by this approach may also be used, for example, as nematicides, insecticides, or therapeutics to treat a parasitic nematode infection. Compounds which are identified as binding to a MLT polypeptide with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to a nucleic acid sequence or polypeptide of the invention (e.g., MLT polypeptide) and thereby decrease its activity. Potential antagonists also include small molecules that bind to and occupy the binding site of the polypeptide thereby preventing binding to cellular binding molecules, such that normal biological activity is prevented.

Each of the DNA sequences provided herein may also be used in the discovery and development of a nematicide, insecticide, or therapeutic compound for the treatment of parasitic nematode infection. The encoded protein, upon expression, can be used as a target for the screening of molt-disrupting drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest. Such sequences may be isolated by standard techniques (Ausubel et al., supra).

The antagonists of the invention may be employed, for instance, as nematicides, insecticides, or therapeutics for the treatment of a parasitic nematode infection.

Optionally, compounds identified in any of the above-described assays may be confirmed as useful in a C. elegans molting assay.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of disrupting molting are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their molt-disrupting activity should be employed whenever possible.

When a crude extract is found to have a molt-disrupting activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having molt-disrupting activity. Methods of fractionation and purification of such heterogenous extracts are known in the art. If desired, compounds shown to be useful as insecticides, nematicides, or therapeutics for the treatment of a parasitic nematode infection are chemically modified according to methods known in the art.

Pharmaceutical Therapeutics

The invention provides a simple means for identifying compounds (including peptides, small molecule inhibitors, and mimetics) capable of acting as therapeutics for the treatment of a parasitic nematode infection. Accordingly, a chemical entity discovered to have medicinal value using the methods described herein is useful as a drug or as information for structural modification of existing insecticides or nematicides compounds, e.g., by rational drug design. Such methods are useful for screening compounds having an effect on a variety of conditions involving parasitic nematode infections in animals, for example, mammals, including humans and domestic animals (e.g., virtually any bovine, canine, caprine, feline, ovine, or porcine species). Parasitic nematodes that infect animals include, but are not limited to, any ascarid, filarid, or rhabditid (e.g., Dioctophymatida, Dioctophyme renale, Eustrongylides tubifex, Trichurida, Capillaria hepatica, Capillaria philippinensis, Trichinella spiralis, Trichuris muris, Trichuris, Trichuris trichiura, Trichuris vulpis. Ancylostoma, Ancylostoma caninum, Ancylostoina duodenal, Ancylostoma braziliense, Necator, Necator americanus, Placoconus, Angiostrongylus cantonensis, Cooperia, Haemonchus, Nematodirus, Obeliscoides cuniculi, Ostertagia, Trichostongylus, Ascaris, Ascaris lumbricoides, Ascaris suum, Toxocara canis, Baylisascaris procyonis, Anisakis, Oxyurida, Enterobius vennicularis, Cosmocerella, Onchocercidae, Brugia malayi, Dirofilaria, Dirofilaria immitis, Loa boa, Onchocerca volvulus, Wuchereria bancrofti, Spinitectus, Camallanus, Camallanus oxycephalus, Dracunculus, Dracunculus medinensis, Philometra cylindracea, Heterorhabditis bacteriophora, Parastrongyloides trichosuri, Pristionchus pacificus, Steinernema, Strongyloides stercoralis, or Strongyloides ratti).

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a parasite inhibitory agent in a physiologically-acceptable carrier. Suitable carriers and their formulation are described, for example, in Remington's Pharmaceutical Sciences by E. W. Martin. The amount of the nematicide agent to be administered varies depending upon the manner of administration, the age and body weight of the patient, and with the type of parasite the extensiveness of the infection. Generally, amounts will be in the range of those used for other agents used in the treatment of other diseases associated with parasite infection, although in certain instances lower amounts will be needed because of the increased specificity of the compound. In some applications, higher concentrations of the agent may be used given that the compound is highly specific to nematodes, and is therefore less likely to have adverse side effects in humans. A compound is administered at a dosage that induces larval arrest, disrupts nematode molting, or inhibits nematode viability.

Formulation of Pharmaceutical Compositions

The administration of an anti-parasitic compound may be by any suitable means that results in a concentration of the anti-parasitic that, combined with other components, is anti-parasitic upon reaching the parasite target. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the anti-parasitic within the body over an extended period of time; (ii) formulations that after a predetermined lag time create a substantially constant concentration of the anti-parasitic within the body over an extended period of time; (iii) formulations that sustain anti-parasitic action during a predetermined time period by maintaining a relatively, constant, effective anti-parasitic level in the body with concomitant minimization of undesirable side effects associated with fluctuations in the plasma level of the active anti-parasitic substance (sawtooth kinetic pattern); (iv) formulations that localize anti-parasitic action by, e.g., spatial placement of a controlled release composition adjacent to or in the infected tissue or organ; (v) formulations that allow for convenient dosing, such that doses are administered, for example, once every one or two weeks; and (vi) formulations that target a parasite by using carriers or chemical derivatives to deliver the anti-parasitic to a particular parasite or parasite infected cell type. Administration of anti-parasitic compounds in the form of a controlled release formulation is especially preferred for anti-parasitics having a narrow absorption window in the gastro-intestinal tract or a very short biological half-life. In these cases, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the anti-parasitic is formulated with appropriate excipients into a pharmaceutical composition that, upon administration, releases the anti-parasitic in a controlled manner. Examples include single or multiple unit tablet or capsule compositions, oil solutions, suspensions, emulsions, microcapsules, microspheres, molecular complexes, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active anti-parasitic (s), the composition may include suitable parenterally acceptable carriers and/or excipients. The active anti-parasitic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in the form suitable for sterile injection. To prepare such a composition, the suitable active anti-parasitic (s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active anti-parasitic (s) may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Solid Dosage Forms for Oral Use

Formulations for oral use of interferon include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active anti-parasitic substance in a predetermined pattern (e.g., in order to achieve a controlled release formulation) or it may be adapted not to release the active anti-parasitic substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active anti-parasitic substance). The coating may be applied on the solid dosage form in a similar manner as that described in Encyclopedia of Pharmaceutical Technology, supra.

The two anti-parasitics may be mixed together in the tablet, or may be partitioned. In one example, the first anti-parasitic is contained on the inside of the tablet, and the second anti-parasitic is on the outside, such that a substantial portion of the second anti-parasitic is released prior to the release of the first anti-parasitic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active anti-parasitic by controlling the dissolution and/or the diffusion of the active anti-parasitic substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the anti-parasitic (s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Combination Therapies

Anti-parasitics may be administered in combination with any other standard anti-parasitic therapy; such methods are known to the skilled artisan and described in Remington's Pharmaceutical Sciences by E. W. Martin.

Insecticides and Nematicides

Insecticides and nematicides are also provided by the methods described herein to control insects and nematodes. Such insecticides and nematicides are expected to be superior to existing insecticides and nematicides: (i) because they are specific to insect or nematode proteins and therefore unlikely to have adverse effects on humans; (ii) because they arrest development during molting, a non-feeding stage, in contrast to juvenile hormone insecticides which arrest development during a feeding stage; and/or (iii) because they result in an agriculturally desirable insect kill or “knockdown.” Methods for the production and application of insecticides or nematicides are standard in the art and described herein.

A method of controlling an insect, nematode, or other Ecdysozoan population is provided by the invention. The method involves contacting an insect or nematode with a biocidally effective amount of a MLT polypeptide, mlt nucleic acid, or RNA mlt nucleic acid inhibitor. Such methods may be used to kill or reduce the numbers of insects or nematodes in a given area, or may be prophylactically applied to an area to prevent infestation by a susceptible Ecdysozoan. Preferably the insect or nematode ingests, or is contacted with, an biocidally-effective amount of the MLT polypeptide, mlt nucleic acid, or RNA mlt nucleic acid inhibitor.

Insect Pests

Virtually all field crops, plants, and commercial farming areas are susceptible to attack by one or more insect pests. Such insect pests may be targeted with an insecticide containing a MLT polypeptide, mlt nucleic acid, or RNA mlt nucleic acid inhibitor. For example, vegetable and cole crops such as artichokes, kohlrabi, arugula, leeks, asparagus, lentils, beans, lettuce (e.g. head, leaf, romaine), beets, bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, peas, Chinese cabbage, peppers, collards, potatoes, cucumber, pumpkins, cucurbits, radishes, dry bulb onions, rutabaga, eggplant, salsify, escarole, shallots, endive, soybean, garlic, spinach, green onions, squash, greens, sugar beets, sweet potatoes, turnip, swiss chard, horseradish, tomatoes, kale, turnips, and a variety of spices are sensitive to infestation by one or more of the following insect pests: alfalfa looper, armyworm, beet armyworm, artichoke plume moth, cabbage budworm, cabbage looper, cabbage webworm, corn earworm, celery leafeater, cross-striped cabbageworm, european corn borer, diamondback moth, green cloverworm, imported cabbageworm, melonworm, omnivorous leafroller, pickleworm, rindworm complex, saltmarsh caterpillar, soybean looper, tobacco budworm, tomato fruitworm, tomato hornworm, tomato pinworm, velvetbean caterpillar, and yellowstriped armyworm.

Likewise, pasture and hay crops such as alfalfa, pasture grasses and silage are often attacked by such pests as armyworm, beef armyworm, alfalfa caterpillar, European skipper, a variety of loopers and webworms, as well as yellowstriped armyworms.

Fruit and vine crops such as apples, apricots, cherries, nectarines, peaches, pears, plums, prunes, quince almonds, chestnuts, filberts, pecans, pistachios, walnuts, citrus, blackberries, blueberries, boysenberries, cranberries, currants, loganberries, raspberries, strawberries, grapes, avocados, bananas, kiwi, persimmons, pomegranate, pineapple, tropical fruits are often susceptible to attack and defoliation by achema sphinx moth, amorbia, armyworm, citrus cutworm, banana skipper, blackheaded fireworm, blueberry leafroller, cankerworm, cherry fruitworm, citrus cutworm, cranberry girdler, eastern tent caterpillar, fall webworm, fall webworm, filbert leafroller, filbert webworm, fruit tree leafroller, grape berry moth, grape leaffolder, grapeleaf skeletonizer, green fruitworm, gummosos-batrachedra commosae, gypsy moth, hickory shuckworm, hornworms, loopers, navel orangeworm, obliquebanded leafroller, omnivorous leafroller. omnivorous looper, orange tortrix, orangedog, oriental fruit moth, pandemis leafroller, peach twig borer, pecan nut casebearer, redbanded leafroller, redhumped caterpillar, rougliskinned cutworm, saltmarsh caterpillar, spanworm, tent caterpillar, thecla-thecla basillides, tobacco budworm, tortrix moth, tufted apple budmoth, variegated leafroller, walnut caterpillar, western tent caterpillar, and yellowstriped armyworm.

Field crops such as canola/rape seed, evening primrose, meadow foam, corn (field, sweet, popcorn), cotton, hops, jojoba, peanuts, rice, safflower, small grains (barley, oats, rye, wheat, etc.), sorghum, soybeans, sunflowers, and tobacco are often targets for infestation by insects including armyworm, asian and other corn borers, banded sunflower moth, beet armyworm, bollworm, cabbage looper, corn rootworm (including southern and western varieties), cotton leaf perforator, diamondback moth, european corn borer, green cloverworm, headmoth, headworm, imported cabbageworm, loopers (including Anacamptodes spp.), obliquebanded leafroller, omnivorous leaftier, podworm, podworm, saltmarsh caterpillar, southwestern corn borer, soybean looper, spotted cutworm, sunflower moth, tobacco budworm, tobacco hornworm, velvetbean caterpillar,

Bedding plants, flowers, ornamentals, vegetables and container stock are frequently fed upon by a host of insect pests such as armyworm, azalea moth, beet armyworm, diamondback moth, ello moth (hornworm), Florida fern caterpillar, Io moth, loopers, oleander moth, omnivorous leafroller, omnivorous looper, and tobacco budworm.

Forests, fruit, ornamental, and nut-bearing trees, as well as shrubs and other nursery stock are often susceptible to attack from diverse insects such as bagworm, blackheaded budworm, browntail moth, California oakworm, douglas fir tussock moth, elm spanworm, fall webworm, fuittree leafroller, greenstriped mapleworm, gypsy moth, jack pine budworm, mimosa webworm, pine butterfly, redhumped caterpillar, saddleback caterpillar, saddle prominent caterpillar, spring and fall cankerworm, spruce budworm, tent caterpillar, tortrix, and western tussock moth. Likewise, turf grasses are often attacked by pests such as armyworm, sod webworm, and tropical sod webworm.

Nematode Agricultural Pests

Virtually all field crops, plants, and commercial farming areas are susceptible to attack by one or more nematode pests. Examples of plants subject to nematode attack include, but are not limited to, rice, wheat, maize, cotton, potato, sugarcane, grapevines, cassava, sweet potato, tobacco, soybean, sugar beet, beans, banana, tomato, lettuce, oilseed rape and sunflowers. Nematodes to be controlled using a nematicide containing a mlt nucleic acid or MLT polypeptide include, but are not limited to, plant parasites belonging to the Orders Dorylaimida and Tylenchida. Nematodes which may be controlled by this invention include, but are not limited to Families Longidoridae (e.g., Xiphinema spp. and Longidorus spp.) or Trichodoridae, (e.g., Trichodorus spp. and Paratrichodorus spp), migratory ectoparasites belonging to the Families Anguinidae (e.g., Ditylenchus spp.), Dolichodoridae (Dolichodorus spp.) and Belenolaimidae (e.g., Belenolaimus spp. and Trophanus spp).; obligate parasites belonging to the -Families Pratylenchidae (e.g., Pratylenchus spp., Radopholus spp. and Nacobbus spp), Hoplolaimidae (e.g., Helicotylenchus spp., Scutellonema spp. and Rotylenchulus spp.), Heteroderidae (e.g., Heterodera spp., Globodera spp., Meloidogyne spp. and Meloinema spp.), Criconematidae (e.g., Croconema spp., Criconemella spp. Hemicycliophora spp.), and Tylenchulidae (e.g., Tylenchulus spp., Paratylenchulus spp. and Tylenchocriconema spp.); and parasites belonging to the Families Aphelenchoididae (e.g., Aphelenchoides spp., Bursaphelenchus spp. and Rhadinaphelenchus spp.) and Fergusobiidae (e.g., Fergusobia spp.).

Insecticidal or Nematicidal Compositions and Methods of Use

In one preferred embodiment, the MLT polypeptide, mlt nucleic acid, or RNA mlt nucleic acid inhibitor compositions disclosed herein are useful as insecticides or nematicides for topical and/or systemic application to field crops, grasses, fruits and vegetables, lawns, trees, and/or ornamental plants. Alternatively, a MLT polypeptide, mlt nucleic acid, or RNA mlt nucleic acid inhibitor disclosed herein may be formulated as a spray, dust, powder, or other aqueous, atomized or aerosol for killing an Ecdysozoan (e.g., an insect, or nematode) or controlling an Ecdysozoan population. The MLT polypeptide, mlt nucleic acid, or RNA mlt nucleic acid inhibitor compositions disclosed herein may be used prophylactically, or alternatively, may be administered to an environment once target Ecdysozoans have been identified in the particular environment to be treated.

Regardless of the method of application, the amount of the active polypeptide component(s) is applied at a biocidally-effective amount, which will vary depending on such factors as, for example, the specific target Ecdysozoan to be controlled, the specific environment, location, plant, crop, or agricultural site to be treated, the environmental conditions, and the method, rate, concentration, stability, and quantity of application of the biocidally-active polypeptide composition. The formulations may also vary with respect to climatic conditions, environmental considerations, and/or frequency of application and/or severity of insect infestation.

The insecticide and nematicide compositions described may be made by formulating the isolated MLT protein with the desired agriculturally-acceptable carrier. The compositions may be formulated prior to administration in an appropriate means such as lyophilized, freeze-dried, desiccated, or in an aqueous carrier, medium or suitable diluent, such as saline or other buffer. The formulated compositions may be in the form of a dust or granular material, a suspension in oil (vegetable or mineral), water, or oil/water emulsion, or as a wettable powder, or in combination with any other carrier material suitable for agricultural application. Suitable agricultural carriers can be solid or liquid and are well known in the art. An agriculturally-acceptable carrier includes but is not limited to, for example, adjuvants, inert components, dispersants, surfactants, tackifiers, and binders, that are ordinarily used in insecticide or nematicide formulation technology. Such carriers are well known to those skilled in insecticide or nematicide formulation. The formulations may be mixed with one or more solid or liquid adjuvants and prepared by various means, e.g., by homogeneously mixing, blending and/or grinding the insecticidal composition with suitable adjuvants using conventional formulation techniques.

Oil Flowable Suspensions

In a preferred embodiment, the insecticide or nematicide composition comprises an oil flowable suspension comprising a MLT polypeptide, mlt nucleic acid, or RNA mlt nucleic acid inhibitor, or bacterial cell expressing a MLT polypeptide, mlt nucleic acid, or RNA mlt nucleic acid inhibitor. In one preferred embodiment, the bacterial cells are B. thuringiensis or E. coli, but any bacterial host cell expressing a MLT polypeptide, mlt nucleic acid, or RNA mlt nucleic acid inhibitor may be useful. Exemplary bacterial species include B. thuringiensis, B. megaterium, B. subtilis, B. cereus, E. coli, Salmonella spp., Agrobacterium spp., or Pseudomonas spp.

Water-Dispersible Granules

In another important embodiment, the insecticide composition comprises a water dispersible granule. This granule comprises a MLT polypeptide, mlt nucleic acid, or RNA mlt nucleic acid inhibitor, or bacterial cell expressing a MLT polypeptide, mlt nucleic acid, or RNA mlt nucleic acid inhibitor. In one preferred embodiment, the bacterial cells are B. thuringiensis or E. coli, but other bacteria such as B. megaterium, B. subtilis, B. cereus, E. coli, Salmonella spp., Agrobacterium spp., or Pseudomonas spp. cells transformed with a DNA segment disclosed herein and expressing a MLT polypeptide, mlt nucleic acid, or RNA mlt nucleic acid inhibitor are also contemplated to be useful.

Powders, Dusts, and Spore Formulations

For some applications, the insecticide composition comprises a wettable powder, dust, spore crystal formulation, cell pellet, or colloidal concentrate. This powder comprises a MLT polypeptide, mlt nucleic acid, or RNA mlt nucleic acid inhibitor, or a bacterial cell expressing a MLT polypeptide, mlt nucleic acid, or RNA mlt nucleic acid inhibitor. Preferred bacterial cells are B. thuringiensis or E. coli, however, bacterial cells such as those of other strains of B. thuringiensis, or cells of strains of bacteria such as B. megaterium, B. subtilis, B. cereus, E. coli, Salmonella spp., Agrobacterium spp., or Pseudomonas spp., may also be transformed with one or more mlt nucleic acid. Such dry forms of the insecticidal compositions may be formulated to dissolve immediately upon wetting, or alternatively, dissolve in a controlled-release, sustained-release, or other time-dependent manner. Such compositions may be applied to, or ingested by, the target insect, and as such, may be used to control the numbers of insects, or the spread of such insects in a given environment.

Aqueous Suspensions and Bacterial Cell Filtrates or Lysates

For some applications, the insecticide or nematicide composition comprises an aqueous suspension of bacterial cells, or an aqueous suspension of bacterial cell lysates or filtrates, etc., containing a MLT polypeptide, mlt nucleic acid, or RNA mlt nucleic acid inhibitor. Such aqueous suspensions may be provided as a concentrated stock solution which is diluted prior to application, or alternatively, as a diluted solution ready-to-apply.

The insecticidal or nematicidal compositions comprise intact bacterial cells expressing a mlt nucleic acid or polypeptide. These compositions may be formulated in a variety of ways. They may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, suspensions, emulsifiable concentrates, or the like. The ingredients may include Theological agents, surfactants, emulsifiers, dispersants, or polymers.

Alternatively, the novel insecticidal or nematicidal polypeptides may be prepared by native or recombinant bacterial expression systems in vitro and isolated for subsequent field application. Such protein may be either in crude cell lysates, suspensions, colloids, etc., or alternatively may be purified, refined, buffered, and/or further processed, before formulating in an active biocidal formulation. Likewise, under certain circumstances, it may be desirable to isolate a MLT polypeptide, mlt nucleic acid, or RNA mlt nucleic acid inhibitor from the bacterial cultures expressing the MLT polypeptide, mlt nucleic acid, or RNA mlt nucleic acid inhibitor and apply solutions, suspensions, or colloidal preparations of such nucleic acids or proteins as the active bioinsecticidal composition.

Multitfunctional Formulations

In some embodiments, when the control of multiple Ecdysozoan species is desired, the insecticidal or nematicidal formulations described herein may comprise one or more chemical pesticides, (such as chemical pesticides, nematicides, fungicides, virucides, microbicides, amoebicides, insecticides, etc.), and/or one or MLT polypeptides, mlt nucleic acids, or RNA mlt nucleic acid inhibitors. The insecticidal polypeptides may also be used in conjunction with other treatments such as fertilizers, weed killers, cryoprotectants, surfactants, detergents, insecticidal soaps, dormant oils, polymers, and/or time-release or biodegradable carrier formulations that permit long-term dosing of a target area following a single application of the formulation. In addition, the formulations may be prepared in edible baits or fashioned into insect or nematode traps to permit feeding or ingestion by a target Ecdysozoan of the biocide formulation.

The insecticidal compositions of the invention may also be used in consecutive or simultaneous application to an environmental site singly or in combination with one or more additional insecticides, pesticides, chemicals, fertilizers, or other compounds.

Application Methods and Effective Rates

The insecticidal or nematicidal compositions of the invention are applied to the environment of the target Ecdysozoan, typically onto the foliage of the plant or crop to be protected, by conventional methods, preferably by spraying. The strength and duration of application will be set with regard to conditions specific to the particular pest(s), crop(s) to be treated and particular environmental conditions. The proportional ratio of active ingredient to carrier will naturally depend on the chemical nature, solubility, and stability of the insecticidal composition.

Other application techniques, including dusting, sprinkling, soil soaking, soil injection, seed coating, seedling coating, foliar spraying, aerating, misting, atomizing, fumigating, aerosolizing, and the like, are also feasible and may be required under certain circumstances such as e.g., insects that cause root or stalk infestation, or for application to delicate vegetation or ornamental plants. These application procedures are also well-known to those of skill in the art.

The insecticidal or nematicidal compositions of the present invention may also be formulated for preventative or prophylactic application to an area, and may in certain circumstances be applied to pets, livestock, animal bedding, or in and around farm equipment, barns, domiciles, or agricultural or industrial facilities, and the like.

The concentration of an insecticidal or nematicidal composition that is used for environmental, systemic, topical, or foliar application will vary widely depending upon the nature of the particular formulation, means of application, environmental conditions, and degree of biocidal activity. Typically, the biocidal, insecticidal, or nematicidal composition will be present in the applied formulation at a concentration of at least about 1%, 5%, 10%, 20%, 30%, 40%, 50%, 60%, 70%, 80%, 90%, or 99% by weight. Dry formulations of MLT polypeptide, mlt nucleic acid, or RNA mlt nucleic acid inhibitor compositions may be from about 1% to about 99% or more by weight of the nucleic acid or polypeptide composition, while liquid formulations may generally comprise from about 1%, 5%, 10%, 20%, 30%, 40%, 50%, 60%, 70%, 80%, 90%, 99% or more of the active ingredient by weight.

In the case of compositions in which intact bacterial cells that contain at least one MLT polypeptide, mlt nucleic acid, or RNA mlt nucleic acid inhibitor are included, preparations will generally contain from about 10⁴ to about 10⁸ cells/mg, although in certain embodiments it may be desirable to utilize formulations comprising from about 10² to about 10⁴ cells/mg, or when more concentrated formulations are desired, compositions comprising from about 10⁸ to about 10¹⁰ or 10¹¹ cells/mg may also be formulated. Alternatively, cell pastes, spore concentrates, MLT polypeptide, mlt nucleic acid, or RNA mlt nucleic acid inhibitor concentrates may be prepared that contain the equivalent of from about 10¹² to 10¹³ cells/mg of the active polypeptide, and such concentrates may be diluted prior to application.

The insecticidal or nematicidal formulation described above may be administered to a particular plant or target area in one or more applications as needed, with a typical field application rate per hectare ranging on the order of about 50, 100, 200, 300, 400, or 500 g/hectare of active ingredient, or alternatively, 600, 700, 800, 900, or 1000 g/hectare may be utilized. In certain instances, it may even be desirable to apply the insecticidal or nematicidal formulation to a target area at an application rate of about 1000, 2000, 3000, 4000, 5000 g/hectare or even as much as 7500, 10,000, or 15,000 g/hectare of active ingredient.

MLT Polypeptide Insecticides and Nematicides

As discussed above, MLT polypeptide, mlt nucleic acid, and RNA mlt nucleic acid inhibitor are useful, for example, for inhibiting molting in an Ecdysozoan (e.g., a parasitic insect or nematode). Such nucleic acids and polypeptides may be, for example, applied ectopically or administered systemically to a plant at a level that is sufficient to inhibit insect or nematode infestation in the plant. Evaluation of the level of insect or nematode protection conferred to a plant by application or administration of a MLT polypeptide, mlt nucleic acid, or RNA mlt nucleic acid inhibitor is determined according to conventional methods and assays.

In one embodiment, a plant is contacted with a MLT polypeptide, mlt nucleic acid, or RNA mlt nucleic acid inhibitor present in an excipient, such that a MLT polypeptide, mlt nucleic acid, or RNA mlt nucleic acid inhibitor is present in or on the plant (e.g., in or on the roots, leaves, stems, fruit, flowers, or vegetative tissues). A parasitic insect or nematode is introduced to the plant under controlled conditions (for example, standard levels of temperature, humidity, and/or soil conditions). After a period of incubation sufficient to allow the growth and reproduction of a harmful insect or nematode on a control plant not contacted with a MLT polypeptide, mlt nucleic acid, or RNA mlt nucleic acid inhibitor, insects, nematodes, or their progeny are evaluated for their level of growth, viability, or reproduction according to conventional experimental methods. For example, the number of insects, nematodes, or their progeny is recorded every twenty-four hours for seven days, fourteen days, twenty-one days, or twenty-eight days or longer after inoculation. From these data, levels of inhibition of harmful insects or nematodes are determined. MLT polypeptide, mlt nucleic acid, or RNA mlt nucleic acid inhibitors that inhibit the growth, viability, or reproduction of a harmful insect or nematode are taken as being useful in the invention. In another embodiment, the level of plant damage is determined according to standard methods on the plant contacted with the MLT polypeptide, mlt nucleic acid, or RNA mlt nucleic acid inhibitor relative to a control plant not contacted with the MLT polypeptide, mlt nucleic acid, or RNA mlt nucleic acid inhibitor. MLT polypeptides, mlt nucleic acids, or RNA mlt nucleic acid inhibitors that inhibit plant damage are taken to be useful in the methods of the invention.

### Other Embodiments

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

What is claimed is:

1. A method for identifying a candidate compound that disrupts Ecdysozoan molting, said method comprising:
(a) providing a cell expressing a mlt nucleic acid molecule or an ortholog of a mlt nucleic acid molecule;
(b) contacting said cell with a candidate compound; and
(c) comparing the expression of said nucleic acid molecule in said cell contacted with said candidate compound with the expression of said nucleic acid molecule in a control cell not contacted with said candidate compound, wherein an alteration in said expression identifies said candidate compound as a candidate compound that disrupts molting.

2. The method of claim 1, wherein said cell expresses a mlt nucleic acid molecule selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, F10C1.5.

3. The method of claim 1, wherein said ortholog of a mlt nucleic acid is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916 AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

4. The method of claim 1, wherein said cell is
a nematode cell.

5. (canceled)

6. (canceled)

7. The method of claim 1, wherein said method identifies a compound that decreases transcription of said mlt nucleic acid molecule.

8. The method of claim 1, wherein said method identifies a compound that decreases translation of an mRNA transcribed from said mlt nucleic acid molecule.

9. The method of claim 1, wherein said compound is a member of a chemical library.

10. The method of claim 1, wherein said method is carried out in a nematode.

11. (canceled)

12. A method for identifying a candidate compound that disrupts molting in an Ecdysozoan, said method comprising:
(a) providing a cell expressing a MLT polypeptide;
(b) contacting said cell with a candidate compound; and
(c) comparing the biological activity of said MLT polypeptide in said cell contacted with said candidate compound to a control cell not contacted with said candidate compound, wherein an alteration in said biological activity of said MLT polypeptide identifies said candidate compound as a candidate compound that disrupts molting in an Ecdysozoan.

13. The method of claim 12, wherein said cell is a nematode cell.

14. The method of claim 12, wherein said cell is a mammalian cell.

15. The method of claim 12, wherein said MLT polypeptide is a protease.

16. The method of claim 12, wherein said biological activity is monitored with an enzymatic assay or an immunological assay.

17. (canceled)

18. The method of claim 12, wherein said cell is in a nematode and said biological activity is monitored by detecting molting. BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM 057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

21. A method for identifying a candidate compound that disrupts molting, said method comprising:
(a) contacting a nematode with a candidate compound; and
(b) comparing molting in said nematode contacted with said candidate compound to a control nematode not contacted with said candidate compound, wherein an alteration in said molting identifies said candidate compound as a candidate compound that disrupts molting in a nematode.

22. A method of identifying a candidate compound that disrupts Ecdysozoan molting, said method comprising:
a) contacting a cell comprising a mlt nucleic acid regulatory region fused to a detectable reporter gene with an candidate compound;
b) detecting the expression of the reporter gene; and
c) comparing said reporter gene expression in said cell contacted with said candidate compound with a control cell not contacted with said candidate compound, wherein an alteration in the expression of the reporter gene identifies the candidate compound as a compound that disrupts molting in an Ecdysozoan.

23. The method of claim 22, wherein said alteration is an alteration of at least 10% in the timing or level of expression of said reporter gene relative to the timing of expression in a control nematode not contacted with said candidate compound.

24. (canceled)

25. The method of claim 22, wherein said alteration is an alteration in the cellular expression pattern of said reporter gene relative to the cellular expression pattern in a control nematode not contacted with said candidate compound.

26. A method for identifying a candidate compound that disrupts Ecdysozoan molting, said method comprising:
(a) contacting a MLT polypeptide with a candidate compound; and
(b) detecting binding of said candidate compound to said MLT polypeptide, wherein said binding identifies said candidate compound as a candidate compound that disrupts molting in an Ecdysozoan.

27. The method of claim 26, wherein said compound is a member of a chemical library.

28. An isolated RNA mlt nucleic acid inhibitor comprising at least a portion of a naturally occurring mlt nucleic acid molecule of an organism, or its complement, said mlt nucleic acid molecule being selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B 11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E 11A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of said mlt nucleic acid molecule, wherein said RNA mlt nucleic acid inhibitor is capable of hybridizing to a naturally occurring mlt nucleic acid molecule and decreasing expression of said mlt nucleic acid molecule in said organism.

29. The RNA mlt nucleic acid inhibitor of claim 28, wherein said RNA is a double stranded RNA molecule that decreases expression in said organism by at least 10%.

30. The RNA mlt nucleic acid inhibitor of claim 28, wherein said RNA molecule is an antisense nucleic acid molecule that is complementary to at least six nucleotides of said mlt nucleic acid molecule and decreases expression in said organism by at least 10%.

31. The RNA mlt nucleic acid inhibitor of claim 28, wherein said RNA molecule is an siRNA molecule that comprises at least 20 nucleic acids of said mlt nucleic acid molecule and decreases expression in said organism by at least 10%.

32. The RNA mlt nucleic acid inhibitor of claim 27, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709 AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

33. A vector comprising the nucleic acid of claim 32 positioned for expression.

34-36. (canceled)

37. A method for reducing a parasitic nematode infection in an organism, said method comprising contacting said organism with an RNA mlt nucleic acid inhibitor that comprises at least a portion of a mlt nucleic acid molecule, or its complement, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G1.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of said nucleic acid molecule, in an amount sufficient to reduce said parasitic nematode infection in said organism.

38. The method of claim 37, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM 057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

39. The method of claim 37, wherein said RNA mlt nucleic acid inhibitor is a double stranded RNA molecule that comprises at least 20 nucleic acids of a mlt nucleic acid molecule of claim 37 and is capable of hybridizing to a mlt nucleic acid molecule under high stringency conditions, and is capable of decreasing expression of the nucleic acid molecule in said organism with which it shares identity by at least 10%.

40. The method of claim 37, wherein said RNA mlt nucleic acid inhibitor is an antisense nucleic acid molecule that is complementary to at least six nucleotides of a mlt nucleic acid molecule of claim 37, and is capable of hybridizing to a mlt nucleic acid molecule under high stringency conditions and is capable of decreasing expression by at least 10% from the nucleic acid molecule to which it is complementary.

41. The method of claim 37, wherein said RNA mlt nucleic acid inhibitor is an siRNA molecule that comprises at least 20 nucleic acids of a mlt nucleic acid molecule of claim 37, and is capable of hybridizing to a mlt nucleic acid molecule under high stringency conditions and is capable of decreasing expression by at least 10% from the nucleic acid molecule with which it shares identity

42. The method of claim 37, wherein said organism is a mammal.

43. The method of claim 37, wherein said mammal is a domestic mammal or human.

44-49. (canceled)

50. An insecticide including an insecticide excipient and an ortholog of a MLT polypeptide or portion thereof, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, that disrupts insect molting by at least 10%.

51. The insecticide of claim 50, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

52. An insecticide including an insecticide excipient and an ortholog of a mlt nucleic acid molecule or portion thereof, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B 11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, that disrupts insect molting by at least 10%.

53. The composition of claim 52, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM_{—}079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

54. An insecticide including an insecticide excipient and an RNA mlt nucleic acid inhibitor comprising at least a portion of an insect ortholog of a mlt nucleic acid molecule, or its complement, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, that disrupts insect molting by at least 10%.

55. The composition of claim 52, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AY060635, NM_{—}143476, AC008339, L02793, NM_{—}079167, J02727, NM_{—}139674, NM 079763, NM_{—}057268, NM_{—}137449, NM_{—}079419, NM_{—}080092, AAF51201, NM_{—}057698, NM_{—}080132, NM_{—}132335, AJ487018, NM_{—}080072, AY094832, NM_{—}057520, NM_{—}136653, NM_{—}078644, AY075331, M90806, NM_{—}079419, NM_{—}080092, AAF51201, NM_{—}057698, NM_{—}134578, AY071265, AY060235, NM_{—}078577, NM_{—}057621, AY089504, NM_{—}135238, X78577, AY118647, NM_{—}140652, AY113364, NM_{—}079972, X58374, NM_{—}132550, AY052122 AY060893, AY058709, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

56. A nematicide including a nematicide excipient and a MLT polypeptide or portion thereof, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B 12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y11B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77 μl A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of said polypeptide that disrupts nematode molting by at least 10%.

57. The nematicide of claim 56, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

58. A nematicide including a nematicide excipient and a mlt nucleic acid molecule or portion thereof, selected from the group consisting B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y11B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of said nucleic acid molecule that disrupts nematode molting by at least 10%.

59. The nematicide of claim 58, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

60. A nematicide including a nematicide excipient and an RNA mlt nucleic acid inhibitor comprising at least a portion of a mlt nucleic acid molecule, or its complement, selected from the group consisting of B0024.14, C01H6.5, C09G5.6, C11H1.3, C17G1.6, C23F12.1, B0272.5, C34G6.6, C37C3.3, C42D8.5, C45B2.7, CD4.4, CD4.6, D1054.15, F08C6.1, F09B12.1, F11C1.6, F16H9.2, F18A1.3, F18C12.2, F20G4.1, F25B4.6, F29D11.1, F33A8.1, F33C8.3, F38H4.9, F40G9.1, F41C3.4, F41H10.7, F45G2.5, F49C12.12, F52B11.3, F53B8.1, F53G12.3, F54A5.1, F54C9.2, F56C11.1, F57B9.2, H04M03.4, H19M22.1, K04F10.4, K05C4.1, K06B4.5, K07C5.6, K07D8.1, K08B4.1, K09H9.6, M03F4.7, M03F8.3, M162.6, M6.1, M88.6, R05D11.3, R07E4.6, R11G11.1, T01C3.1, T01H3.1, T05C12.10, T14F9.1, T19B10.2, T23F2.1, T24H7.2, T27F2.1, W01F3.3, W08F4.6, W09B6.1, W10G6.3, Y111B2A.14, Y37D8A.10, Y38F2AL.3, Y48B6A.3, ZC101.2, ZK1073.1, ZK1151.1, ZK262.8, ZK270.1, ZK430.8, ZK686.3, ZK783.1, ZK970.4, C09F12.1, C09H10.2, C17H12.14, C37C3.2, C37C3.3, D2085.1, EEED8.5, F10E9.7, F19F10.9, F28F8.5, F32D1.2, F35H10.4, F41E7.1, F42A8.1, F54B3.3, F55A3.3, F56F3.5, H06I04.4a, K06A4.6, K10D6.1, R06A10.1, T07D10.1, Y17G7A.2, Y23H5A.7, Y38F2AL.3, Y41D4B.21, Y41D4B.5, Y41D4B.5, Y45F10B.5, Y55H10A.1, ZK1236.3, ZK265.5, ZK265.6, ZK652.1, Y54E10BR.5, B0513.1, R06A4.9, Y105E8B.1, Y47D3B.1, Y54F10AL.2, T17H7.3, H27M09.5, F45E10.2, F25H8.6, K04A8.6, ZC13.3, T19A5.3, F32D8.6, F53F4.3, F56C9.12, T25B9.10, ZK154.3, Y37D8A.19, Y37D8A.21, Y71F9AL.7, Y51H1A.3, W03F9.10, ZK945.2, ZK637.4, C30F8.2, F32H2.9, Y87G2A.5, Y53F4B.22, Y77E11A.13, C15H11.7, Y113G7B.23, C53H9.1, W09C5.6, T24B8.1, Y71A12B.1, C26C6.3, C42D8.5, F53G12.3, Y41D4B.10, and F10C1.5, or an ortholog of said nucleic acid molecule that disrupts nematode molting by at least 10%.

61. The nematicide of claim 60, wherein said ortholog is selected from the group consisting of M90806, NM_{—}134578, AY075331, BG310588, BE758466, BG227161, BM346811, BG226227, BF169279, BE580288, BG893621, BQ625515, BI746672, AA471404, BE579677, BI500192, BI782938, BI073876, BF060055, AI723670, BI746256, BM882137, BM277122, BM880769, BI501765, BE581131, AI539970, BE580231, BE238916, AA161577, CAAC01000016, BI744615, BG224680, AW114337, BM281377, BU585500, BG577863, BQ091075, AW257707, BF014893, BQ613344, CAAC01000088, BG735742, CAAC01000028, AA110597, BI863834, AI987143, BI782814, BI744849, and BG735807.

62-67. (canceled)
